(19) United States
(12) Patent Application Publication
(10) Pub. No.: US 2026/0014270 A1
(43) Pub. Date: Jan. 15, 2026

(54) ANTI-CEACAM5 ANTIBODY-DRUG CONJUGATE AND METHOD FOR PREPARATION AND USE THEREOF

(71) Applicant: SICHUAN KELUN-BIOTECH BIOPHARMACEUTICAL CO., LTD., Chengdu (CN)

(72) Inventors: Shuntao LUO, Chengdu (CN); Yajun HE, Chengdu (CN); Qiang TIAN, Chengdu (CN); Lirong LI, Chengdu (CN); Yue FAN, Chengdu (CN); Yitao ZHANG, Chengdu (CN); Ruihong LIU, Chengdu (CN); Shasha ZHAO, Chengdu (CN); Qin ZHOU, Chengdu (CN); Hu LONG, Chengdu (CN); Hongmei SONG, Chengdu (CN); Miao TAN, Chengdu (CN); Xiangyang TAN, Chengdu (CN); Junyou GE, Chengdu (CN)

(73) Assignee: SICHUAN KELUN-BIOTECH BIOPHARMACEUTICAL CO., LTD., Chengdu (CN)

(21) Appl. No.: 19/261,667

(22) Filed: Jul. 7, 2025

(30) Foreign Application Priority Data
Jul. 9, 2024 (WO) PCT/CN2024/104405
Jun. 16, 2025 (WO) PCT/CN2025/101241

(51) Int. Cl. A61K 47/68; A61P 35/00; C07K 16/28

(52) U.S. Cl. CPC A61K 47/6853; A61K 47/68037; A61P 35/00; C07K 16/2803; C07K 2317/92

(57) ABSTRACT

The present application relates to an antibody-drug conjugate and a method for preparation and use thereof, and specifically relates to an antibody-drug conjugate for treating CEACAM5-positive cancer, including an anti-CEACAM5 antibody, and drug-linker molecules conjugated to the antibody. In some embodiments, the antibody is humanized. In some embodiments, the antibody has excellent binding activity to CEACAM5-positive cells, and can efficiently deliver drugs to CEACAM5-positive cells. In some embodiments, the drug includes a DNA topoisomerase inhibitor. The antibody-drug conjugate of the present application has a better drug-antibody conjugation ratio, and has a good targeted killing effect on certain cancers.

### RELATED APPLICATIONS

This application claims the benefit of International Application No. PCT/CN2025/101241, filed Jun. 16, 2025 and International Application No. PCT/CN2024/104405, filed Jul. 9, 2024, the disclosure of each of which is incorporated herein by reference in its entirety.

### SEQUENCE LISTING

This application contains an electronic Sequence Listing which has been submitted in XML file format with this application, the entire content of which is incorporated by reference herein in its entirety. The Sequence Listing XML file submitted with this application is entitled “14463-704-999 SEQ_LISTING.xml”, was created on Jun. 25, 2024, and is 35,765 bytes in size.

### FIELD OF THE INVENTION

The present application relates to the field of targeted therapy, and specifically relates to an antibody-drug conjugate for treating CEACAM5-positive cancers. Specifically, the antibody-drug conjugate comprises an anti-CEACAM5 antibody, and drug-linker molecules conjugated to the antibody. In some embodiments, the antibody is humanized. In some embodiments, the antibody has excellent binding activity to CEACAM5, and can efficiently deliver drugs to CEACAM5. In some embodiments, the drug comprises a DNA topoisomerase inhibitor. The antibody-drug conjugate of the present application has excellent drug-antibody conjugation ratio, and has good targeted killing effect on rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer). Therefore, the present application further provides a method for preparing the antibody-drug conjugate and use thereof in treating CEACAM5-positive cancers.

### BACKGROUND OF THE INVENTION

CEACAM5 (Carcinoembryonic antigen-related cell adhesion molecule 5), also known as CD66e, belongs to a large CEACAM subfamily of the immunoglobulin superfamily, is a cell adhesion molecule that, when expressed at a high level, may affect the genesis and development of a tumor. The CEACAM subfamily includes 12 glycoprotein members (CEACAM1, CEACAM3, CEACAM4, CEACAM5, CEACAM6, CEACAM7, CEACAM8, CEACAM16, CEACAM18, CEACAM19, CEACAM20, and CEACAM21). The CEACAM5 is mainly expressed on cytomembranes in a GPI-anchored manner, can be enzymatically hydrolyzed by GPI-PLD (glycosylphosphatidylinositol phospholipase D), and is then shed to form the CEACAM5 free in blood. CEACAM5 glycoprotein comprises 1 Ig-like V-type (IgV) domain at the N-terminus, followed by 6 Ig-like C2-type (IgC2) domains and a GPI anchor. Its main function is adhesion to epithelial cells in embryonic intestinal and colon tumors. In addition, it also plays an important role in inhibiting colonic cell differentiation and apoptosis. The CEACAM5 can prevent tumor cells from anoikis. CEACAM5 expression is limited in normal adult tissues. In normal tissues, the CEACAM5 expression can be detected in kidney, bladder, larynx, epiglottis, skin, submaxillary gland, colon, esophagus, duodenum, parotid gland, and sublingual gland tissues. In contrast, the CEACAM5 expression is not detected in stomach, pancreas, and many other normal tissues. The CEACAM5 mainly serves as a cell adhesion molecule in normal tissues, to mediate homogeneous and heterogeneous adhesion. The CEACAM5 is expressed in rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer). The CEACAM5 is found to be distributed throughout the cell surface and cytoplasm of cancer cells. The CEA is a well-known biomarker for many types of malignancies, such as colorectal cancer and non-small cell lung cancer. High CEACAM5 expression, detectable by IHC (immunohistochemistry), is also found in approximately 25% of patients with advanced non-squamous (NSq) non-small cell lung cancer (NSCLC). In addition to the increase of the CEACAM5 shown in tumor cells, some articles show changes of the level of free CEACAM5 in plasma, and some literatures show that sCEA levels in 64.7% (101/156) of CRC patients are higher than the normal range. Among drugs targeting the CEACAM5, ADCs currently in clinical stages include the antibody SAR408701. In previously published clinical studies on lung cancer treatment, the SAR408701 has good efficacy for NSCLC (non-small cell lung cancer). Overall response rate (ORR) is between 20-30%.

An ADC drug is composed of a monoclonal antibody and a potent toxic drug conjugated through a bioactive linker, and is a potent anti-cancer drug targeting cancer cells at specific sites. Due to its accurate identification of targets, it greatly improves efficacy and reduces toxic and side effects. It is one of the main research directions for anti-tumor drugs in the future. At present, no CEACAM5-targeting antibody drugs are available on the market. Therefore, it is urgent and necessary to develop a CEACAM5-targeting antibody/ADC with higher specificity, lower toxic and side effects, better clinical efficacy, and more convenient administration mode, which will provide patients with more drug use options. Biomacromolecular drugs have become an important part of anti-tumor drugs, and are showing a rapid growth trend.

ADC drugs are composed of antibodies, bioactive molecules and linkers. Biologically active molecules are covalently conjugated to the antibodies through the linkers; the antibodies (for example monoclonal antibodies) can specifically recognize specific targets on the surfaces of tumor cells, and then guide ADCs to the surfaces of cancer cells, and enable ADCs to enter the cancer cells through endocytosis; and then the bioactive molecules are released in the cancer cells to kill the cancer cells without damaging the normal tissue cells as much as possible. Developing antibodies with better targeting, hydrophilicity, and endocytic activity for conjugating cytotoxic small molecules to prepare ADC drugs with a larger therapeutic window will provide patients with better treatment options.

### BRIEF SUMMARY OF THE INVENTION

The present application relates to an antibody-drug conjugate for treating CEACAM5-positive cancers, and exemplarily discloses an antibody-drug conjugate using antibodies Ab-hzDPmut and tusamitamab, as targeting portions and having a structure represented by a general formula HA-(Y-M-L-E-D)ₓ′. The results show that the conjugate has a better drug-antibody conjugation ratio, and the conjugate has excellent binding activity to CEACAM5, and has a good target killing effect on CEACAM5, for example rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer). Therefore, the present application provides an antibody-drug conjugate for treating cancers with high expression of CEACAM5, a pharmaceutical composition comprising the antibody-drug conjugate, and use thereof in treatment of cancers with high expression of CEACAM5.

### Antibody-Drug Conjugate

In one aspect, the present application provides an antibody-drug conjugate having a structure shown as a formula: HA-(Y-M-L-E-D)ₓ′,
wherein HA-(Y-represents an antibody or antigen-binding fragment thereof that specifically binds to CEACAM5, where-(Y-represents a linkage from an amino acid residue of the antibody or antigen-binding fragment thereof to M;
M-L-E is a linker connecting -(Y-to D, wherein M is the portion of the linker connecting to Y, L is the central portion of the linker, and E is the portion of the linker connecting to D;
D is a cytotoxic drug; and
x′ is selected from 1-20.

In one embodiment, the amino acid residue of the antibody or antigen-binding fragment thereof is a cysteine, lysine, serine or threonine residue.

In one embodiment, HA-(Y-(or HA-Y-) is HA-(S-, HA-(NH—, or HA-(O-(or HA-S, HA-NH, or HA-O—).

In one embodiment, -(Y— is —(S-, -(NH— or -(O—.

In one embodiment, x′ is 1-16, 1-8, 4-8, 4 or 8.

In one embodiment, x′ is 1-16.

In one embodiment, x′ is 1-8.

In one embodiment, x′ is 4-8.

In one embodiment, x′ is 8.

In some embodiments, the antibody-drug conjugates provided herein comprise a humanized anti-CEACAM5 antibody. In some embodiments, the antibody has excellent binding activity to CEACAM5. In some embodiments, the antibody-drug conjugates provided herein can efficiently deliver drugs to CEACAM5.

In some embodiments, the antibody or antigen-binding fragment thereof comprises:
(1) the following heavy chain variable regions (VH) and/or light chain variable regions (VL), wherein CDRs are defined according to a Chothia numbering system:
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
   or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement.

In some embodiments, the antibody or antigen-binding fragment thereof comprises:
(a) a V_{H} shown as SEQ ID NO: 1 or a variant thereof, and/or, a V_{L} shown as SEQ ID NO: 2 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived; and preferably, the replacement is a conservative replacement.

In some embodiments, the antibody or antigen-binding fragment thereof further comprises:
(a) a human mutant immunoglobulin (IgG1) heavy chain constant region (CH) or a variant thereof, wherein the variant has replacement, deletion or addition of one, two, or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human mutant immunoglobulin (IgG1) light chain constant (CL) or a variant, wherein the variant has replacement, deletion or addition of one, two, or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In some embodiments, the heavy chain constant region is a human mutant IgG1 heavy chain constant region. In some embodiments, the antibody or antigen-binding fragment thereof comprises a heavy chain constant region (CH) shown as SEQ ID NO: 3 or a variant thereof, and the variant has a conservative replacement of up to 20 amino acids (for example, a conservative replacement of up to 15, up to 10 or up to 5 amino acids; for example, a conservative replacement of 1, 2, 3, 4 or 5 amino acids) as compared to SEQ ID NO: 3.

In some embodiments, the light chain constant region is a human IgG1 light chain constant region. In some embodiments, the antibody or antigen-binding region thereof comprises a light chain constant region (CL) shown as SEQ ID NO: 4 or a variant thereof, and the variant has a conservative replacement of up to 20 amino acids (for example, a conservative replacement of up to 15, up to 10 or up to 5 amino acids; for example, a conservative replacement of 1, 2, 3, 4 or 5 amino acids) as compared to SEQ ID NO: 4.

In some embodiments, the antibody or antigen-binding fragment thereof comprises a heavy chain constant region (CH) shown as SEQ ID NO: 3, and a light chain constant region (CL) shown as SEQ ID NO: 4.

In some embodiments, the antibody or antigen-binding fragment thereof comprises a heavy chain comprising a V_{H} sequence shown as SEQ ID NO: 1 and heavy chain constant region (CH) shown as SEQ ID NO: 3, and a light chain comprising a V_{L} sequence shown as SEQ ID NO: 2 and light chain constant region (CL) shown as SEQ ID NO: 4.

In a further embodiment, the antibody comprises a heavy chain (HC) having the amino acid sequence as set forth in SEQ ID NO: 20, and a light chain (LC) having the amino acid sequence as set forth in SEQ ID NO: 21.

In the antibody-drug conjugate, the cytotoxic drug may be linked to the antibody or antigen-binding fragment through the linker (like “M-L-E” fragment shown in the present application).

In some embodiments, M is

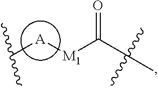

wherein ring A is a 5-6 membered aliphatic heterocyclic ring, or 5-20 membered aromatic ring system, and the aliphatic heterocyclic ring and aromatic ring systems are optionally substituted by one or more members independently selected from the group consisting of oxo (═O), halogen, cyano, amino, carboxyl, mercapto and C₁₋₆ alkyl; and M₁ is selected from a single bond, C₁₋₂₀ alkylene, C₂₋₂₀ alkenylene and C₂₋₂₀ alkynylene.

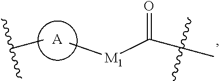

In some embodiments, M is wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted by one or more members independently selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M is

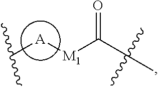

wherein ring A is selected from

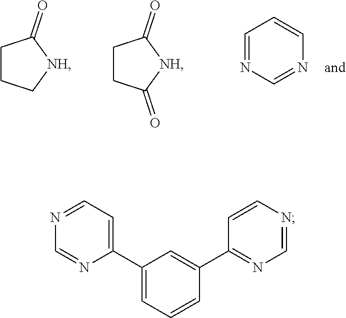 [SMILES: O=C1CCCN1 ; O=C1CCC(=O)N1 ; c1cncnc1 ; c1cc(-c2ccncn2)cc(-c2ccncn2)c1]

and M₁ is selected from a single bond, C₅₋₈ alkylene, C₅₋₈ alkenylene and C₅₋₈ alkynylene.

In some embodiments, M is selected from the following structures:

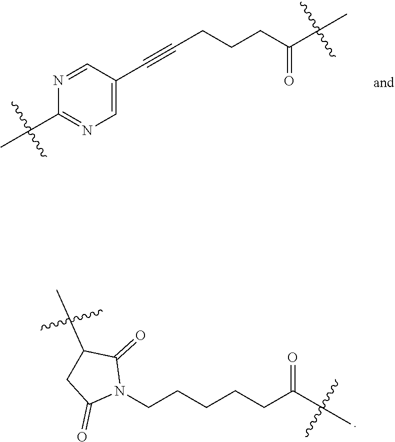 [SMILES: *C(=O)CCCC#Cc1cnc(*)nc1 ; *C(=O)CCCCCN1C(=O)CC(*)C1=O]

In some embodiments, M is:

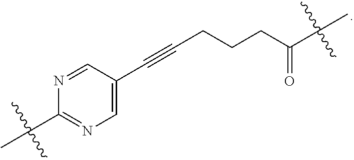 [SMILES: *C(=O)CCCC#Cc1cnc(*)nc1]

In some embodiments, L is selected from a structure comprising one or more of C₁₋₆ alkylene, -N(R′)—, carbonyl, -O—, Val, Cit, Phe, Lys, Lys(COCH₂CH₂ (OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 25), Gly-Phe-Leu-Gly (SEQ ID NO: 26), Gly-Gly-Val-Ala (SEQ ID NO: 27), Gly-Gly-Gly-Gly-Gly (SEQ ID NO: 28),

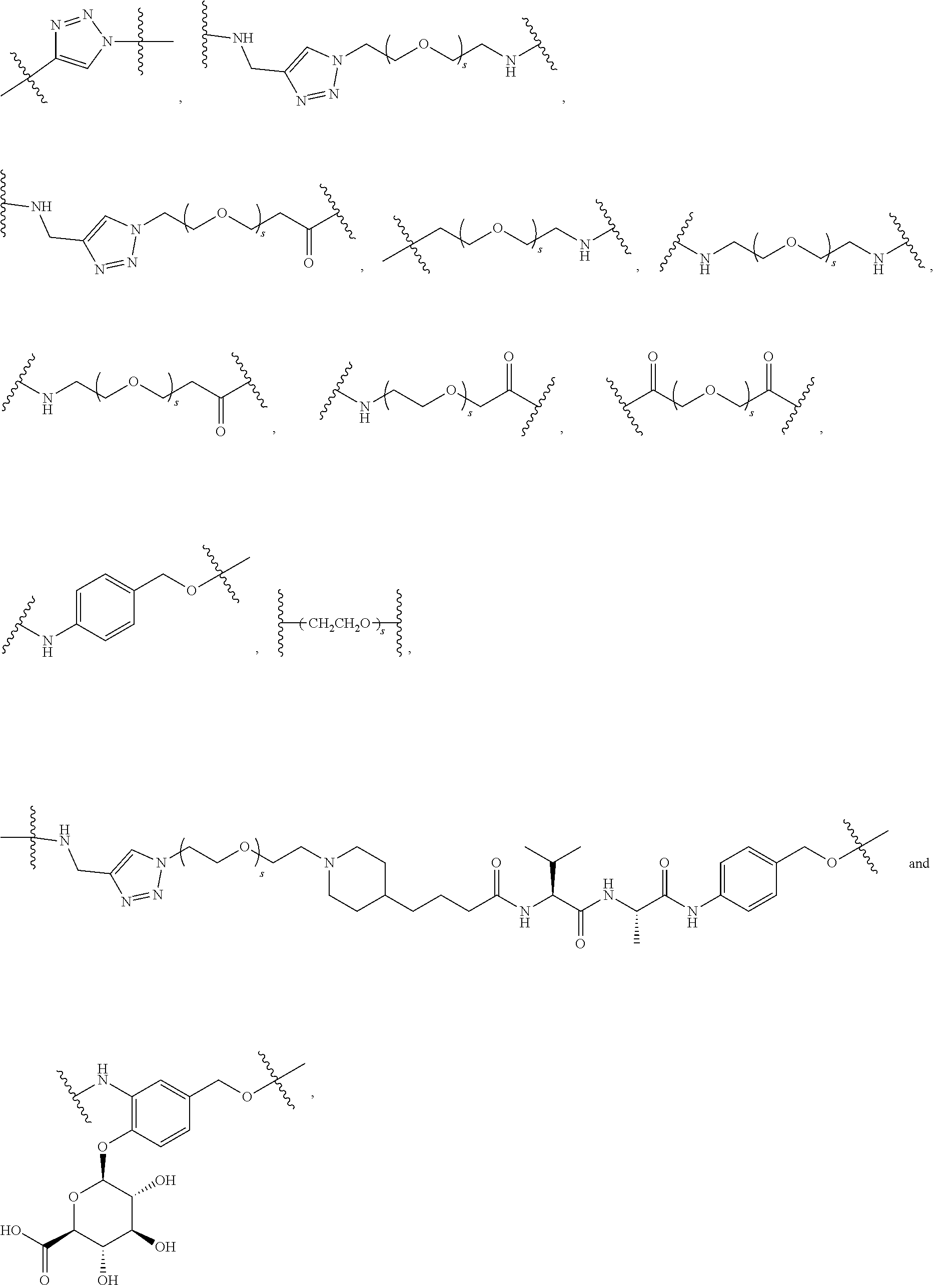

or a combination thereof, wherein R′ represents hydrogen, C₁₋₆ alkyl or alkyl containing —(CH₂CH₂O)ᵣ-; r is selected from an integer from 1-10; and s is selected from an integer from 1-20.

In some embodiments, L is selected from a structure comprising one or more of C₁₋₆ alkylene, -N(R′)—, carbonyl, -O—, Val, Cit, Phe, Lys, Lys(COCH₂CH₂ (OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 25), Gly-Phe-Leu-Gly (SEQ ID NO: 26), Gly-Gly-Val-Ala (SEQ ID NO: 27), Gly-Gly-Gly-Gly-Gly (SEQ ID NO: 28),

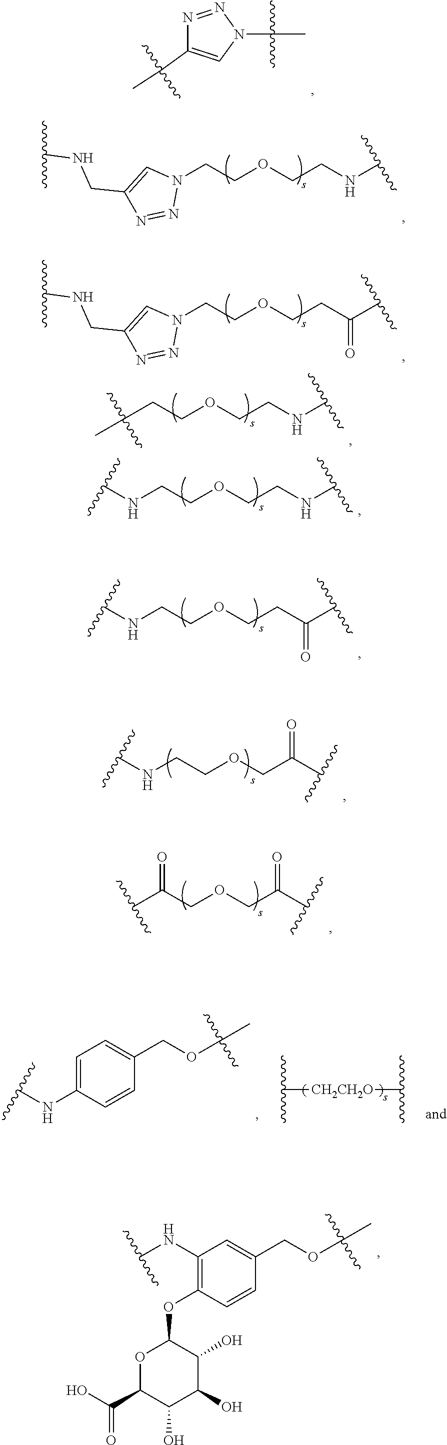

or a combination thereof, wherein R′ represents hydrogen, C₁₋₆ alkyl or alkyl containing—(CH₂CH₂O)ᵣ-; r is selected from an integer from 1-10; and s is selected from an integer from 1-20.

In some embodiments, L is selected from a structure comprising one or more of C₁₋₆ alkylene, —NH—, Val, Cit, Phe, Lys, Lys(COCH₂CH₂ (OCH₂CH₂)ₛOCH₃), Gly, Val-Cit, Gly-Gly-Phe-Gly (SEQ ID NO: 25),

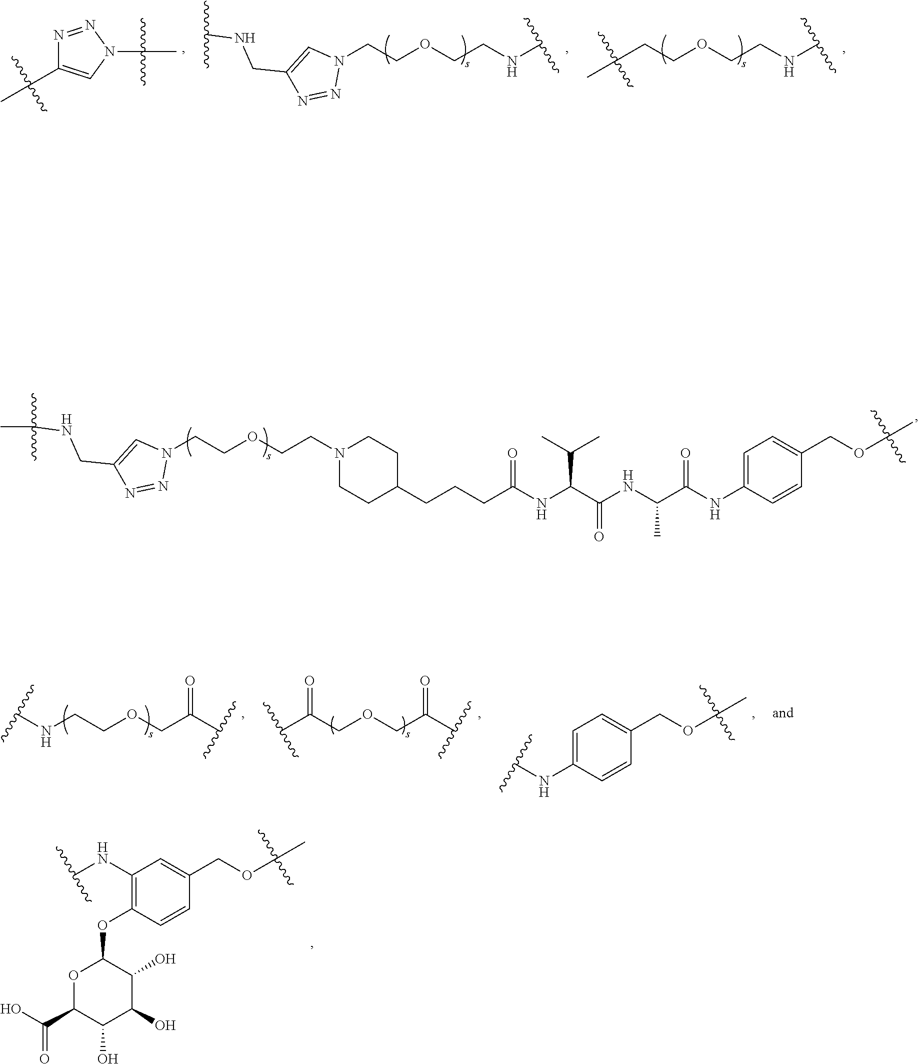

or a combination thereof; where s is selected from an integer from 1-20.

In some embodiments, L is selected from a structure comprising one or more of C₁₋₆ alkylene, —NH—, Val, Cit, Phe, Lys, Lys(COCH₂CH₂ (OCH₂CH₂)ₛOCH₃), Gly, Val-Cit, Gly-Gly-Phe-Gly (SEQ ID NO: 25),

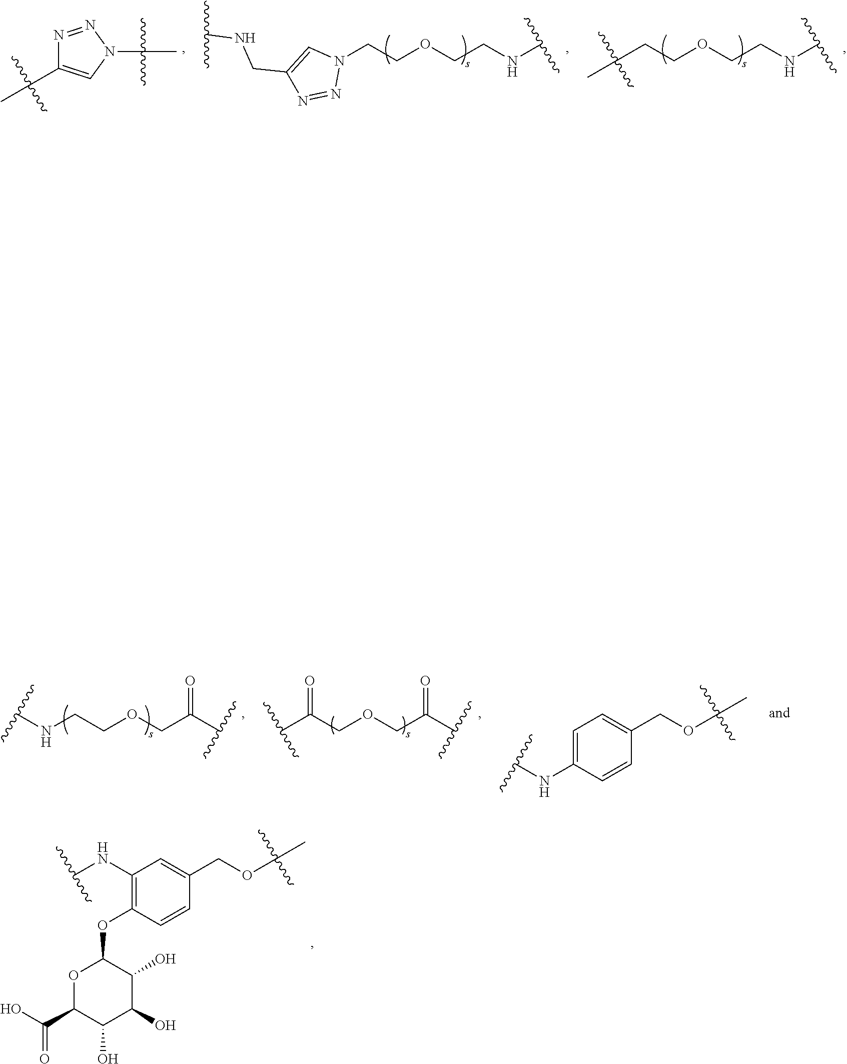

or a combination thereof; where s is selected from an integer from 1-20.

In some embodiments, L is selected from the following structures:

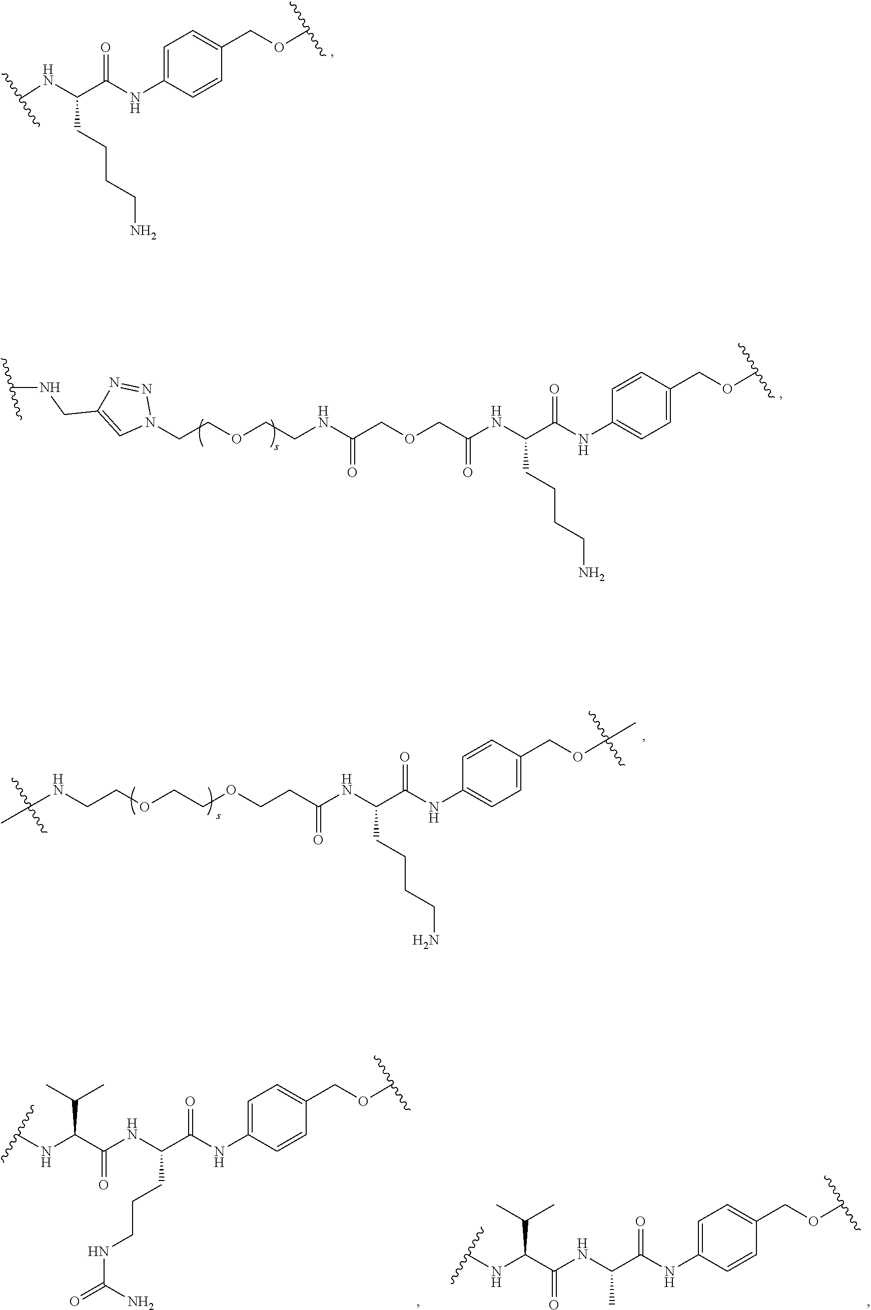

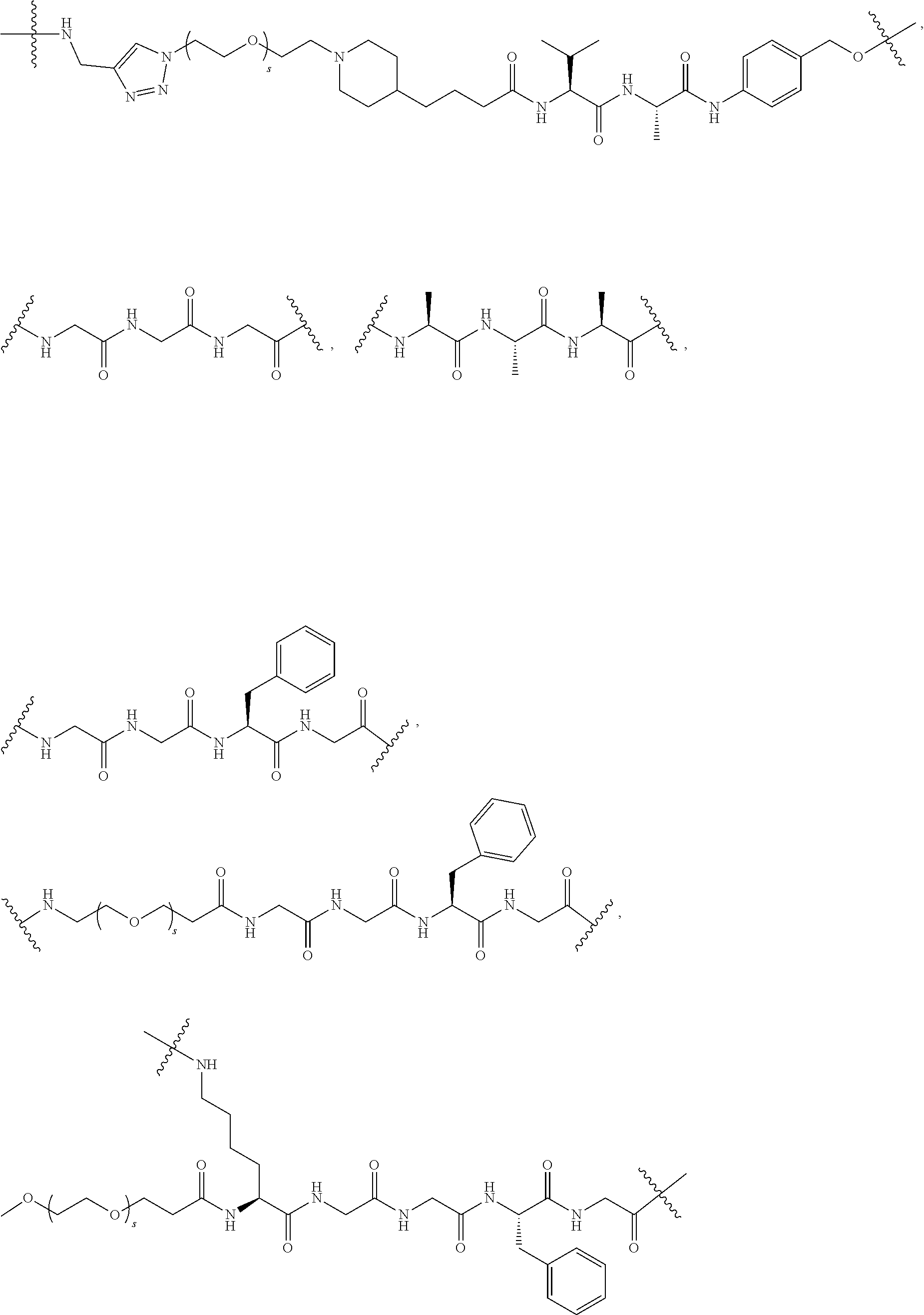

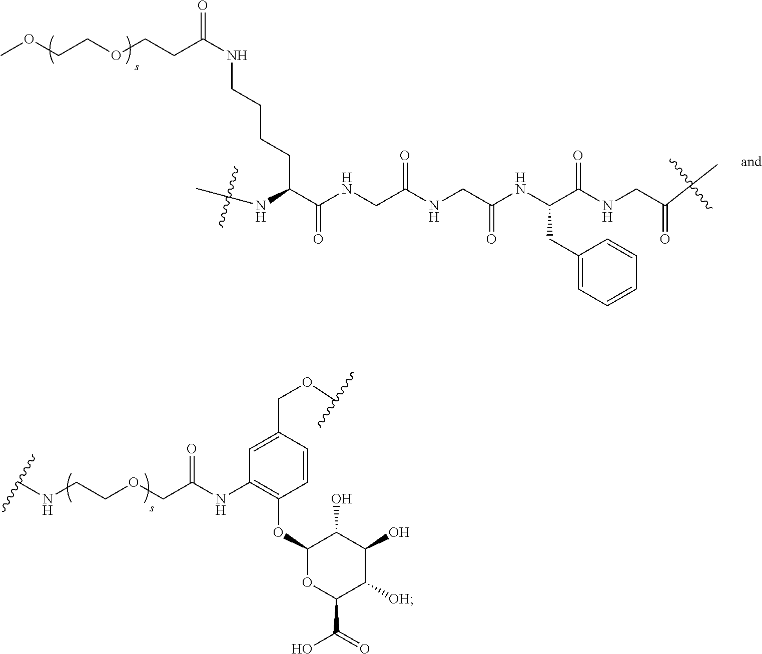

where s is selected from an integer from 1-20.

In some embodiments, L is selected from the following structures:

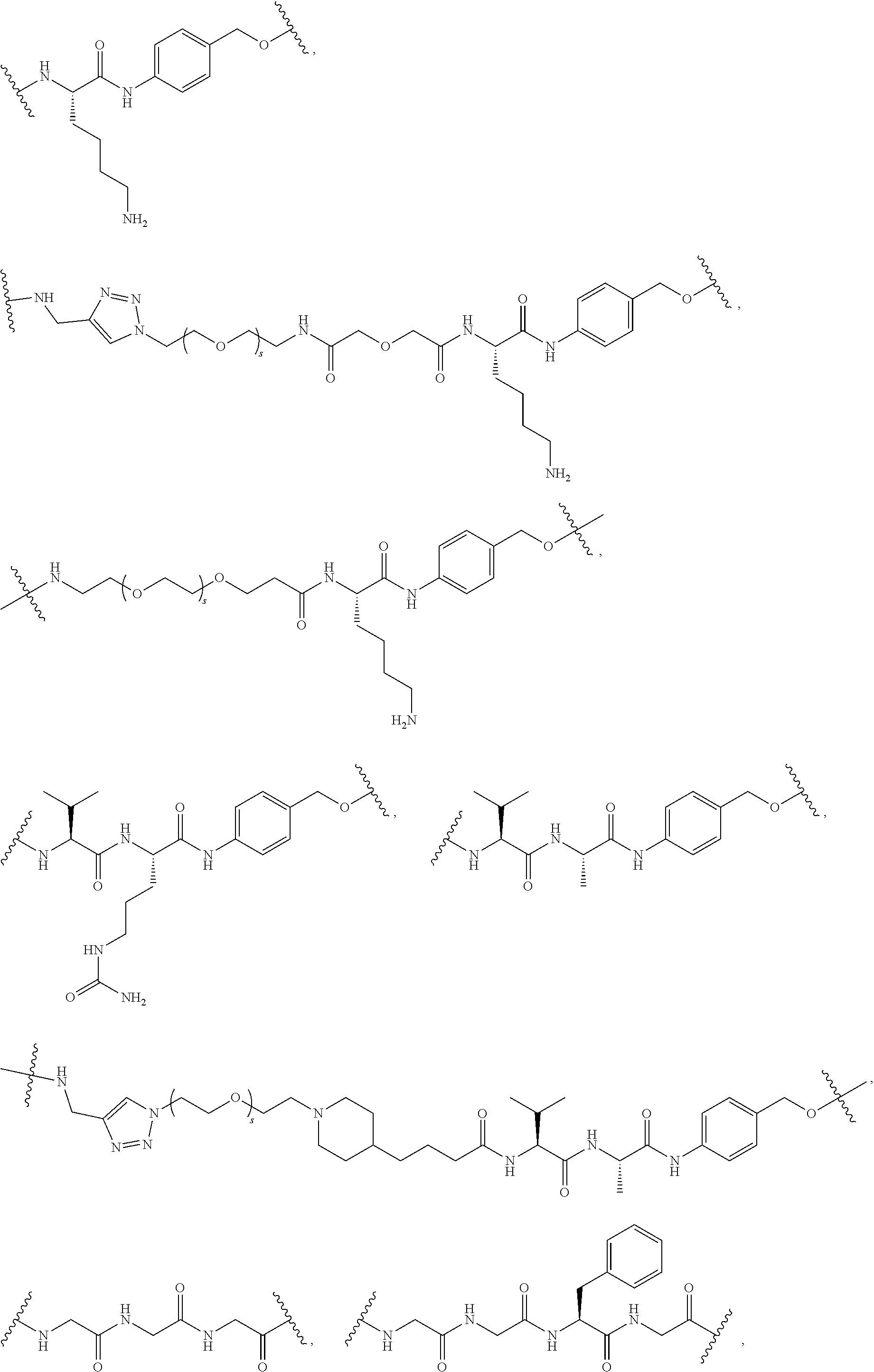

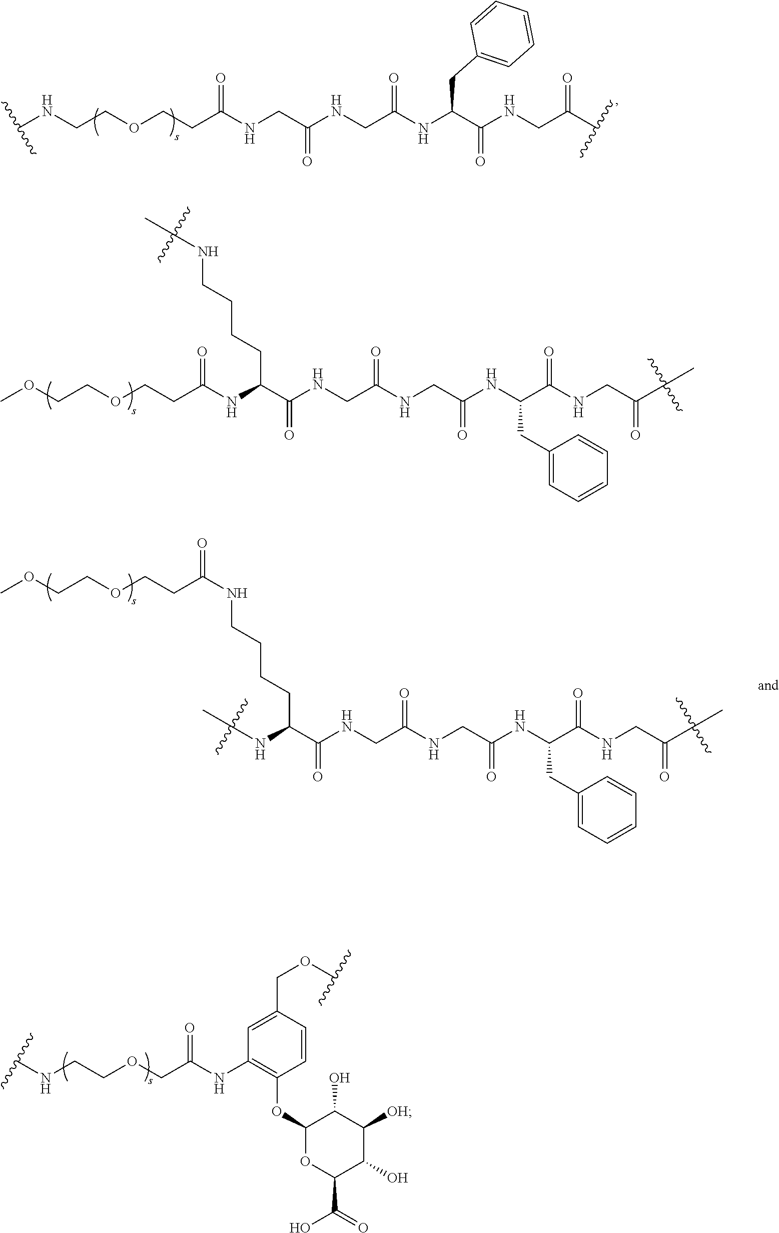

where s is selected from an integer from 1-20.

In some embodiments, L is selected from the following structures:

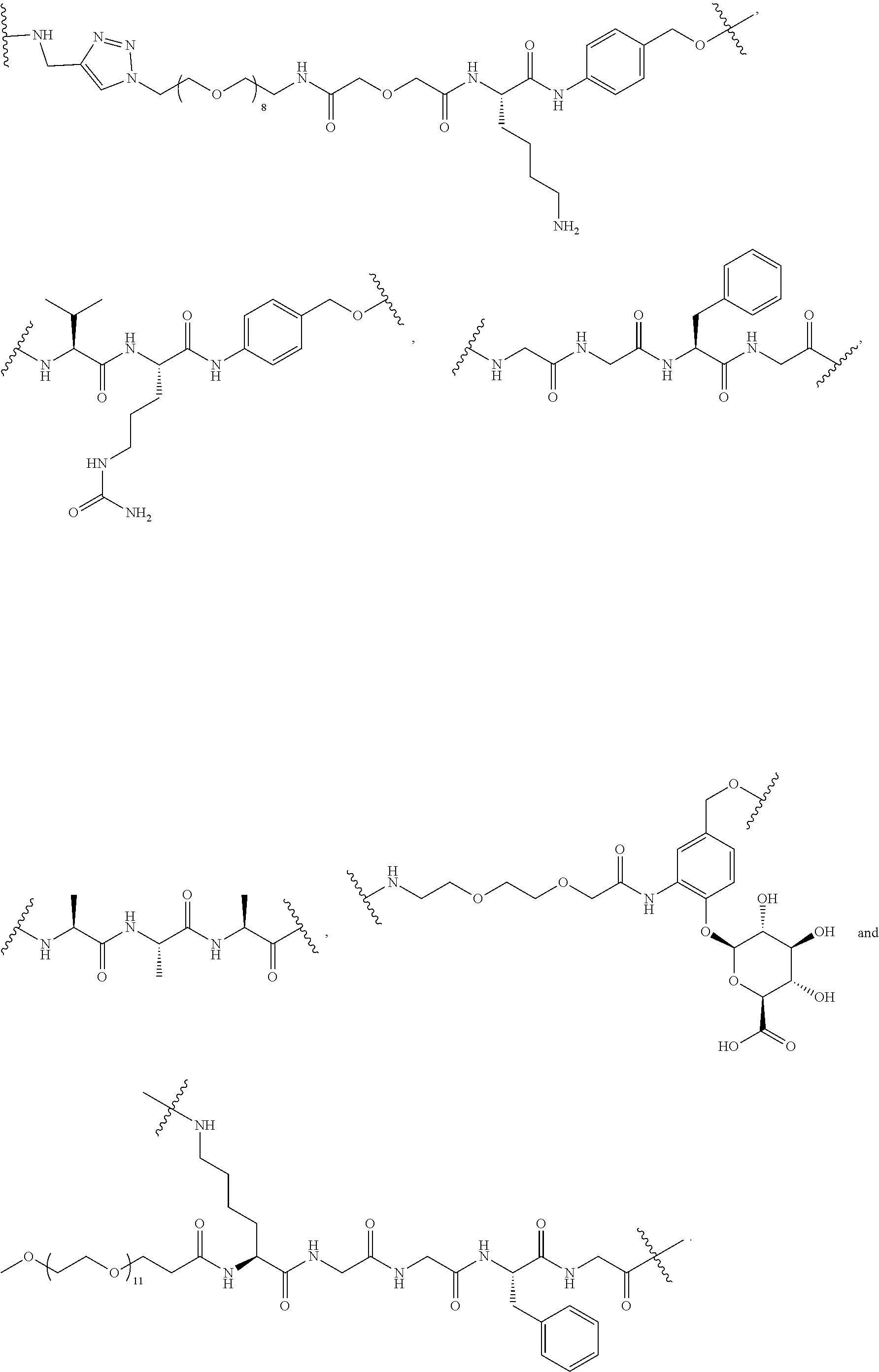

In some embodiments, L is selected from the following structures:

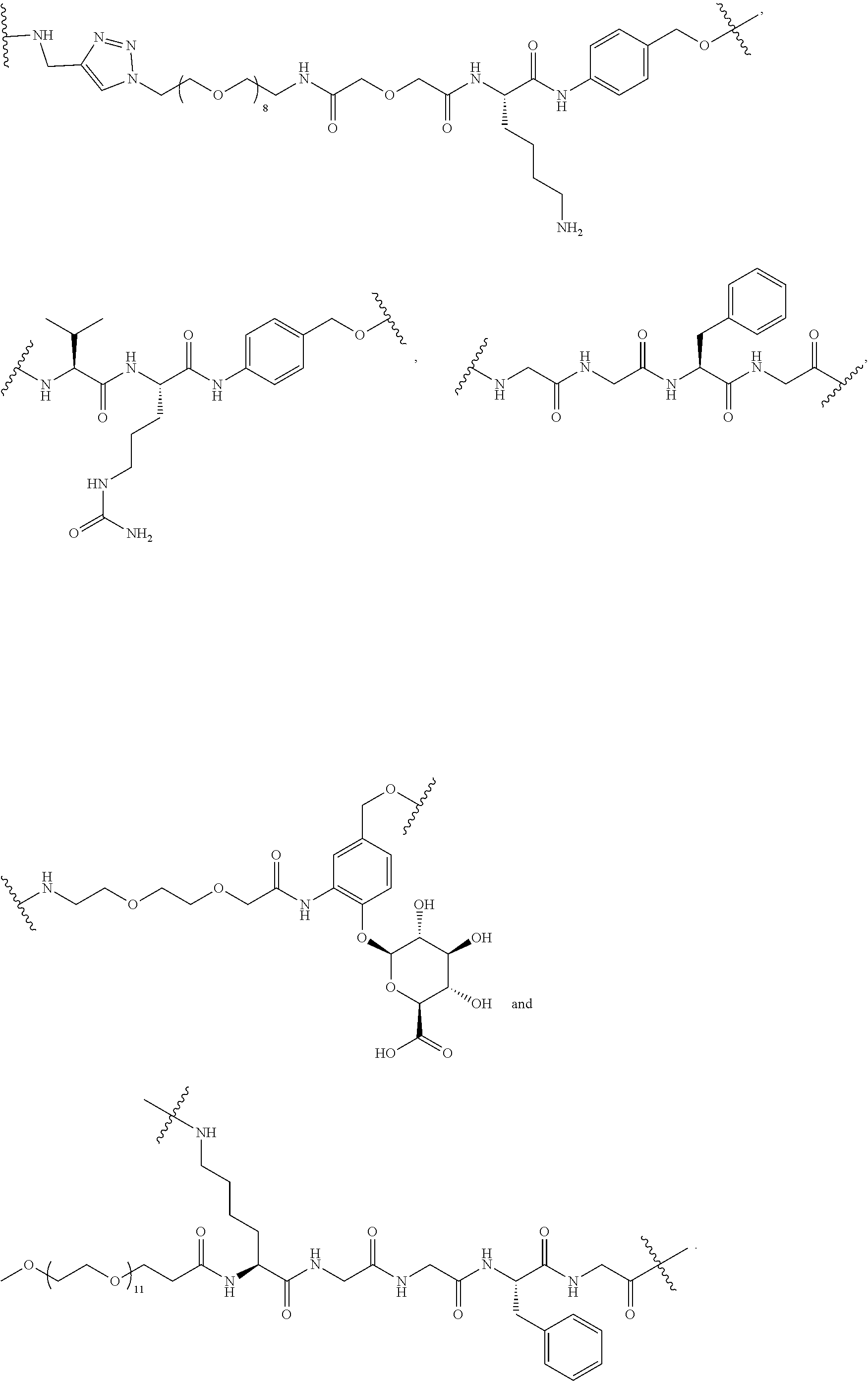

In some embodiments, L is selected from the following structures:

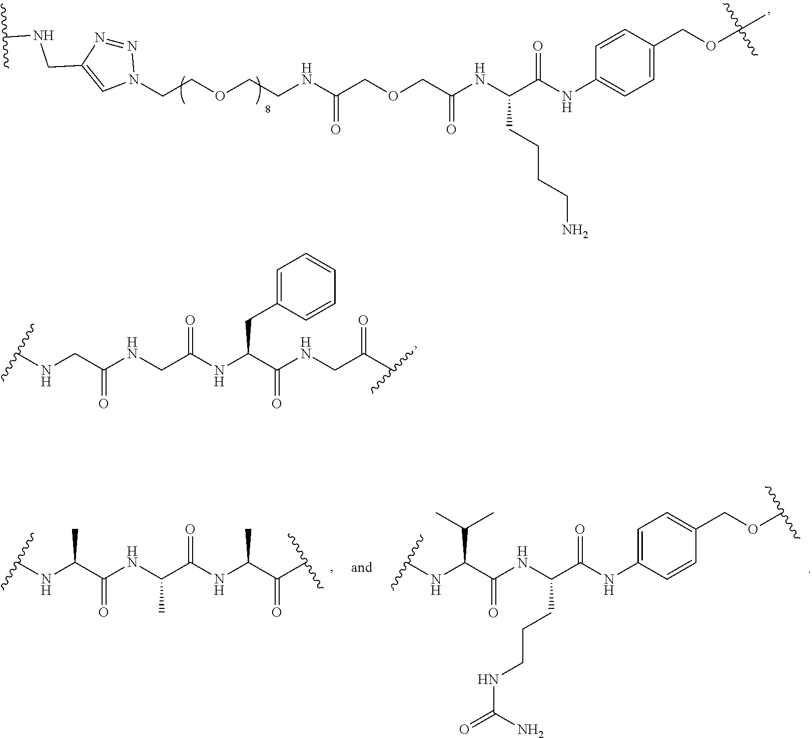

In some embodiments, L is selected from the following structures:

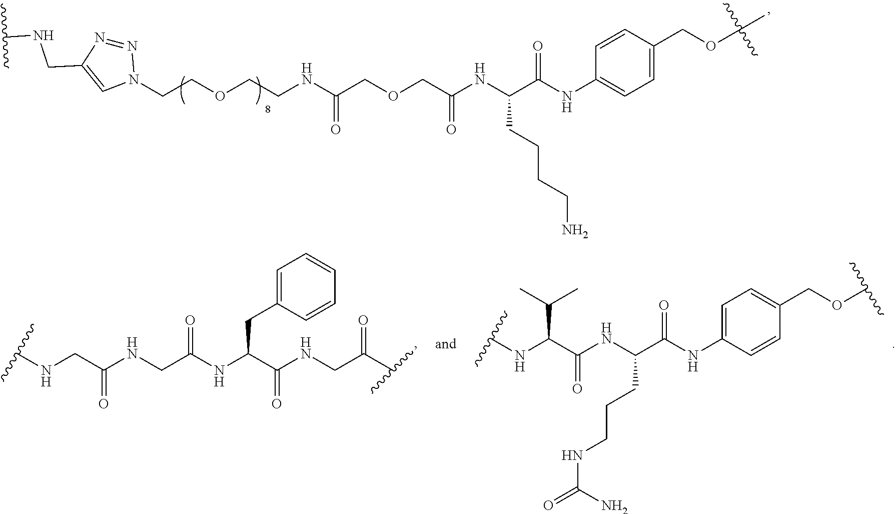

In some embodiments, L is selected from the following structures:

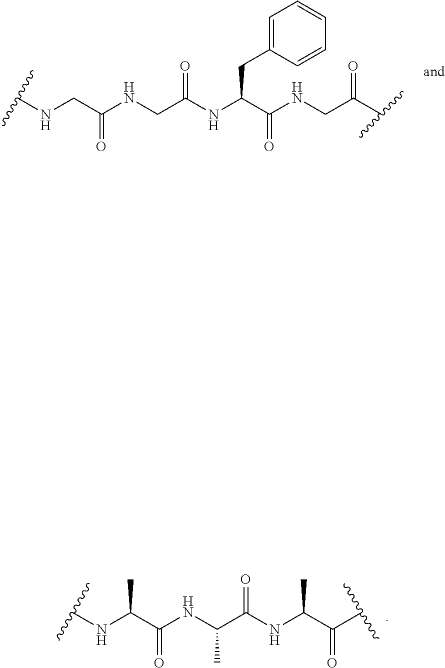 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O ; "."]

In some embodiments, L is

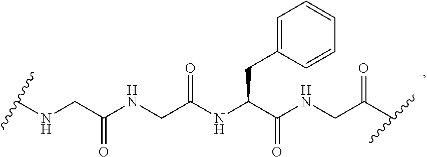 [SMILES: *NCC(=O)NCC(=O)N[C@@H](Cc1ccccc1)C(=O)NCC(*)=O]

In some embodiments, L is

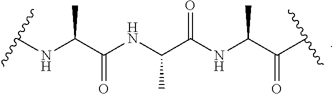 [SMILES: *N[C@@H](C)C(=O)N[C@@H](C)C(=O)N[C@@H](C)C(*)=O ; "."]

In some embodiments, E is a single bond, —NH—CH₂-, —NH—CH₂-O-CH₂—CO—,

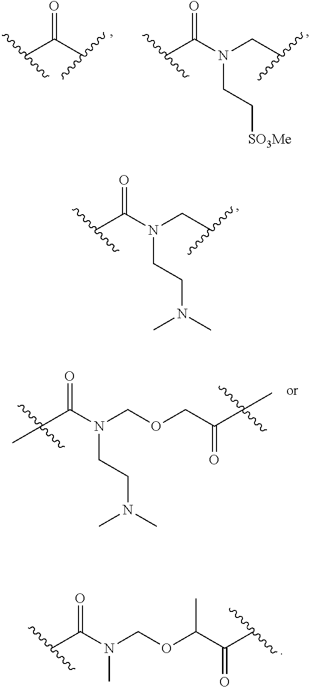

In some embodiments, E is a single bond, —NH—CH₂-, —NH—CH₂-O-CH₂—CO—,

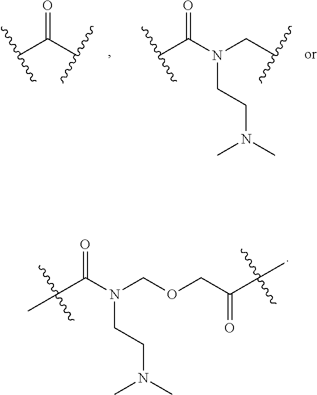

In some embodiments, E is —NH—CH₂-,

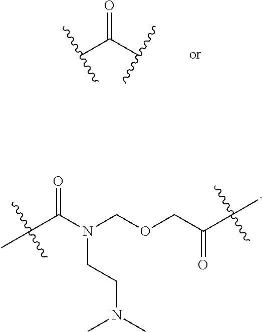

In some embodiments, E is —NH—CH₂— or

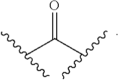 [SMILES: *C(*)=O]

In some embodiments, E is —NH—CH₂— or —NH—CH₂-O-CH₂—CO-.

In some embodiments, E is —NH—CH₂-O-CH₂—CO-.

In some embodiments, M is:

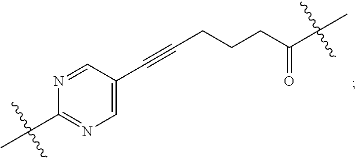 [SMILES: *C(=O)CCCC#Cc1cnc(*)nc1]

L is selected from the following structures:

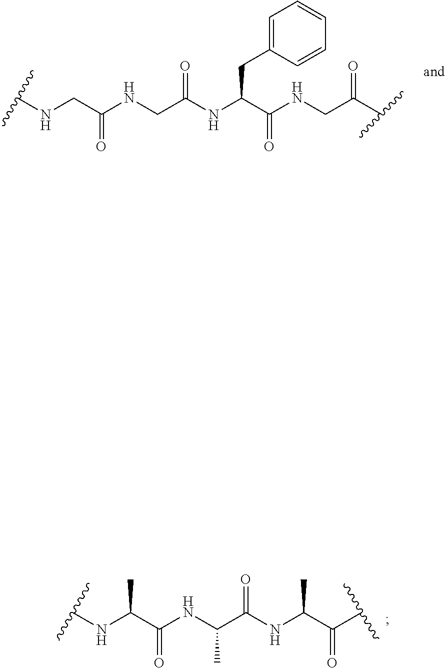 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O ; ";"]

E is —NH—CH₂— or —NH—CH₂-O-CH₂—CO-.

In some embodiments,

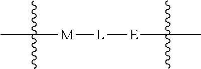

is selected from the following structures:

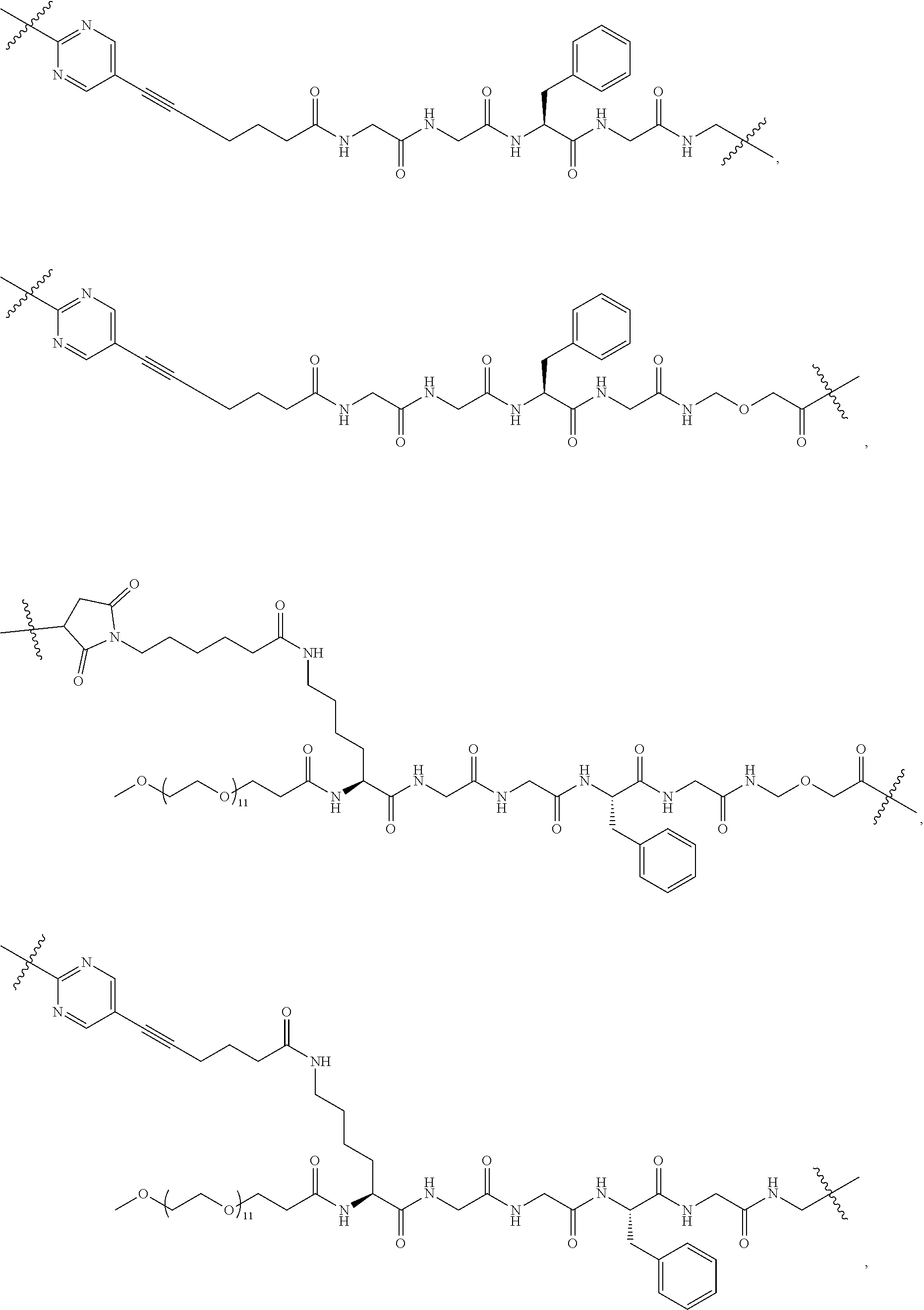

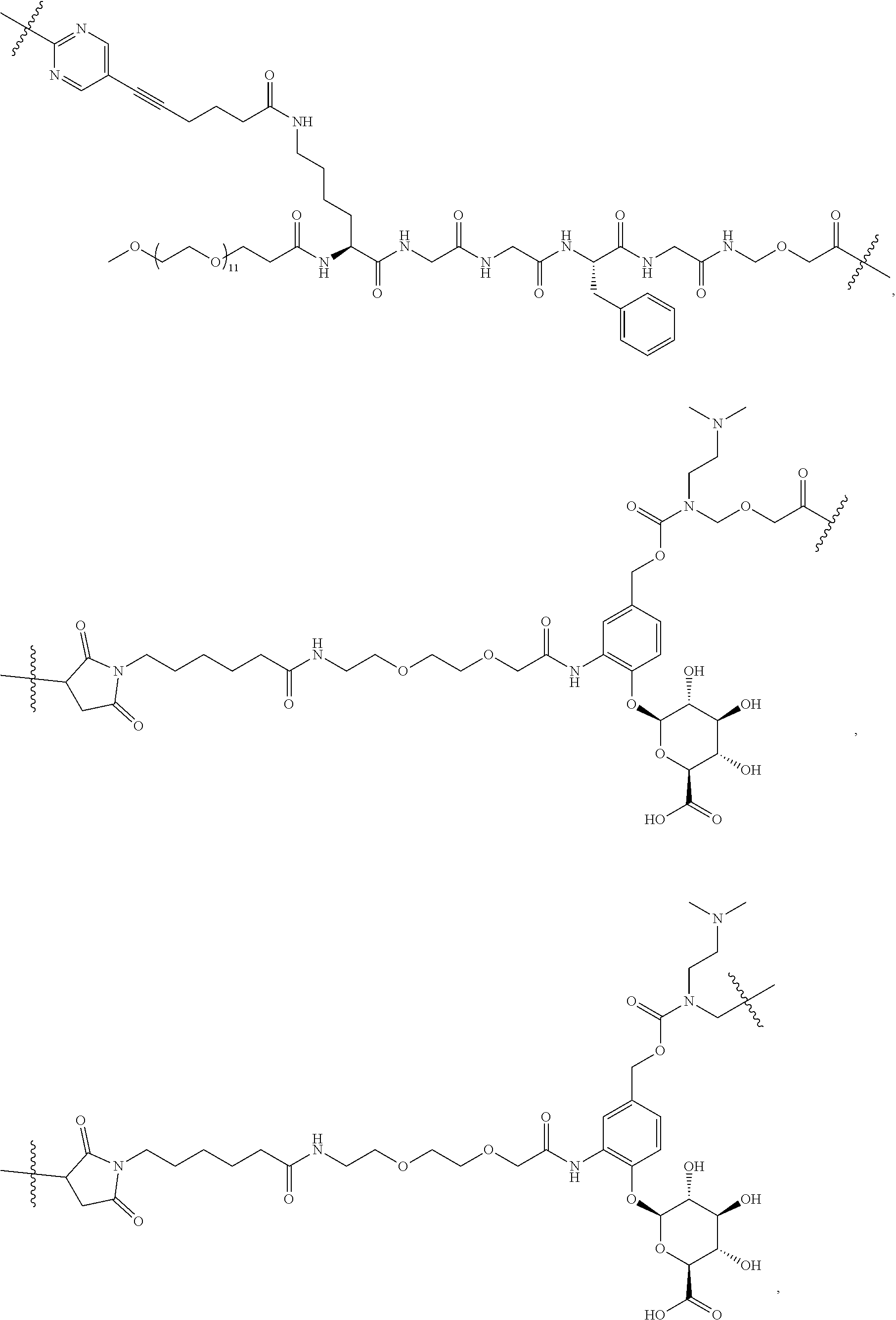

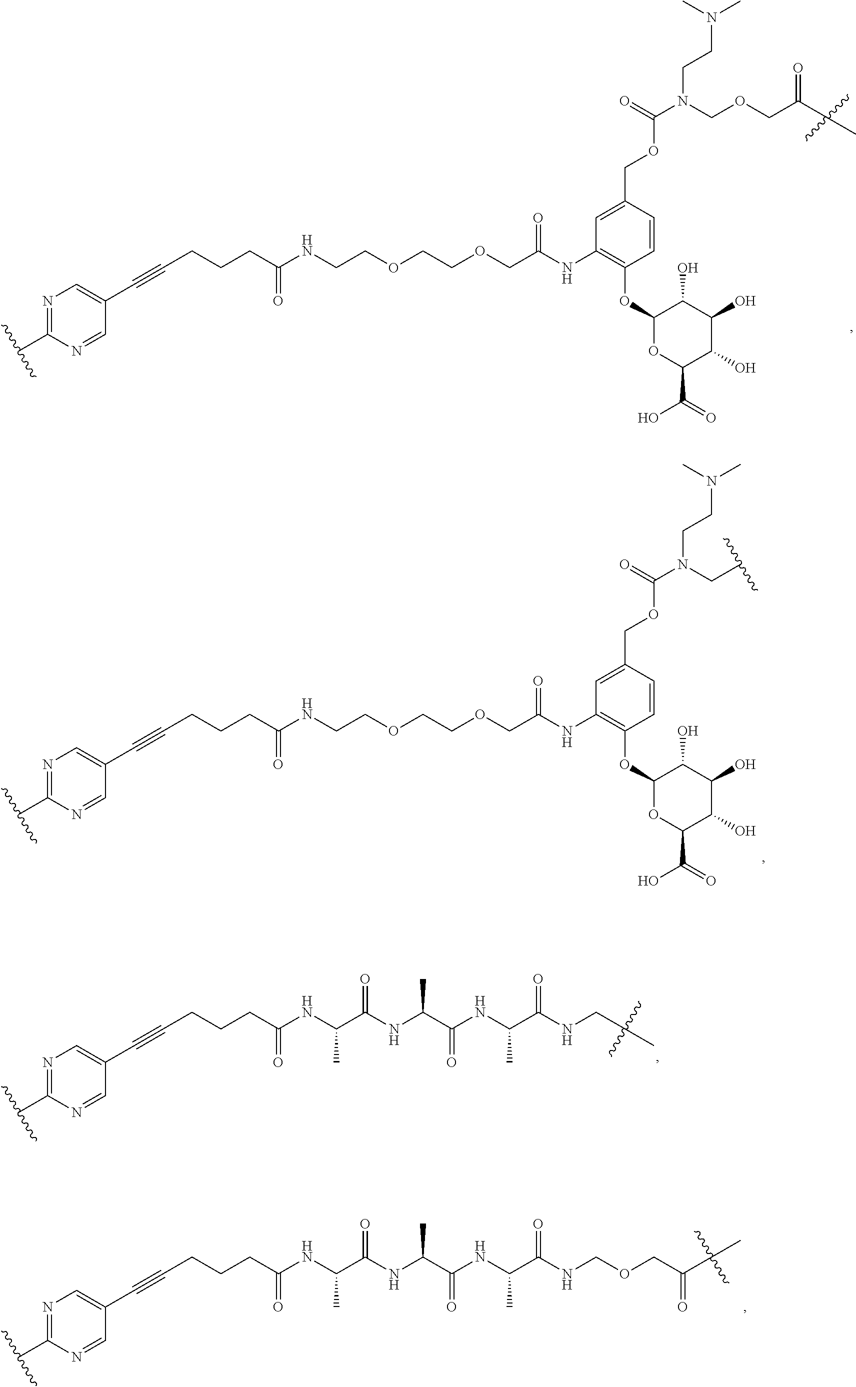 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

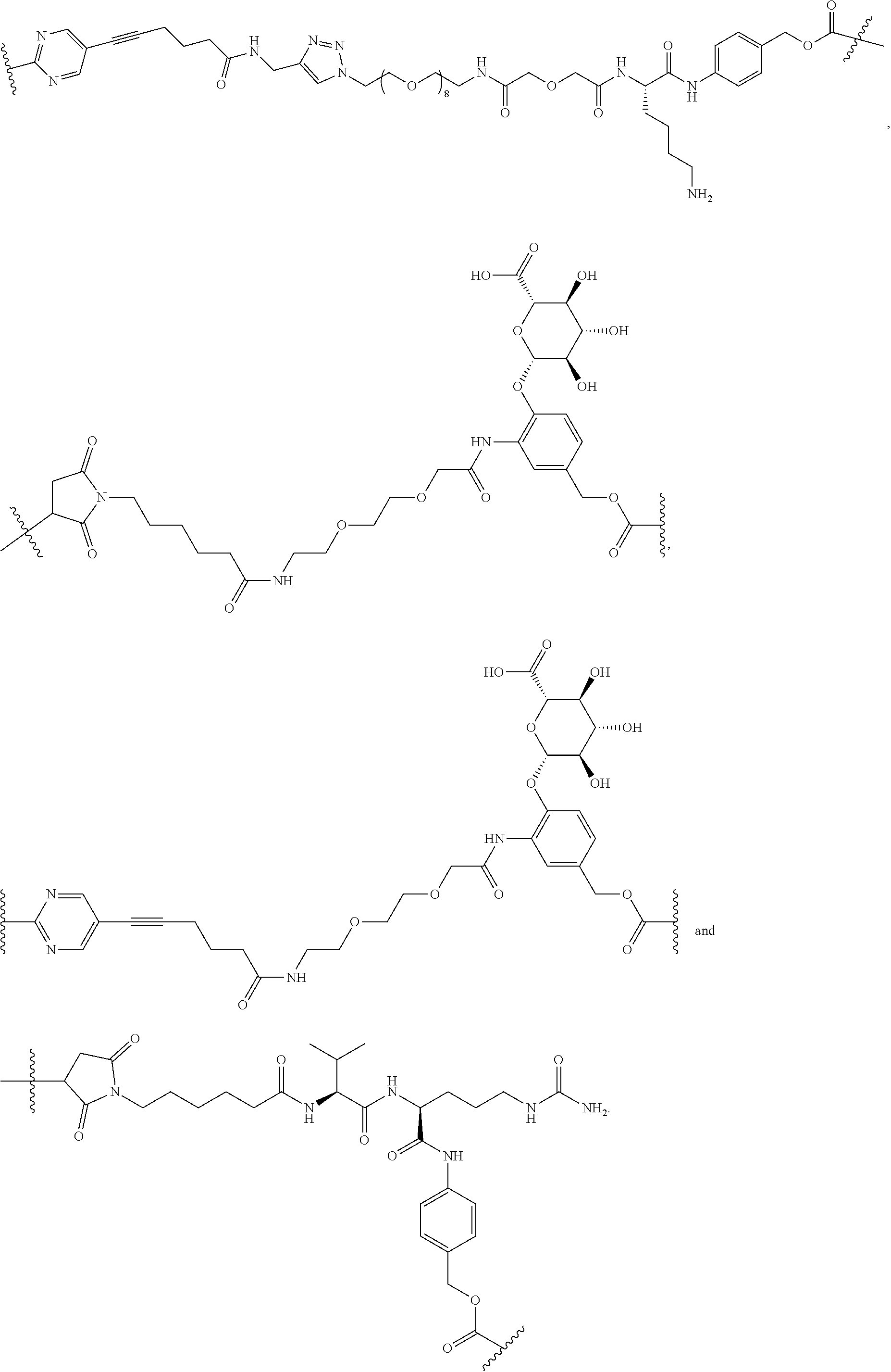

In some embodiments,

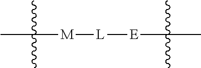

is selected from the following structures:

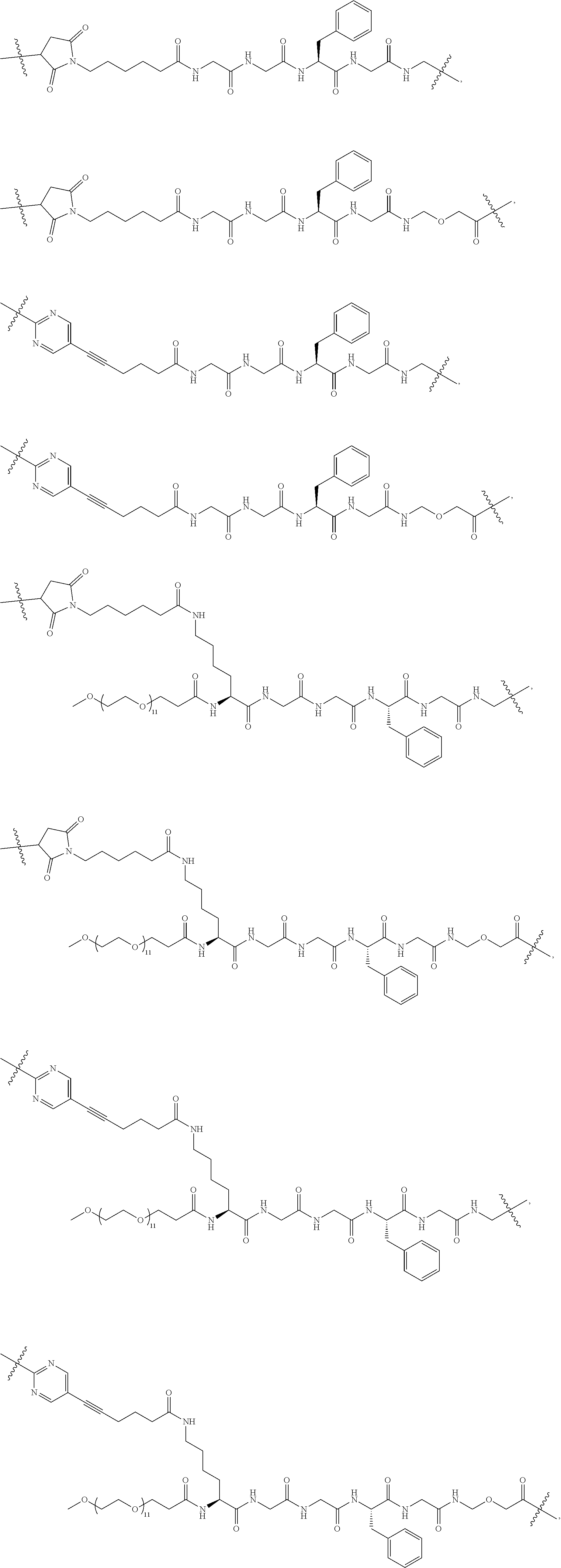

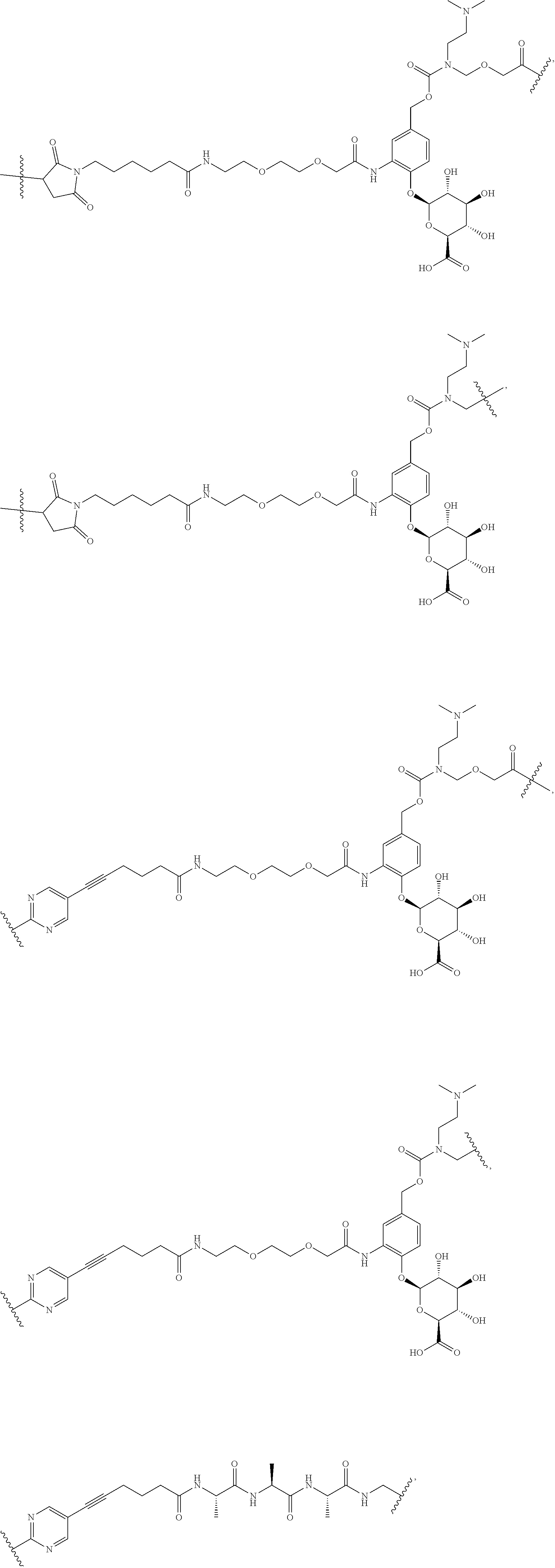

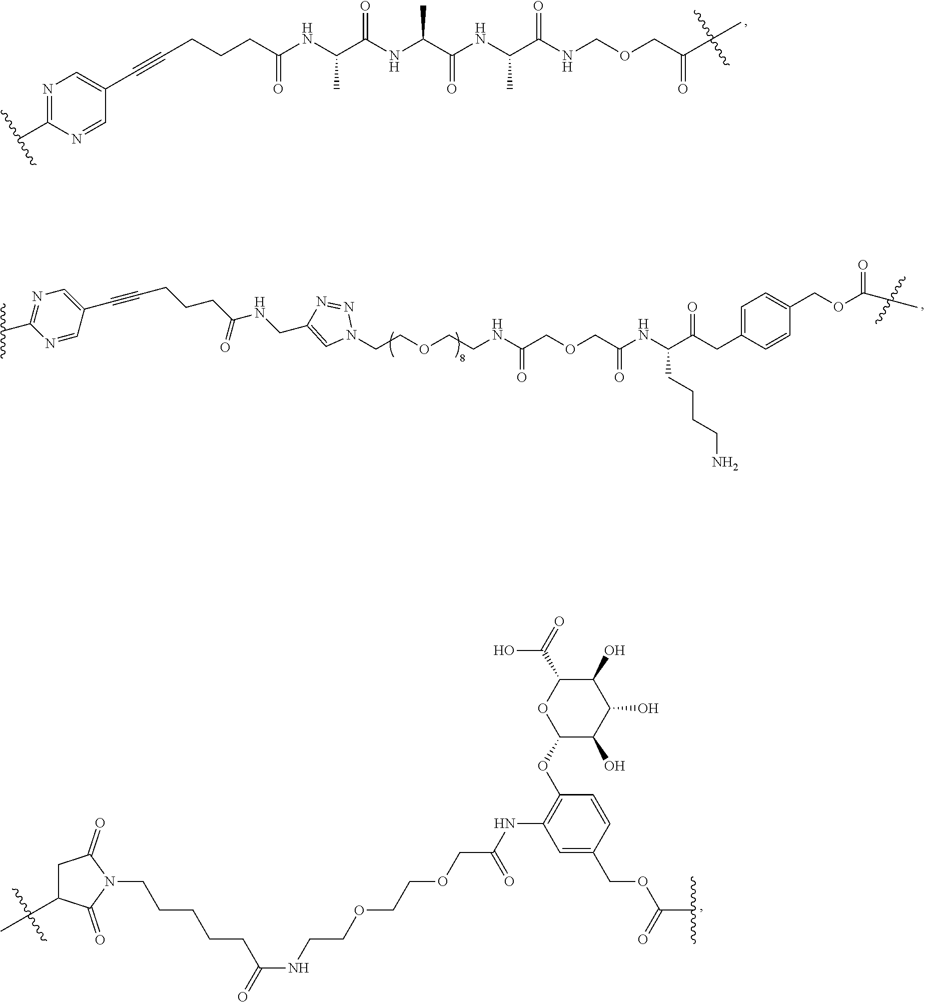

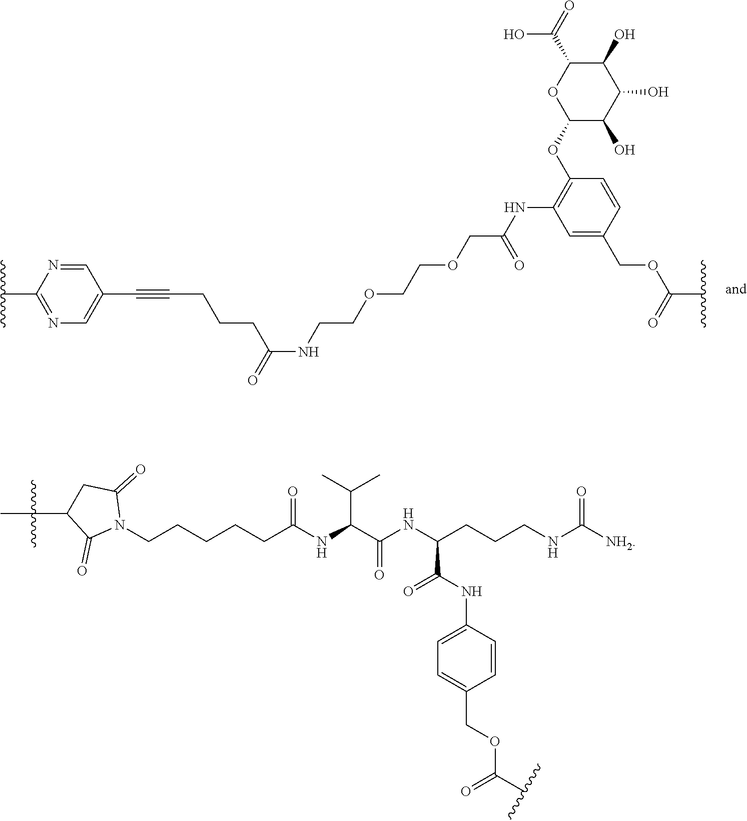 [SMILES: *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments,

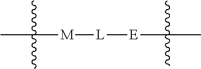

is selected from the following structures:

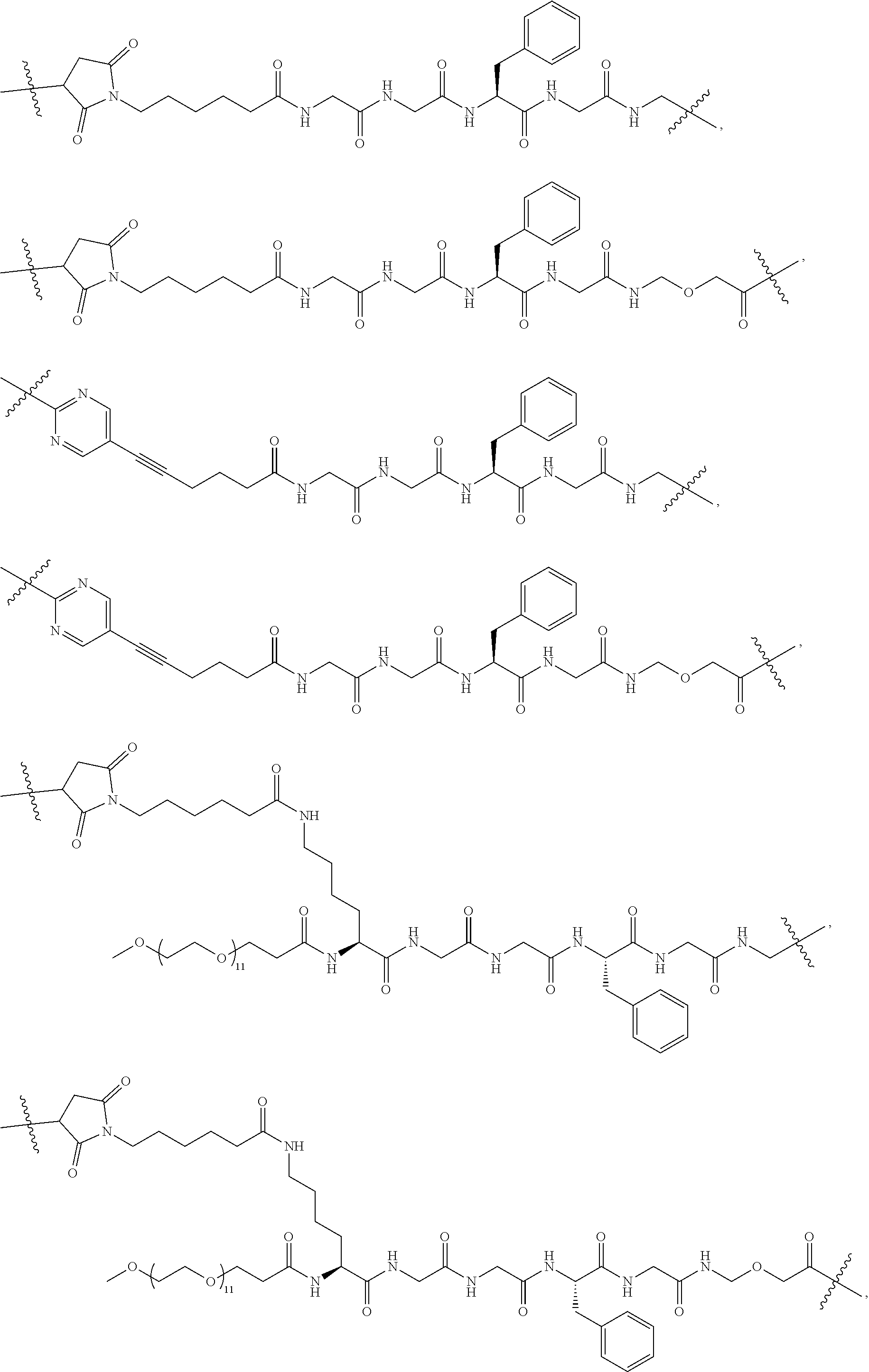

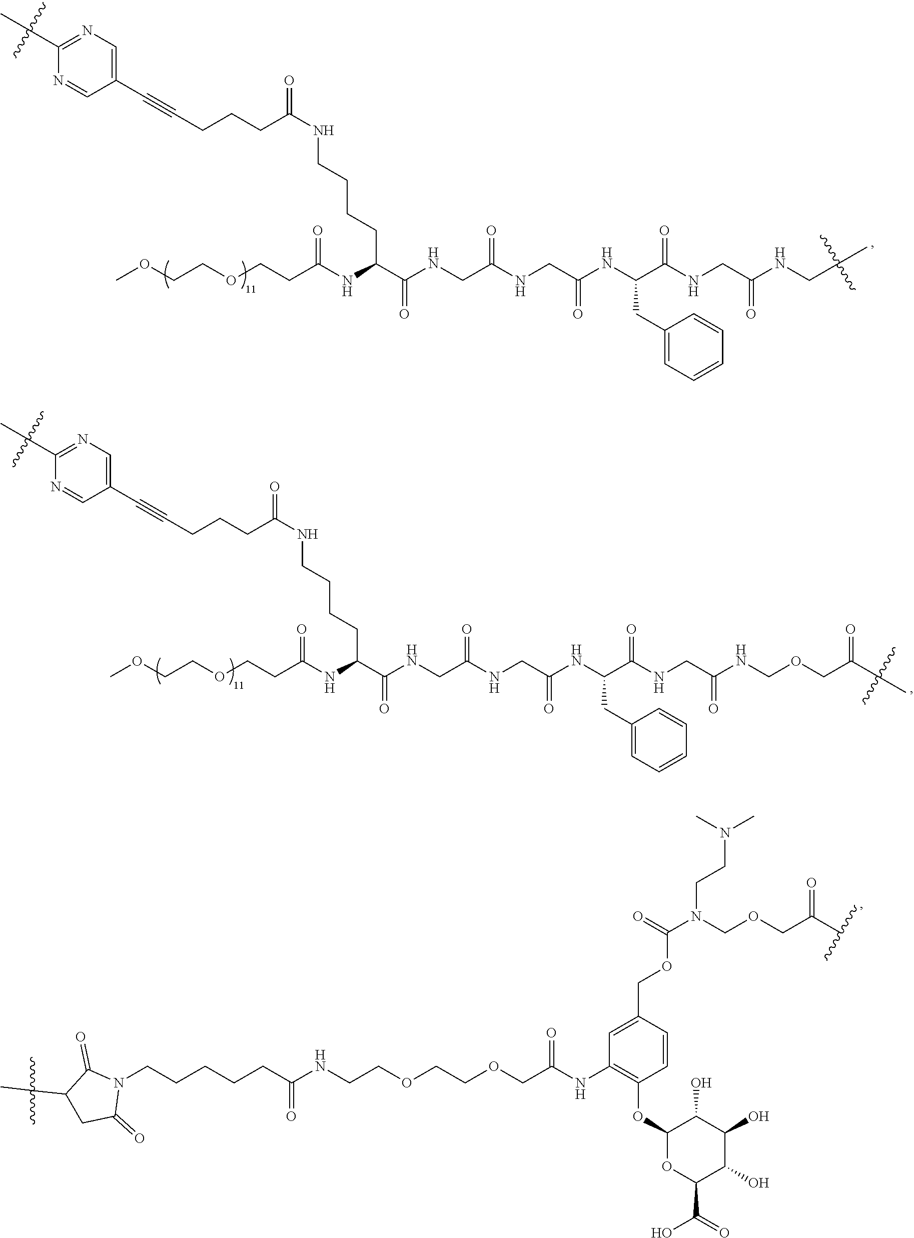

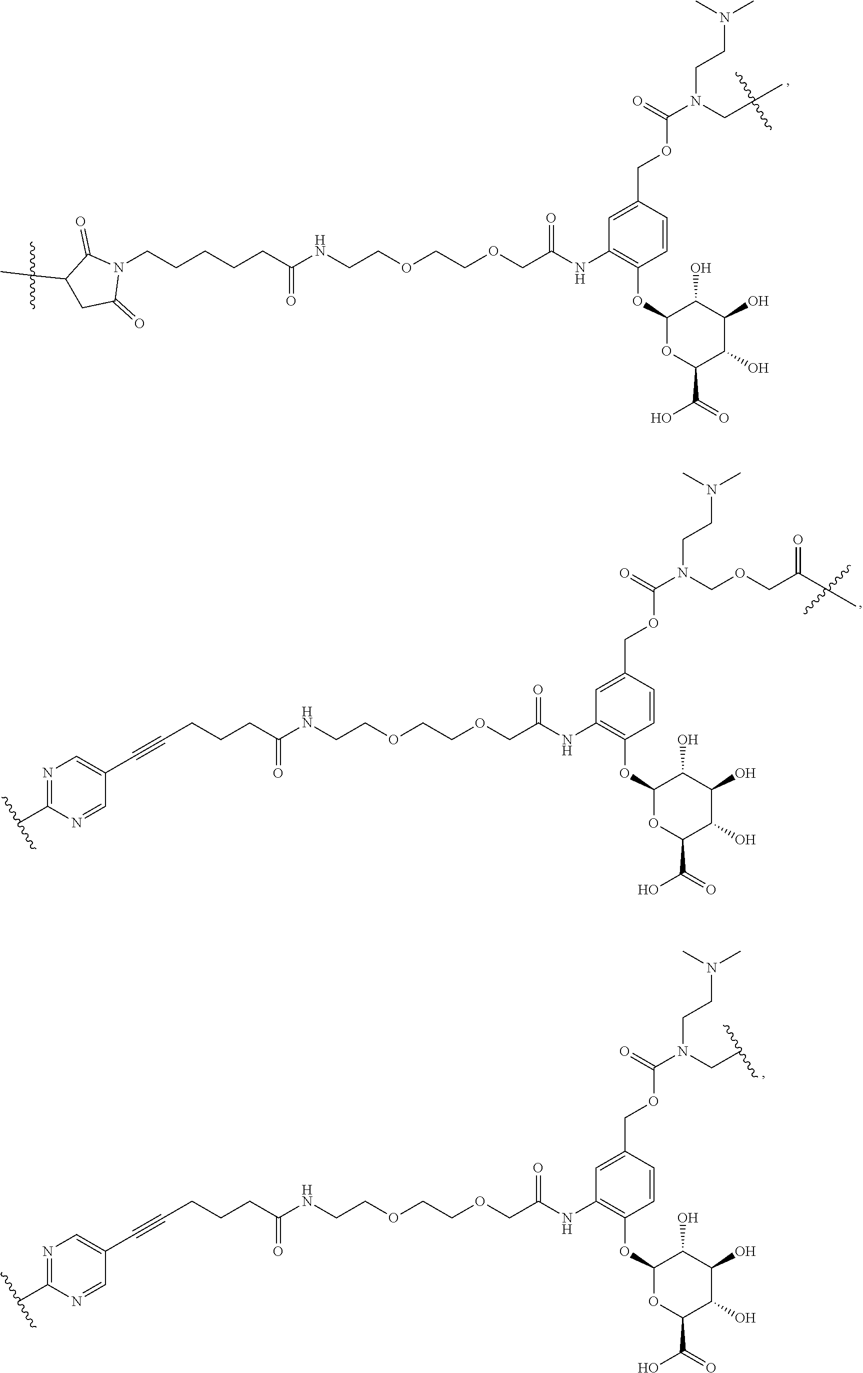 [SMILES: *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

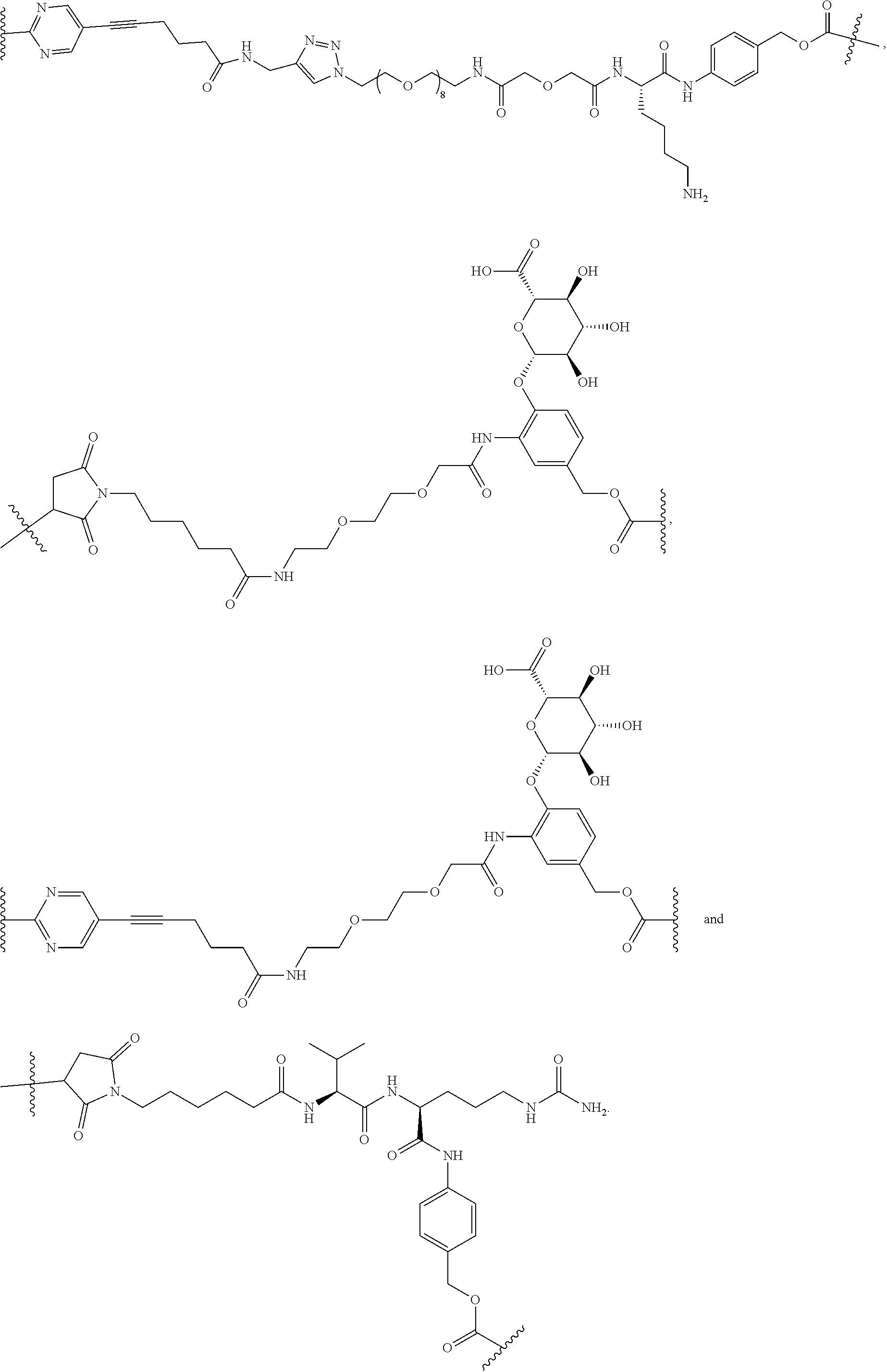

In some embodiments,

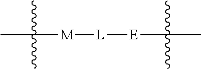

is selected from the following structures:

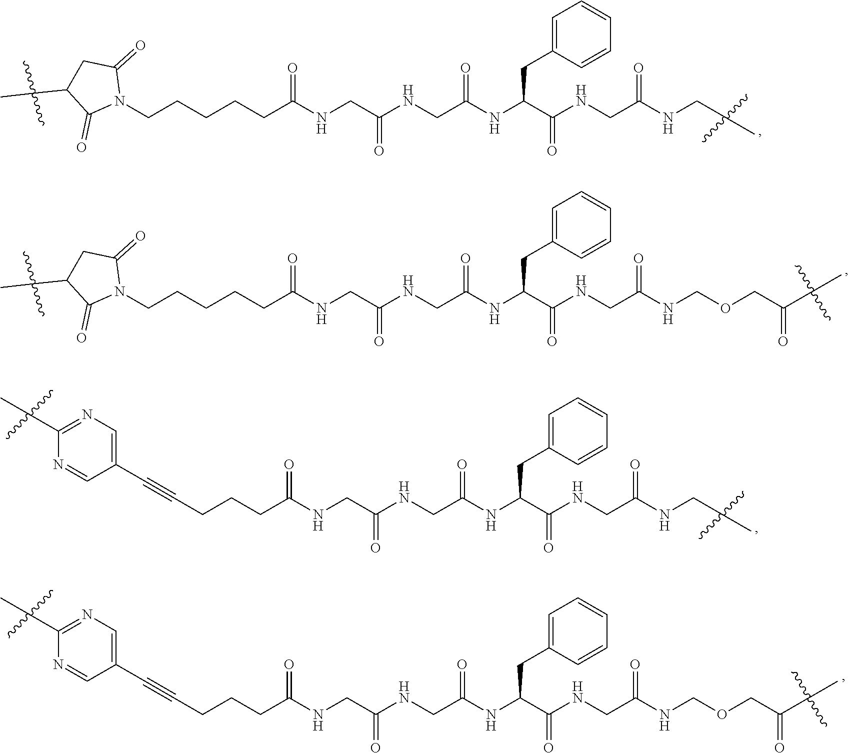 [SMILES: *CNC(=O)CNC(=O)[C@H](Cc1ccccc1)NC(=O)CNC(=O)CNC(=O)CCCCCN1C(=O)CC(*)C1=O ; *C(=O)COCNC(=O)CNC(=O)[C@H](Cc1ccccc1)NC(=O)CNC(=O)CNC(=O)CCCCCN1C(=O)CC(*)C1=O ; *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1]

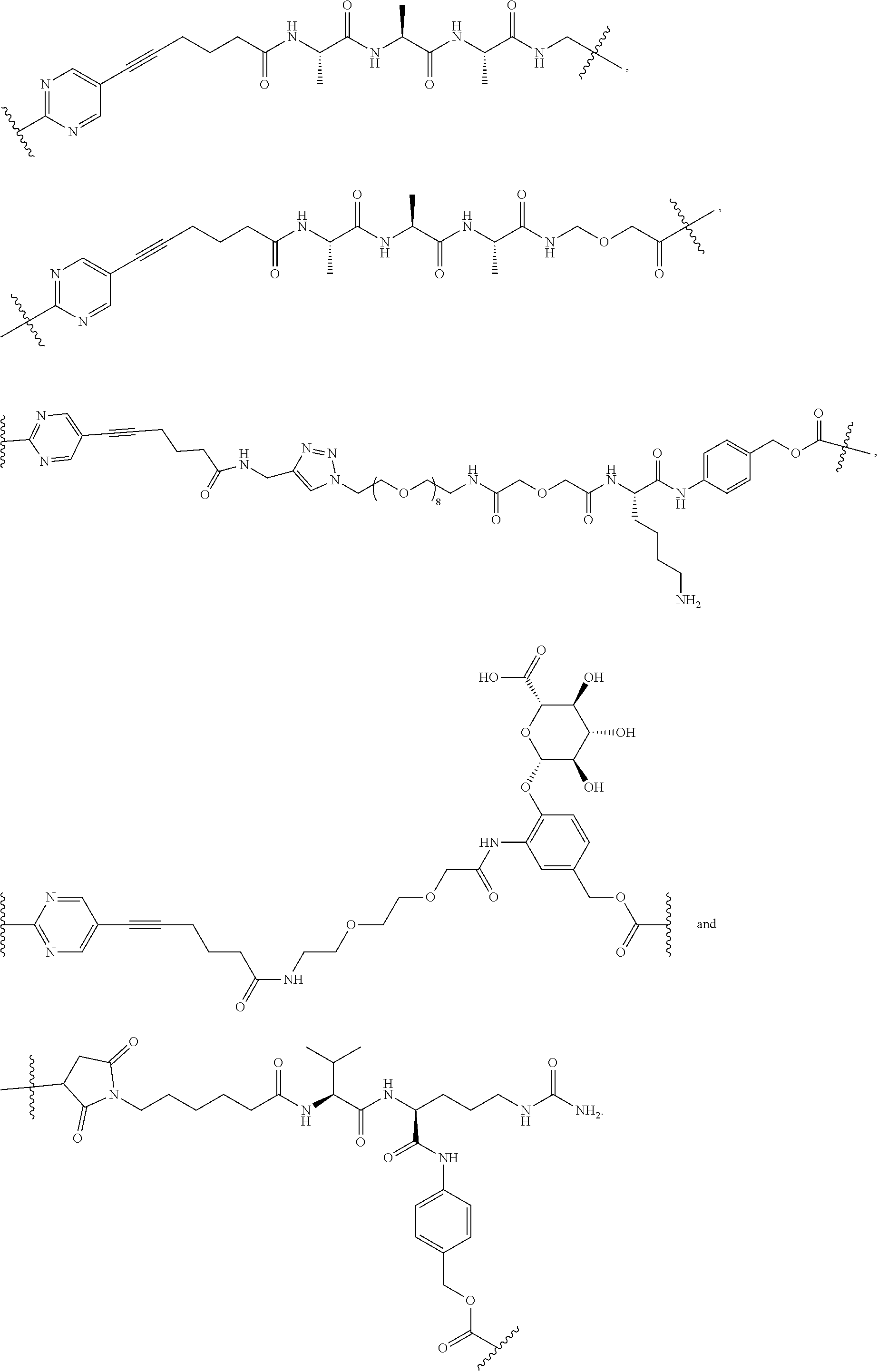

In some embodiments,

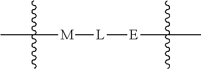

is selected from the following structures:

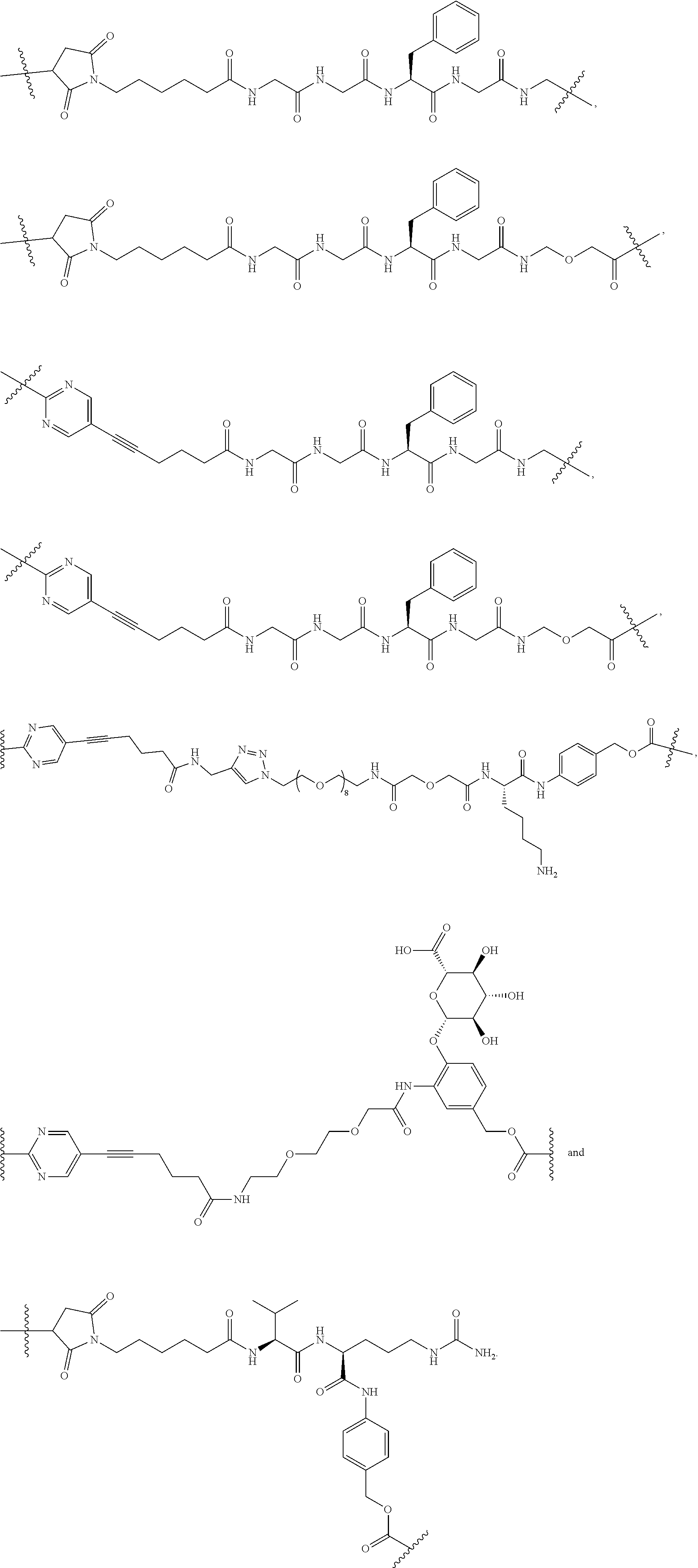

In some embodiments,

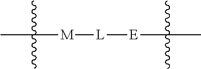

is selected from the following structures:

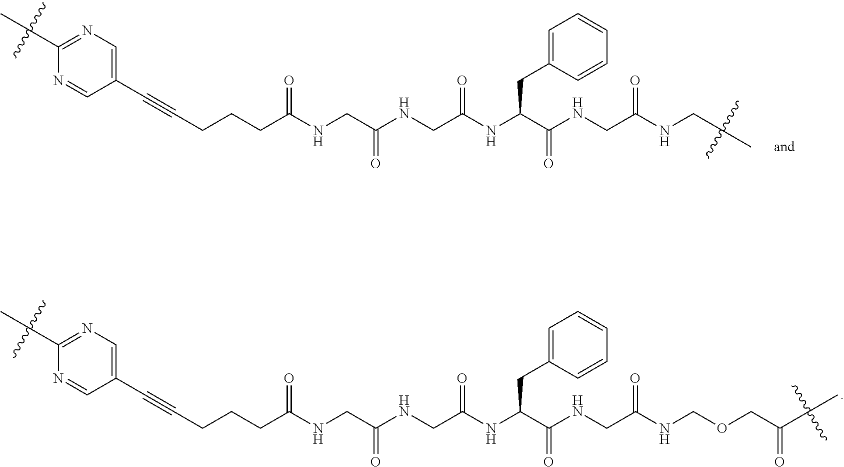 [SMILES: *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1]

In some embodiments,

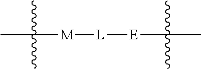

is selected from the following structures:

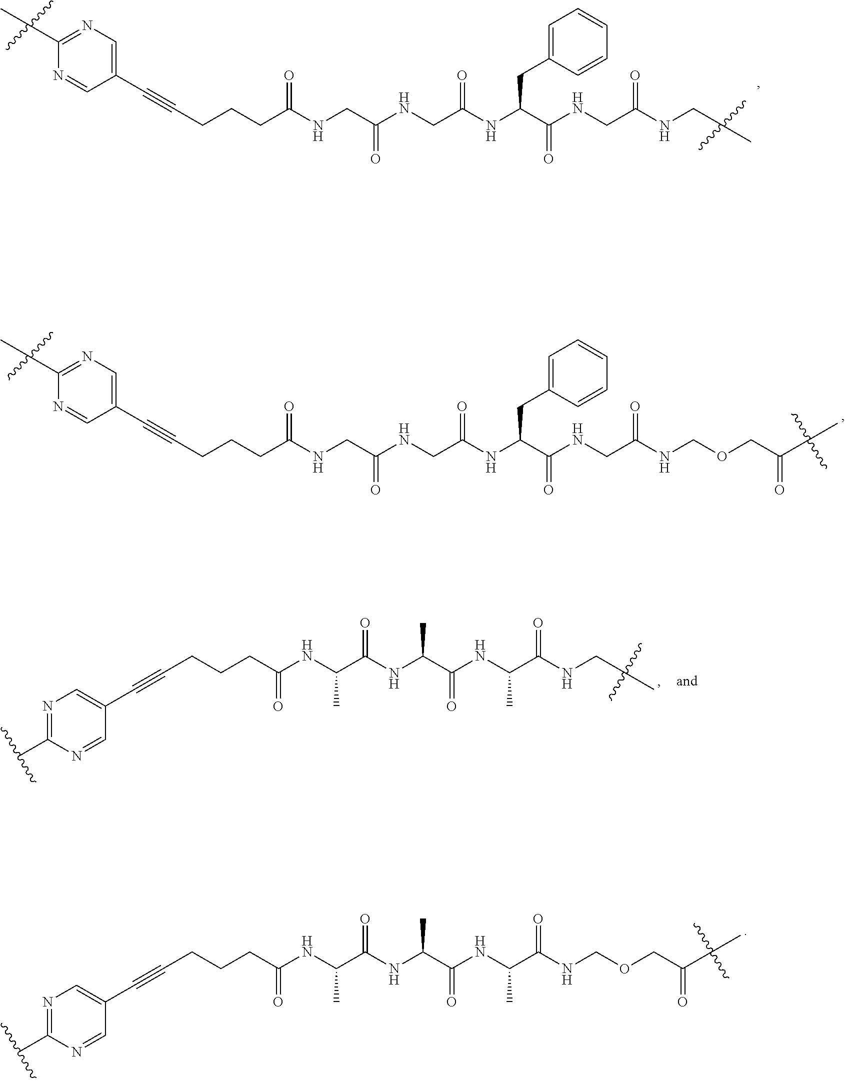 [SMILES: *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

In some embodiments, the cytotoxic drug is selected from a tubulin inhibitor, a DNA intercalator, a DNA topoisomerase inhibitor and a RNA polymerase inhibitor. In some embodiments, the tubulin inhibitor is an auristatin compound or a maytansinoid compound. In some embodiments, the DNA intercalator is pyrrolobenzodiazepine (PBD). In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is a-amanitin or a pharmaceutically acceptable salt, ester or analog thereof.

The cytotoxic drug disclosed in the present application usually contains a variety of functional groups, for example hydroxyl(—OH), carboxyl(—COOH), sulfhydryl(—SH), primary amine (—NH₂), secondary amine (—NR_{A}H) or a tertiary amine group (—NR_{B}R_{C}), and R_{A}, R_{B} and R_{C} herein only represent non-hydrogen substitutes on N; and the cytotoxic drug can be linked to the linker in the conjugate through these functional groups.

In some embodiments, the cytotoxic drug (D) is linked to E in the antibody-drug conjugate through —OH, —SH, primary amine group, secondary amine group or tertiary amine group thereon.

In some embodiments, the cytotoxic drug is selected from the following formulas I and II:

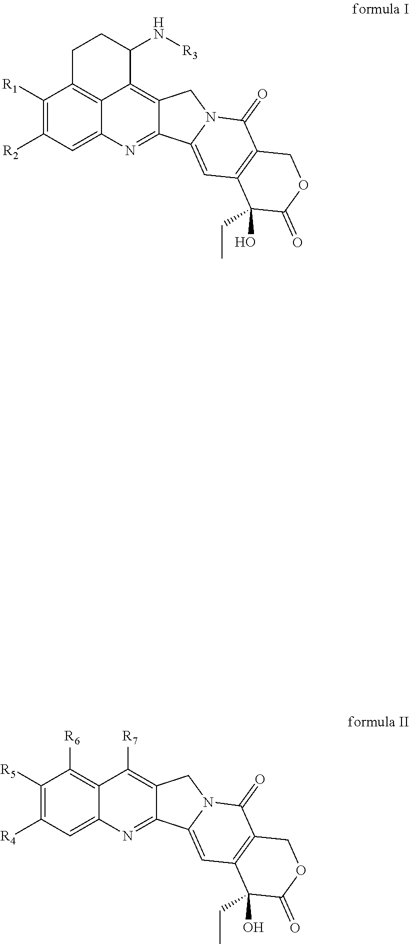

wherein R₁ and R₂ are each independently selected from C₁₋₆ alkyl and halogen;
R₃ is selected from H and -CO-CH₂OH;
R₄ and R₅ are each independently selected from H, halogen and hydroxyl; or R₄ and μs are linked to associated carbon atoms to form 5-6 membered oxygen- containing heterocyclic ring;
R₆ is selected from hydrogen and -C₁₋₄ alkylene-NRₐR_{b};
R₇ is selected from hydrogen, C₁₋₆ alkyl and -C₁₋₄ alkylene-NRₐR_{b};
wherein Rₐ and R_{b} are each independently selected from H, C₁₋₆ alkyl, -SO₂-C₁₋₆ alkyl and -CO-C₁₋₆ alkyl at each occurrence.

In some embodiments, the cytotoxic drug is selected from the following formulas I and II:

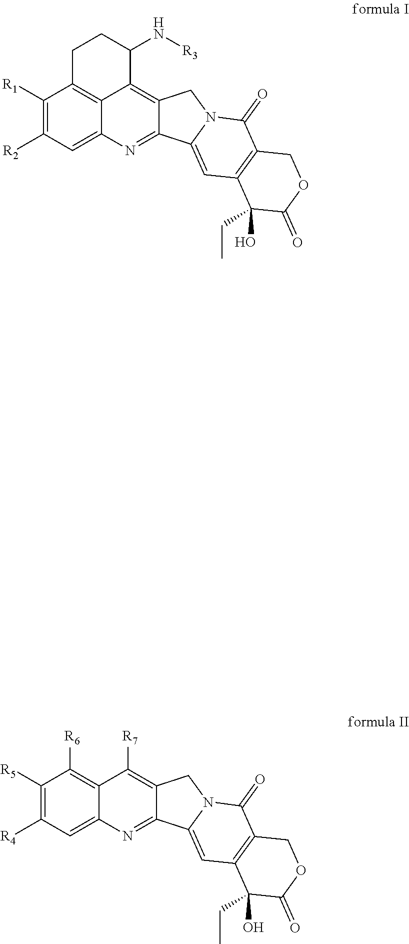

wherein R₁ and R₂ are each independently selected from C₁₋₆ alkyl and halogen;
R₃ is selected from H and -CO-CH₂OH;
R₄ and R₅ are each independently selected from H, halogen and hydroxyl; or R₄ and μs are linked to associated carbon atoms to form 5-6 membered oxygen- containing heterocyclic ring;
R₆ is selected from hydrogen and -C₁₋₄ alkylene-NRₐR_{b};
R₇ is selected from C₁₋₆ alkyl and -C₁₋₄ alkylene-NRₐR_{b};
wherein Rₐ and R_{b} are each independently selected from H, C₁₋₆ alkyl, -SO₂-C₁₋₆ alkyl and -CO-C₁₋₆ alkyl at each occurrence.

In some embodiments, the cytotoxic drug is selected from the following compounds:

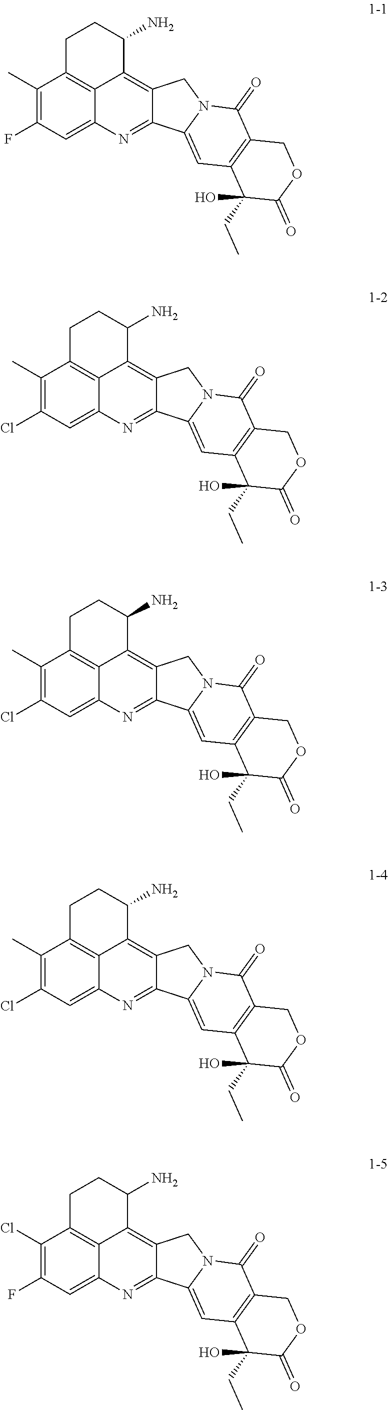 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

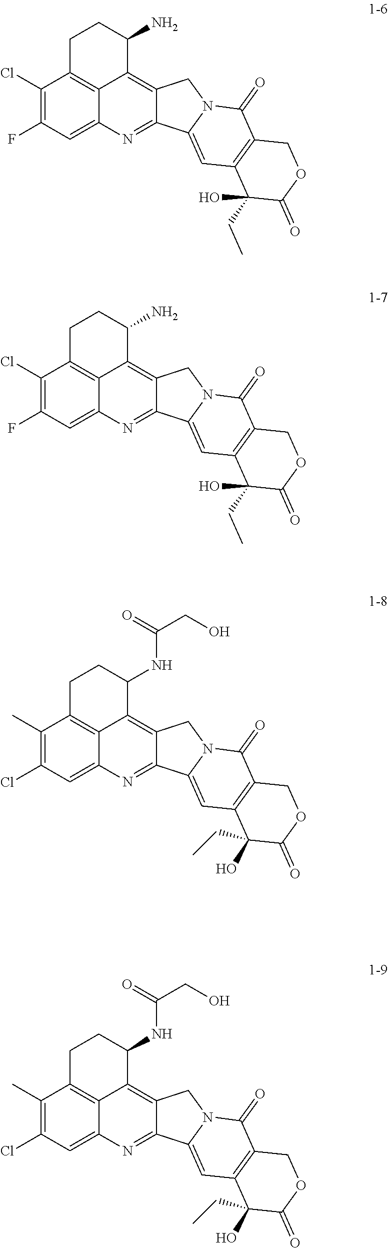 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3]

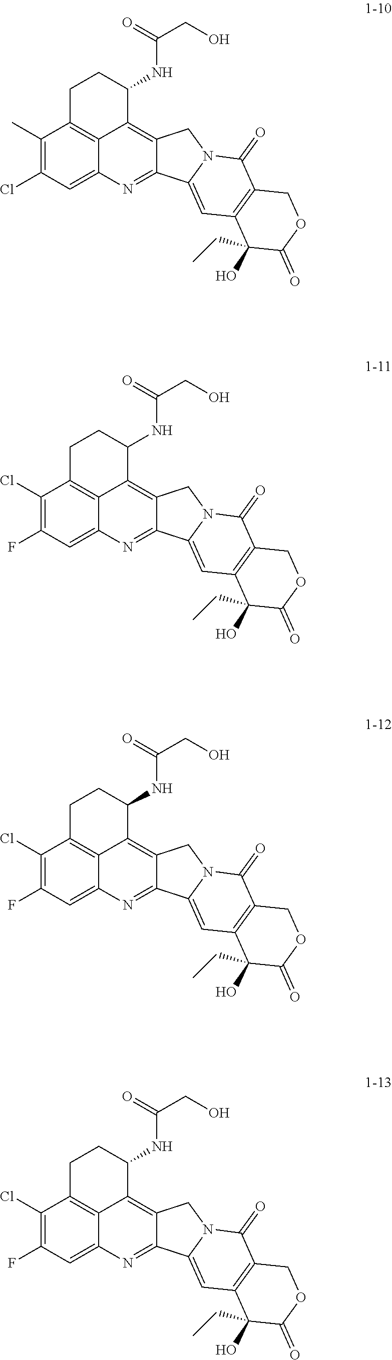 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

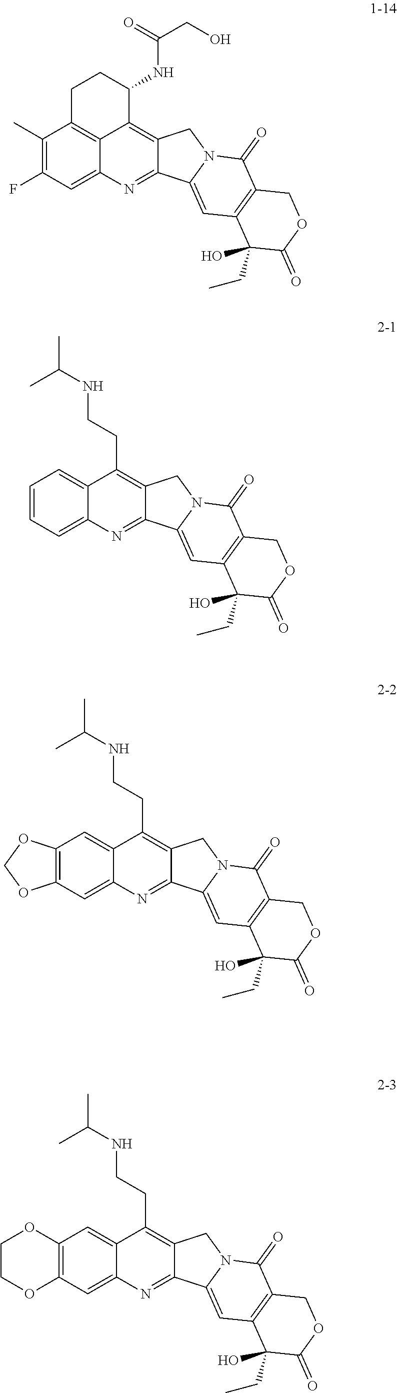 [SMILES: CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3]

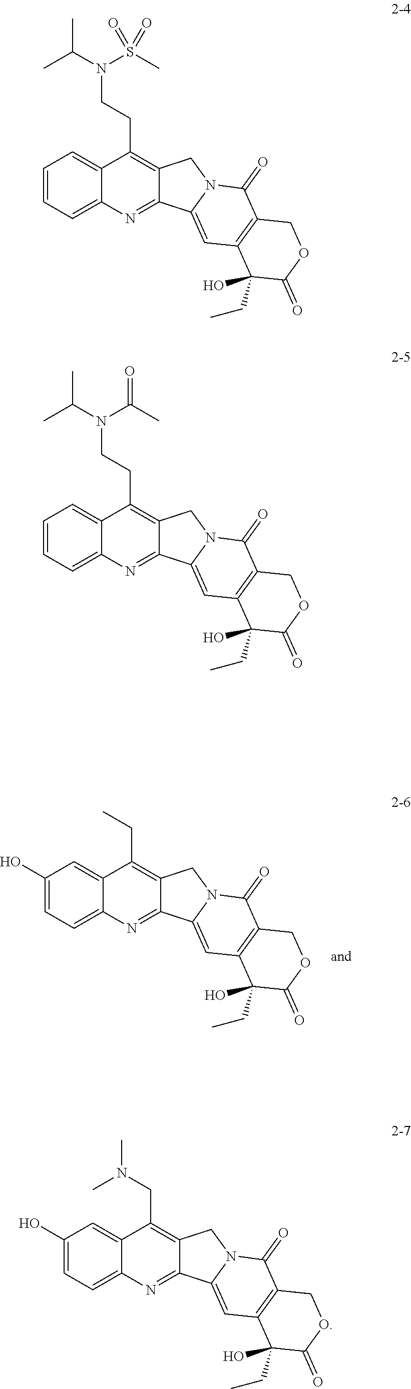 [SMILES: CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

In some embodiments, the cytotoxic drug is selected from the following compounds:

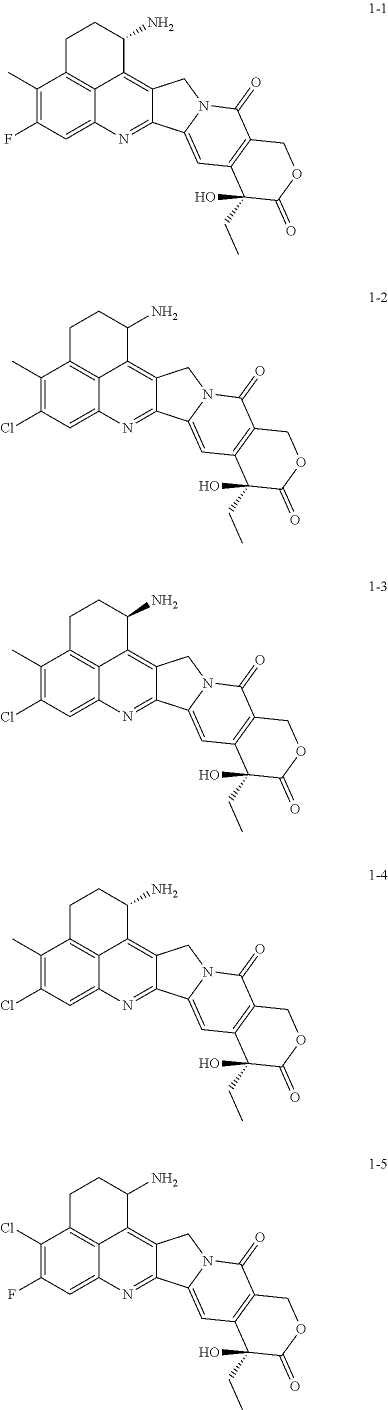 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

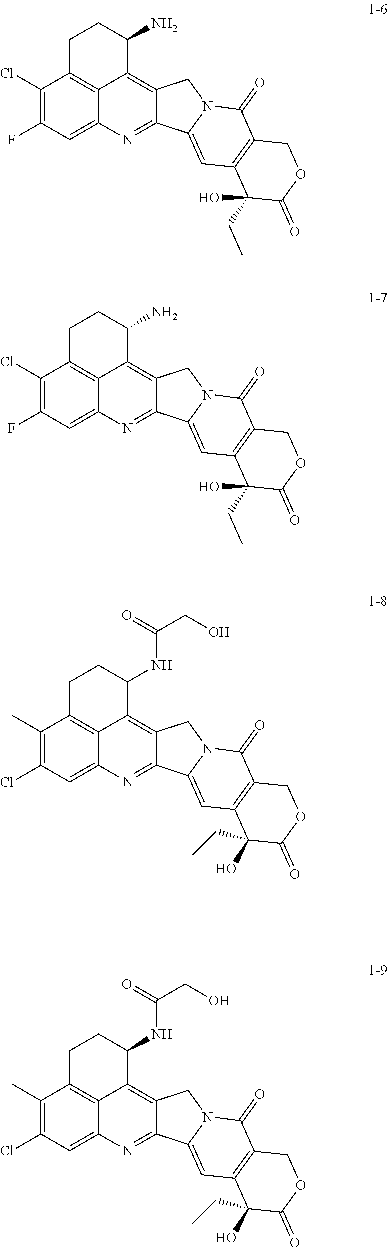 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3]

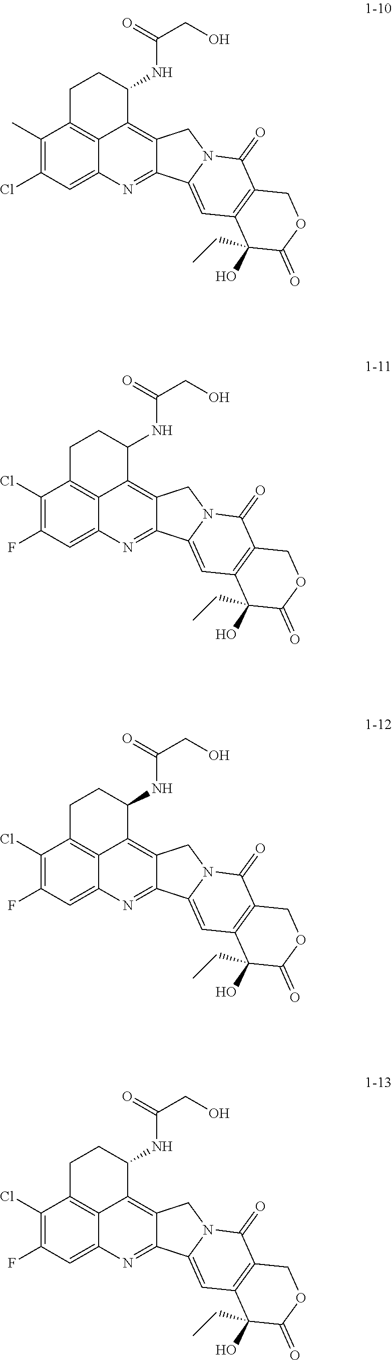 [SMILES: CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3]

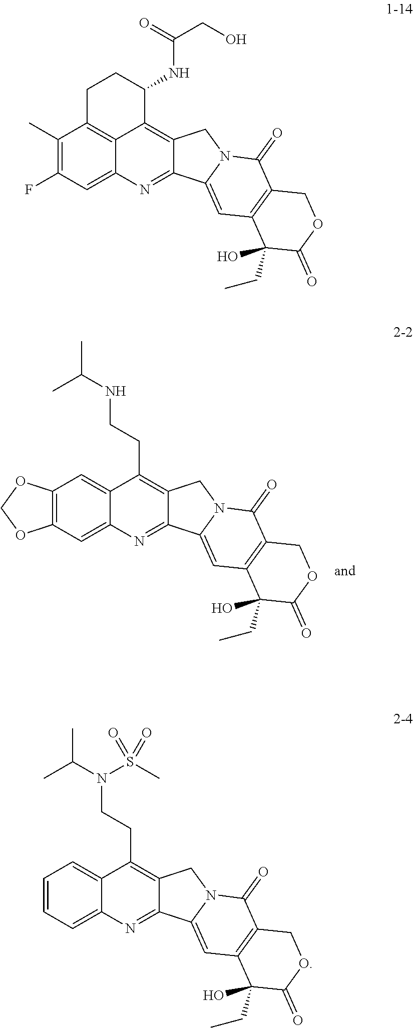 [SMILES: CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1ccccc1c2CCN(C(C)C)S(C)(=O)=O]

In some embodiments, D is selected from the following structures:

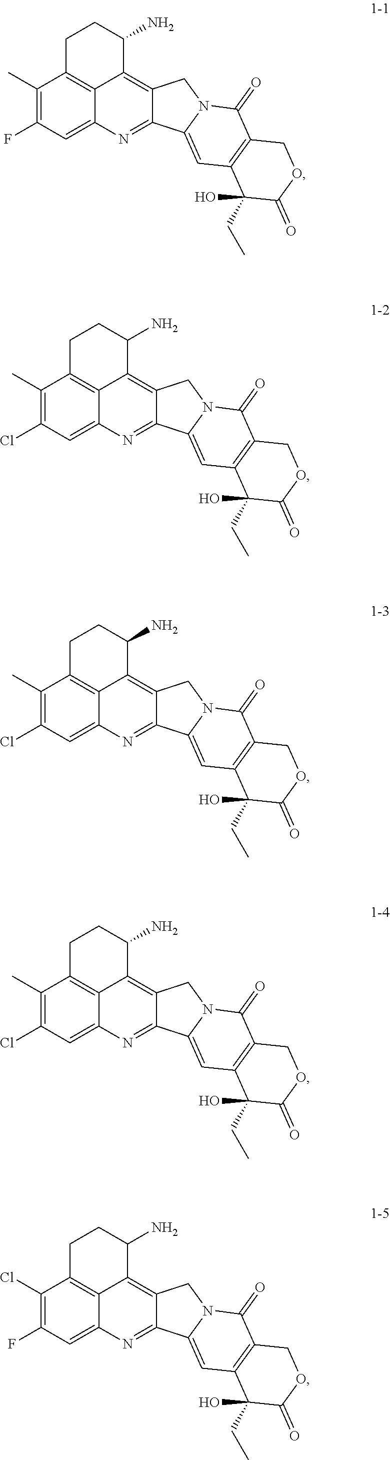 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

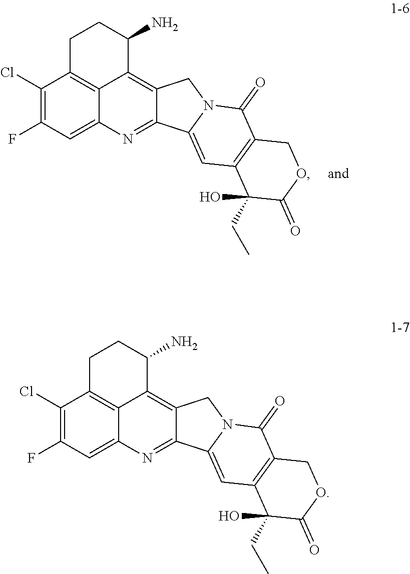 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3]

In some embodiments, the cytotoxic drug (D) is linked to E by the loss of one H from —OH, —NH₂, or secondary amine groups on the cytotoxic drug.

In some embodiments, -D is selected from the following structures:

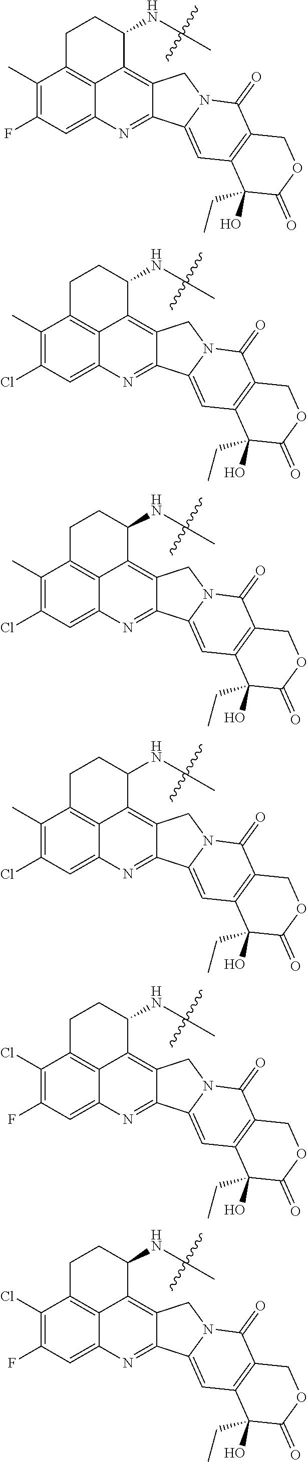 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC ; *N[C@@H]1CCc2c(Cl)c(F)cc3nc4c(c1c23)Cn1c-4cc2c(c1=O)COC(=O)[C@]2(O)CC]

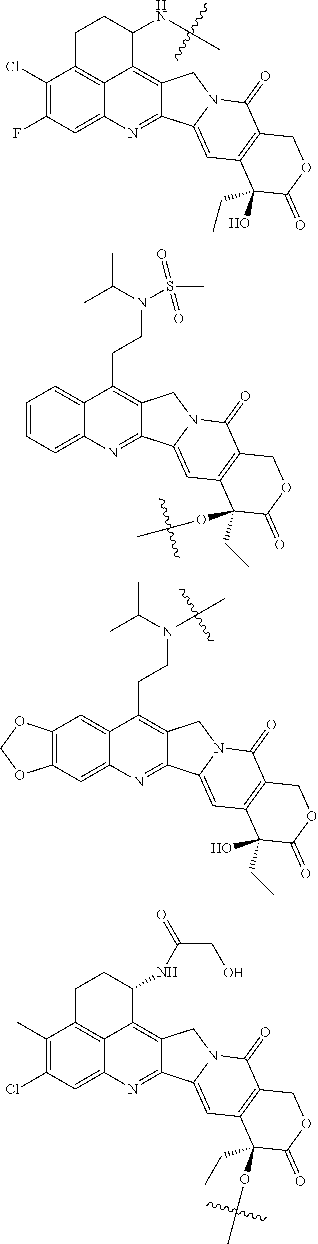 [SMILES: *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C ; *O[C@]1(CC)C(=O)OCc2c1cc1n(c2=O)Cc2c-1nc1cc(Cl)c(C)c3c1c2[C@@H](NC(=O)CO)CC3]

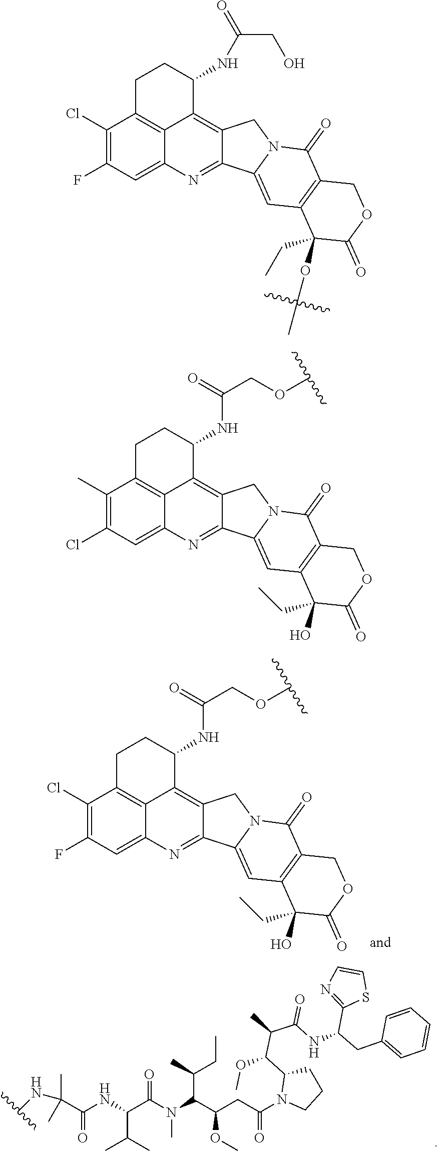

In some embodiments, the cytotoxic drug is

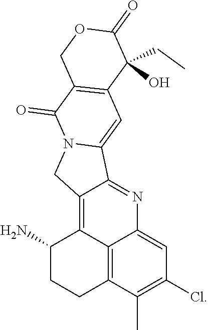 [SMILES: CC[C@@]1(O)C(=O)OCc2c1cc1n(c2=O)Cc2c-1nc1cc(Cl)c(C)c3c1c2[C@@H](N)CC3]

In some embodiments, the cytotoxic drug is

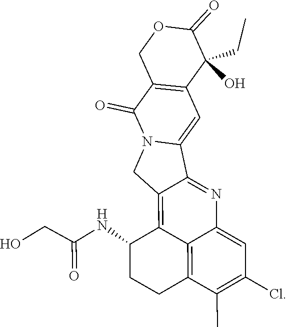 [SMILES: CC[C@@]1(O)C(=O)OCc2c1cc1n(c2=O)Cc2c-1nc1cc(Cl)c(C)c3c1c2[C@@H](NC(=O)CO)CC3]

In some embodiments, (—M-L-E-D) is selected from

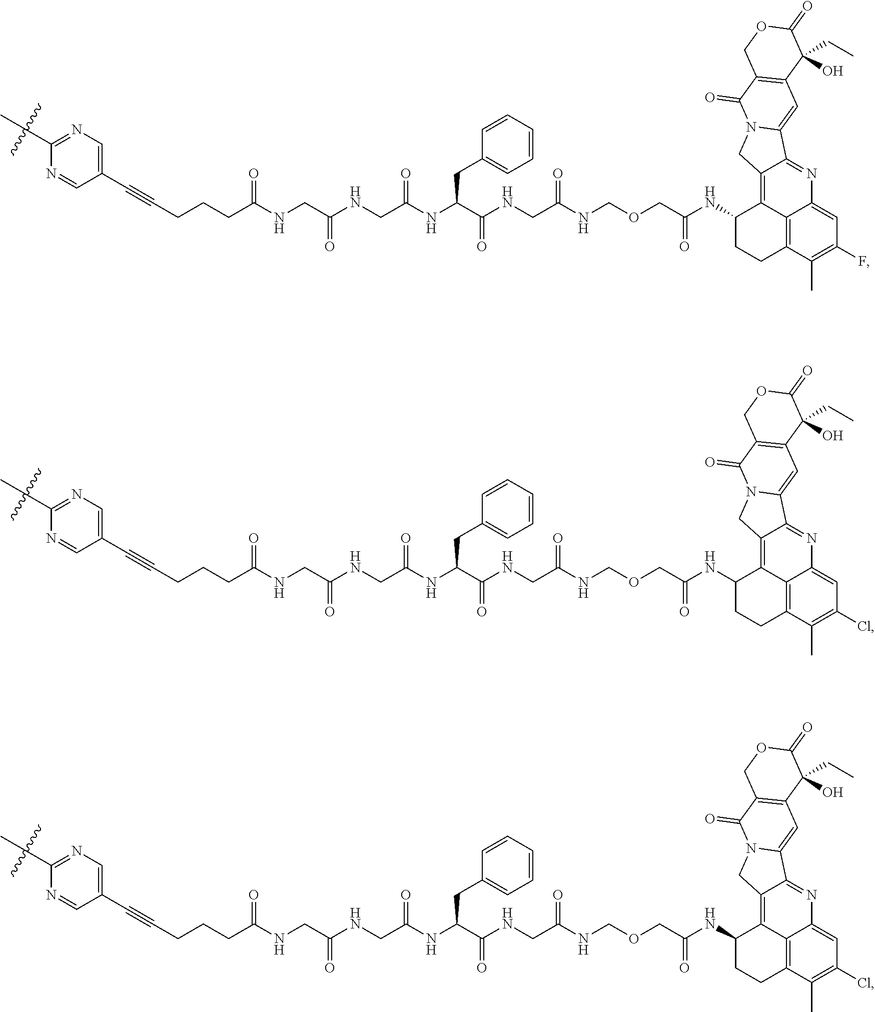 [SMILES: *c1ncc(C#CCCCC(=O)NCC(=O)NCC(=O)N[C@@H](Cc2ccccc2)C(=O)NCC(=O)NCOCC(=O)N[C@H]2CCc3c(C)c(F)cc4nc5c(c2c34)Cn2c-5cc3c(c2=O)COC(=O)[C@]3(O)CC)cn1 ; *c1ncc(C#CCCCC(=O)NCC(=O)NCC(=O)N[C@@H](Cc2ccccc2)C(=O)NCC(=O)NCOCC(=O)NC2CCc3c(C)c(Cl)cc4nc5c(c2c34)Cn2c-5cc3c(c2=O)COC(=O)[C@]3(O)CC)cn1 ; *c1ncc(C#CCCCC(=O)NCC(=O)NCC(=O)N[C@@H](Cc2ccccc2)C(=O)NCC(=O)NCOCC(=O)N[C@@H]2CCc3c(C)c(Cl)cc4nc5c(c2c34)Cn2c-5cc3c(c2=O)COC(=O)[C@]3(O)CC)cn1]

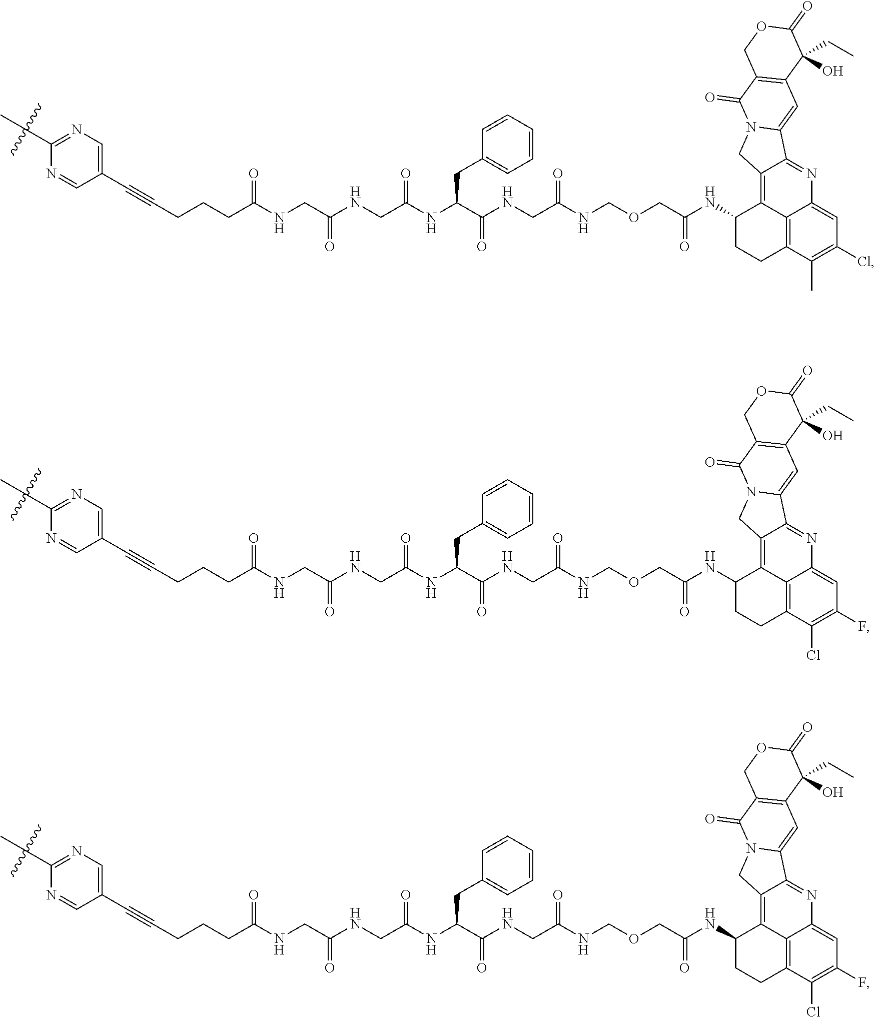 [SMILES: *c1ncc(C#CCCCC(=O)NCC(=O)NCC(=O)N[C@@H](Cc2ccccc2)C(=O)NCC(=O)NCOCC(=O)N[C@H]2CCc3c(C)c(Cl)cc4nc5c(c2c34)Cn2c-5cc3c(c2=O)COC(=O)[C@]3(O)CC)cn1 ; *c1ncc(C#CCCCC(=O)NCC(=O)NCC(=O)N[C@@H](Cc2ccccc2)C(=O)NCC(=O)NCOCC(=O)NC2CCc3c(Cl)c(F)cc4nc5c(c2c34)Cn2c-5cc3c(c2=O)COC(=O)[C@]3(O)CC)cn1 ; *c1ncc(C#CCCCC(=O)NCC(=O)NCC(=O)N[C@@H](Cc2ccccc2)C(=O)NCC(=O)NCOCC(=O)N[C@@H]2CCc3c(Cl)c(F)cc4nc5c(c2c34)Cn2c-5cc3c(c2=O)COC(=O)[C@]3(O)CC)cn1]

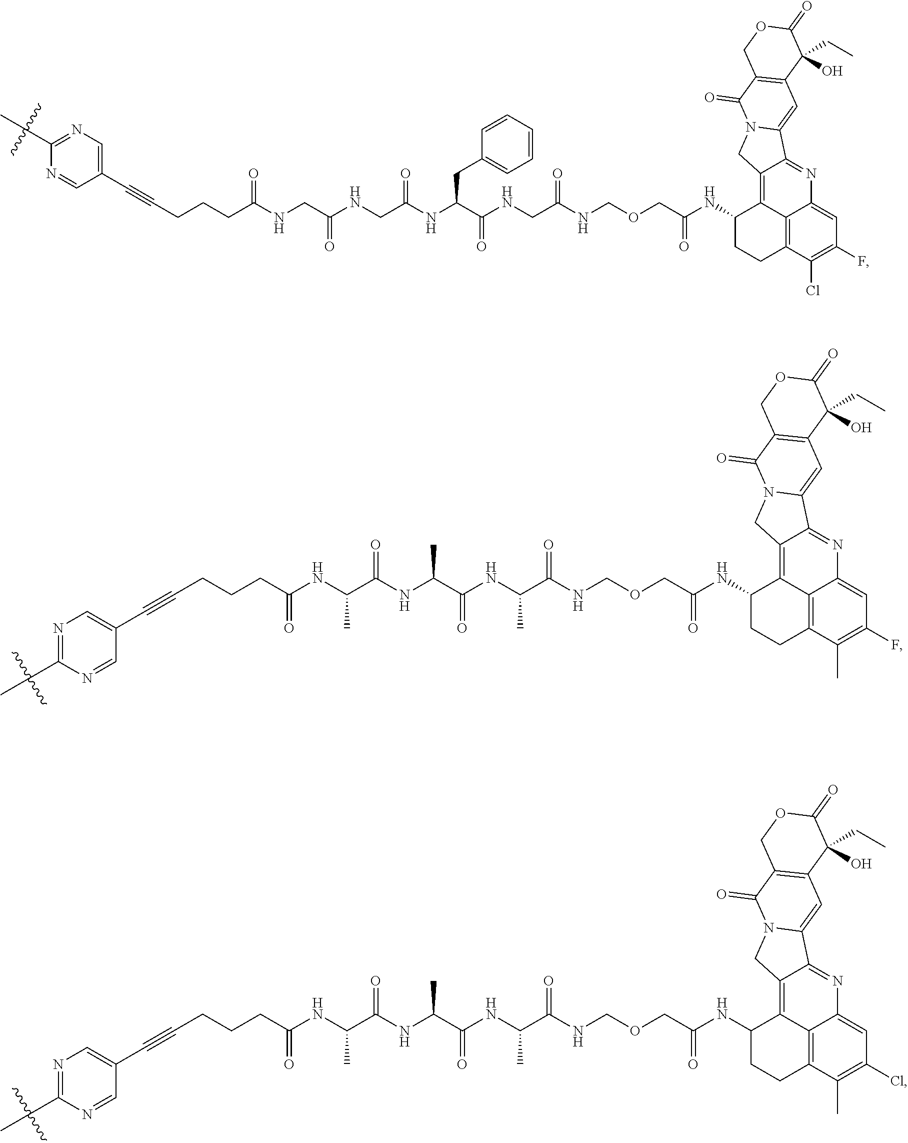 [SMILES: *c1ncc(C#CCCCC(=O)NCC(=O)NCC(=O)N[C@@H](Cc2ccccc2)C(=O)NCC(=O)NCOCC(=O)N[C@H]2CCc3c(Cl)c(F)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)N[C@H]2CCc3c(C)c(F)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)NC2CCc3c(C)c(Cl)cc4nc5c(c2c34)Cn2c-5cc3c(c2=O)COC(=O)[C@]3(O)CC)cn1]

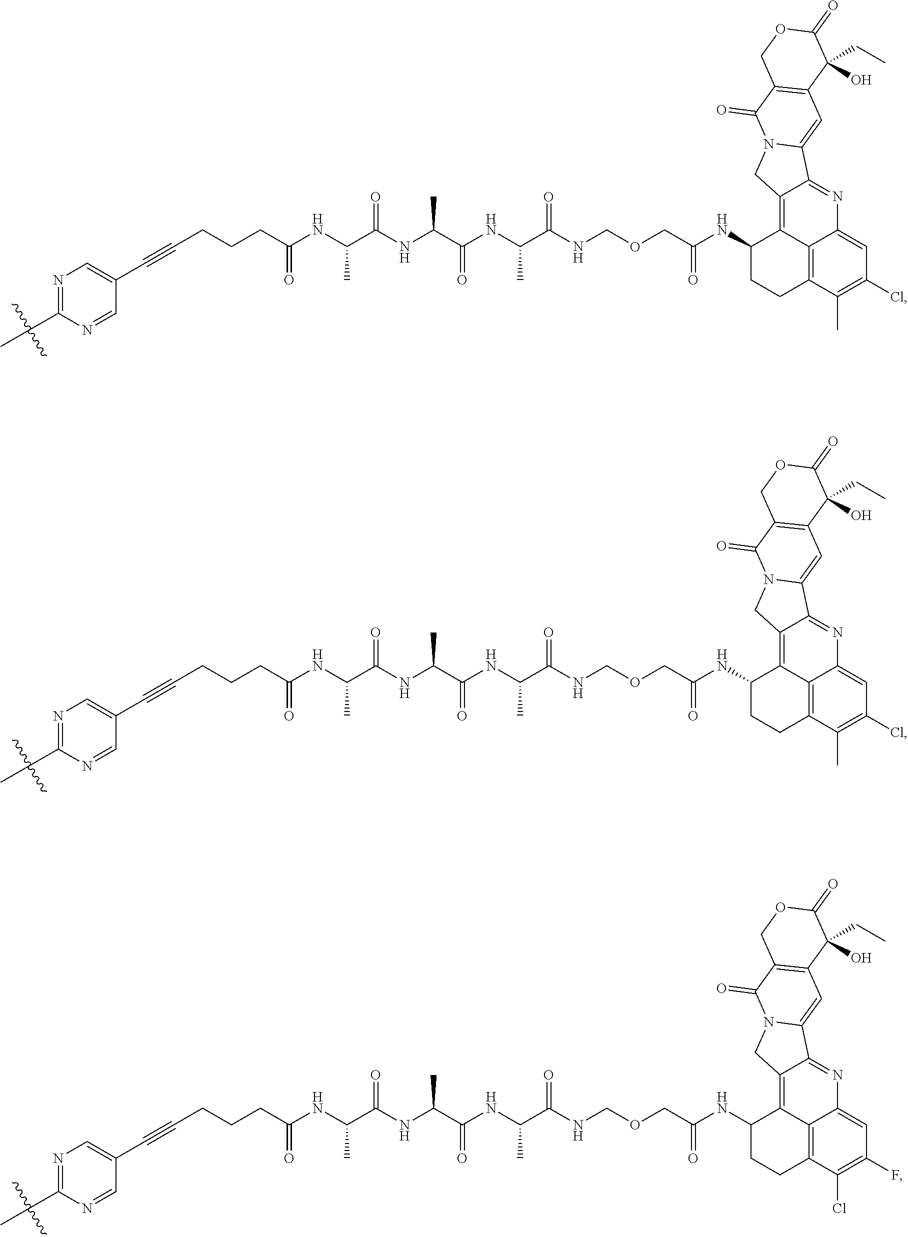 [SMILES: *c1ncc(C#CCCCC(=O)N[C@@H](C)C(=O)N[C@@H](C)C(=O)N[C@@H](C)C(=O)NCOCC(=O)N[C@@H]2CCc3c(C)c(Cl)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)N[C@H]2CCc3c(C)c(Cl)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)NC2CCc3c(Cl)c(F)cc4nc5c(c2c34)Cn2c-5cc3c(c2=O)COC(=O)[C@]3(O)CC)cn1]

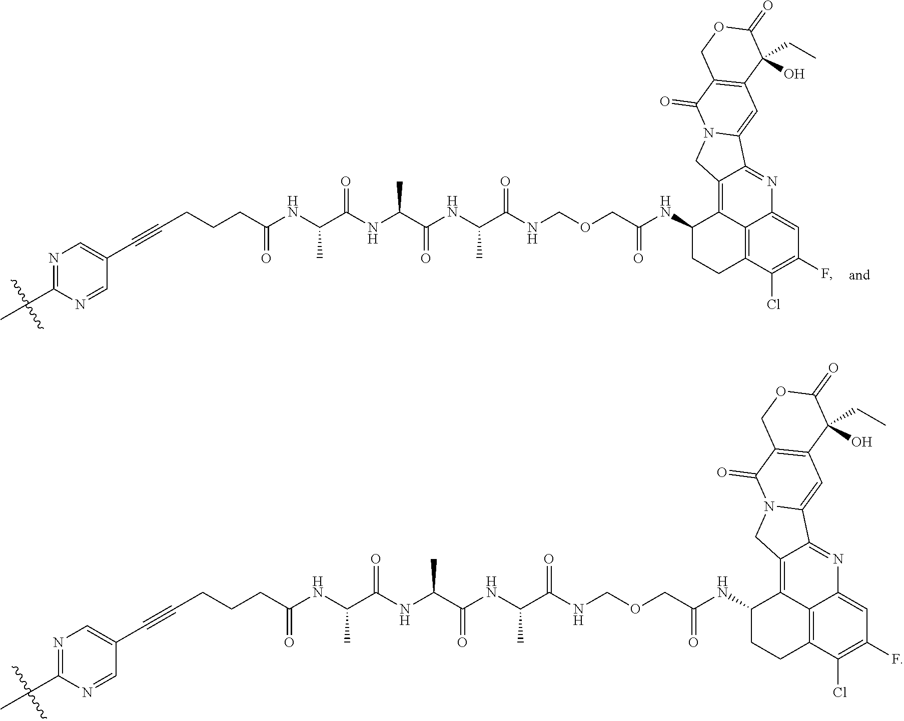 [SMILES: *c1ncc(C#CCCCC(=O)N[C@@H](C)C(=O)N[C@@H](C)C(=O)N[C@@H](C)C(=O)NCOCC(=O)N[C@@H]2CCc3c(Cl)c(F)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)N[C@H]2CCc3c(Cl)c(F)cc4nc5c(c2c34)Cn2c-5cc3c(c2=O)COC(=O)[C@]3(O)CC)cn1]

In some embodiments, the antibody-drug conjugate is selected from ADC A-01 to ADC A-04, ADC A-08, ADC A-10, ADC A-12, ADC A-13, ADC A-16, ADC A-18, ADC A-20, ADC A-22, ADC A-24, ADC B-02, and ADC C-01 shown as follows:

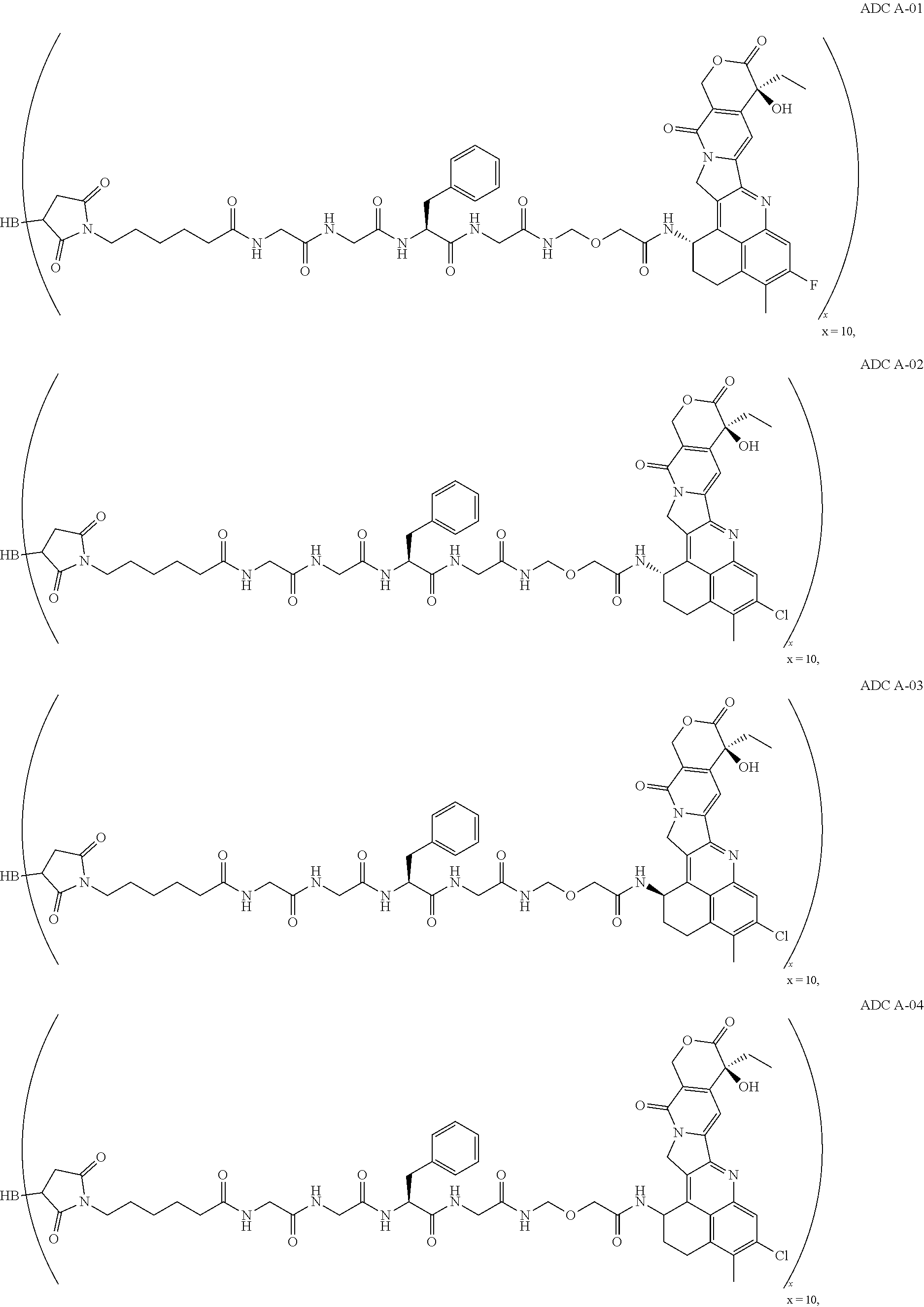

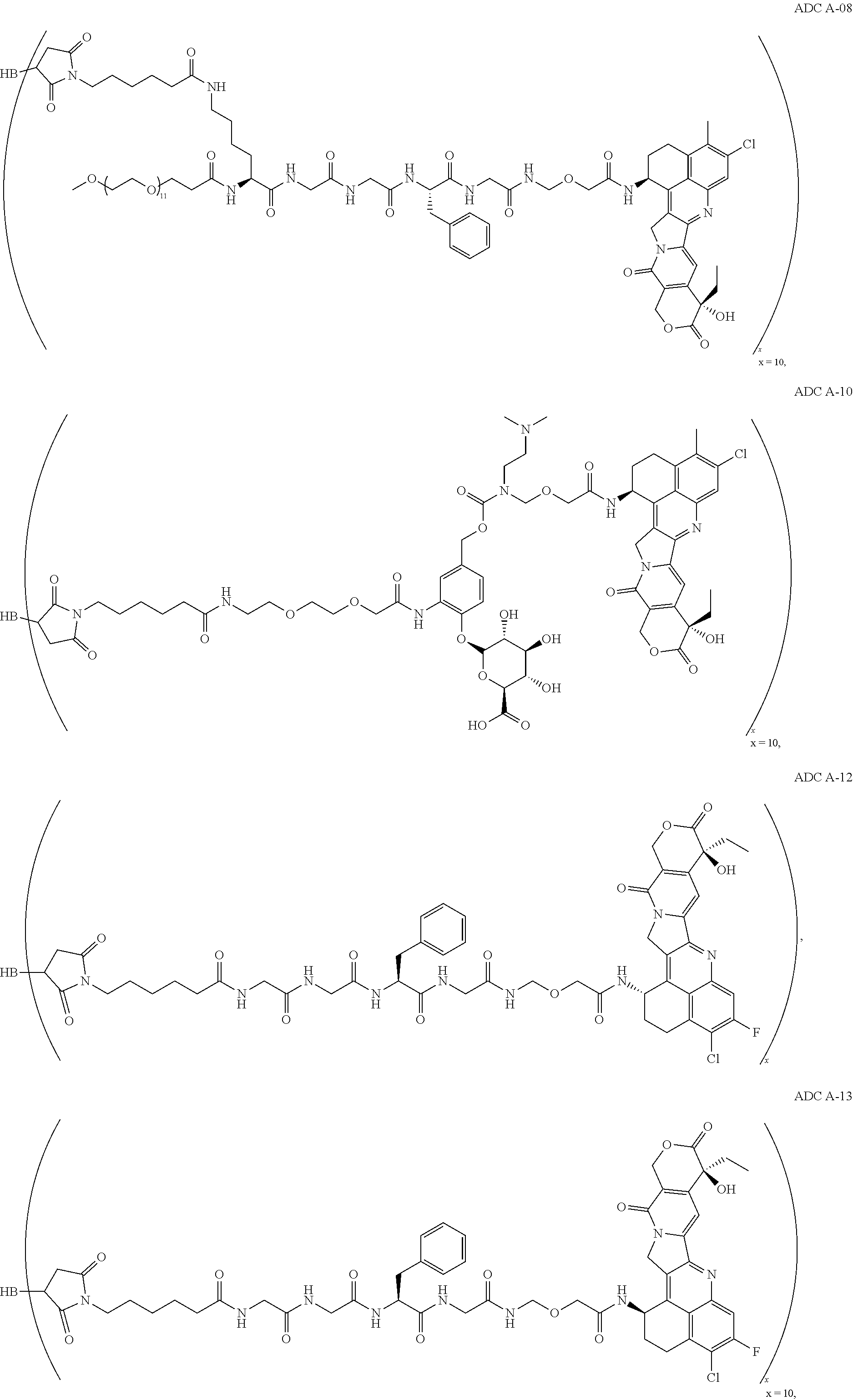

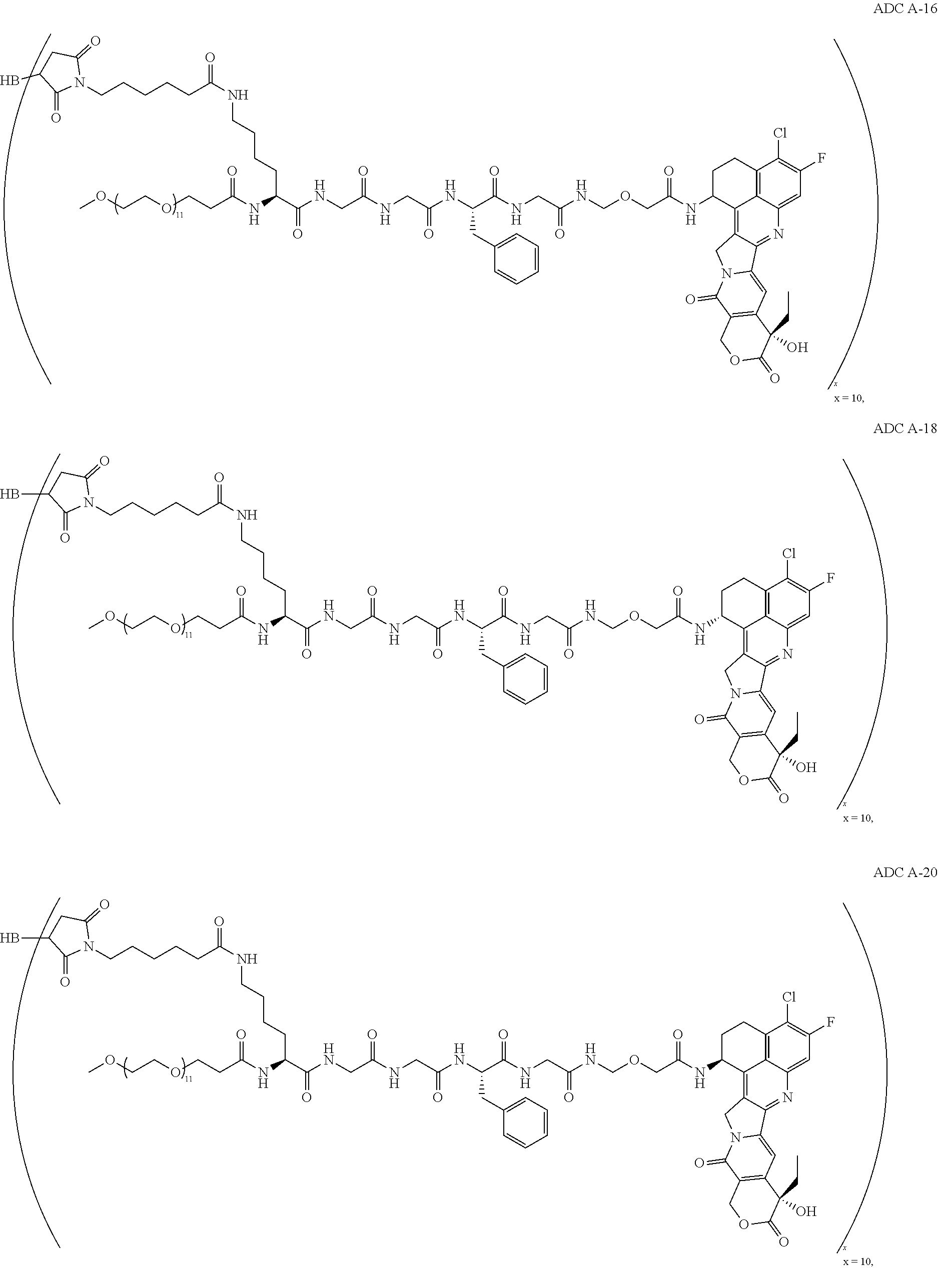

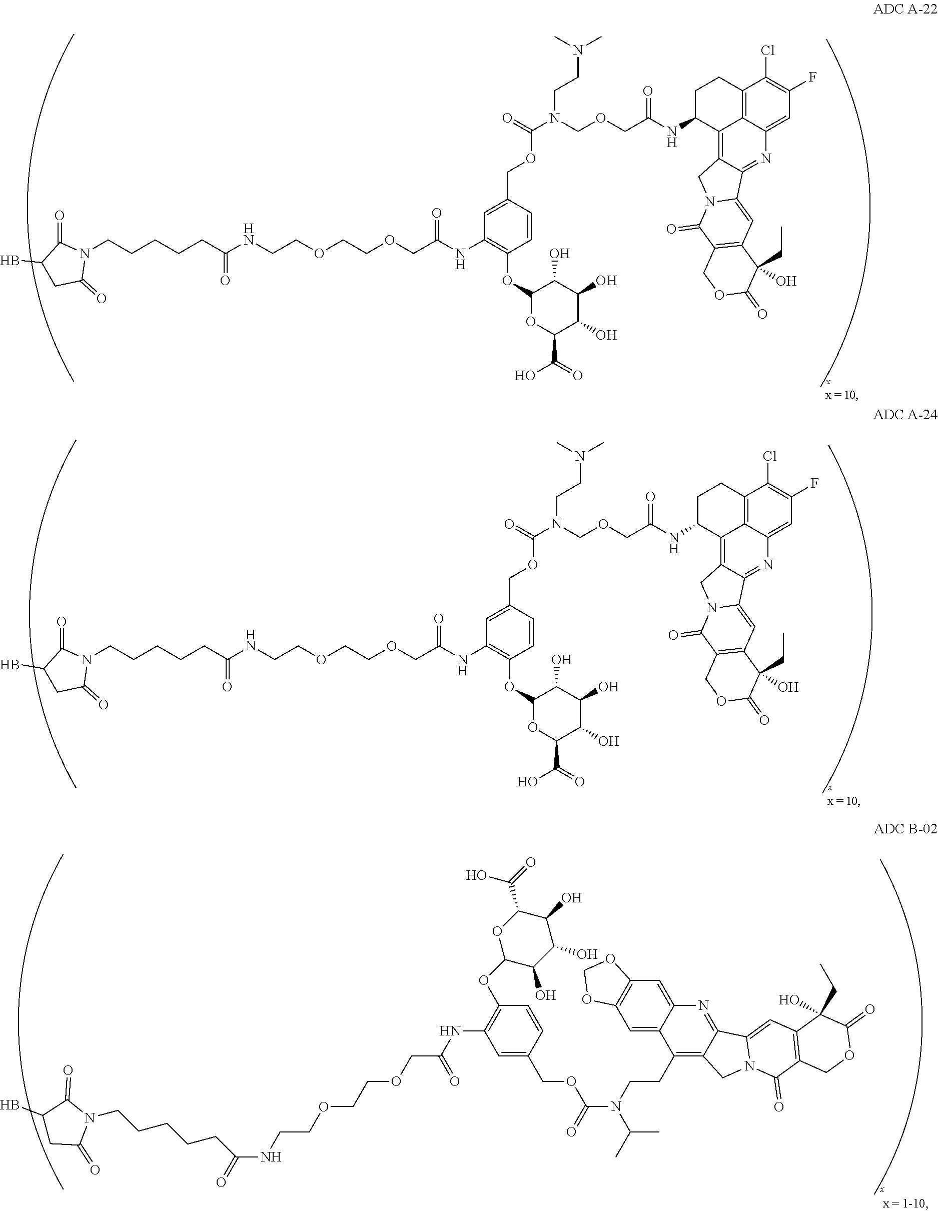

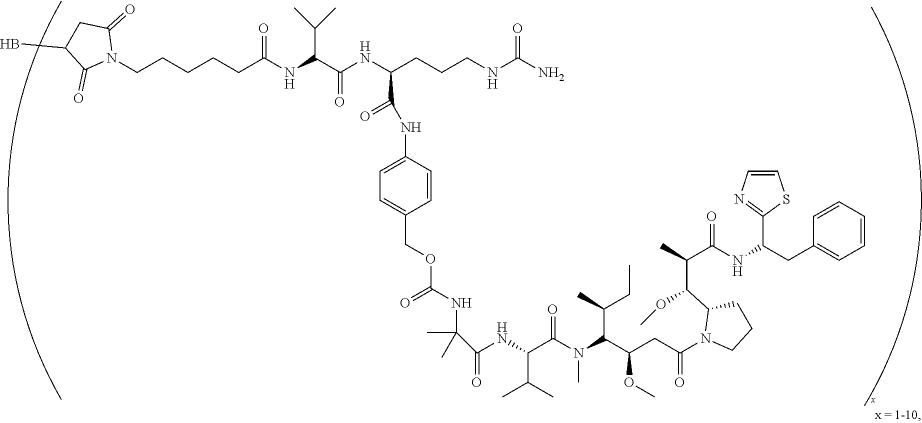

wherein HB-(- is HA-(Y-, wherein HA-(Y-represents an antibody or antigen-binding fragment thereof that specifically binds to CEACAM5, where-(Y-represents a linkage from an amino acid residue of the antibody or antigen-binding fragment thereof to each succinimidyl group in formulas ADC A-01 to ADC A-04, ADC A-08, ADC A-10, ADC A-12, ADC A-13, ADC A-16, ADC A-18, ADC A-20, ADC A-22, ADC A-24, ADC B-02 and ADC C-01.

In some embodiments, HA-(Y— comprises a heavy chain having the amino acid sequence as set forth in SEQ ID NO: 20 and a light chain having the amino acid sequence as set forth in SEQ ID NO: 21.

In some embodiments, HA-(Y— comprises a heavy chain having the amino acid sequence as set forth in SEQ ID NO: 22 and a light chain having the amino acid sequence as set forth in SEQ ID NO: 21.

In some embodiments, HA-(Y— comprises a heavy chain having the amino acid sequence as set forth in SEQ ID NO: 23 and a light chain having the amino acid sequence as set forth in SEQ ID NO: 21.

In some embodiments, HA-(Y— comprises a heavy chain having the amino acid sequence as set forth in SEQ ID NO: 24 and a light chain having the amino acid sequence as set forth in SEQ ID NO: 21.

In some embodiments, -(Y— is -(S-.

In some embodiments, the antibody-drug conjugate is selected from ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06 shown as follows:

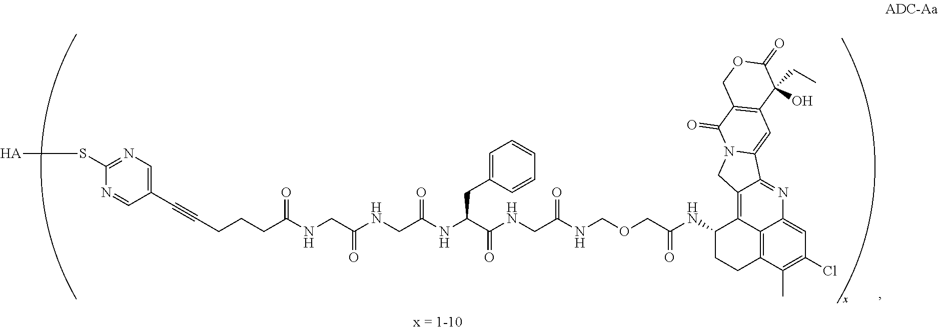

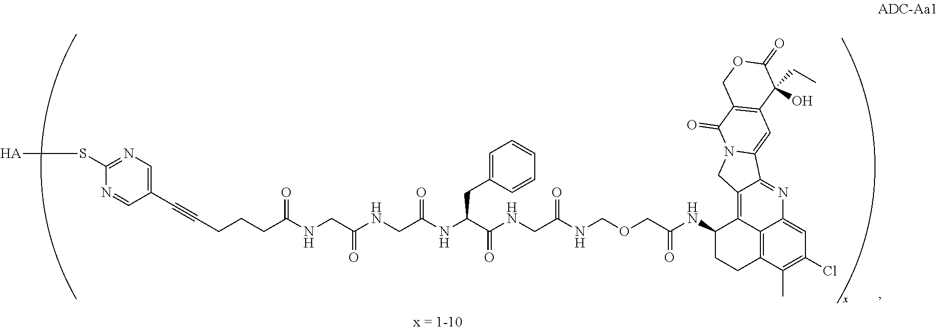

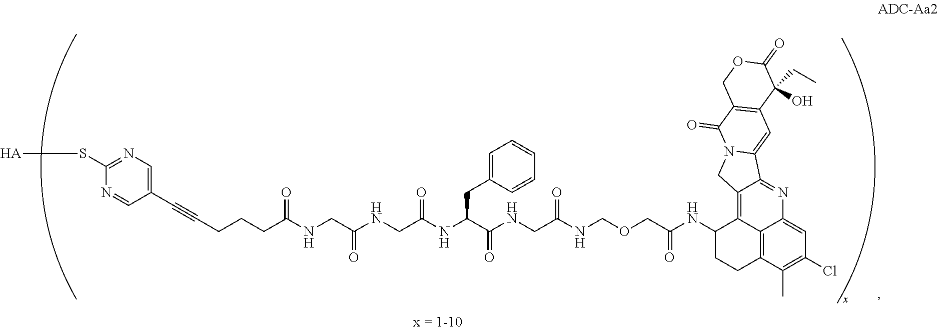

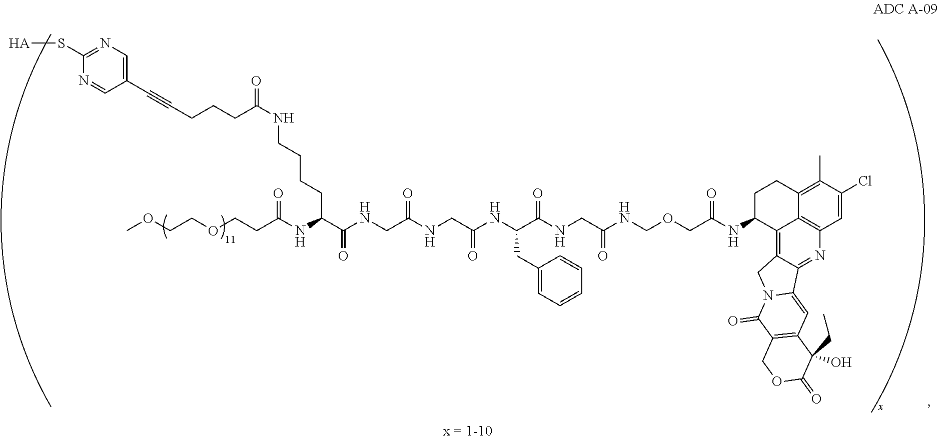

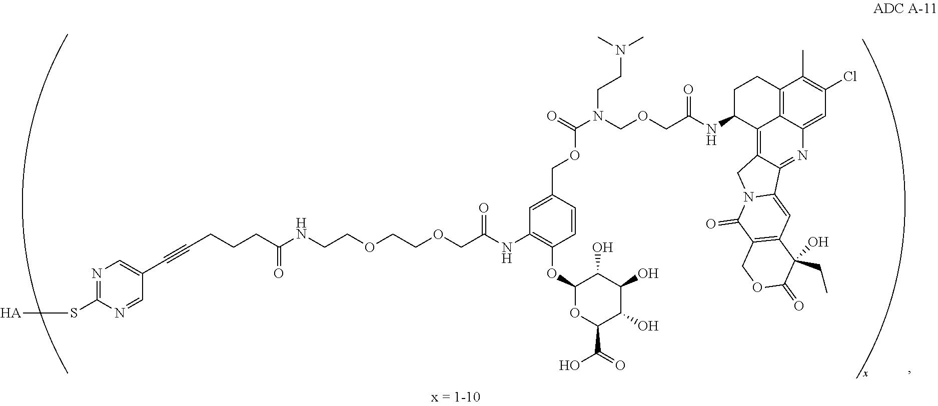

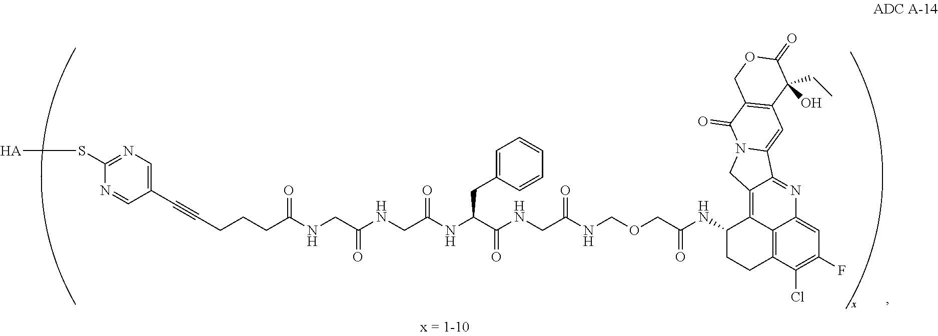

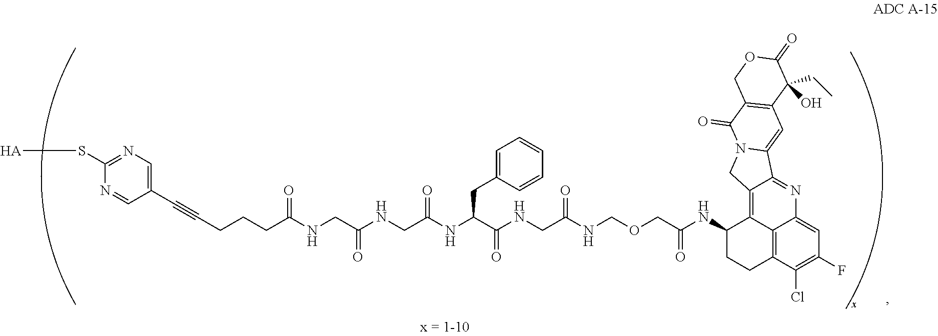

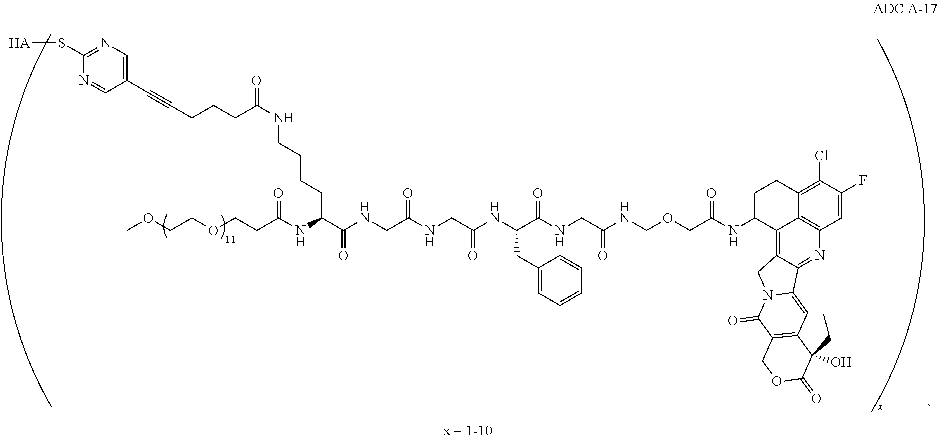

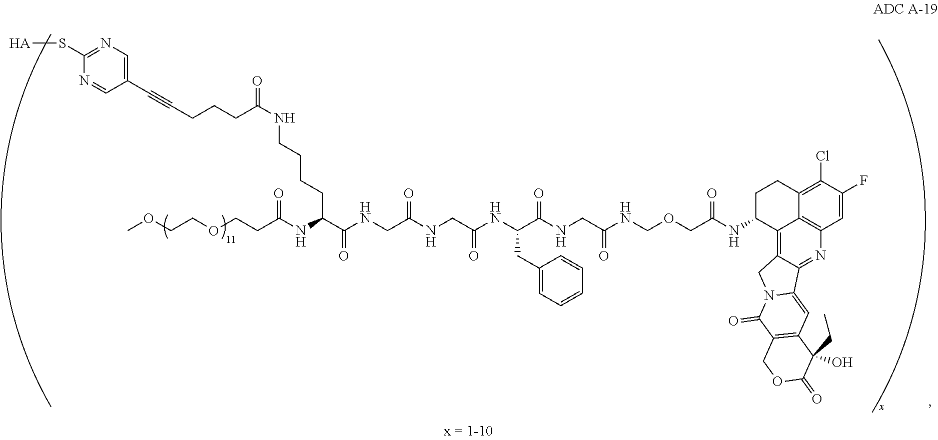

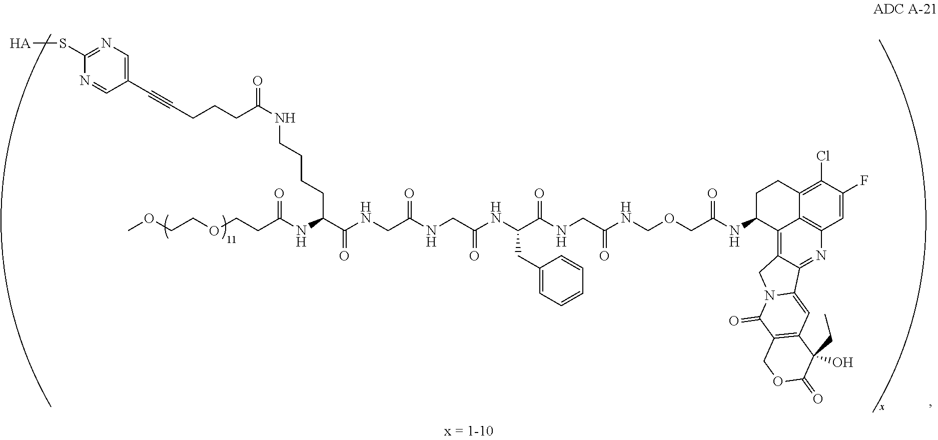

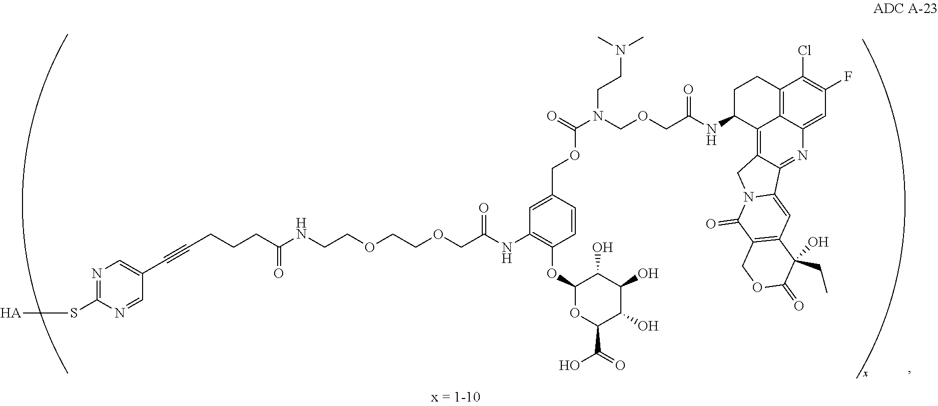

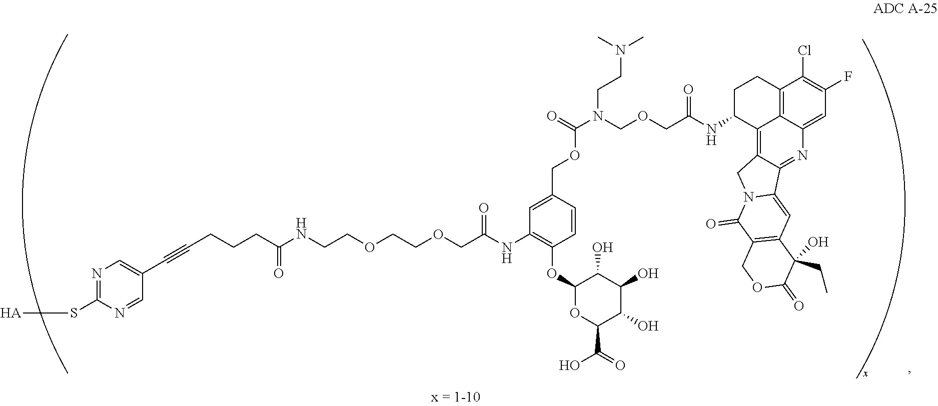

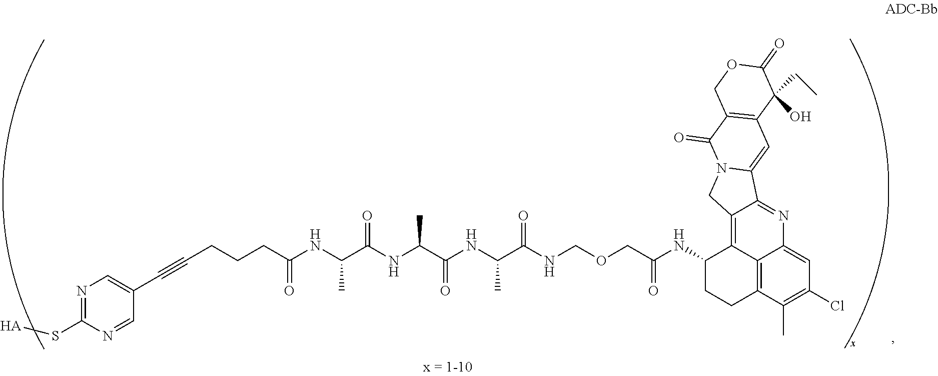

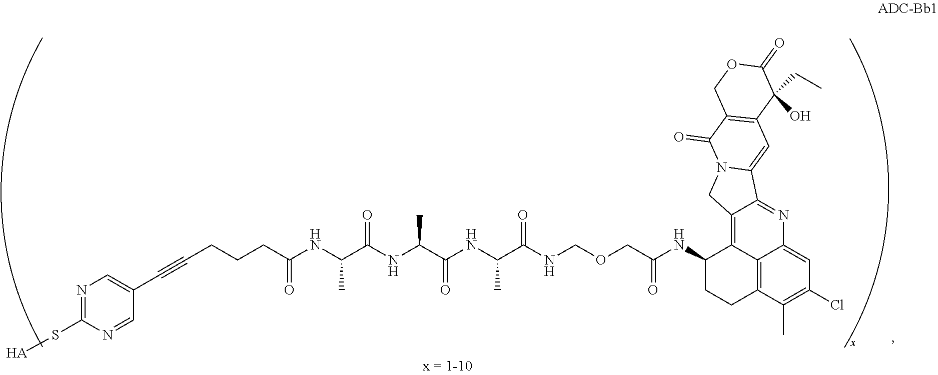

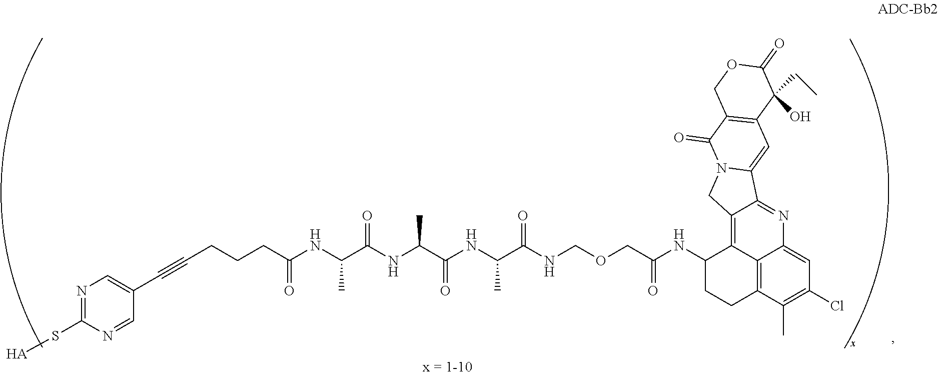

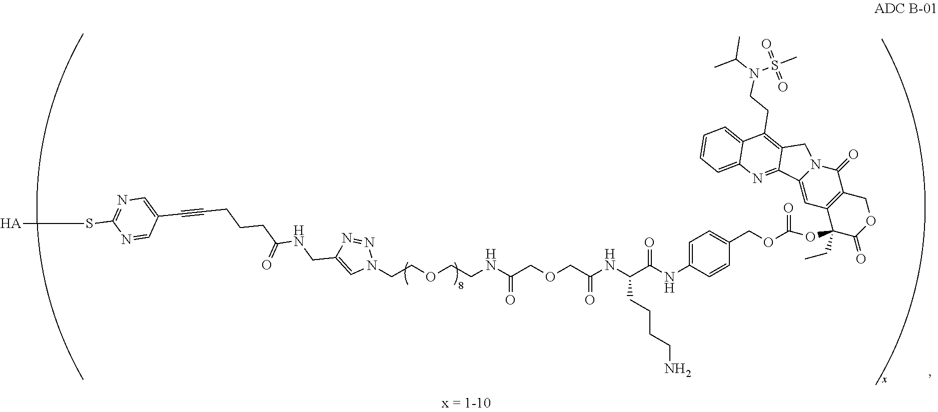

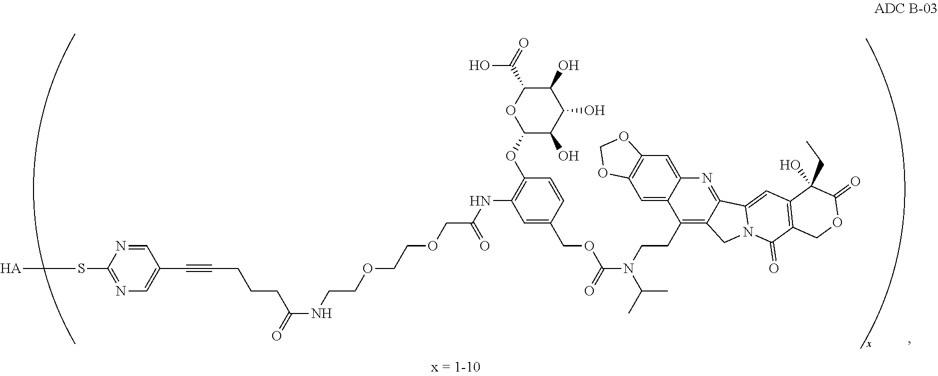

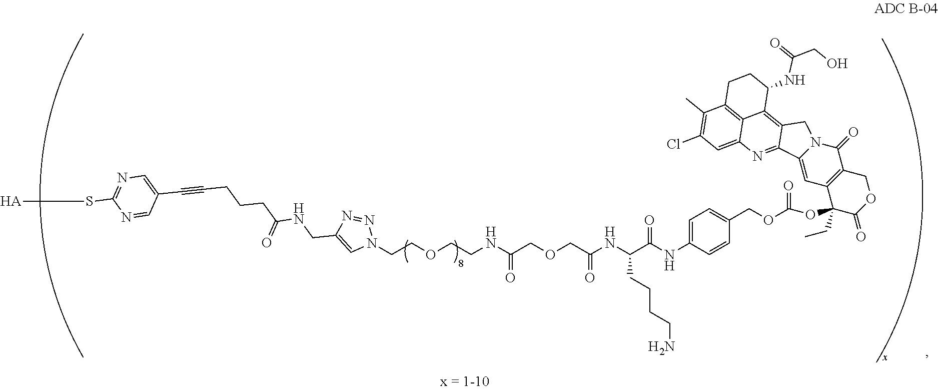

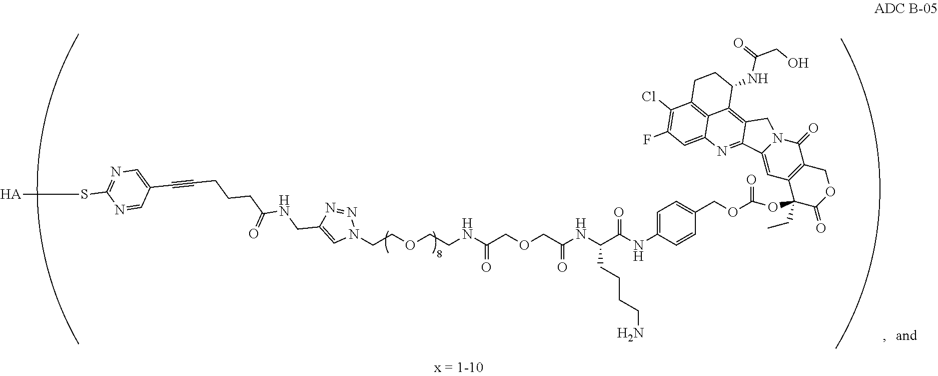

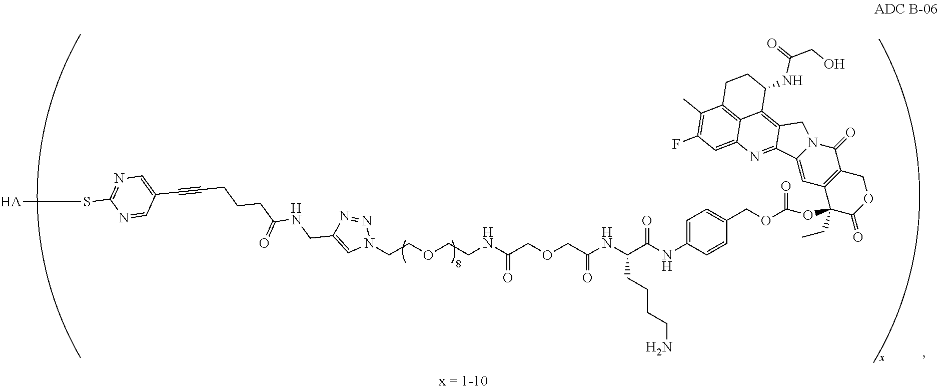

wherein HA-(S-represents an antibody or antigen-binding fragment thereof disclosed herein, and each -(S-represents a linkage from a sulfhydryl in the antibody or antigen-binding fragment thereof to each pyrimidinyl group in formulas ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06.

In some embodiments, the antibody-drug conjugate is selected from ADC-Aa, ADC-Aa1, ADC-Aa2, ADC-Bb, ADC-Bb1, and ADC-Bb2 shown as follows:

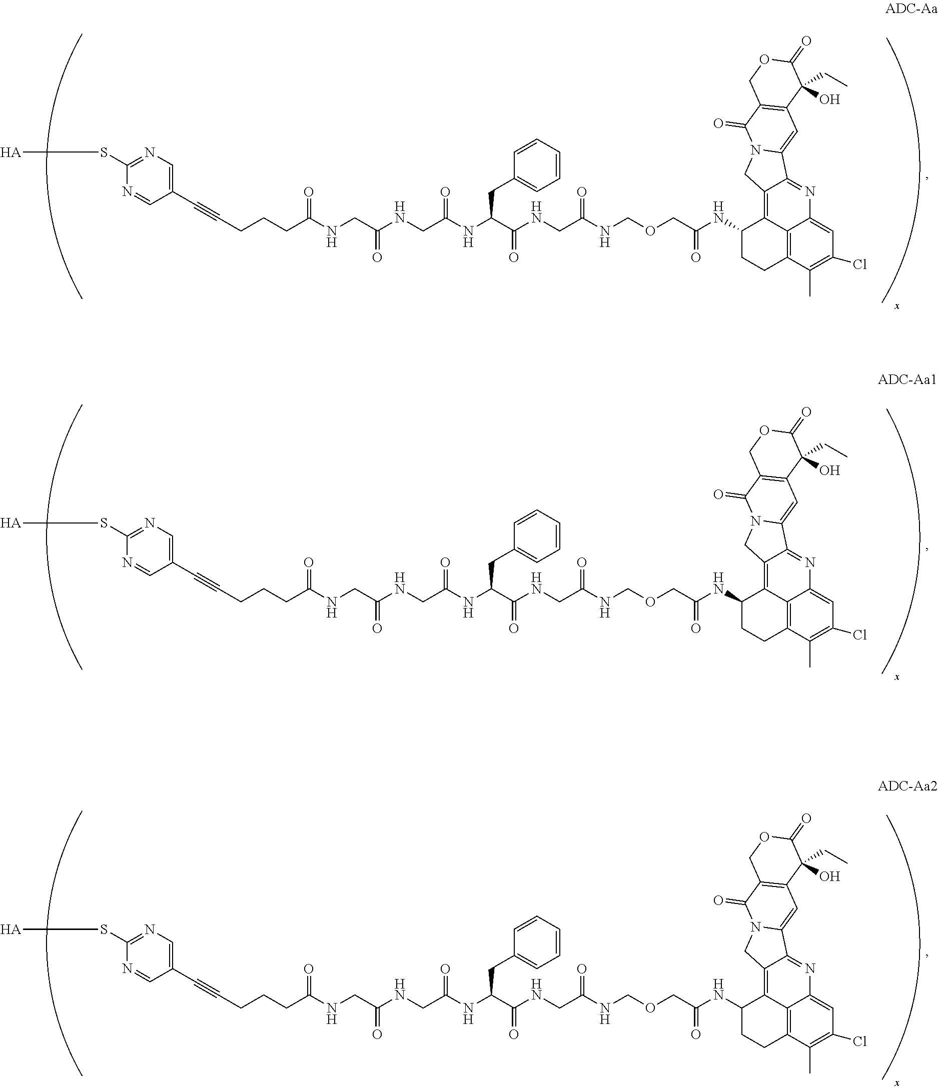

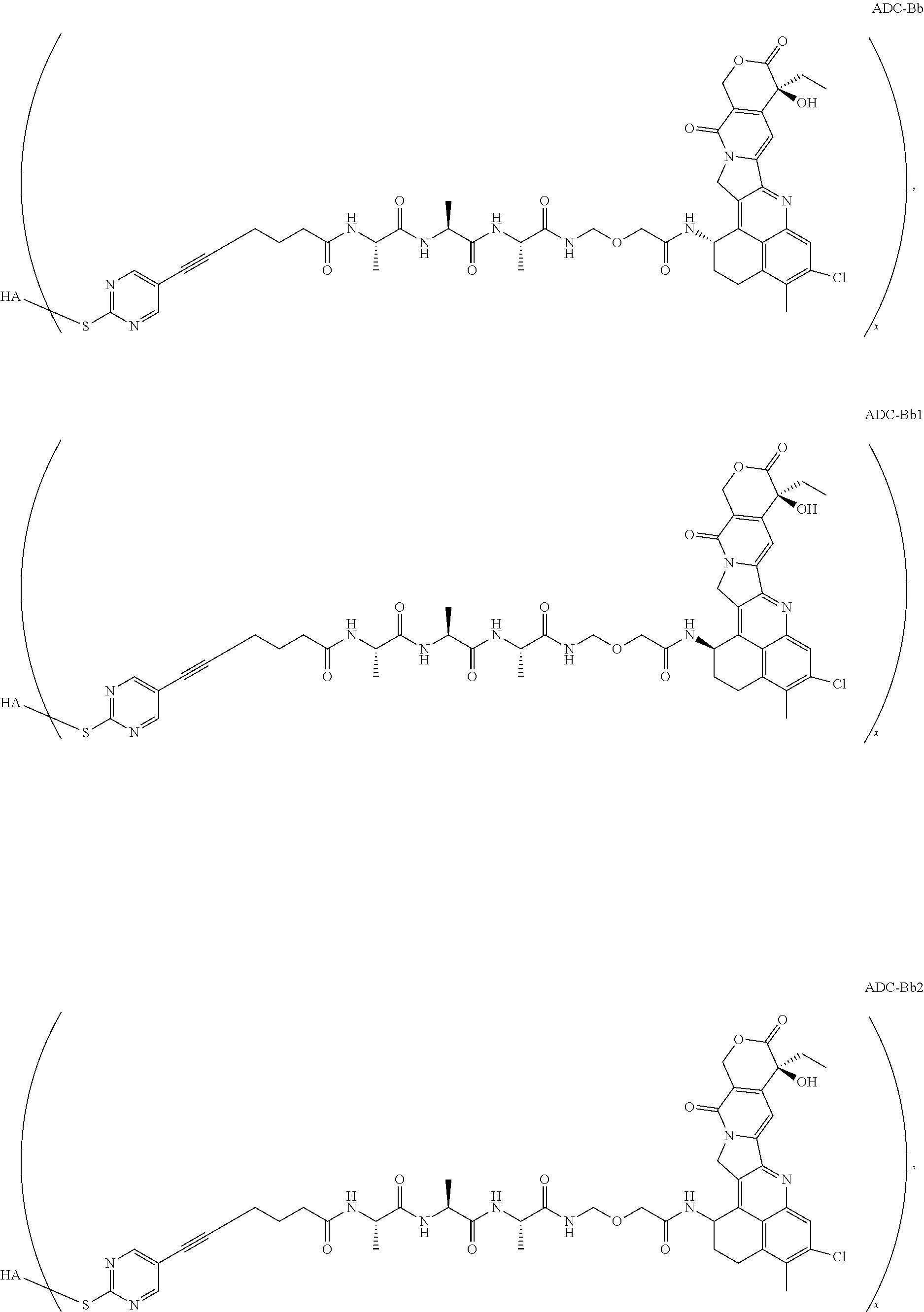

wherein HA-(S— comprises any one of the antibodies disclosed herein; each -(S-represents a linkage from a sulfhydryl in the antibody to each pyrimidinyl group; and x=1-16, 1-10, 1-8, 4-8, 4 or 8. In certain embodiments, each -(S-represents a linkage from a sulfhydryl of a cysteine residue in the antibody to each pyrimidinyl group in formulas ADC-Aa, ADC-Aa1, ADC-Aa2, ADC-Bb, ADC-Bb1, and ADC-Bb2.

In the foregoing embodiments for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-16. In one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa, ADC-Aa1, ADC-Aa2, ADC-Bb, ADC-Bb1, and ADC-Bb2, wherein HA-(S— comprises a V_{H} having the amino acid sequence as set forth in SEQ ID NO: 1, and a V_{L} having the amino acid sequence as set forth in SEQ ID NO: 2; -(S-represents a linkage from a sulfhydryl in the antibody to each pyrimidinyl group; and x=1-16, 1-10, 1-8, 4-8, 4 or 8.

In the foregoing embodiments for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-16. In one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from formulas ADC-Aa, ADC-Aa1, ADC-Aa2, ADC-Bb, ADC-Bb1, and ADC-Bb2, wherein HA-(S— comprises a heavy chain (HC) having the amino acid sequence as set forth in SEQ ID NO: 20 and a light chain (LC) having the amino acid sequence as set forth in SEQ ID NO: 21; -(S-represents a linkage from a sulfhydryl in the antibody to each pyrimidinyl group; and x=1-10, 1-8, 4-8, 4 or 8.

In the foregoing embodiments for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa and ADC-Bb shown as follows:

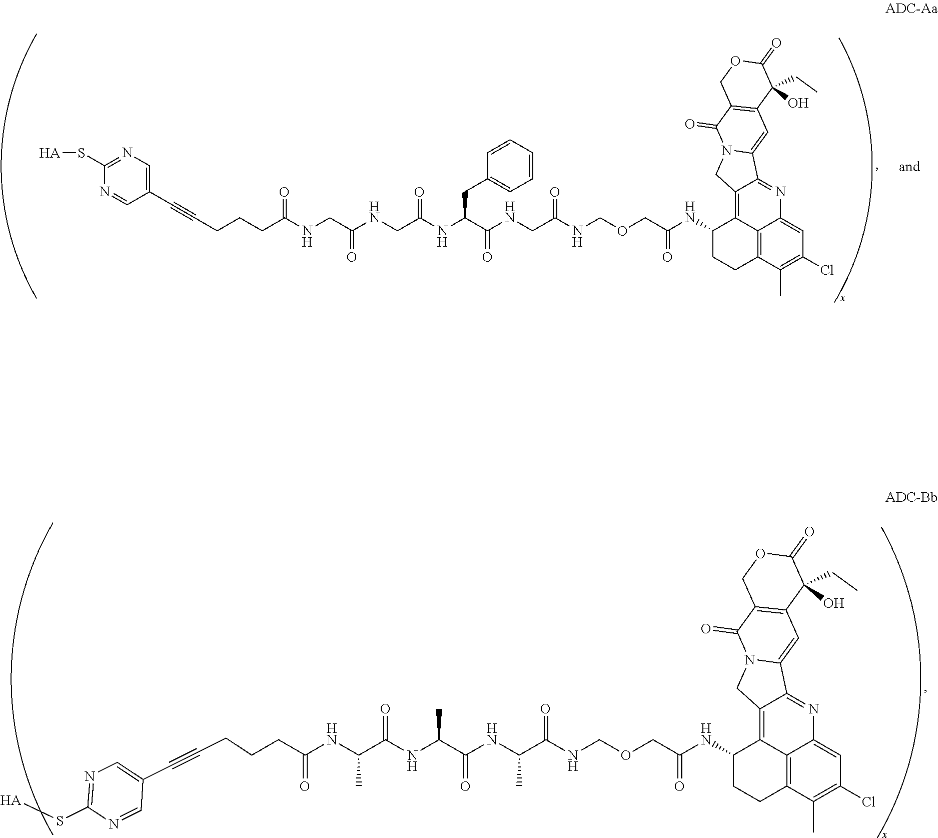

wherein HA-(S— comprises any one of the antibodies disclosed herein; each -(S-represents a linkage from a sulfhydryl in the antibody to each pyrimidinyl group in above formulas ADC-Aa and ADC-Bb; and x=1-10, 1-8, 4-8, 4 or 8. In certain embodiments, each -(S-represents a linkage from a sulfhydryl of a cysteine residue in the antibody to each pyrimidinyl group in above formulas ADC-Aa and ADC-Bb.

In the foregoing embodiments for antibody drug conjugates, for example, for antibody drug conjugates of formulas ADC-Aa and ADC-Bb, in one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa and ADC-Bb, wherein HA-(S— comprises a V_{H} having the amino acid sequence as set forth in SEQ ID NO: 1, and a V_{L} having the amino acid sequence as set forth in SEQ ID NO: 2; (S-represents a linkage from a sulfhydryl in the antibody to each pyrimidinyl group in above formulas ADC-Aa and ADC-Bb; and x=1-16, 1-10, 1-8, 4-8, 4 or 8.

In the foregoing embodiments for antibody drug conjugates, for example, for antibody drug conjugates of formulas ADC-Aa and ADC-Bb, in one embodiment, x is 1-16. In one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa and ADC-Bb, wherein HA-(S— comprises a heavy chain (HC) having the amino acid sequence as set forth in SEQ ID NO: 20 and a light chain (LC) having the amino acid sequence as set forth in SEQ ID NO: 21; -(S-represents a linkage from a sulfhydryl in the antibody to each pyrimidinyl group in above formulas ADC-Aa and ADC-Bb; and x=1-10, 1-8, 4-8, 4 or 8.

In the foregoing embodiments for antibody drug conjugates, for example, for antibody drug conjugates of formulas ADC-Aa and ADC-Bb, in one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa and ADC-Bb:

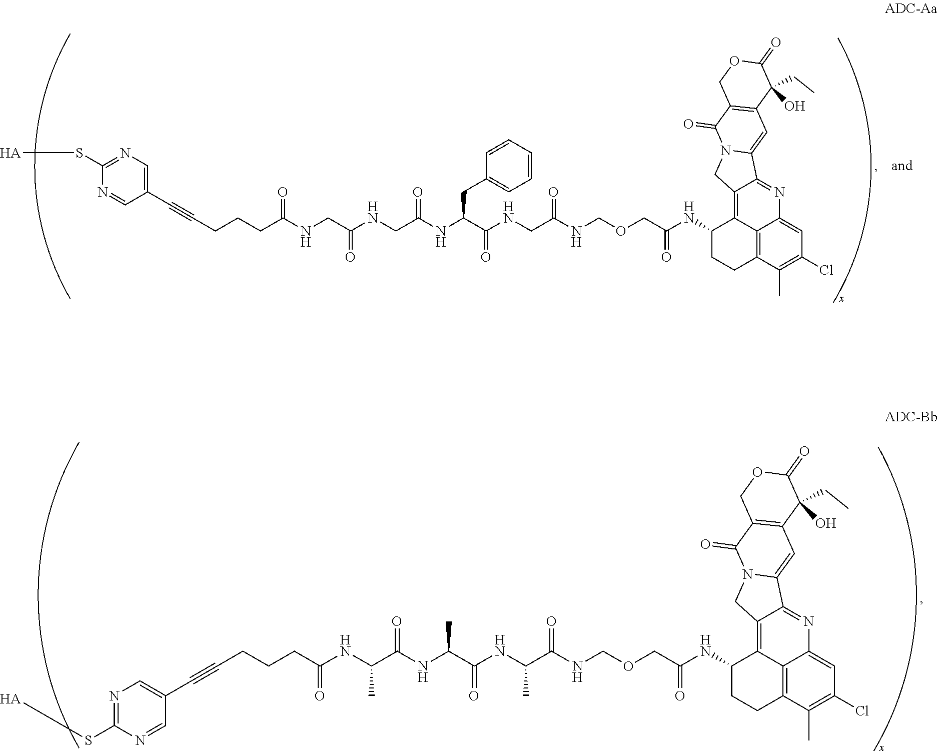

wherein x=1-8, 4-8, 4 or 8;
HA-(S— comprises any one of the antibodies disclosed herein, and each -(S-represents a linkage from a sulfhydryl in the antibody to each pyrimidinyl group in formulas ADC-Aa and ADC-Bb.

In some embodiments, the—(S-represents a linkage from a sulfhydryl of a cysteine residue in the antibody to each pyrimidinyl group in formulas ADC-Aa and ADC-Bb.

In the foregoing embodiments for antibody drug conjugates, for example, for antibody drug conjugates of formulas ADC-Aa and ADC-Bb, in one embodiment, x is 1-8. In one embodiment, x is 4-8. In one embodiment, x is 8. In one embodiment, x is an integer.

In some embodiments, the antibody-drug conjugate is selected from ADC-1, ADC-2, ADC-3, ADC-4 and ADC-5 as follows:

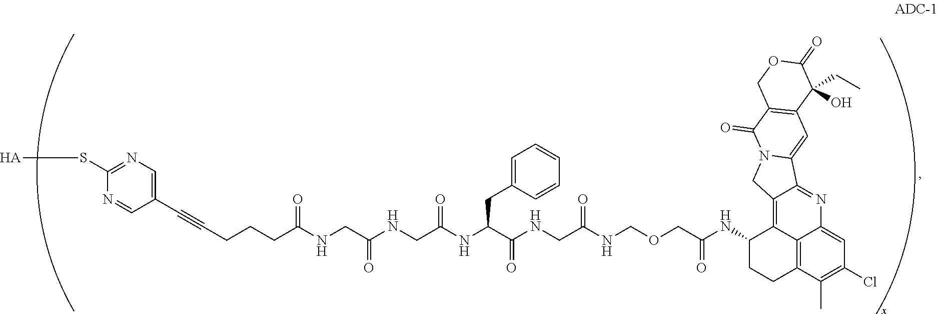

wherein x=8,
HA-(S— comprises antibody Ab-hzDPmut, and each -(S-represents a linkage from a sulfhydryl of a cysteine residue in the antibody to each pyrimidinyl group in formula ADC-1;

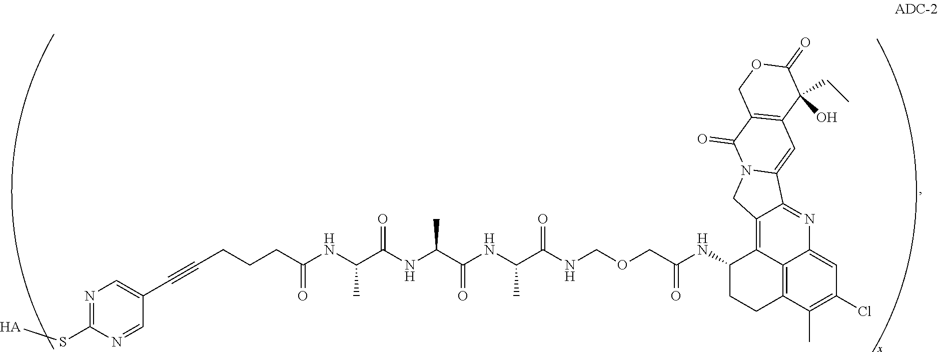

wherein x=8,
HA-(S— comprises antibody Ab-hzDPmut, and each -(S-represents a linkage from a sulfhydryl of a cysteine residue in the antibody to each pyrimidinyl group in formula ADC-2;

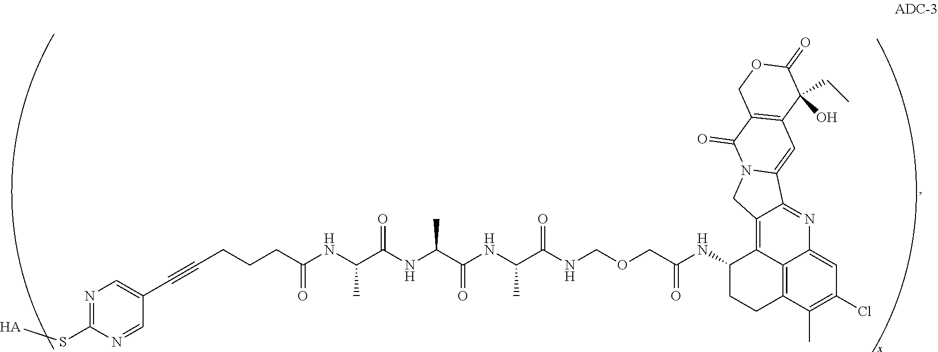

wherein x=4,
HA-(S— comprises antibody Ab-hzDPmut, and each -(S-represents a linkage from a sulfhydryl of a cysteine residue in the antibody to each pyrimidinyl group in formula ADC-3;

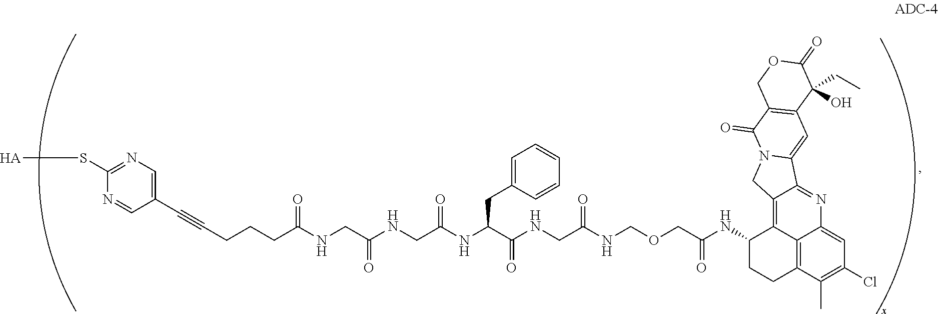

wherein x=8,
HA-(S— comprises antibody tusamitamab, and each -(S-represents a linkage from a sulfhydryl of a cysteine residue in the antibody to each pyrimidinyl group in formula ADC-4, and

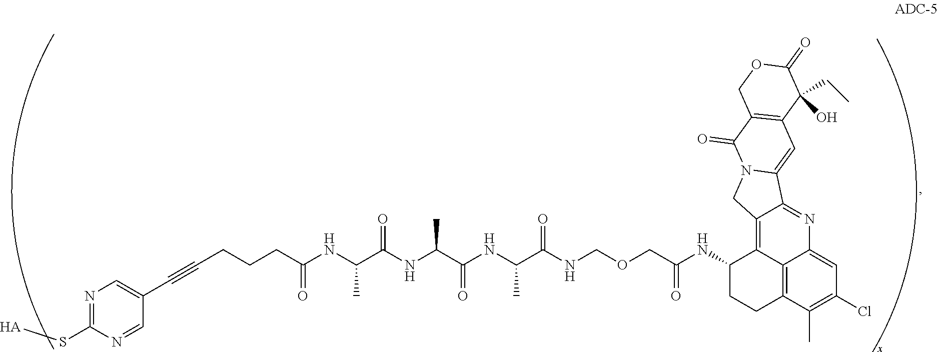

wherein x=8,
HA-(S— comprises antibody tusamitamab, and each -(S-represents a linkage from a sulfhydryl of a cysteine residue in the antibody to each pyrimidinyl group in formula ADC-5.

In a further embodiment, HA-(S— comprises:
(1) a heavy chain variable region (VH) comprising the following 3 CDRs: CDR-H1 having the amino acid sequence as set forth in SEQ ID NO: 14, CDR-H2 having the amino acid sequence as set forth in SEQ ID NO: 18, and CDR-H3 having the amino acid sequence as set forth in SEQ ID NO: 6; and/or, a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9;
(2) a heavy chain variable region (VH) comprising the following 3 CDRs: CDR-H1 having the amino acid sequence as set forth in SEQ ID NO: 15, CDR-H2 having the amino acid sequence as set forth in SEQ ID NO: 19, CDR-H3 having the amino acid sequence as set forth in SEQ ID NO: 6; and/or, a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9;
(3) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 17, CDR-H3 having the amino acid sequence as set forth in SEQ ID NO: 11; and/or, a light chain variable region (VL) comprising the following 3 CDRs: CDR-L1 having the amino acid sequence as set forth in SEQ ID NO: 12, CDR-L2 having the amino acid sequence as set forth in SEQ ID NO: 13, and CDR-L3 having the amino acid sequence as set forth in SEQ ID NO: 9; or
(4) a heavy chain variable region (VH) comprising the following 3 CDRs: CDR-H1 having the amino acid sequence as set forth SEQ ID NO: 5, CDR-H2 having the amino acid sequence as set forth in SEQ ID NO: 16, and CDR-H3 having the amino acid sequence as set forth in SEQ ID NO: 6; and/or, a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In a further embodiment, HA-(S— comprises a V_{H} having the amino acid sequence as set forth in SEQ ID NO: 1, and/or, a V_{L} having the amino acid sequence as set forth in SEQ ID NO: 2, and each -(S-represents a linkage from a sulfhydryl in the antibody to each pyrimidinyl group in formulas ADC-1, ADC-2, ADC-3, ADC-4 and ADC-5.

In a further embodiment, HA-(S— comprises a heavy chain constant region (CH) having the amino acid sequence as set forth in SEQ ID NO: 3, and a light chain constant region (CL) having the amino acid sequence as set forth in SEQ ID NO: 4; and each -(S-represents a linkage from a sulfhydryl in the antibody to each pyrimidinyl group in formulas ADC-1, ADC-2, ADC-3, ADC-4 and ADC-5.

In a further embodiment, HA-(S— comprises a heavy chain comprising a V_{H} having the amino acid sequence as set forth in SEQ ID NO: 1 and a heavy chain constant region (CH) having the amino acid sequence as set forth in SEQ ID NO: 3, and, a light chain comprising a VL having the amino acid sequence as set forth in SEQ ID NO: 2 and a light chain constant region (CL) having the amino acid sequence as set forth in SEQ ID NO: 4; and each -(S-represents a linkage from a sulfhydryl in the antibody to each pyrimidinyl group in formulas ADC-1, ADC-2, ADC-3, ADC-4 and ADC-5.

In a further embodiment, HA-(S— comprises a heavy chain (HC) having the amino acid sequence as set forth in SEQ ID NO: 20, and a light chain (LC) having the amino acid sequence as set forth in SEQ ID NO: 21; and each -(S-represents a linkage from a sulfhydryl in the antibody to each pyrimidinyl group in formulas ADC-1, ADC-2, ADC-3, ADC-4 and ADC-5. In certain embodiments, each -(S-represents a linkage from a sulfhydryl of a cysteine residue in the antibody to each pyrimidinyl group in formulas ADC-1, ADC-2, ADC-3, ADC-4 and ADC-5.

In some embodiments, the antibody or antigen-binding fragment thereof further comprises:
(a) a human immunoglobulin heavy chain constant region (CH) or a variant thereof, wherein the variant has replacement, deletion or addition of one, two, or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human immunoglobulin light chain constant (CL) or a variant, wherein the variant has replacement, deletion or addition of one, two, or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In one embodiment, HA-(S— comprises a V_{H} shown as SEQ ID NO: 1 and V_{L} shown as SEQ ID NO: 2, for example the antibodies or antigen-binding fragments of V_{H} shown as SEQ ID NO: 1 and CH shown as SEQ ID NO: 3, V_{L} shown as SEQ ID NO: 2 and CL shown as SEQ ID NO: 4; and each -(S-representes a linkage from a sulfhydryl of a cysteine residue in the antibody to each pyrimidinyl group in formulas ADC-1, ADC-2, ADC-3, ADC-4 and ADC-5.

In one embodiment, provided herein is an antibody-drug conjugate of formula ADC-Aa:

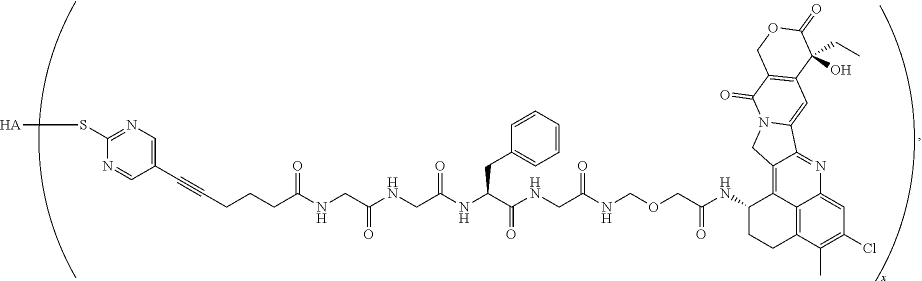

wherein HA-(S— comprises a heavy chain (HC) having the amino acid sequence as set forth in SEQ ID NO: 20 and a light chain (LC) having the amino acid sequence as set forth in SEQ ID NO: 21; each -(S-representes a linkage from a sulfhydryl of a cysteine residue in the antibody to each pyrimidinyl group in formula ADC-Aa; and x is an integer from 1-8, 4-8, 4 or 8.

In the foregoing embodiment for antibody drug conjugate, for example, for antibody drug conjugates of formula ADC-Aa, in one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Bb:

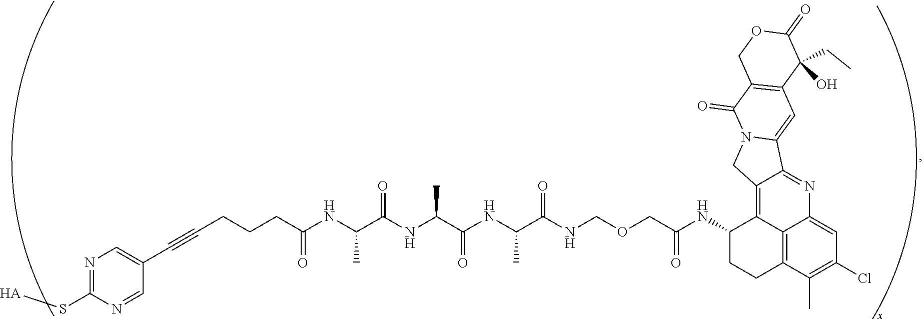

wherein HA-(S— comprises a heavy chain (HC) having the amino acid sequence as set forth in SEQ ID NO: 20 and a light chain (LC) having the amino acid sequence as set forth in SEQ ID NO: 21; each -(S-representes a linkage from a sulfhydryl in the antibody to each pyrimidinyl group in formula ADC-Bb; and x is an integer from 1-8, 4-8, 4 or 8.

In the foregoing embodiment for antibody drug conjugate, for example, for antibody drug conjugates of formula ADC-Bb, in one embodiment, x is 1-8. In one embodiment, x is 4-8. In one embodiment, x is 4. In one embodiment, x is 8. In one embodiment, x is an integer.

In one embodiment, each -(S-represents a linkage from a sulfhydryl of a cysteine residue in the antibody to each pyrimidinyl group in formula ADC-Bb.

In one embodiment, provided herein is an antibody-drug conjugate of formula ADC-Aa′ or ADC-Bb′:

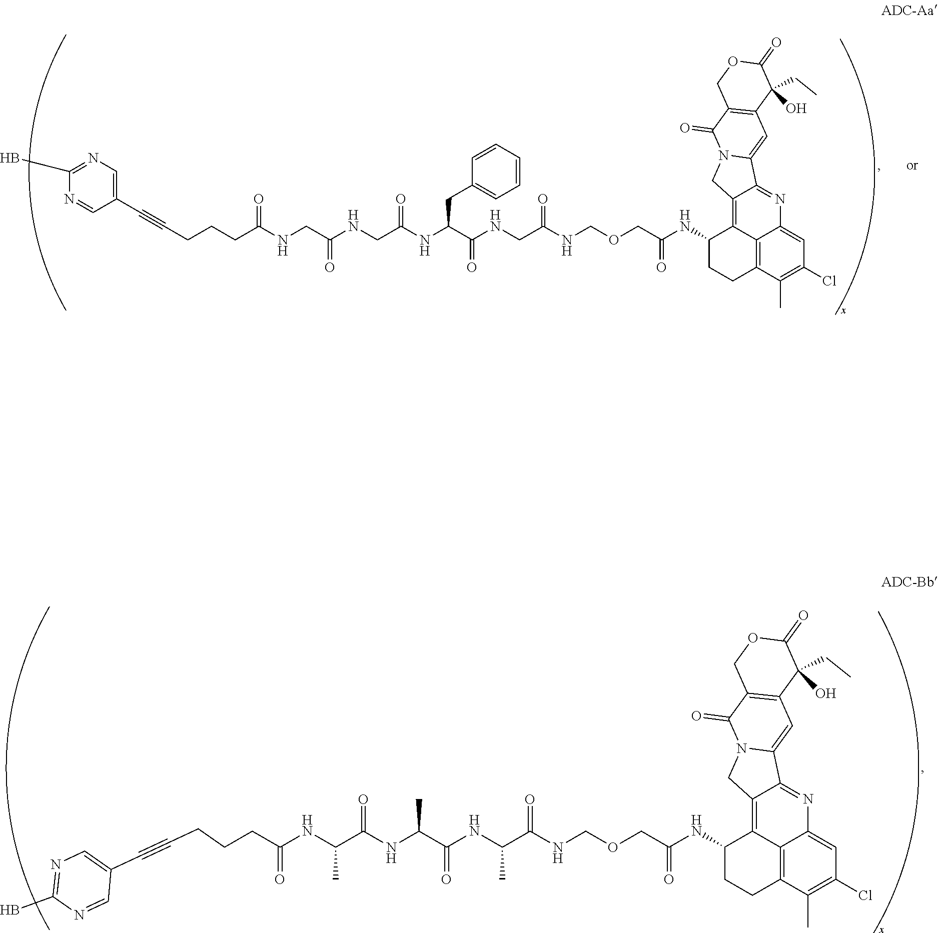

wherein HB—(- comprises any one of the antibodies disclosed herein, and x is an integer from 1-20, 1-16, 1-8, 4-8, 4 or 8.

In the foregoing embodiment for antibody drug conjugates, for example, for antibody drug conjugates of formulas ADC-Aa′ and ADC-Bb′, in one embodiment, x is 1-20. In one embodiment, x is 1-16. In one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Aa′ or ADC-Bb′:

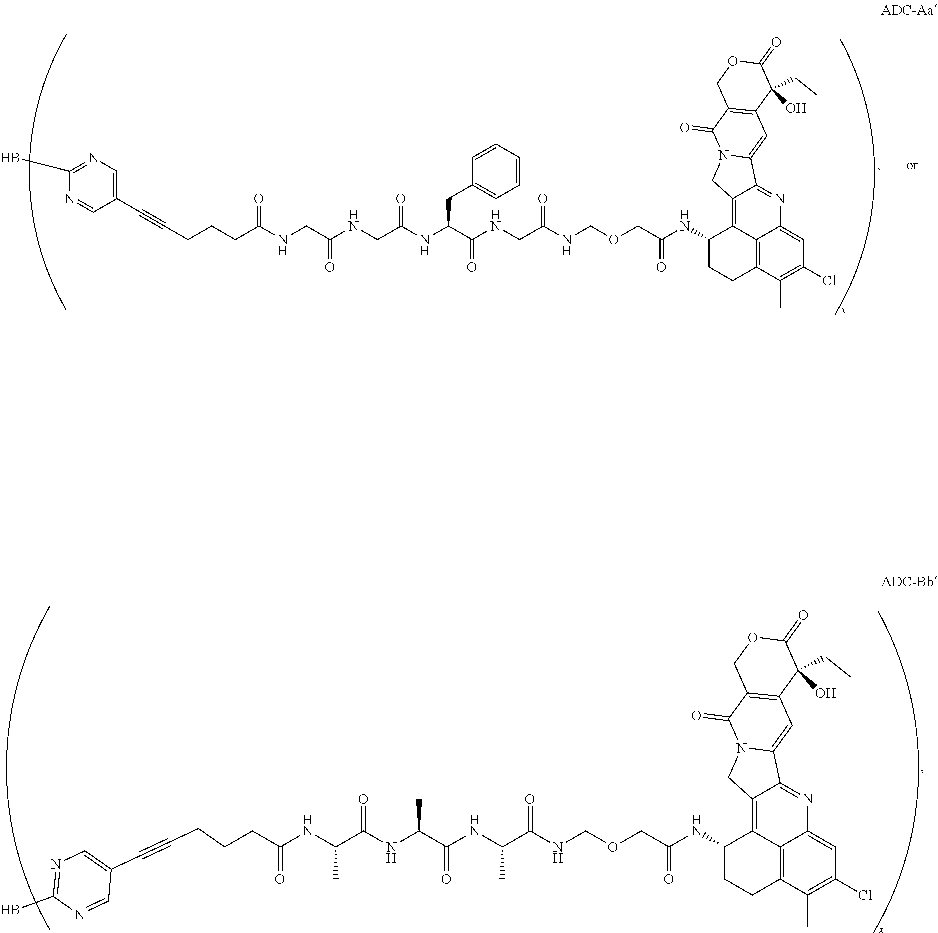

wherein HB-(- comprises a V_{H} having the amino acid sequence as set forth in SEQ ID NO: 1, and a V_{L} having the amino acid sequence as set forth in SEQ ID NO: 2, and x=1-20, 1-16, 1-10, 1-8, 4-8, 4 or 8.

In the foregoing embodiment for antibody drug conjugates, for example, for antibody drug conjugates of formulas ADC-Aa′ and ADC-Bb′, in one embodiment, x is 1-20. In one embodiment, x is 1-16. In one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Aa′ or ADC-Bb′:

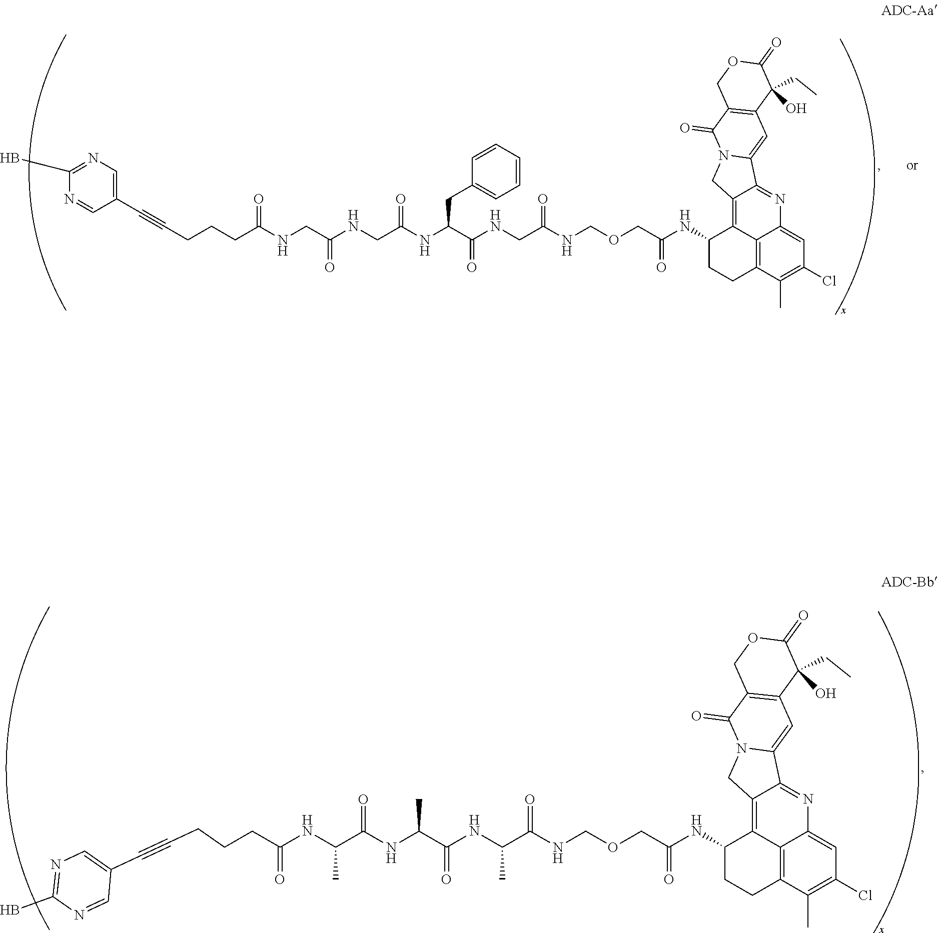

wherein HB-(- is a conjugated anti-CEACAM5 monoclonal antibody comprising a heavy chain (HC) having the amino acid sequence as set forth in SEQ ID NO: 20 and a light chain (LC) having the amino acid sequence as set forth in SEQ ID NO: 21, and x is an integer from 1-8, 4-8, 4 or 8.

In the foregoing embodiment for antibody drug conjugates, for example, for antibody drug conjugates of antibody drug conjugates of formulas ADC-Aa′ and ADC-Bb′, in one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Aa′:

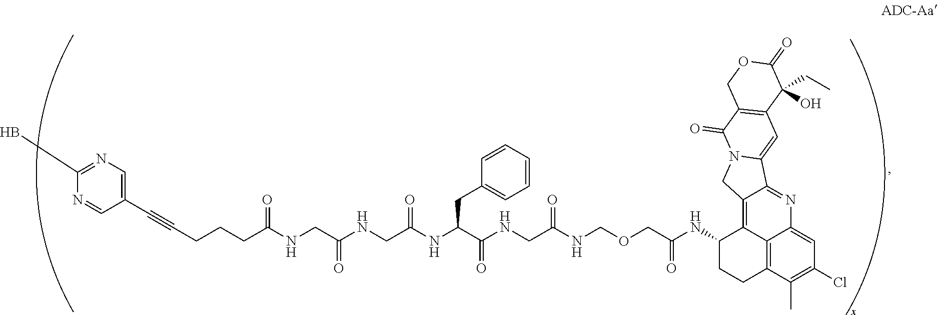

wherein HB-(- is a conjugated anti-CEACAM5 monoclonal antibody comprising a heavy chain (HC) having the amino acid sequence as set forth in SEQ ID NO: 20 and a light chain (LC) having the amino acid sequence as set forth in SEQ ID NO: 21, and x is an integer from 1-8, 4-8, 4 or 8.

In the foregoing embodiment for antibody drug conjugate, for example, for antibody drug conjugate of formula ADC-Aa′, in one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC Bb′:

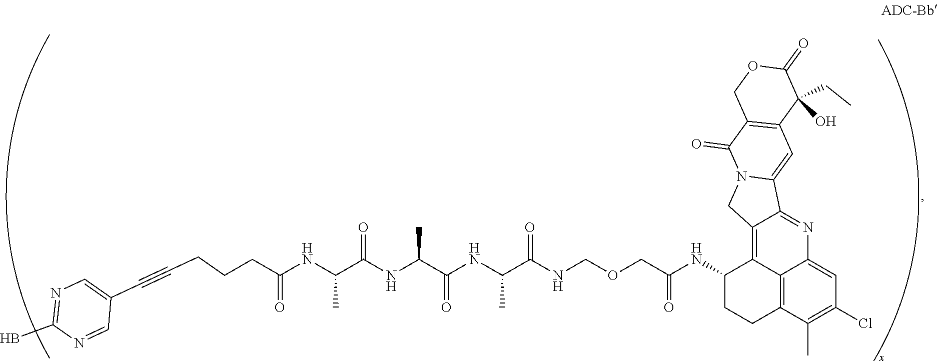

wherein HB-(- is a conjugated anti-CEACAM5 monoclonal antibody comprising a heavy chain (HC) having the amino acid sequence as set forth in SEQ ID NO: 20 and a light chain (LC) having the amino acid sequence as set forth in SEQ ID NO: 21, and x is an integer from 1-8, 4-8, 4 or 8.

In the foregoing embodiment for antibody drug conjugate, for example, for antibody drug conjugate of formula ADC-Bb′, in one embodiment, x is 1-8. In one embodiment, x is 4-8. In one embodiment, x is 4. In one embodiment, x is 8. In one embodiment, x is an integer.

In one embodiment, HB-(- is linked via one or more sulfhydryl group(s) of cysteine residue(s), one or more amino group(s) of lysine residue(s), one or more hydroxy group(s) of threonine residue(s) or one or more hydroxy group(s) of serine residue(s) in the antibody to form the conjugate.

In one embodiment, HB-(- is linked via x number of sulfhydryl group(s) of cysteine residue(s) in the antibody.

In particular embodiments of the antibody-drug conjugate disclosed herein, the heavy chain constant domains may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof may undergo cyclization to pyroglutamate.

In some embodiments of the antibody or antigen binding fragment thereof, the N-terminal amino acid of the antibody or antigen binding fragment thereof may undergo cyclization to pyroglutamatic acid.

Thus, a composition may comprise a population of antibody-drug conjugate species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamate.

In certain embodiments, the composition may comprise a population of antibody-drug conjugate species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamatic acid.

Therefore, in particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminal amino acid is glutamine, glutamic acid, or pyroglutamate; or, (iii) antibodies in which the heavy chain C-terminus lacks a lysine residue and the heavy chain N-terminal amino acid is glutamine, glutamic acid, or pyroglutamate.

In particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminal amino acid is glutamine, glutamic acid, or pyroglutamic acid; or, (iii) antibodies in which the heavy chain C-terminus lacks a lysine residue and the heavy chain N-terminal amino acid is glutamine, glutamic acid, or pyroglutamic acid.

As known to those skilled in the art, pyroglutamic acid is the conjugate acid of pyroglutamate, and is in equilibrium with pyroglutamate in solution.

### Compositions

In another aspect, the present application provides a composition of antibody-drug conjugates (ADCs) as described herein. Such composition may comprise a plurality of ADCs as described herein, wherein each ADC comprises a drug-linker as described herein. Therefore, a composition may be characterized by a “drug-to-antibody” ratio (DAR) ranging from about 1 to about 10. Methods to determine DAR are well known to the skilled person and include methods using Reverse Phase Chromatography, or HPLC-MS.

In some embodiments, the DAR value (drug-antibody conjugation ratio) of the composition of drug-antibody conjugate is 1-10, for example: 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10, preferably 1-8, preferably 3-9, for example, 3.0-3.5, 3.0-4.0, 3.0-4.5, 3.0-5.0, 3.0-5.5, 3.0-6.0, 3.5-4.0, 3.5-4.5, 3.5-5.0, 3.5-5.5, 3.5-6.0, 3.5-6.5, 3.5-7.0, 3.5-7.5, 3.5-8.0, 4.0-4.5, 4.0-5.0, 4.0-5.5, 4.0-6.0, 4.0-6.5, 4.0-7.0, 4.0-7.5, 4.0-8.0, 4.5-5.0, 4.5-5.5, 4.5-6.0, 4.5-6.5, 4.5-7.0, 4.5-7.5, 4.5-8.0, 5.0-5.5, 5.0-6.0, 5.0-6.5, 5.0-7.0, 5.0-7.5, 5.0-8.0, 5.5-6.0, 5.5-6.5, 5.5-7.0, 5.5-7.5, 5.5-8.0, 6.0-6.5, 6.0-7.0, 6.0-7.5, 6.0-8.5, 6.5-7.0, 6.5-7.5, 6.5-8.5, 7.0-7.5, 7.0-9.0 or 7.5-9.0.

In one embodiment, the DAR value is 8. In one embodiment, the DAR value is 7 to 9. In one embodiment, the DAR value is 7 to 8. In other embodiments, the DAR value of a composition of antibody drug conjugate comprising ADC-1 is about 8. In other embodiments, the DAR value of a composition of antibody drug conjugate comprising ADC-1 is 7 to 9. In other embodiments, the DAR value of a composition of antibody drug conjugate comprising ADC-1 is 7 to 8. In other embodiments, the DAR value of a composition of antibody drug conjugate comprising ADC-2 is about 8. In other embodiments, the DAR value of a composition of antibody drug conjugate comprising ADC-2 is 7 to 9. In other embodiments, the DAR value of a composition of antibody drug conjugate comprising ADC-2 is 7 to 8. In other embodiments, the DAR value of a composition of antibody drug conjugate comprising ADC-3 is about 4. In other embodiments, the DAR value of a composition of antibody drug conjugate comprising ADC-3 is 4 to 6. In other embodiments, the DAR value of a composition of antibody drug conjugate comprising ADC-3 is 4 to 5. In other embodiments, the DAR value of a composition of antibody drug conjugate comprising ADC-4 is about 8. In other embodiments, the DAR value of a composition of antibody drug conjugate comprising ADC-4 is 7 to 9. In other embodiments, the DAR value of a composition of antibody drug conjugate comprising ADC-4 is 7 to 8. In other embodiments, the DAR value of a composition of antibody drug conjugate comprising ADC-5 is about 8. In other embodiments, the DAR value of a composition of antibody drug conjugate comprising ADC-5 is 7 to 9. In other embodiments, the DAR value of a composition of antibody drug conjugate comprising ADC-5 is 7 to 8.

### Drug-Linker

Those skilled in the art should understand that the antibody-drug conjugate described in the present application can be prepared in a modularized manner. For example, the “drug-linker” in a pre- conjugated form (which can be understood as M′-L-E-D, wherein M′ is a structural form before covalent linkage of M to the antibody or antigen binding fragment thereof) is obtained, and then the ‘drug-linker’ in the pre- conjugated form is covalently linked to the antibody or antigen binding fragment thereof to obtain the antibody-drug conjugate according to the present application. Correspondingly, M′ in the pre- conjugated form of ‘drug-linker’ is linked to one or more sulfhydryl(—SH), amino (-NH₂) or carboxyl(—COOH) on the antibody or antigen binding fragment thereof through a substitution reaction (for example, removal of—SO₂Me or -Br and the like structures thereon) or through an addition reaction and other manners.

In another aspect, the present invention provides a drug-linker having a structure shown as the formula M′-L-E-D, where
M′ is

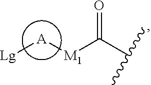

and Lg is a leaving group for a nucleophilic substitution reaction (for example, halogen, methanesulfonyl, fluorophenol, or

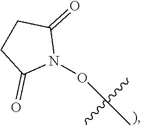 [SMILES: *ON1C(=O)CCC1=O]

or hydroxyl(—OH), sulfhydryl(—SH) or amino (-NH₂); or, Lg forms an unsaturated double bond with an adjacent atom on ring A; ring A is a 5-6 membered aliphatic heterocyclic ring, or a 5-20 membered aromatic ring system, and the aliphatic heterocyclic ring and the aromatic ring system are optionally substituted by one or more groups selected from oxo (═O), halogen, cyano, amino, carboxyl, sulfhydryl and C₁₋₆ alkyl; and M₁ is selected from a single bond, C₁₋₂₀ alkylene, C₂₋₂₀ alkenylene and C₂₋₂₀ alkynylene; and
L, E and D structures are defined according to any one of above antibody-drug conjugate.

In some embodiments, M′ is

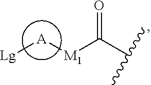

Lg is methanesulfonyl, or Lg and adjacent atoms on the ring A form carbon-carbon double bonds; the ring A is a 5-membered alicyclic heterocyclic ring, a 6-membered heteroaromatic ring, or a polycyclic ring formed by linking more than one 6-membered heteroaromatic ring and a benzene ring through a single bond, and the alicyclic heterocyclic ring is optionally substituted by one, two, or more groups independently selected from oxo (═O), halogen and C₁₋₄ alkyl; and M₁ is selected from a single bond, C₃₋₁₀ alkylene, C₃₋₁₀ alkenylene and C₃₋₁₀ alkynylene.

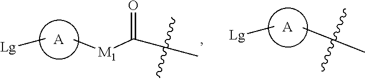

In some embodiments, M′ is

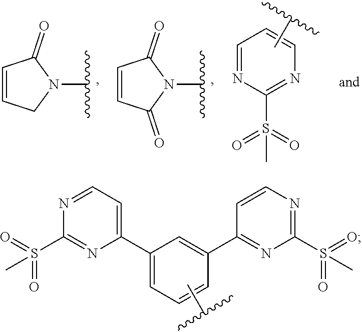

is selected from and M₁ is selected from a single bond, C₅₋₈alkylene, C₅₋₈alkenylene and C₅₋₈alkynylene.

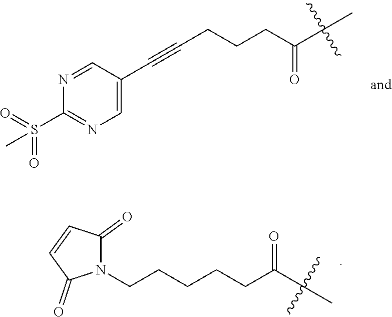 [SMILES: *C(=O)CCCC#Cc1cnc(S(C)(=O)=O)nc1 ; "and" ; *C(=O)CCCCCN1C(=O)C=CC1=O ; "."]

In some embodiments, M′ is selected from In some embodiments, the “drug-linker” in pre- conjugated form is selected from A-01 to A-04, A-1, A-06 to A-25, A-2, B-01 to B-06, and C-01 shown as follows:

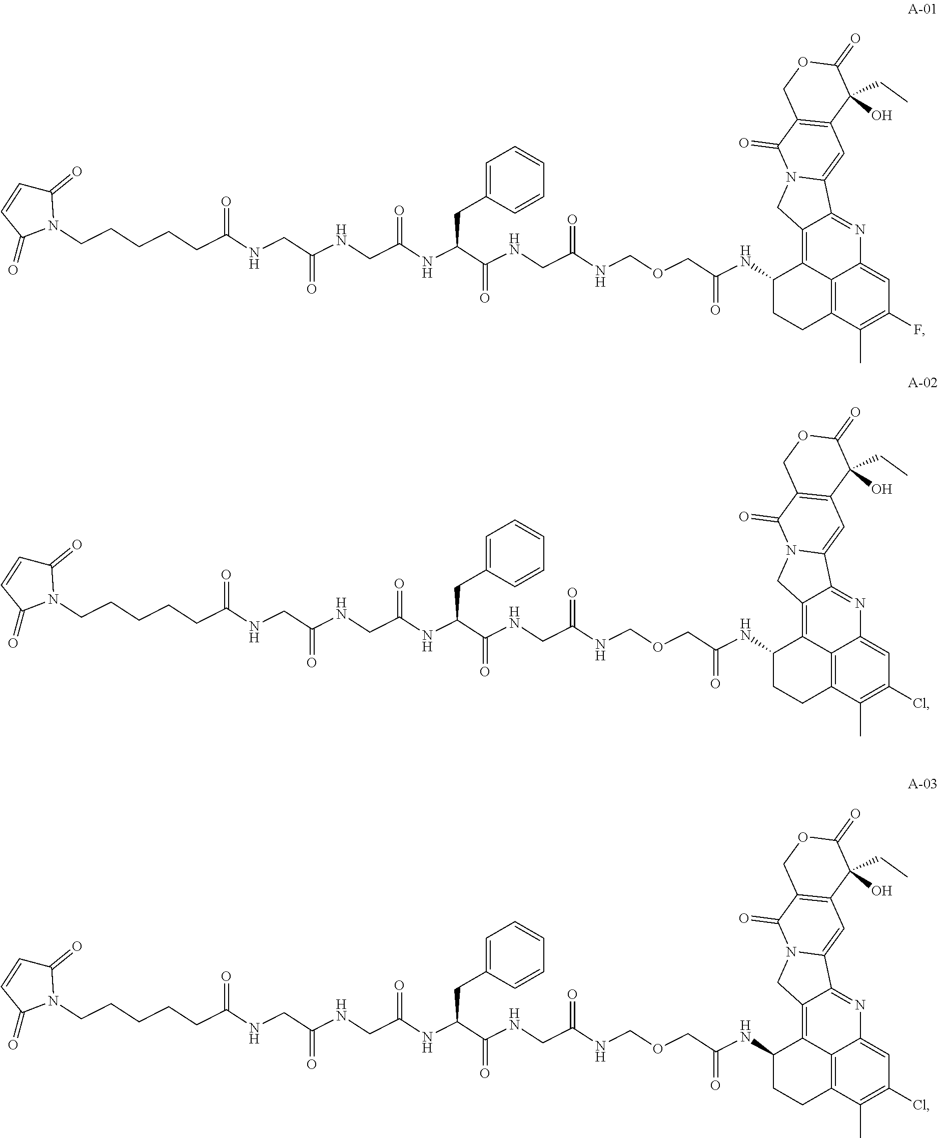 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

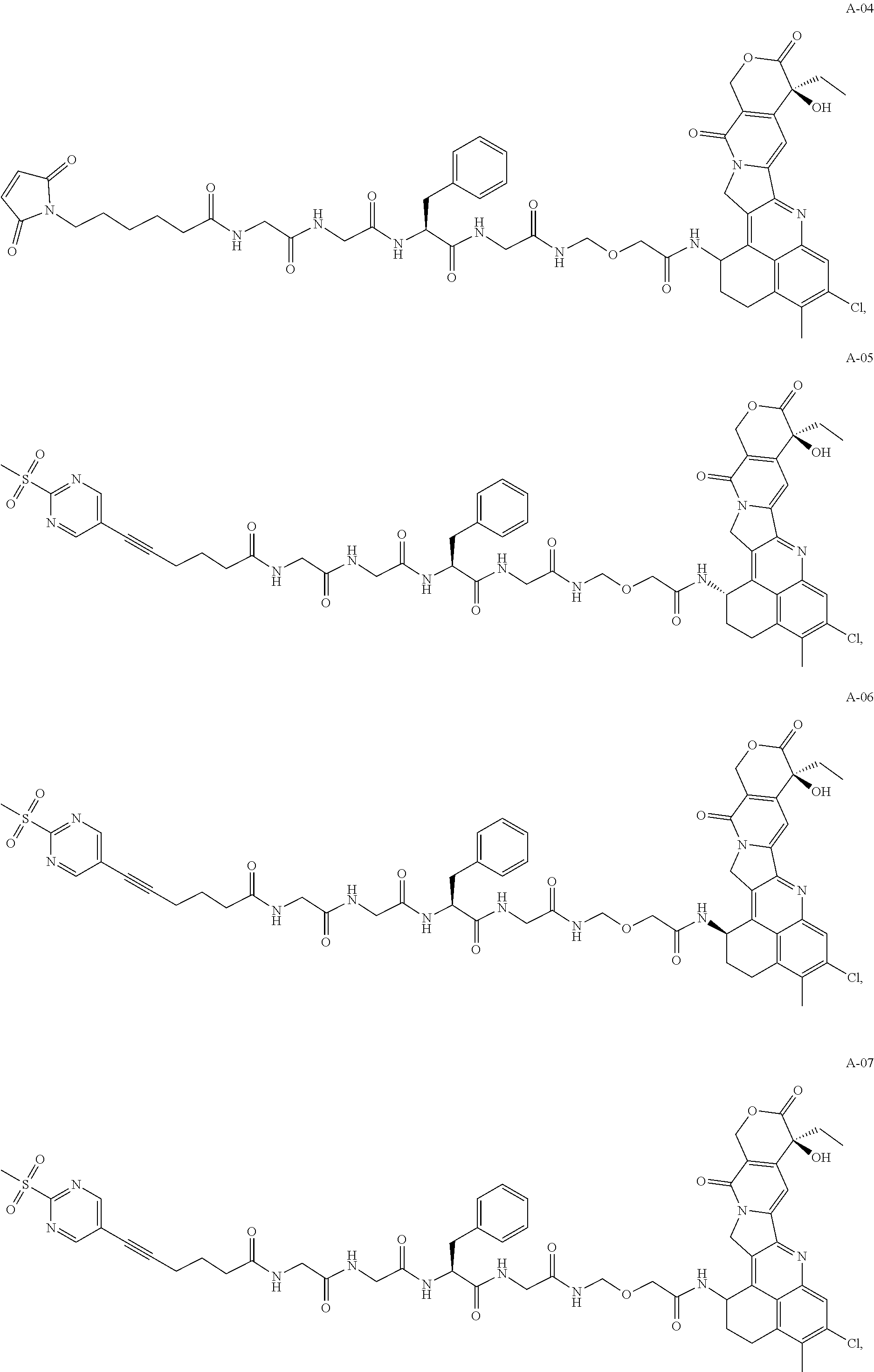

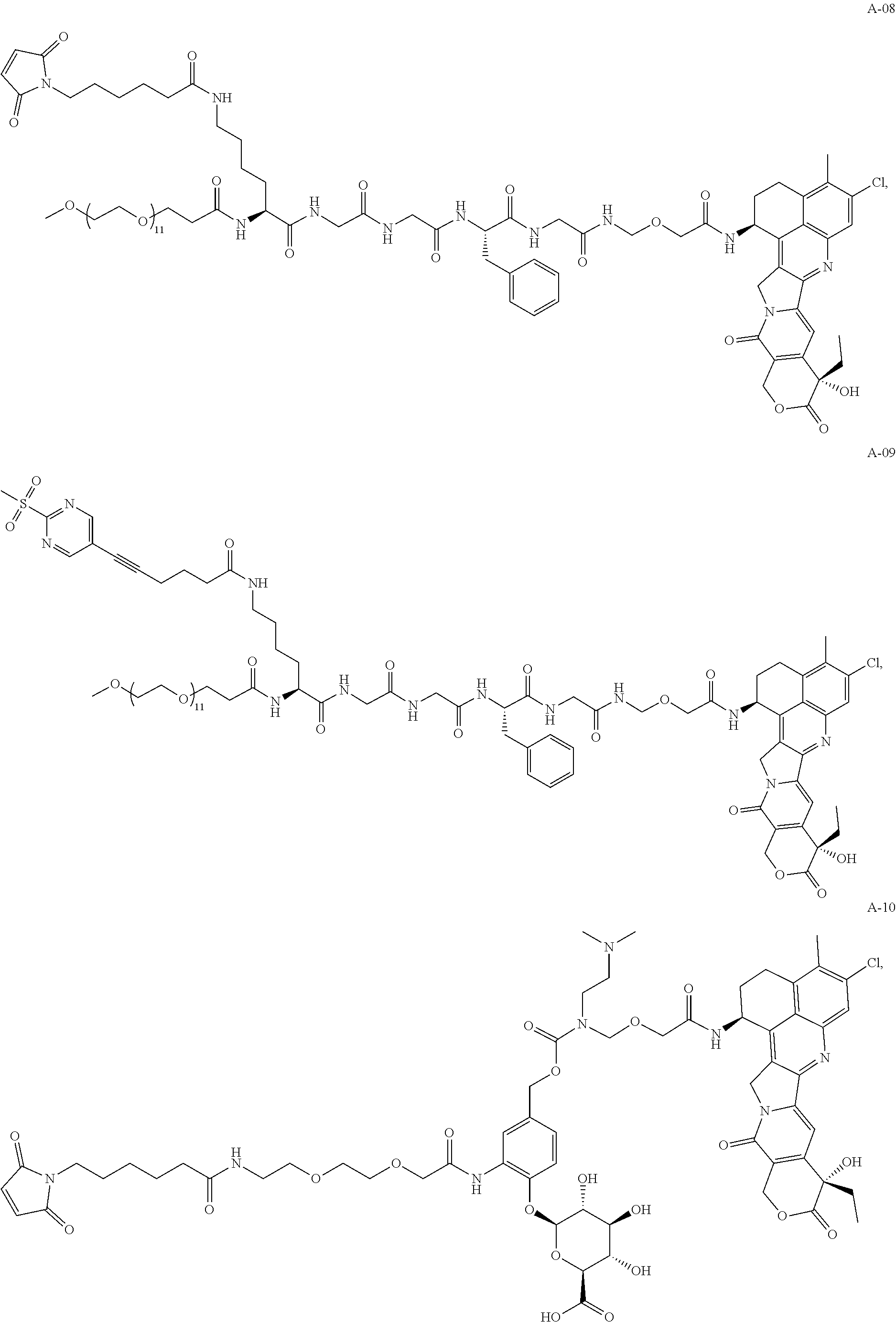

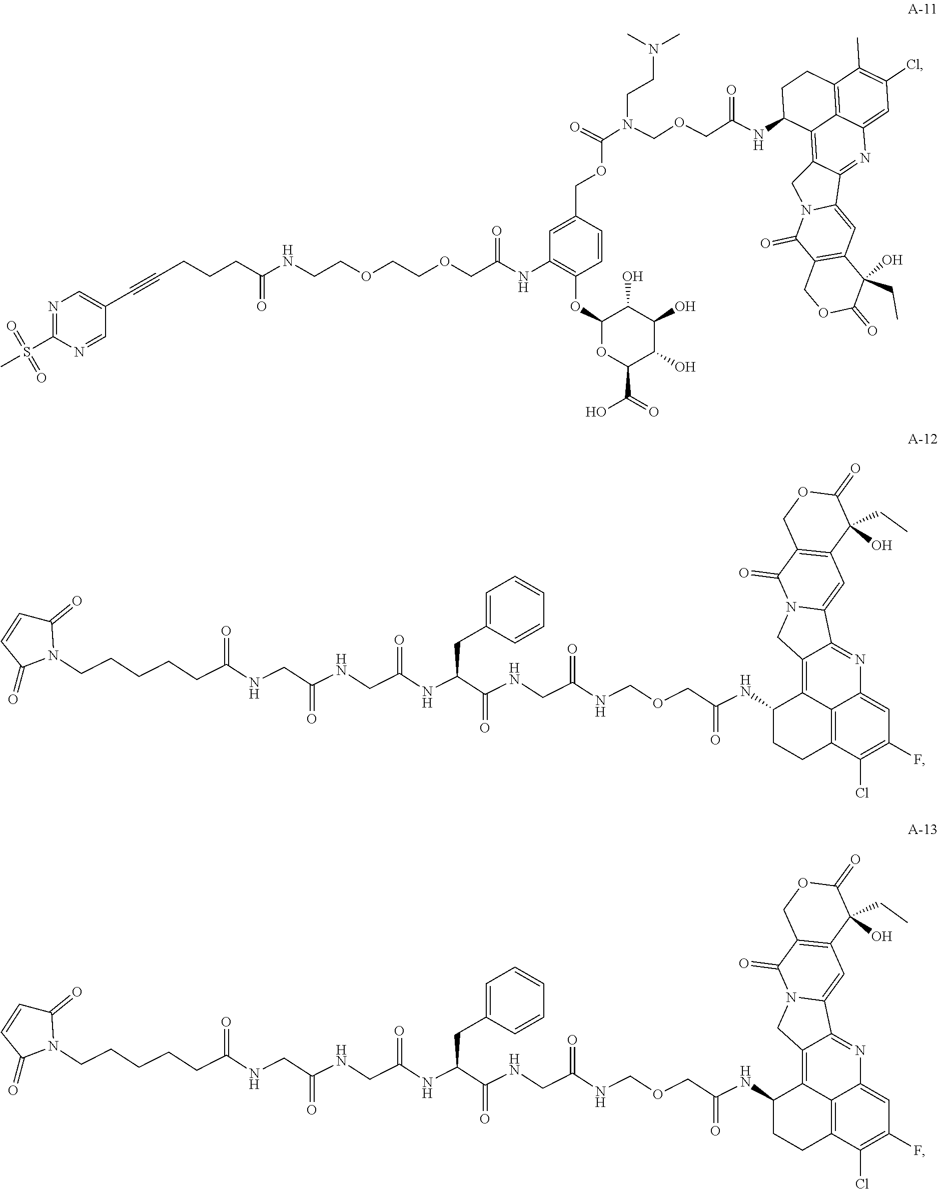 [SMILES: CC[C@@]1(O)C(=O)OCc2c1cc1n(c2=O)Cc2c-1nc1cc(Cl)c(C)c3c1c2[C@@H](NC(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(S(C)(=O)=O)nc2)c1)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

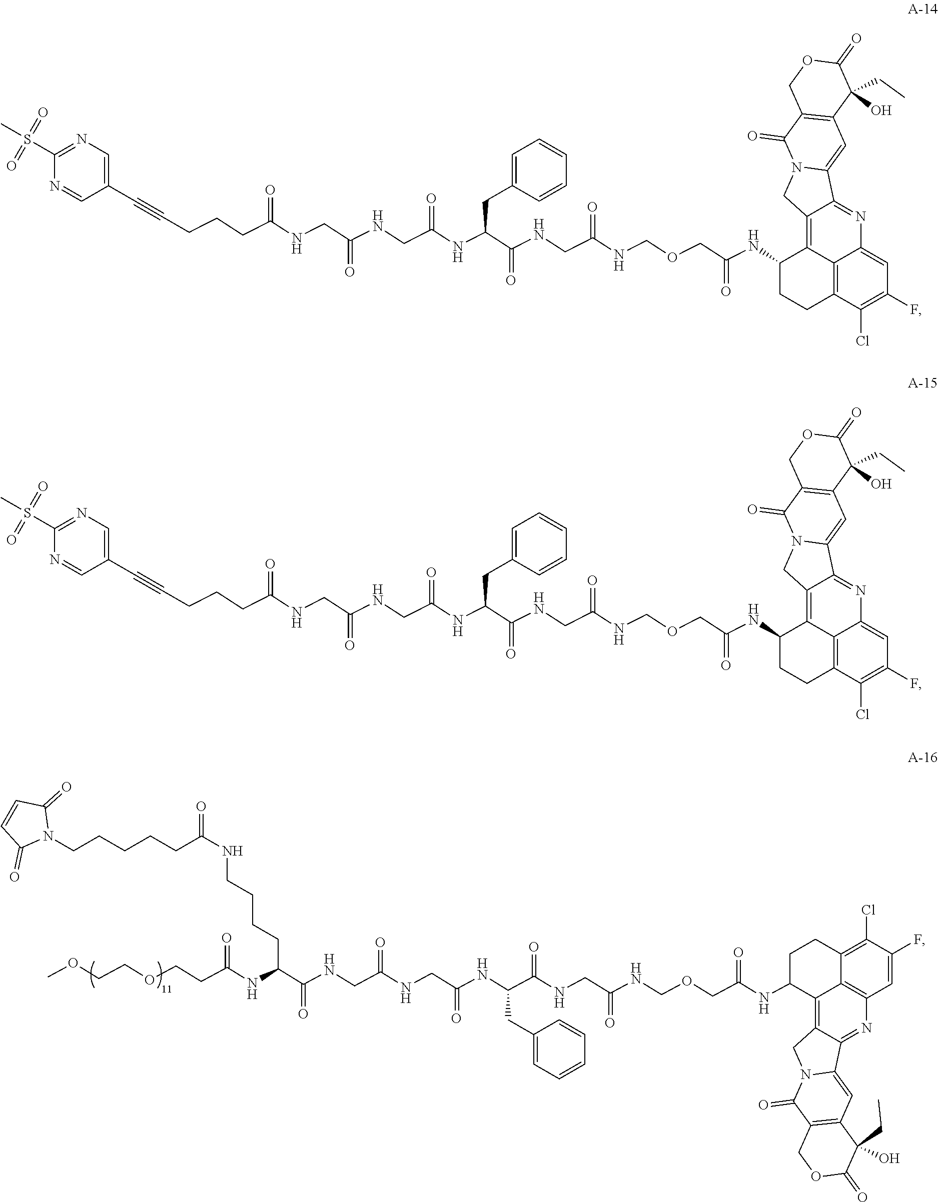

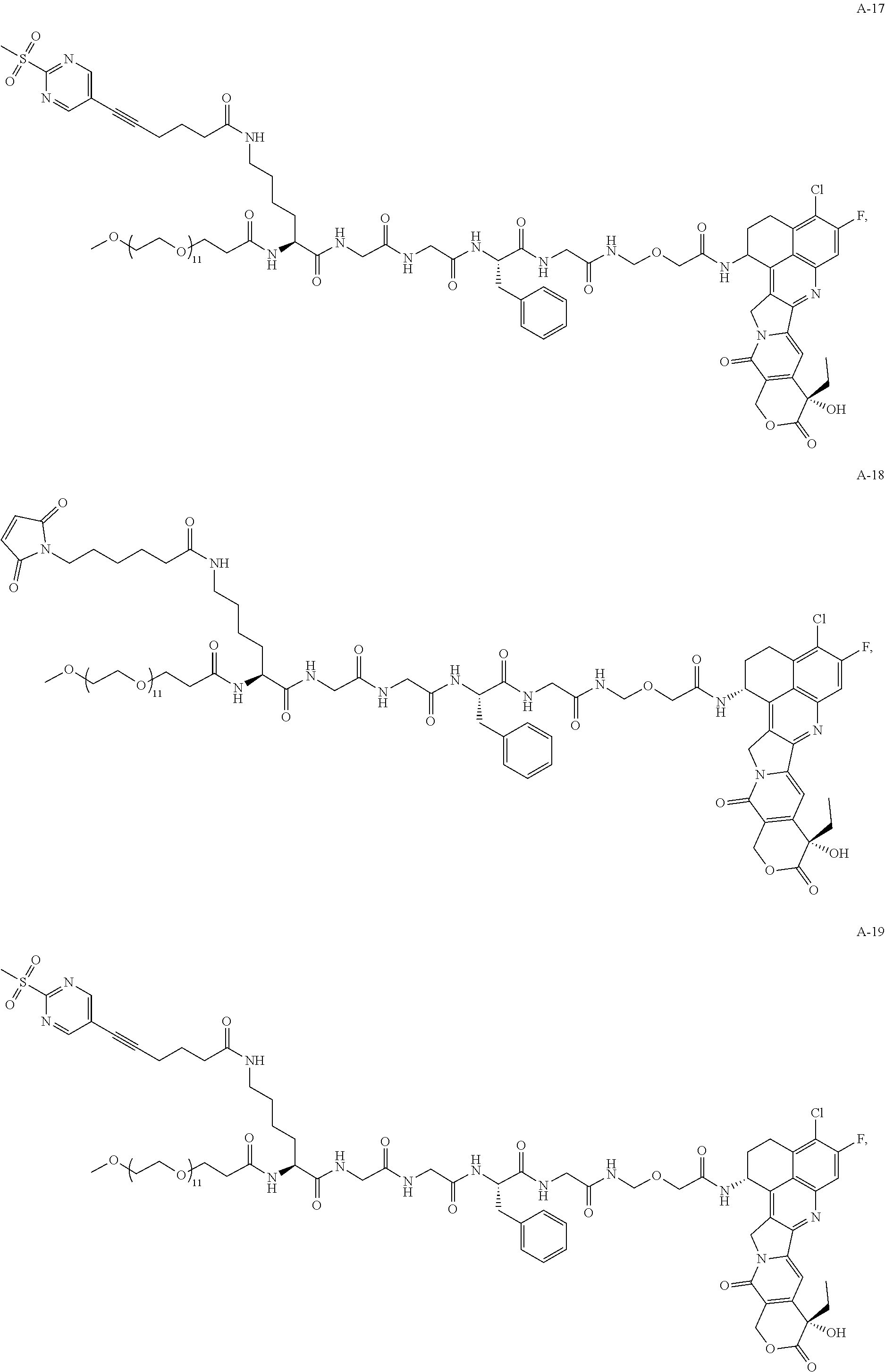

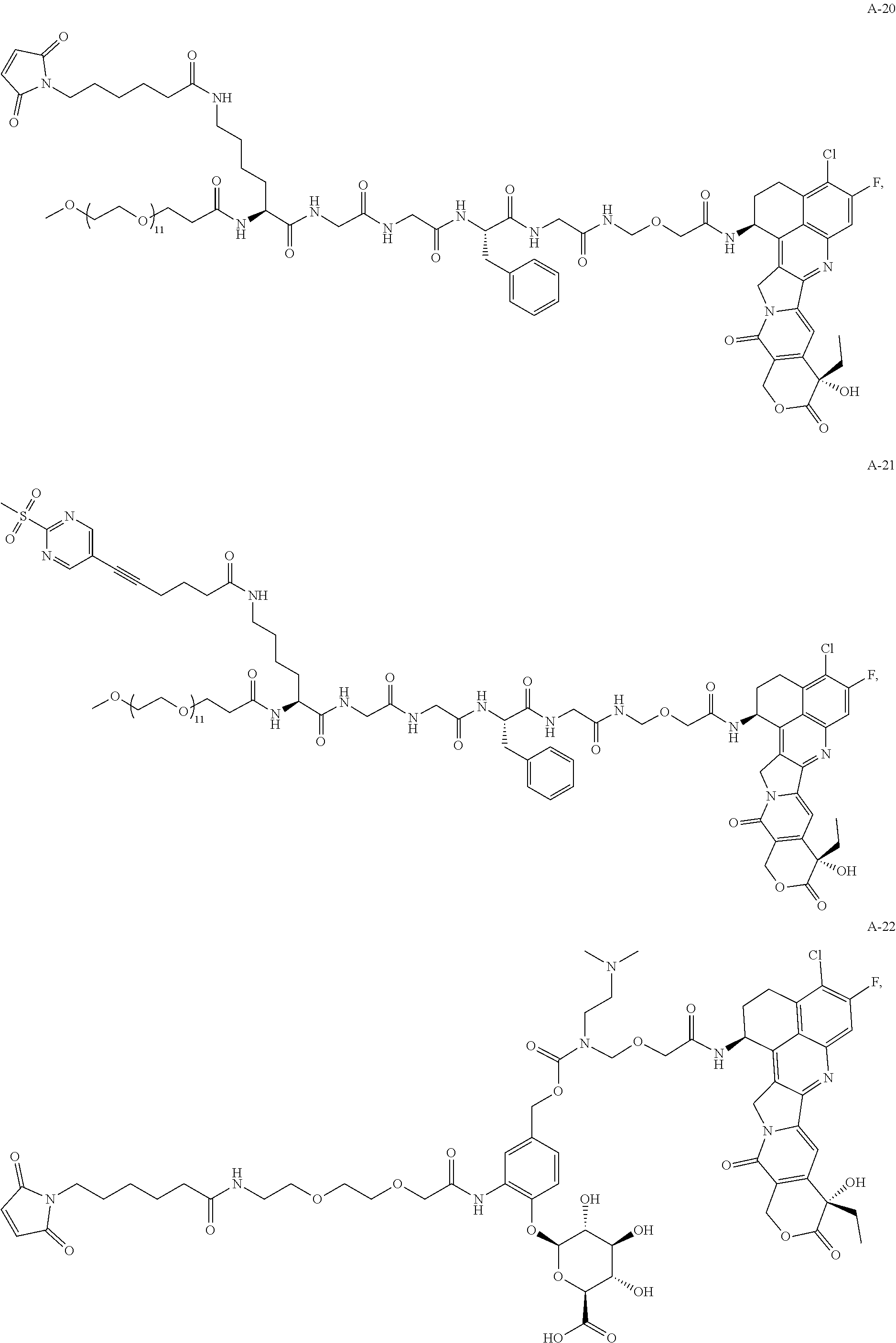

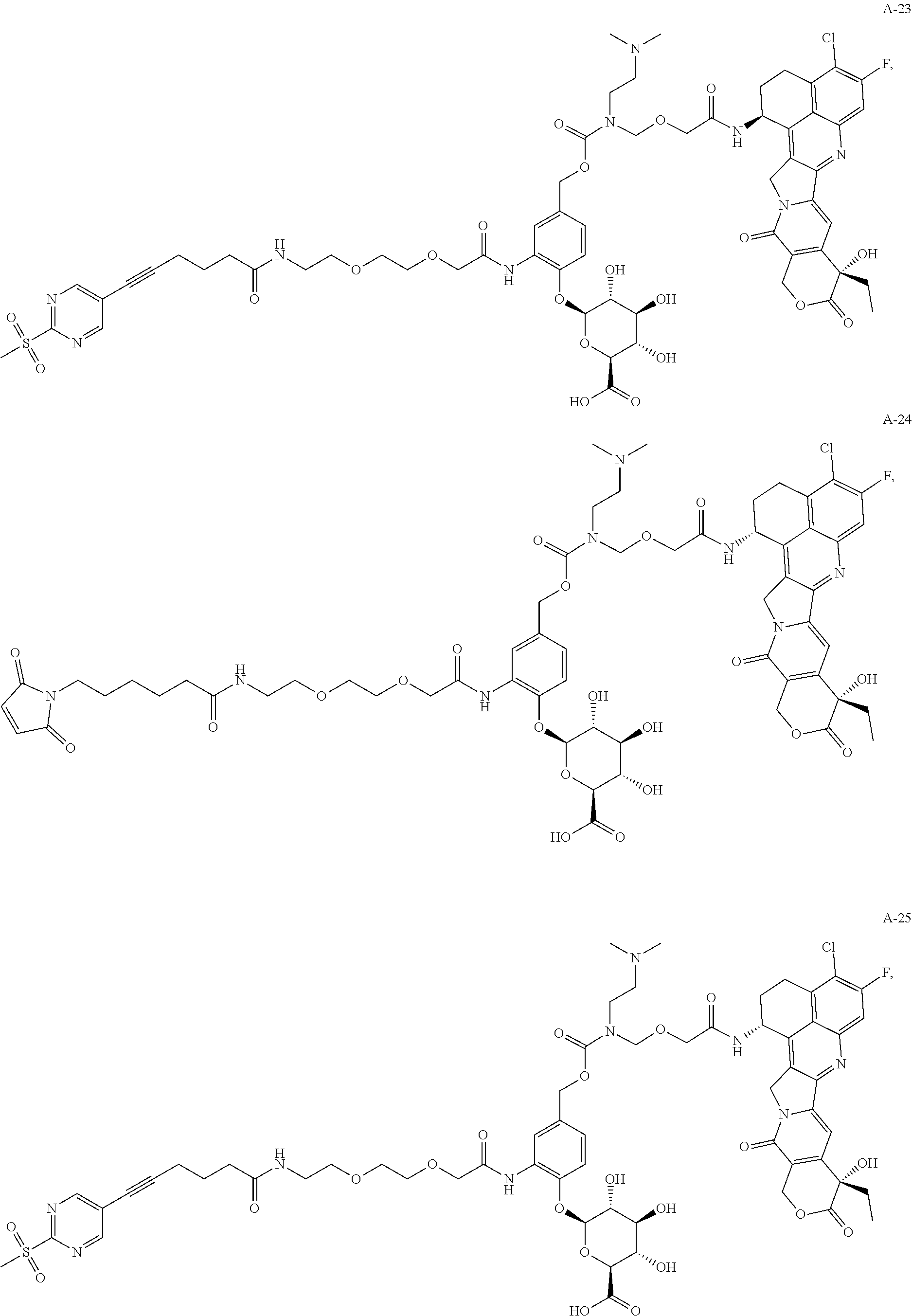

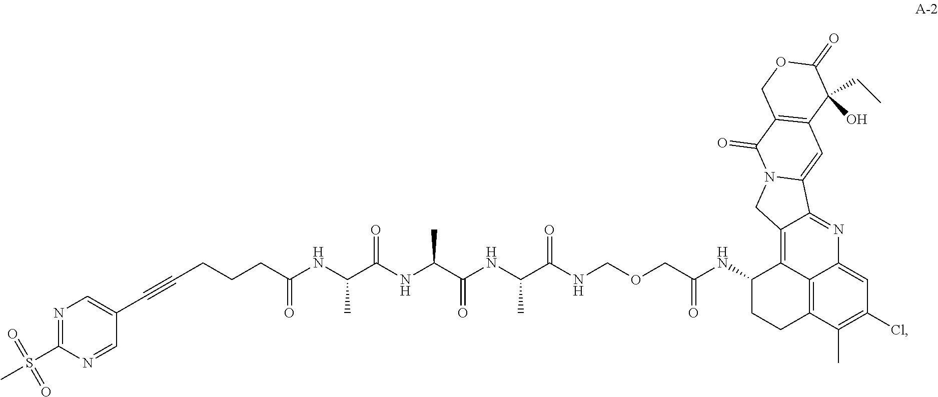 [SMILES: CC[C@@]1(O)C(=O)OCc2c1cc1n(c2=O)Cc2c-1nc1cc(Cl)c(C)c3c1c2[C@@H](NC(=O)COCNC(=O)[C@H](C)NC(=O)[C@H](C)NC(=O)[C@H](C)NC(=O)CCCC#Cc1cnc(S(C)(=O)=O)nc1)CC3]

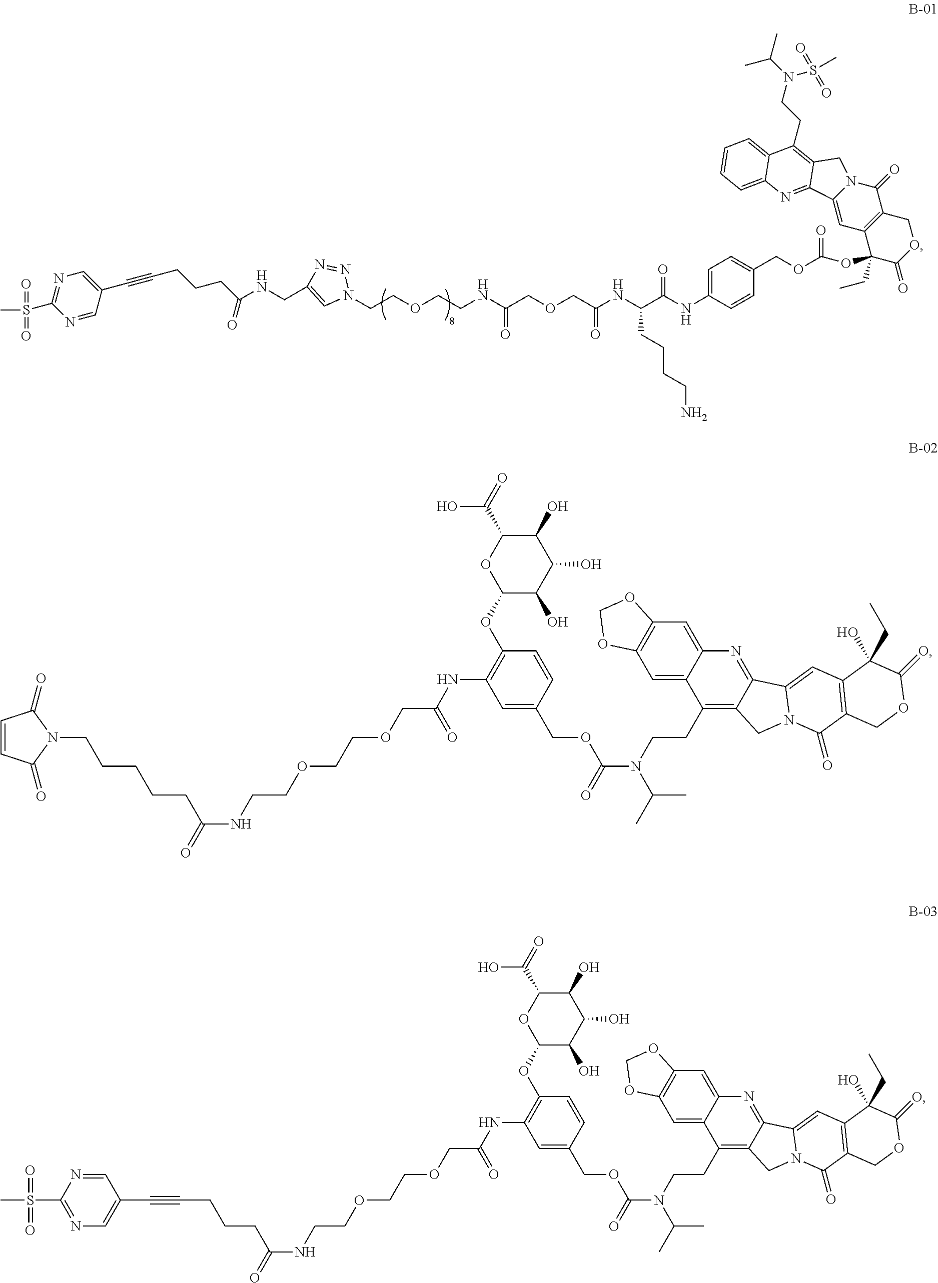

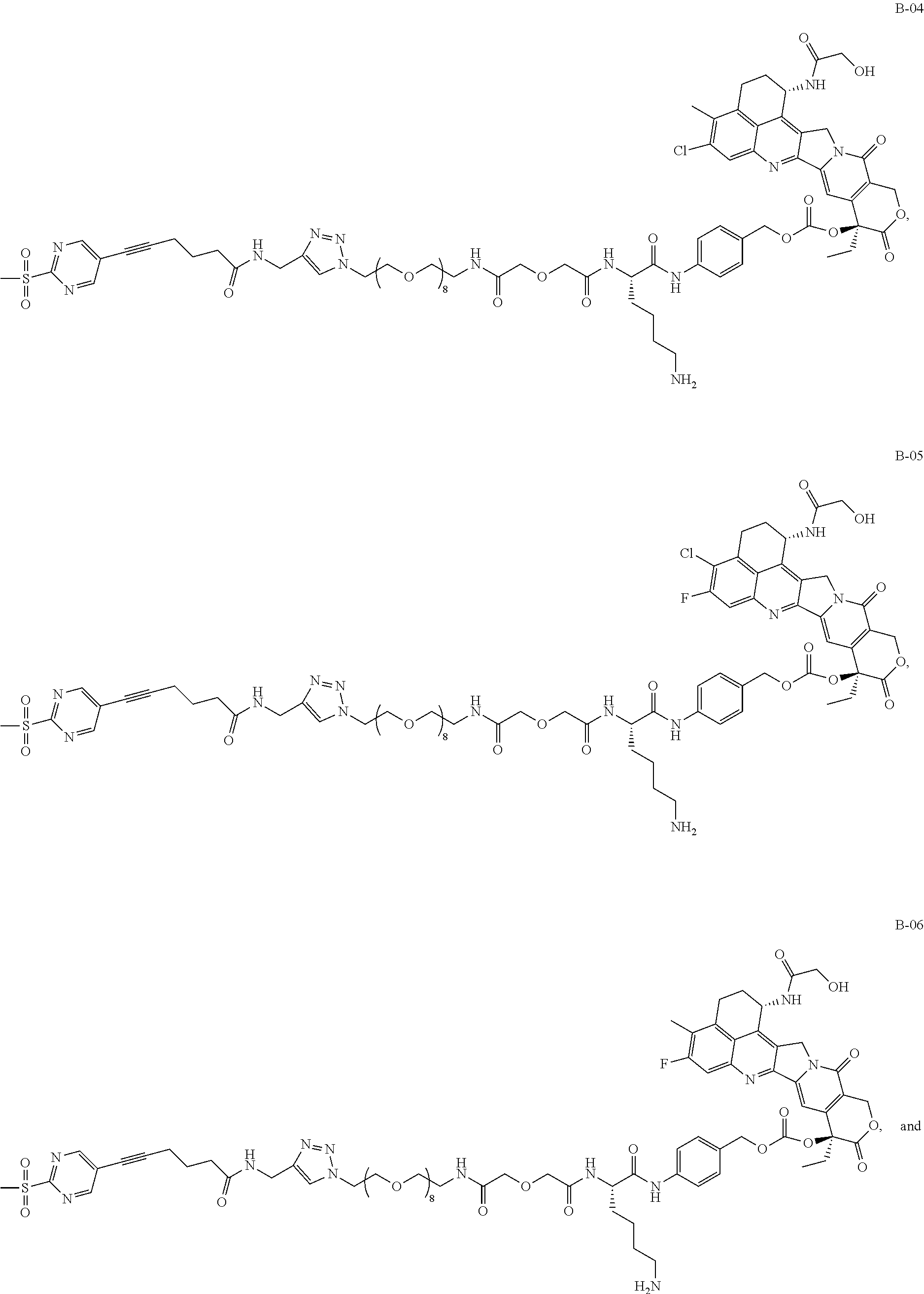

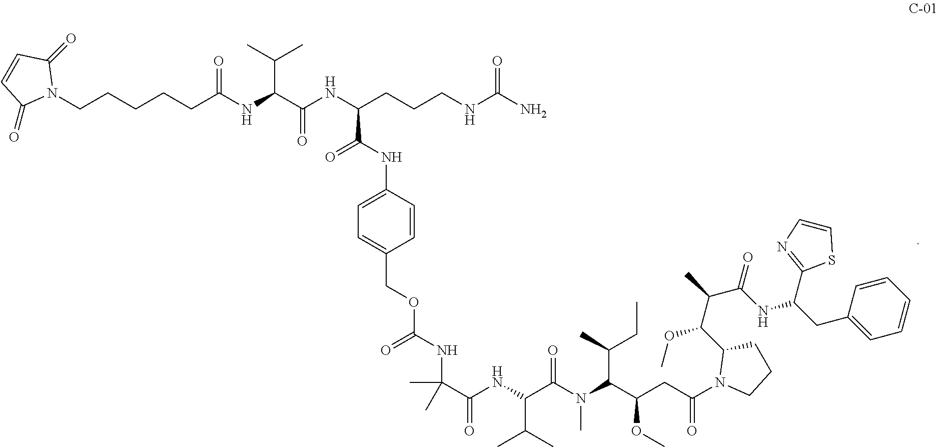

### Pharmaceutical Compositions

In another aspect, the present application provides a pharmaceutical composition, comprising the antibody-drug conjugate according to any one of the foregoing, optionally the drug-linker according to any one of the foregoing, and one or more pharmaceutic adjuvant(s).

The antibody-drug conjugate described herein is typically formulated in a unit injectable form together with a pharmaceutically acceptable parenteral vehicle for parenteral use, for example bolus injection, intravenous injection, intratumoral injection and the like. Optionally, the antibody-drug conjugate with desired purity is mixed with a pharmaceutically acceptable diluent, carrier, excipient or stabilizer in the form of lyophilizate or solution (Remington's Pharmaceutical Sciences (1980) 16th edition, Osol, A. Ed.). The antibody-drug conjugate, or the pharmaceutical composition comprising the antibody-drug conjugate described herein may be administered by any route appropriate for the individual to be treated.

### Use

The antibody-drug conjugate, drug-linker, composition or the pharmaceutical composition thereof described herein may be used for treating various diseases or disorders, for example cancers with high expression of CEACAM5, including solid tumors, for example rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer), and the like, and leukemia, for example, acute myeloid leukemia.

In certain embodiments, the cancer is a cancer of the rectum, colon, stomach, pancreas, esophagus, lung, head and neck, breast, bladder, uterine, prostate, skin, testicle, ovary, liver, thyroid, kidney, or brain. In certain embodiments, the cancer is a lymphoma, a melanoma or a leukemia.

In one embodiment, the antibody-drug conjugate, drug-linker, composition or the pharmaceutical composition thereof described herein may be used for treating rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor or ovarian serous cystadenocarcinoma.

In one embodiment, the antibody-drug conjugate, drug-linker, composition or the pharmaceutical composition thereof described herein may be used for treating a cancer of the rectum, colon, stomach, pancreas, esophagus, lung, head and neck, breast, bladder, uterine, prostate, skin, testicle, ovary, liver, thyroid, kidney, or brain. In certain embodiments, the cancer is a lymphoma, a melanoma or a leukemia.

In one embodiment, the antibody-drug conjugate, drug-linker, composition or the pharmaceutical composition thereof described herein may be used for treating colorectal cancer, gastric cancer or GEJ adenocarcinoma (adenocarcinomas of the esophagogastric junction), non-small cell lung cancer, pancreatic ductal adenocarcinoma, or esophageal carcinoma.

Therefore, the present application provides use of the antibody-drug conjugate, drug-linker, composition, or pharmaceutical composition comprising the same in preparation of a drug for treating cancer with high expression of CEACAM5.

The present application also provides the antibody-drug conjugate, drug-linker, composition or pharmaceutical composition comprising the same, for use in treating cancer with high expression of CEACAM5.

Meanwhile, the present application also provides a method for treating cancers with high expression of CEACAM5, which includes steps of: administering an effective amount of the antibody-drug conjugate, drug linker, composition or pharmaceutical composition comprising the same according to any one of the foregoing to a subject in need.

In particular embodiments, the cancer comprises solid tumors. In a further embodiment, the cancer is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer) and the like.

### BRIEF DESCRIPTION OF THE DRAWINGS

FIG. 1A provides a plot depicting binding affinity of an anti-human CEACAM5 antibody and ADC to gastric cancer cells MKN45.

FIG. 1B provides a plot depicting binding affinity of an anti-human CEACAM5 antibody and ADC to colon cancer cells LS174T.

FIG. 1C provides a plot depicting binding of monkey CEACAM5 antibody and ADC to monkey CEACAM5.

FIG. 2A provides a plot depicting tumor killing activity in MKN45 tumor cells by ADC drugs.

FIG. 2B provides a plot depicting tumor killing activity in SK-CO-1 tumor cells by ADC drugs.

### DETAILED DESCRIPTION

### Definitions

Unless otherwise defined hereinafter, all technical and scientific terms as used herein have the same meaning as commonly understood by those skilled in the art. References to techniques used herein are intended to refer to techniques commonly understood in the art, including those variations of the techniques or replacements of equivalent techniques that are obvious to those skilled in the art. Moreover, the laboratory operation steps such as genomics, nucleic acid chemistry, and molecular biology used herein are all routine steps widely used in the respective fields. While the following terms are believed to be well understood by those skilled in the art, the following definitions are set forth to better explain the present invention.

The term “antibody” generally refers to immunoglobulin molecules that are composed of two pairs of polypeptide chains (each pair having one light chain (LC) and one heavy chain (HC)). The light chain of the antibody can be classified as kappa (κ) and lambda (2) light chains. The heavy chain can be classified as u, 8, y, a or &, and the isotypes of the antibody are defined as IgM, IgD, IgG, IgA, and IgE, respectively. In the light chain and heavy chain, the variable regions and the constant regions are linked by a “J” region having about 12 or more amino acids, and the heavy chain further comprises a “D” region having about 3 or more amino acids. Each heavy chain is comprised of the heavy chain variable region (VH) and the heavy chain constant region (CH). The heavy chain constant region is composed of 3 domains (CH1, CH2, and CH3).

Each light chain comprises the light chain variable region (VL) and the light chain constant region (CL). The light chain constant region is composed of one domain CL. The constant domains do not directly participate in the binding between the antibody and the antigen, but show a variety of effector functions, such as mediating the binding of immunoglobulins to host tissues or factors, including various cells of the immune system (like effector cells) and a first component (Clq) of a classical complement system. The V_{H} and V_{L} regions may also be subdivided into regions with high variability (referred to as complementarity determining regions (CDRs)), with more conservative regions interspersed therebetween, referred to as framework regions (FRs). Each V_{H} and each V_{L} are composed of 3 CDRs and 4 FRs arranged in the following order: FR1, CDR1, FR2, CDR2, FR3, CDR3 and FR4 from the amino terminus to the carboxy terminus. The variable regions (V_{H} and VL) of each heavy chain/light chain pair form antigen-binding sites, respectively. The allocation of amino acids in each region or domain is according to various numbering system known in the art.

The term “antibody” further includes an embodiment in which the heavy chain constant region comprises a C-terminal lysine, or lacks a C-terminal lysine or a C-terminal glycine-lysine dipeptide. The term further includes an embodiment in which an N-terminal amino acid of the antibody variable region has been cyclized into pyroglutamic acid or pyroglutamate. Therefore, in a composition comprising the antibody disclosed herein, each antibody therein may independently comprise a C-terminal lysine, lack a C-terminal lysine, lack a C-terminal glycine-lysine dipeptide, and/or comprise an N-terminal glutamine or glutamic acid, or an N-terminal amino acid is cyclized into pyroglutamic acid or pyroglutamate.

The light and heavy chains of the antibody may comprise one or more cysteine residues having reactive sulfhydryl groups. These sulfhydryl groups may oxidatively react to form one or more inter- and/or intra-chain disulfide bonds, which upon reduction may revert back to the reactive sulfhydryl groups. The light and heavy chains of the antibody may also comprise one or more lysine residues having free amino groups.

The term “complementarity determining region” or “CDR” refers to amino acid residues responsible for antigen-binding in the variable region of the antibody. The variable regions of the heavy chain and the light chain each comprises three CDRs, named as CDR1, CDR2 and CDR3. The exact boundaries of these CDR can be defined according to various numbering systems known in the art, e.g., as defined according to a Kabat numbering system (Kabat et al., Sequences of Proteins of Immunological Interest, 5th Ed. Public Health Service, National Institutes of Health, Bethesda, Md., 1991), a Chothia numbering system (Chothia & Lesk (1987) J. Mol. Biol. 196:901-917; Chothia et al. (1989) Nature 342:878-883), an IMGT numbering system (Lefranc et al., Dev. Comparat. Immunol. 27:55-77, 2003), or an AbM numbering system (Martin ACR, Cheetham J C, Rees A R (1989) Modelling antibody hypervariable loops: A combined algorithm. Proc Natl Acad Sci USA 86:9268-9272). For a given antibody, those skilled in the art will easily identify the CDRs defined according to each numbering system. Moreover, the correspondence between different numbering systems is well-known to those skilled in the art (like, referring to Lefranc et al., Dev. Comparat. Immunol. 27:55-77, 2003).

In the present invention, the CDR in the antibody or antigen-binding fragment can be determined according to various numbering system known in the art, e.g., as determined according to the Kabat, Chothia, IMGT or AbM numbering system. In certain embodiments, the CDR contained in the antibody or antigen-binding fragment is defined according to the Chothia numbering system.

The following general rules disclosed in www.bioinf.org.uk: Prof. Andrew C. R. Martin's Group and reproduced below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L52 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire amino acid sequence of the V_{H} is commonly numbered according to Kabat while the three CDRs within the variable region may be defined according to any one of the aforementioned numbering schemes. In particular embodiments, the numbering of the amino acid positions in the V_{H} may be sequential beginning with amino acid position 1 and continuing sequentially to the end of the sequence or according to Kabat. Unless specified otherwise, the amino acid positions in the V_{H} and V_{L} herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

The term “framework region” or “FR” residue refers to those amino acid residues in the variable region of the antibody other than the CDR residues as defined above.

The term “antigen-binding fragment” of antibody refers to polypeptide of a fragment of the antibody, for example polypeptide of a fragment of a full-length antibody, which retains the capability of specifically binding to the same antigen to which the full-length antibody is bound, and/or competes for specific binding to the antigen with the full-length antibody, which is also referred to as the “antigen-binding fragment”. Generally, refer to Fundamental Immunology, Ch. 7 (Paul, W. ed., 2nd Edition, Raven Press, N.Y. (1989), which is incorporated herein by reference for all purposes in its entirety. The antigen-binding fragment of the antibody may be produced by a recombinant DNA technology or by enzymatic or chemical cleavage of the complete antibody. Non-limiting examples of the antigen-binding fragment include a Fab fragment, a Fab′ fragment, aF(ab)′2 fragment, aF(ab)′3 fragment, Fd, Fv, scFv, di-scFv, (scFv) 2, disulfide bond stabilized Fv protein (“dsFv”), a single-domain antibody (sdAb, nanobody) and similar polypeptides, which comprise at least a part of the antibody sufficient to enable antigen-specific binding ability to the polypeptide. Engineered variants of the antibodies are summarized in Holliger et al., 2005; Nat Biotechnol, 23:1126-1136.

The term “Fd” means an antibody fragment composed of V_{H} and CH1 domains; the term “dAb fragment” means an antibody fragment composed of V_{H} domains (Ward et al., Nature 341:544 546 (1989)); the term “Fab fragment” means an antibody fragment composed of VL, VH, CL, and CH1 domains; the term “F(ab′) 2 fragment” means an antibody fragment comprising two Fab fragments linked by a disulfide bridge on the hinge region; the term “Fab′ fragment” means a fragment obtained after reducing the disulfide bond linking the two heavy chain fragments in the F(ab′) 2 fragment, and the obtained fragment is composed of a complete Fd fragment (composed of V_{H} and CH1 domains) of the light chain and the heavy chain.

The term “Fv” means an antibody fragment composed of V_{L} and V_{H} domains of a single arm of the antibody. The Fv fragment is generally considered to be a minimal antibody fragment that is capable of forming a complete antigen-binding site. It is generally considered that six CDRs enable the antigen-binding specificity to the antibody. However, one variable region (e.g., an Fd fragment, which contains only three CDRs specific to the antigen) also can be used for recognizing and binding to the antigen, although its affinity may be lower than the complete binding site.

The term “Fc” means an antibody fragment formed by binding second and third constant regions of the first heavy chain of the antibody to second and third constant regions of the second heavy chain through disulfide bonds. The Fc fragment of the antibody has a variety of different functions, but does not participate in antigen-binding.

The term “scFv” refers to a single polypeptide chain comprising VL and VH domains, where the VL and VH are linked by the linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, edited by Roseburg and Moore, Springer-Verlag, New York, Pages 269-315 (1994)). Such scFv molecules may have general structure: NH₂-VL-linker-VH—COOH or NH₂-VH-linker-VL-COOH. Suitable linker in the prior art comprises repeated amino acid sequences GGGGS (SEQ ID NO: 29) or variants thereof. For example, a linker having the amino acid sequence (GGGGS)+ (SEQ ID NO: 30) can be used, and the variants thereof can also be used (Holliger, et al. (1993), Proc. Natl. Acad. Sci. USA 90:6444-6448). Other linkers applicable to the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. In some cases, there may be a disulfide bond between V_{L} and V_{H} of scFv. In certain embodiments, the V_{H} and V_{L} domains may be oppositely positioned to each other in any suitable arrangement. For example, scFv comprising NH₂-VH—VH—COOH and NH₂-VL-VL-COOH.

The term “single-domain antibody (sdAb)” has the meaning generally understood by those skilled in the art, which refers to an antibody fragment composed of a single monomer variable antibody domain (like a single heavy chain variable region), and such antibody fragment retains the capability of specifically binding the same antigen to which the full-length antibody binds (Holt, L. et al., Trends in Biotechnology, 21 (11): 484-490, 2003). The single-domain antibody is also called nanobody.

Each of the above antibody fragments retains the capability of specifically binding to the same antigen to which the full-length antibody binds, and/or competes with the full-length antibody for specific binding to the antigen.

The antigen-binding fragment of the antibody (like the antibody fragment above) can be obtained from a given antibody (like the antibody provided by the present invention) using conventional techniques known to those skilled in the art (like recombinant DNA techniques or enzymatic or chemical cleavage methods), and the antigen-binding fragment of the antibody is screened for specificity in the same manner as for the complete antibody.

The term “murine antibody” refers to an antibody obtained by the following methods: fusing B cells and myeloma cells of immunized mice, screening murine hybrid fusion cells that can both immortalize and secrete antibodies, and then performing screening, antibody preparation and antibody purification; or refers to the antibody secreted by plasma cells formed by the differentiation and proliferation of B cells after the antigen invades the mouse body.

The term “humanized antibody” refers to a genetically engineered non-human antibody of which the amino acid sequence has been modified to increase the sequence homology with a human antibody. Generally, all or part of the CDR regions of a humanized antibody are derived from a non-human antibody (donor antibody), and all or part of the non-CDR regions (for example, variable region FR and/or constant region) are derived from human Immunoglobulin (receptor antibody). The humanized antibody generally retains the expected properties of the donor antibody, including but not limited to, antigen specificity, affinity, reactivity, capability of enhancing immune cell activity, capability of enhancing immune response, and the like. The donor antibody can be mouse, rat, rabbit or non-human primate (like cynomolgus monkey) antibodies with desired properties (for example, antigen specificity, affinity, reactivity, capability of enhancing immune cell activity and/or capability of enhancing immune response).

As used herein, the term “identity” refers to the matching of sequences between two polypeptides or between two nucleic acids. When a position in two to-be- compared sequences is occupied by a same base or amino acid monomeric subunit (e.g., a position in each of two DNA molecules is occupied by adenine, or a position in each of the two polypeptides is occupied by lysine), the molecules are the same at that position. The “percentage identity” between the two sequences refers to a function obtained by the formula: the number of matched positions common to the two sequences/the number of to-be- compared positions×100. For example, if there are 6 matches in the 10 positions of the two sequences, the two sequences have 60% identity. For example, DNA sequences CTGACT (SEQ ID NO: 31) and CAGGTT (SEQ ID NO: 32) have 50% identity in total (3 matches in the total of 6 positions). Typically, comparison is performed when two sequences are aligned to achieve the maximum identity. Such alignment can be achieved by using, for example, a method proposed by Needleman et al. (1970) J. Mol. Biol. 48:443-453 and conveniently performed by a computer program, such as an Align program (DNAstar, Inc.). The percentage identity between two amino acid sequences can also be determined through E. Meyers and W. Miller (Comput. Appl Biosci. 4:11-17 (1988)) algorithms that are integrated into an ALIGN program (Version 2.0) by using a PAM120 weight residue table, a gap length penalty of 12, and a gap penalty of 4. In addition, the percentage identity between two amino acid sequences can be determined through Needleman and Wunsch (J Mol Biol. 48:444-453 (1970)) algorithms in a GAP program that is integrated into a GCG software package (available at www.gcg.com) by using a Blossum 62 matrix or PAM250 matrix and gap weight of 16, 14, 12, 10, 8, 6 or 4 and length weights of 1, 2, 3, 4, 5 or 6.

The term “conservative replacement” means amino acid replacement that does not adversely affect or change the intended properties of proteins/polypeptides comprising the amino acid sequence. For example, the conservative replacement can be introduced by standard techniques known in the art, for example site-directed mutagenesis and PCR-mediated mutagenesis. Conservative amino acid replacement includes replacement of amino acid residues with amino acid residues having similar side chains, e.g., replacement with residues that are physically or functionally similar to the corresponding amino acid residues (e.g., having similar size, shape, charge, chemical properties, including the capability of forming covalent or hydrogen bonds). Families of the amino acid residues having similar side chains have been defined in the art. These families include amino acids having basic side chains (for example, lysine, arginine, and histidine), acidic side chains (for example, aspartic acid, and glutamic acid), uncharged polar side chains (for example, glycine, asparagine, glutamine, serine, threonine, tyrosine, cysteine, and tryptophan), non-polar side chains (for example, alanine, valine, leucine, isoleucine, proline, phenylalanine, and methionine), B-branched side chains (for example, threonine, valine, and isoleucine), and aromatic side chains (for example, tyrosine, phenylalanine, tryptophan, and histidine). Thus, the corresponding amino acid residue is preferably replaced with another amino acid residue from the same side chain family. Methods for identifying amino acid conservative replacements are well-known in the art (see, for example, Brummell et al., Biochem. 32:1180-1187 (1993); Kobayashi et al. Protein Eng. 12 (10): 879-884 (1999); and Burks et al. Proc. Natl Acad. Set USA 94:412-417 (1997), which is incorporated herein by reference).

The compilation of the twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, the amino acid is generally shown as a single letter or three letter abbreviation well known in the art. For example, alanine may be shown as A or Ala.

The term “pharmaceutically acceptable carrier and/or excipient” refers to a carrier and/or excipient that is pharmacologically and/or physiologically compatible with the subject and the active ingredient. It is well known in the art (see e.g. Remington's Pharmaceutical Sciences. Edited by Gennaro AR, 19th ed. Pennsylvania: Mack Publishing Company, 1995), and includes but is not limited to: pH adjusters, surfactants, adjuvants, ionic strength enhancers, diluents, agents to maintain osmotic pressure, agents to delay absorption, preservatives. For example, pH adjusting agents include, but are not limited to, phosphate buffers. Surfactants include but are not limited to cationic, anionic or nonionic surfactants such as Tween-80. Ionic strength enhancers include, but are not limited to, sodium chloride. Preservatives include, but are not limited to, various antibacterial and antifungal agents, such as parabens, chlorobutanol, phenol, sorbic acid, and the like. Agents to maintain osmotic pressure include, but are not limited to, sugars, NaCl, and the like. Agents that delay absorption include, but are not limited to, monostearates and gelatin. Diluents include, but are not limited to, water, aqueous buffers (such as buffered saline), alcohols and polyols (such as glycerol), and the like. Preservatives include, but are not limited to, various antibacterial and antifungal agents, such as thimerosal, 2-phenoxyethanol, parabens, chlorobutanol, phenol, sorbic acid, and the like. Stabilizer has the meaning generally understood by those skilled in the art, and it can stabilize the desired activity of the active ingredient in the medicine, including but not limited to sodium glutamate, gelatin, SPGA, sugars (such as sorbitol, mannitol, starch, sucrose, lactose, dextran, or glucose), amino acids (such as glutamic acid, glycine), proteins (such as dry whey, albumin or casein) or their degradation products (such as lactalbumin hydrolyzate), etc.

The terms “including”, “comprising”, “having”, “containing” or “involving” and other variations thereof herein are inclusive or open-ended and do not exclude other unlisted elements or method steps.

The term “effective amount” refers to the amount sufficient to achieve a desired clinical effect in an individual being treated. For instance, this may be the amount necessary to alleviate any particular disease symptom or inhibit or reduce the severity of a disease, or its complications in an individual. Determining such an effective amount is well within the capability of those skilled in the art. For example, amounts effective for therapeutic use will depend on the severity of the disease to be treated, the general state of the patient's own immune system, the general condition of the patient such as age, weight and sex, the mode of administration of the drug, and other treatments administered concomitantly etc. A therapeutically effective amount of an ADC may vary according to the following factors: The severity of the disease to be treated, the general state of the patient's own immune system, the general condition of the patient such as age, weight and sex, the mode of administration of the drug, and other treatments administered at the same time, etc.

The term “treatment” refers to a method implemented to obtain a beneficial or desired clinical outcome in a subject (human or animal individual) that is exhibiting a disease symptom or diagnosed as having a disease (“in need thereof”). For the purposes of this invention, a beneficial or desired clinical outcome includes, but is not limited to, relief of symptoms, reduction of the extent of the disease, stabilization (i.e., no longer worsening) of the disease state, delay or slowing of the progression of the disease, amelioration or palliation of the disease status, and relief of symptoms (whether partial or total), whether detectable or not. Additionally, “treating” can also refer to prolonging survival as compared to expected survival if not receiving treatment.

As used herein, the term “subject” refers to a mammal, such as a primate mammal, such as a human. In certain embodiments, the subject (e.g., a human) has a tumor, or is at risk of having a disease as described above.

The terms “cancer” and “tumor” are used interchangeably and refer to a broad class of diseases characterized by the uncontrolled growth of abnormal cells in the body. Unregulated cell division may lead to the formation of malignant tumors or cells that invade adjacent tissues and may metastasize to distant sites in the body through the lymphatic system or bloodstream. Cancer includes benign and malignant cancers as well as dormant tumors or micrometastases.

The term “alkyl” means a group obtained by the loss of one hydrogen atom from a straight or branched hydrocarbon group, for example “C₁₋₂₀ alkyl”, “C₁₋₁₀ alkyl”, “C₁₋₆ alkyl”, “C₁₋₄ alkyl”, and “C₁₋₃ alkyl”. Specific examples include but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” means a group obtained by the loss of two hydrogen atoms from a straight or branched hydrocarbon group, for example “C₁₋₂₀ alkylene”, “C₁₋₁₀ alkylene”, “C₃₋₁₀ alkylene”, “C₅₋₈ alkylene”, “C₁₋₆ alkylene”, “C₁₋₄ alkylene”, and “C₁₋₃ alkylene”. Specific examples include but are not limited to: methylene, ethylene, 1,3-propylene, 1,4-butylene, 1,5-pentylene, 1,6-hexylene, or the like.

The term “alkenylene” refers to a divalent group obtained by losing two hydrogen atoms from a linear or branched hydrocarbon group containing at least one carbon-carbon double bond, including, for example, “C₂₋₂₀ alkenylene”, “C₃₋₁₀ alkenylene”, “C₅₋₈ alkenylene”, etc. Examples include, but are not limited to: ethenylene, 1-propenylene, 2-propenylene, 1-butenylene, 2-butenylene, 1,3-butadienylene, 1-pentenylene, 2-pentenylene, 3-pentenylene, 1,3-pentadienylene, 1,4-pentadienylene, 1-hexenylene, 2-hexenylene, 3-hexenylene, 1,4-hexadienylene and the like.

The term “alkynylene” refers to a divalent group obtained by the loss of two hydrogen atoms from a linear or branched hydrocarbon group containing at least one carbon-carbon triple bond, including, for example, “C₂₋₂₀ alkynylene”, “C₃₋₁₀ alkynylene”, and “C₅₋₈ alkynylene”. Examples include, but are not limited to: ethynylene, 1-propynylene, 2-propynylene, 1-butynylene, 2-butynylene, 1,3-butadiynylene, 1-pentynyl, 2-pentynyl, 3-pentynyl, 1,3-pentadiynylene, 1,4-pentadiynylene, 1-hexynylene, 2-hexynylene, 3-hexynylene, 1,4-hexadiynylene, etc.

The term “heteroalicyclic ring” refers to a saturated or partially saturated cyclic structure containing at least one ring member selected from N, O and S. Specific examples include, but are not limited to, 5-6 membered aliphatic heterocyclic rings, 5-6 membered nitrogen- containing aliphatic heterocyclic rings, 5-6 membered oxygen- containing aliphatic heterocyclic rings, etc., for example tetrahydrofuran, pyrrolidine, piperidine, tetrahydropyran, etc.

The term “heteroaromatic ring” refers to an aromatic ring structure containing at least one ring member selected from N, O and S. Specific examples include but are not limited to 5-6 membered aromatic heterocyclic rings, 5-6 membered nitrogen- containing aromatic heterocyclic rings, 5-6 membered oxygen- containing aromatic heterocyclic rings, etc., for example furan, thiophene, pyrrole, thiazole, isothiazole, thiadiazole, oxazole, isoxazole, oxadiazole, imidazole, pyrazole, 1,2,3-triazole, 1,2,4-triazole, 1,2,3-oxadiazole, 1,2,4-oxadiazole, 1,2,5-oxadiazole, 1,3,4-oxadiazole, pyridine, pyrimidine, pyridazine, pyrazine, 1,2,3-triazine, 1,3, 5-triazine, 1,2,4,5-tetrazine, etc.

The term “aromatic ring system” refers to a monocyclic or polycyclic ring system comprising at least one aromatic ring (like benzene ring) or heteroaromatic ring (like pyrimidine ring), two or more aromatic rings and/or heteroaromatic aromatic rings can form fused rings or be connected by single bonds (for example dipyrimidinylphenyl, etc.), and the aromatic ring system can be divalent or higher valence state (like trivalent or tetravalent), for example 5-20 membered aromatic ring system.

As used herein, “Cit” is abbreviation for Citrulline.

As used herein, the term “about” or “approximately” when used in conjunction with a numerical variable generally means that the value of the variable is within experimental error (e.g., within a 95% confidence interval for the mean) or within +10%.

It should be noted that if there is a discrepancy between a depicted structure and a name for that structure, the depicted structure is to be accorded more weight.

### Antibody-Drug Conjugate

In one aspect, the present application provides an antibody-drug conjugate having a structure shown as a formula: HA-(Y-M-L-E-D)ₓ′,
wherein HA-(Y-represents an antibody or antigen-binding fragment thereof that specifically binds to CEACAM5, where-(Y-represents a linkage from an amino acid residue of the antibody or antigen-binding fragment thereof to M;
M-L-E is a linker connecting -(Y-to D, wherein M is the portion of the linker connecting to Y, L is the central portion of the linker, and E is the portion of the linker connecting to D;
D is a cytotoxic drug; and
x′ is selected from 1-20.

In one embodiment, the amino acid residue of the antibody or antigen-binding fragment thereof is a cysteine, lysine, serine or threonine residue.

In one embodiment, HA-(Y-(or HA-Y-) is HA-(S-, HA-(NH—, or HA-(O-(or HA-S, HA-NH, or HA-O—).

In one embodiment, -(Y— is -(S-, -(NH— or -(O—.

In one embodiment, x′ is 1-16, 1-8, 4-8, 4 or 8.

In one embodiment, x′ is 1-16.

In one embodiment, x′ is 1-8.

In one embodiment, x′ is 4-8.

In one embodiment, x′ is 8.

In some embodiments, the antibody-drug conjugates provided herein comprises a humanized anti-CEACAM5 antibody. In some embodiments, the antibody has excellent binding activity to CEACAM5. In some embodiments, the antibody-drug conjugates provided herein can efficiently deliver drugs to CEACAM5.

In some embodiments, the antibody or antigen-binding fragment thereof comprises:
(1) the following heavy chain variable regions (VH) and/or light chain variable regions (VL), wherein CDRs are defined according to a Chothia numbering system: a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement.

In some embodiments, the antibody or antigen-binding fragment thereof comprises: (1) the following heavy chain variable regions (VH) and/or light chain variable regions (VL):
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL):
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL):
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
or, regions (VL):
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement.

In some embodiments, the antibody or antigen-binding fragment thereof comprises:
(1) the following heavy chain variable regions (VH) and/or light chain variable regions (VL):
a heavy chain variable region (VH) comprising the following 3 CDRs: CDR-H1 having an amino acid sequence as set forth in SEQ ID NO: 14, CDR-H2 having an amino acid sequence as set forth in SEQ ID NO: 18, and CDR-H3 having an amino acid sequence as set forth in SEQ ID NO: 6; and/or, a light chain variable region (VL) comprising the following 3 CDRs: CDR-L1 having an amino acid sequence as set forth in SEQ ID NO: 7, CDR-L2 having an amino acid sequence as set forth in SEQ ID NO: 8, and CDR-L3 having an amino acid sequence as set forth in SEQ ID NO: 9;
(2) the following heavy chain variable region (VH) and/or light chain variable region (VL):
a heavy chain variable region (VH) comprising the following 3 CDRs: CDR-H1 having an amino acid sequence as set forth in SEQ ID NO: 15, CDR-H2 having an amino acid sequence as set forth in SEQ ID NO: 19, CDR-H3 having an amino acid sequence as set forth in SEQ ID NO: 6; and/or, a light chain variable region (VL) comprising the following 3 CDRs: CDR-L1 having an amino acid sequence as set forth in SEQ ID NO: 7, CDR-L2 having an amino acid sequence as set forth in SEQ ID NO: 8, and CDR-L3 having an amino acid sequence as set forth in SEQ ID NO: 9;
(3) the following heavy chain variable regions (VH) and/or light chain variable regions (VL):
a heavy chain variable region (VH) comprising the following 3 CDRs: CDR-H1 having an amino acid sequence as set forth in SEQ ID NO: 10, CDR-H2 having an amino acid sequence as set forth in SEQ ID NO: 17, and CDR-H3 having an amino acid sequence as set forth in SEQ ID NO: 11; and/or, a light chain variable region (VL) comprising the following 3 CDRs: CDR-L1 having an amino acid sequence as set forth in SEQ ID NO: 12, CDR-L2 having an amino acid sequence as set forth in SEQ ID NO: 13, and CDR-L3 having an amino acid sequence as set forth in SEQ ID NO: 9;
or
(4) the following heavy chain variable regions (VH) and/or light chain variable regions (VL):
a heavy chain variable region (VH) comprising the following 3 CDRs: CDR-H1 having an amino acid sequence as set forth in SEQ ID NO: 5, CDR-H2 having an amino acid sequence as set forth in SEQ ID NO: 16, and CDR-H3 having an amino acid sequence as set forth in SEQ ID NO: 6; and/or, a light chain variable region (VL) comprising the following 3 CDRs: CDR-L1 having an amino acid sequence as set forth in SEQ ID NO: 7, CDR-L2 having an amino acid sequence as set forth in SEQ ID NO: 8, and CDR-L3 having an amino acid sequence as set forth in SEQ ID NO: 9.

In some embodiments, the antibody or antigen-binding fragment thereof comprises (1) a heavy chain variable region (VH) comprising the following 3 CDRs: CDR-H1 having the amino acid sequence as set forth in SEQ ID NO: 14, CDR-H2 having the amino acid sequence as set forth in SEQ ID NO: 18, and CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises (2) a heavy chain variable region (VH) comprising the following 3 CDRs: CDR-H1 having the amino acid sequence as set forth in SEQ ID NO: 15, CDR-H2 having the amino acid sequence as set forth in SEQ ID NO: 19, CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises (3) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 17, CDR-H3 having the amino acid sequence as set forth in SEQ ID NO: 11; and a light chain variable region (VL) comprising the following 3 CDRs: CDR-L1 having the amino acid sequence as set forth in SEQ ID NO: 12, CDR-L2 having the amino acid sequence as set forth in SEQ ID NO: 13, and CDR-L3 having the amino acid sequence as set forth in SEQ ID NO: 9.

In some embodiments, the antibody or antigen-binding fragment thereof comprises (4) a heavy chain variable region (VH) comprising the following 3 CDRs: CDR-H1 having the amino acid sequence as set forth in SEQ ID NO: 5, CDR-H2 having the amino acid sequence as set forth in SEQ ID NO: 16, and CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises a V_{H} having the amino acid sequence as set forth in SEQ ID NO: 1 or a variant thereof, and a VL having the amino acid sequence as set forth in SEQ ID NO: 2 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived; and preferably, the replacement is a conservative replacement.

In some embodiments, the antibody or antigen-binding fragment thereof comprises a V_{H} having the amino acid sequence as set forth in SEQ ID NO: 1, and a V_{L} having the amino acid sequence as set forth in SEQ ID NO: 2.

In some embodiments, the antibody or antigen-binding fragment thereof comprises a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence of SEQ ID NO: 1, respectively; and a light chain variable region (VL) comprising a CDR-L1, a CDR-L2, and a CDR-L3 comprising amino acid sequences of the CDR-L1, the CDR-L2, and the CDR-L3 as set forth in the V_{L} having the amino acid sequence of SEQ ID NO: 2, respectively.

In some embodiments, the antibody or antigen-binding fragment thereof further comprises:
(a) a human immunoglobulin heavy chain constant region (CH) or a variant thereof, wherein the variant has replacement, deletion or addition of one, two, or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human immunoglobulin light chain constant (CL) or a variant, wherein the variant has replacement, deletion or addition of one, two, or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In some embodiments, the heavy chain constant region is an IgG heavy chain constant region, for example an IgG1, IgG2, IgG3 or IgG4 heavy chain constant region, for example a human IgG1 heavy chain constant region or a human IgG4 heavy chain constant region. In some embodiments, the antibody or antigen-binding fragment thereof comprises a heavy chain constant region (CH) shown as SEQ ID NO: 3 or a variant thereof, and the variant has a conservative replacement of up to 20 amino acids (for example, a conservative replacement of up to 15, up to or up to 5 amino acids; for example, a conservative replacement of 1, 2, 3, 4 or 5 amino acids) as compared to SEQ ID NO: 3.

In some embodiments, the light chain constant region is a human IgG1 light chain constant region. In some embodiments, the antibody or antigen-binding region thereof comprises a light chain constant region (CL) shown as SEQ ID NO: 4 or a variant thereof, and the variant has a conservative replacement of up to 20 amino acids (for example, a conservative replacement of up to 15, up to 10 or up to 5 amino acids; for example, a conservative replacement of 1, 2, 3, 4 or 5 amino acids) as compared to SEQ ID NO: 4.

In some embodiments, the antibody or antigen-binding fragment thereof comprises a heavy chain constant region (CH) shown as SEQ ID NO: 3, and a light chain constant region (CL) shown as SEQ ID NO: 4.

In some embodiments, the antibody or antigen-binding fragment thereof comprises a heavy chain comprising a V_{H} having the amino acid sequence as set forth in SEQ ID NO: 1 and heavy chain constant region (CH) having the amino acid sequence as set forth in SEQ ID NO: 3, and, a light chain comprising a V_{L} having the amino acid sequence as set forth in SEQ ID NO: 2 and light chain constant region (CL) having the amino acid sequence as set forth in SEQ ID NO: 4.

In some embodiments, the antibody or antigen-binding fragment thereof comprises a heavy chain (HC) having the amino acid sequence as set forth in SEQ ID NO: 20, and a light chain (LC) having the amino acid sequence as set forth in SEQ ID NO: 21.

In some embodiments, the antibody or antigen-binding fragment thereof comprises:
(i) a first heavy chain comprising the amino acid sequence of SEQ ID NO: 20;
(ii) a first light chain comprising the amino acid sequence of SEQ ID NO: 21;
(iii) a second heavy chain comprising the amino acid sequence of SEQ ID NO: 20; and
(iv) a second light chain comprising the amino acid sequence of SEQ ID NO: 21.

In certain embodiments, the antibody or antigen-binding fragment thereof comprises a heavy chain (HC) having the amino acid sequence as set forth in SEQ ID NO: 22, and a light chain (LC) having the amino acid sequence as set forth in SEQ ID NO: 21.

In some embodiments, the antibody or antigen-binding fragment thereof comprises:
(i) a first heavy chain comprising the amino acid sequence of SEQ ID NO: 22;
(ii) a first light chain comprising the amino acid sequence of SEQ ID NO: 21;
(iii) a second heavy chain comprising the amino acid sequence of SEQ ID NO: 22; and
(iv) a second light chain comprising the amino acid sequence of SEQ ID NO: 21.

In certain embodiments, the antibody or antigen-binding fragment thereof comprises a heavy chain (HC) having the amino acid sequence as set forth in SEQ ID NO: 23, and a light chain (LC) having the amino acid sequence as set forth in SEQ ID NO: 21.

In some embodiments, the antibody or antigen-binding fragment thereof comprises:
(i) a first heavy chain comprising the amino acid sequence of SEQ ID NO: 23;
(ii) a first light chain comprising the amino acid sequence of SEQ ID NO: 21;
(iii) a second heavy chain comprising the amino acid sequence of SEQ ID NO: 23; and
(iv) a second light chain comprising the amino acid sequence of SEQ ID NO: 21.

In certain embodiments, the antibody or antigen-binding fragment thereof comprises a heavy chain (HC) having the amino acid sequence as set forth in SEQ ID NO: 24, and a light chain (LC) having the amino acid sequence as set forth in SEQ ID NO: 21.

In some embodiments, the antibody or antigen-binding fragment thereof comprises:
(i) a first heavy chain comprising the amino acid sequence of SEQ ID NO: 24;
(ii) a first light chain comprising the amino acid sequence of SEQ ID NO: 21;
(iii) a second heavy chain comprising the amino acid sequence of SEQ ID NO: 24; and
(iv) a second light chain comprising the amino acid sequence of SEQ ID NO: 21.

In certain embodiments of antibody or antibody-drug conjugate disclosed herein, the heavy chain constant domains may comprise a C-terminal lysine or lack either a C-terminal lysine or a C-terminal glycine-lysine dipeptide. In certain embodiments of the antibody or antibody-drug conjugate disclosed herein, the N-terminal amino acid of the antibody variable domains may undergo cyclization to pyroglutamate.

In certain embodiments of the antibody or antibody-drug conjugate disclosed herein, the N-terminal amino acid of the antibody variable domains may undergo cyclization to pyroglutamic acid.

In certain embodiments, the antibody or antigen-binding fragment disclosed herein includes the antibody or antigen-binding fragment that specifically binds the antigen, and may include post-translational modifications thereof (e.g., C-terminal Lysine clipping in the heavy chain, conversion of glutamine or glutamic acid to pyroglutamate or pyroglutamic acid) which may occur when recombinantly expressed in host cells (e.g., CHO cells), or during purification/storage.

Thus, a composition may comprise a population of antibody-drug conjugate species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamate.

In certain embodiments, the composition may comprise a population of antibody-drug conjugate species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamic acid.

Therefore, in particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminal amino acid is glutamine, glutamic acid, pyroglutamate or pyroglutamic acid; or, (iii) antibodies in which the heavy chain C-terminus lacks a lysine residue and the heavy chain N-terminal amino acid is glutamine, glutamic acid, pyroglutamate or pyroglutamic acid.

Therefore, in particular embodiments, the present invention further provides compositions comprising an ADC disclosed herein wherein the predominant ADC species in the composition comprises antibodies in which the heavy chain C-terminus lacks a lysine residue and the heavy chain N-terminal amino acid is pyroglutamate or pyroglutamic acid.

In certain embodiments, the antibody or antigen-binding fragment thereof according to the present invention comprises a heavy chain comprising a V_{H} having the amino acid sequence as set forth in SEQ ID NO: 1.

In certain embodiments, the antibody or antigen-binding fragment thereof according to the present invention comprises a heavy chain comprising a V_{H} having the amino acid sequence as set forth in SEQ ID NO: 1, wherein the N-terminal glutamine of the antibody heavy chain variable region has undergone cyclization to pyroglutamate.

In certain embodiments, the antibody or antigen-binding fragment thereof according to the present invention comprises a heavy chain comprising a V_{H} having the amino acid sequence as set forth in SEQ ID NO: 1, wherein the N-terminal glutamine of the antibody heavy chain variable region has undergone cyclization to pyroglutamic acid.

In certain embodiments, the antibody or antigen-binding fragment thereof according to the present invention comprises a heavy chain comprising a V_{H} having the amino acid sequence set forth in SEQ ID NO: 1 and/or a light chain comprising a V_{L} having the amino acid sequence set forth in SEQ ID NO: 2, and wherein and the N-terminal glutamine of SEQ ID NO: 1 has undergone cyclization to pyroglutamate.

In certain embodiments, the antibody or antigen binding fragment comprises:
(a) a heavy chain having the sequence as set forth in SEQ ID NO: 22 and a light chain having the sequence as set forth in SEQ ID NO: 21;
(b) a heavy chain having the sequence as set forth in SEQ ID NO: 23 and a light chain having the sequence as set forth in SEQ ID NO: 21; or
(c) a heavy chain having the sequence as set forth in SEQ ID NO: 24 and a light chain having the sequence as set forth in SEQ ID NO: 21.

In certain embodiments, the antibody or antigen binding fragment comprises:
(a) a heavy chain consisting of the sequence as set forth in SEQ ID NO: 22 and a light chain consisting of the sequence as set forth in SEQ ID NO: 21;
(b) a heavy chain consisting of the sequence as set forth in SEQ ID NO: 23 and a light chain consisting of the sequence as set forth in SEQ ID NO: 21; or
(c) a heavy chain consisting of the sequence as set forth in SEQ ID NO: 24 and a light chain consisting of the sequence as set forth in SEQ ID NO: 21.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 or a variant thereof and/or a V_{L} having the amino acid sequence set forth in SEQ ID NO: 2 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived.

In certain embodiments, the replacement is a conservative replacement. In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 and/or a V_{L} having the amino acid sequence set forth in SEQ ID NO: 2.

In certain embodiments, the antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(1) a heavy chain comprising a V_{H} sequence shown as SEQ ID NO: 1 and heavy chain constant region (CH) shown as SEQ ID NO: 3, and a light chain comprising a V_{L} sequence shown as SEQ ID NO: 2 and light chain constant region (CL) shown as SEQ ID NO: 4;
(2) a heavy chain (HC) having the amino acid sequence as set forth in SEQ ID NO: 20 and a light chain (LC) having the amino acid sequence as set forth in SEQ ID NO: 21; or
(3) a V_{H} having the amino acid sequence set forth in SEQ ID NO: 1 and a V_{L} having the amino acid sequence set forth in SEQ ID NO: 2.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises (a) a V_{H} having the amino acid sequence set forth in SEQ ID NO: 1 or a variant thereof and/or a V_{L} having the amino acid sequence set forth in SEQ ID NO: 2 or a variant thereof.

In particular embodiments of the antibody, the heavy chain constant domains as disclosed herein may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof may undergo cyclization to pyroglutamate.

In some embodiments of the antibody or antigen binding fragment thereof, the N-terminal amino acid of the antibody or antigen binding fragment thereof may undergo cyclization to pyroglutamic acid.

Thus, in a composition comprising a particular antibody disclosed herein, the composition may comprise a population of antibody species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamate.

In certain embodiments, in a composition comprising a particular antibody disclosed herein, the composition may comprise a population of antibody species wherein each species may independently comprise a C-terminal lysine, lack a C-terminal lysine, lack a C-terminal glycine-lysine dipeptide and/or comprise an N-terminal glutamine or glutamic acid or cyclization of the N-terminal amino acid to pyroglutamic acid.

In the antibody-drug conjugate, the cytotoxic drug may be linked to the antibody or antigen-binding fragment through the linker (like “M-L-E” fragment shown in the present application).

In some embodiments, M is

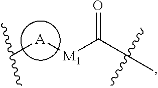

wherein ring A is a 5-6 membered aliphatic heterocyclic ring, or 5-20 membered aromatic ring system, and the aliphatic heterocyclic ring and aromatic ring systems are optionally substituted by one or more members independently selected from the group consisting of oxo (═O), halogen, cyano, amino, carboxyl, mercapto and C₁₋₆ alkyl; and M₁ is selected from a single bond, C₁₋₂₀ alkylene, C₂₋₂₀ alkenylene and C₂₋₂₀ alkynylene.

In some embodiments, M is

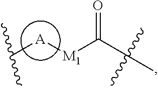

wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted by one or more members independently selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M is

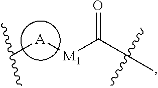

wherein ring A is selected from

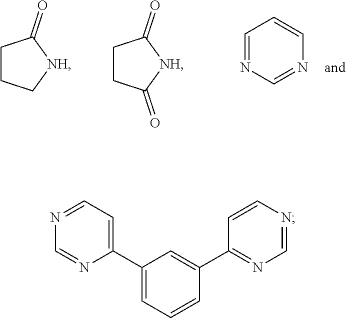 [SMILES: O=C1CCCN1 ; O=C1CCC(=O)N1 ; c1cncnc1 ; c1cc(-c2ccncn2)cc(-c2ccncn2)c1]

and M₁ is selected from a single bond, C₅₋₈ alkylene, C₅₋₈ alkenylene and C₅₋₈ alkynylene.

In some embodiments, M is selected from the following structures:

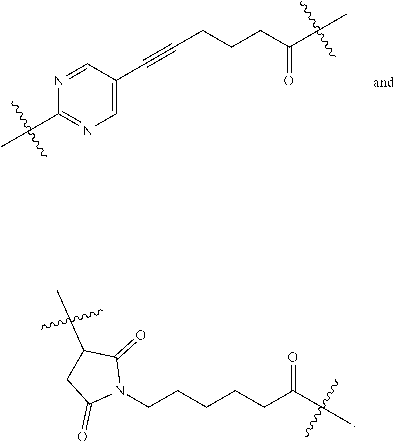 [SMILES: *C(=O)CCCC#Cc1cnc(*)nc1 ; *C(=O)CCCCCN1C(=O)CC(*)C1=O]

In some embodiments, M is:

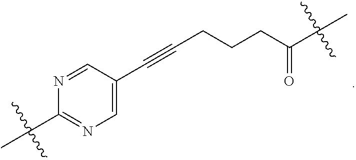 [SMILES: *C(=O)CCCC#Cc1cnc(*)nc1]

In some embodiments, L is selected from a structure comprising one or more of C₁₋₆ alkylene, -N(R′)—, carbonyl, -O—, Val, Cit, Phe, Lys, Lys(COCH₂CH₂ (OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 25), Gly-Phe-Leu-Gly (SEQ ID NO: 26), Gly-Gly-Val-Ala (SEQ ID NO: 27), Gly-Gly-Gly-Gly-Gly (SEQ ID NO: 28),

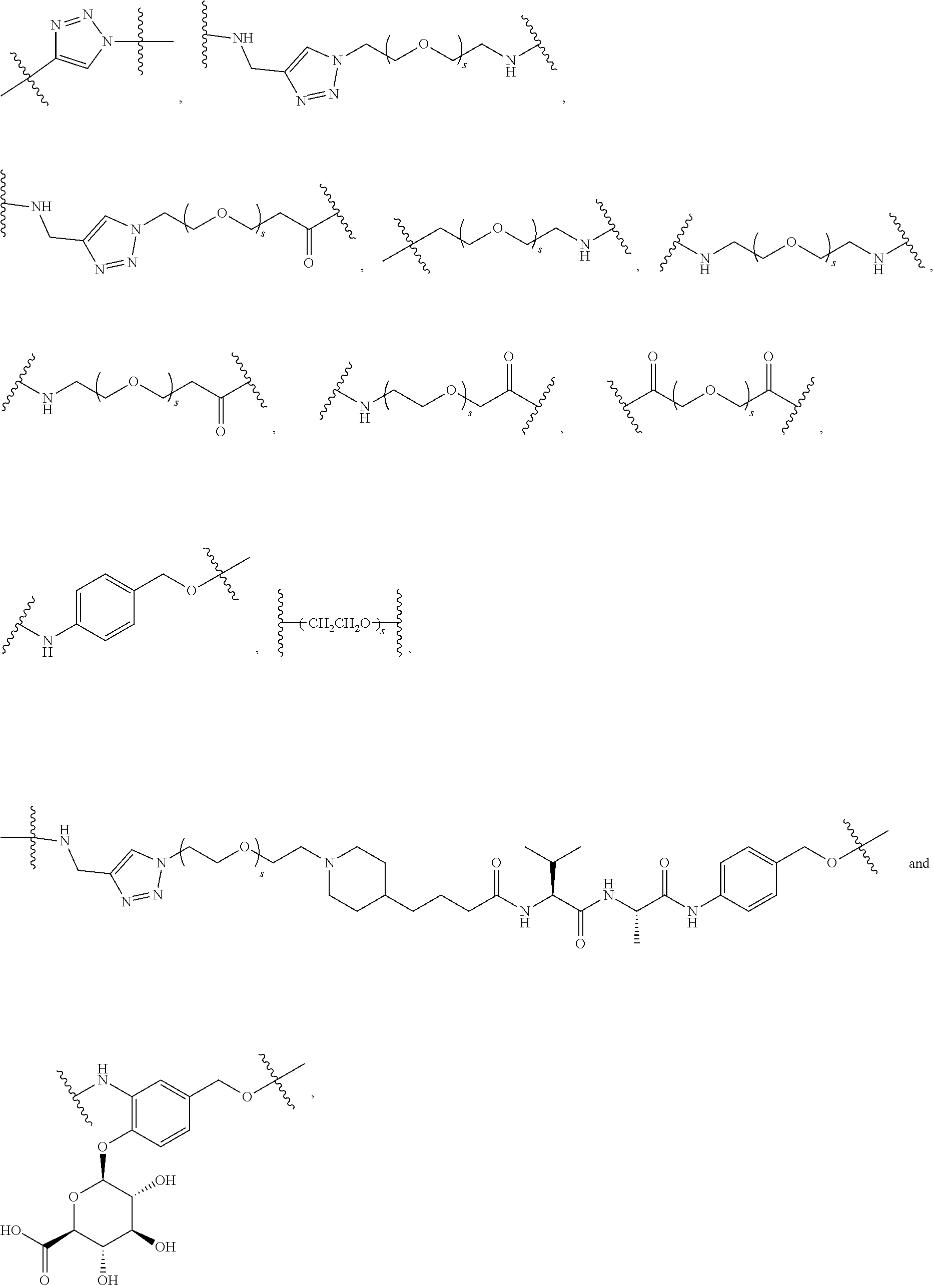

or a combination thereof, wherein R′ represents hydrogen, C₁₋₆ alkyl or alkyl containing—(CH₂CH₂O)ᵣ-; r is selected from an integer from 1-10; and s is selected from an integer from 1-20.

In some embodiments, L is selected from a structure comprising one or more of C₁₋₆ alkylene, —NH—, Val, Cit, Phe, Lys, Lys(COCH₂CH₂ (OCH₂CH₂)ₛOCH₃), Gly, Val-Cit, Gly-Gly-Phe-Gly (SEQ ID NO: 25),

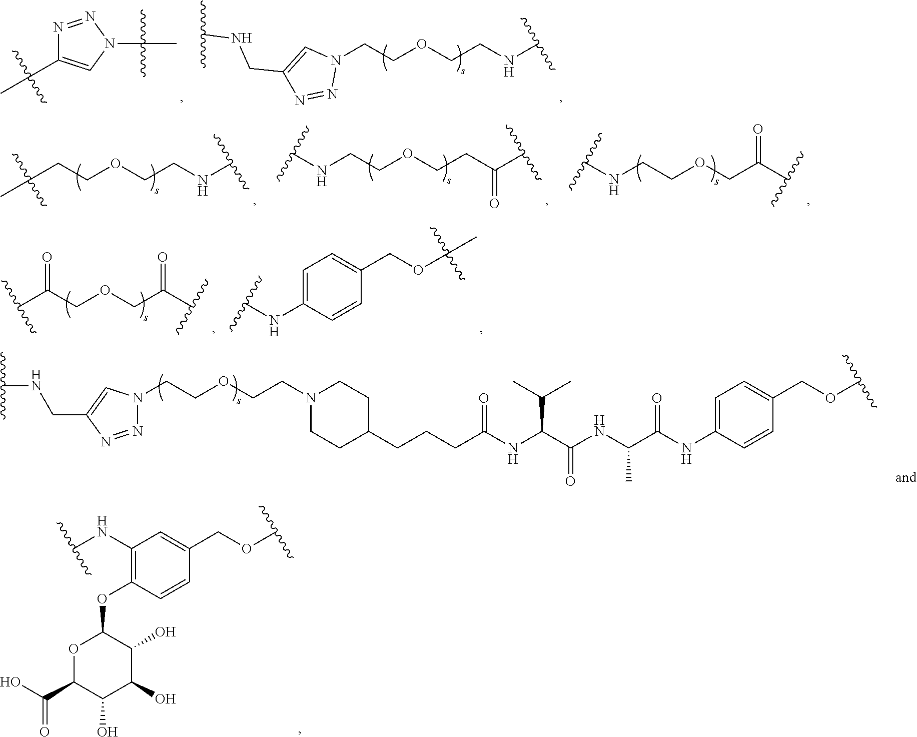

or a combination thereof; where s is selected from an integer from 1-20.

In some embodiments, L is selected from the following structures:

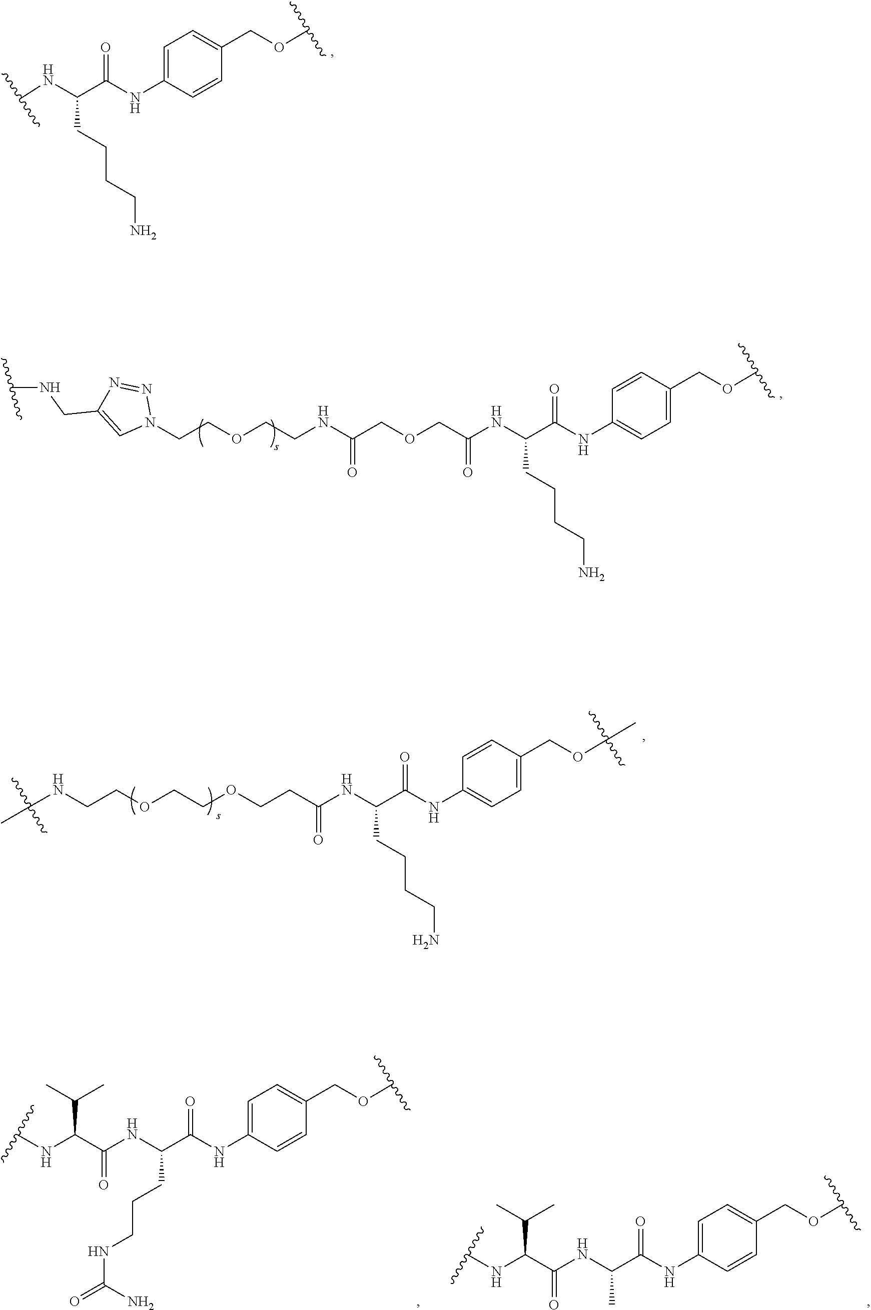

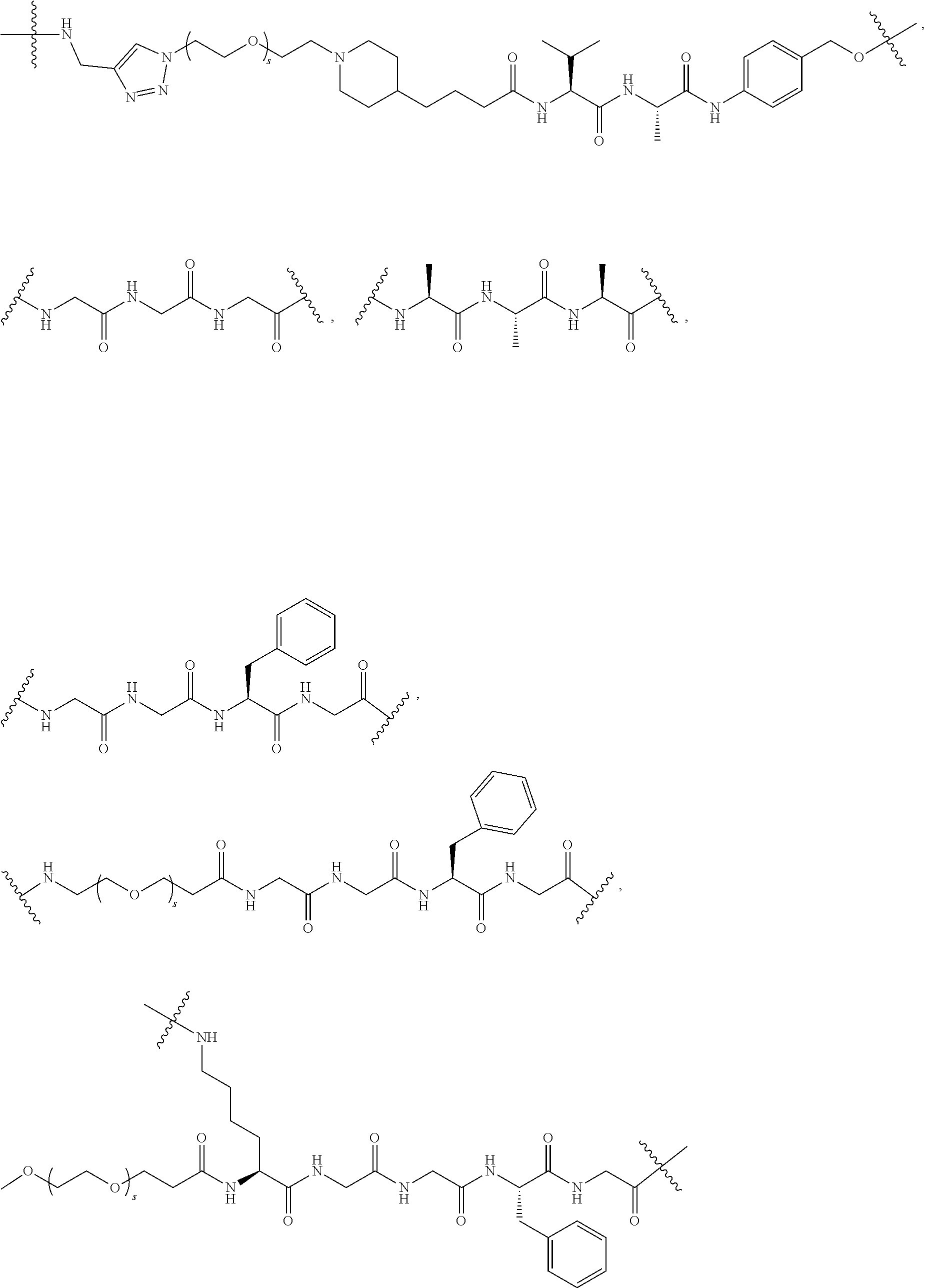

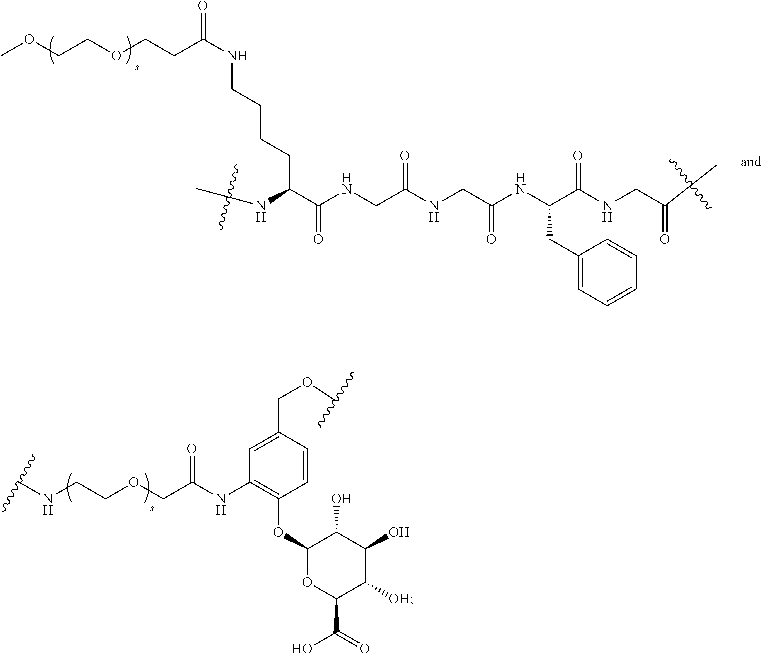

where s is selected from an integer from 1-20.

In some embodiments, L is selected from the following structures:

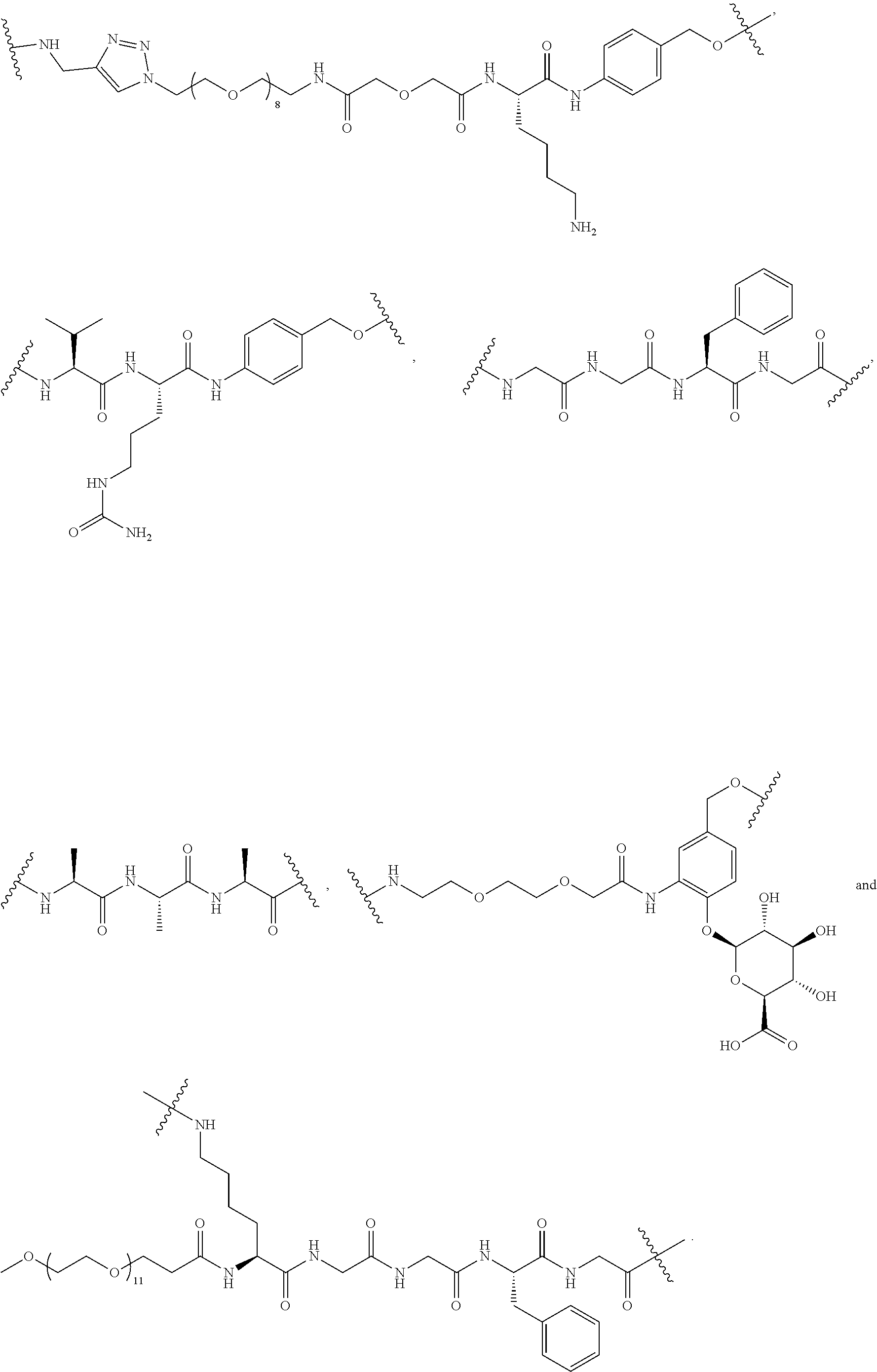

In some embodiments, L is selected from the following structures:

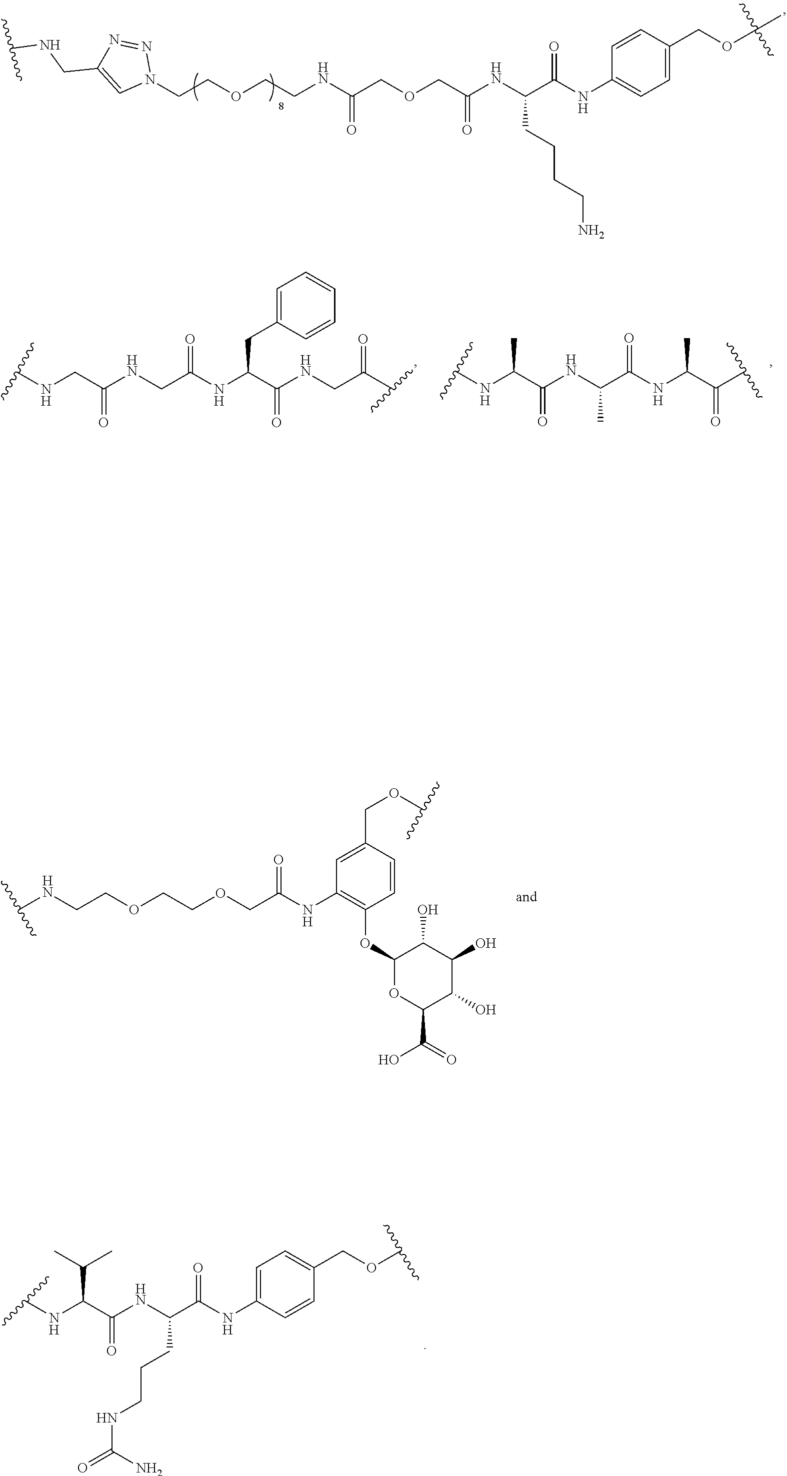

In some embodiments, L is selected from the following structures:

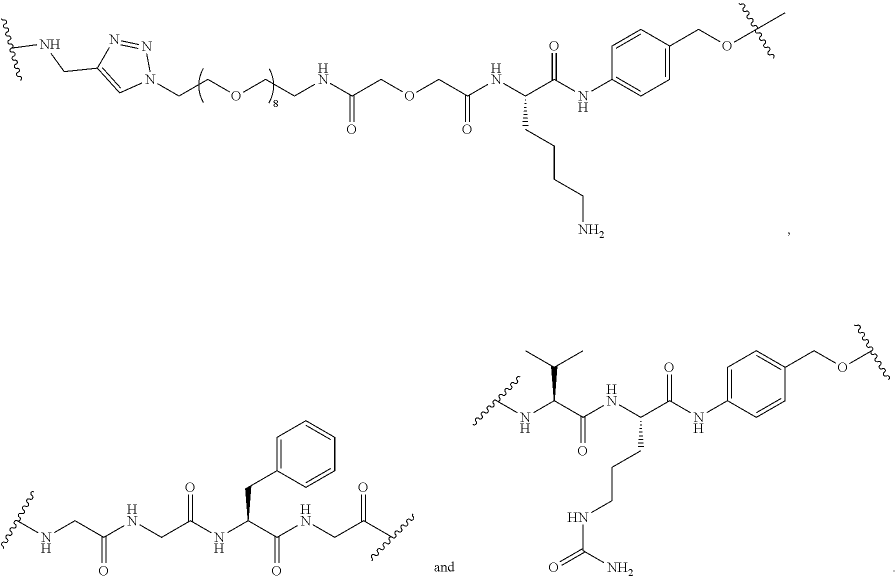

In some embodiments, L is selected from the following structures:

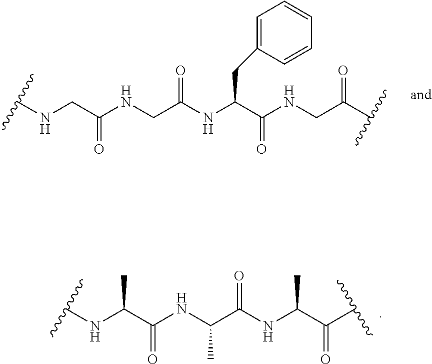 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

In certain embodiments, L is connected to M via amino group depicted on the left hand side in the above groups and to E group via carbonyl group on the right hand side in the above groups.

In some embodiments, E is a single bond, —NH—CH₂-, —NH—CH₂-O-CH₂—CO—,

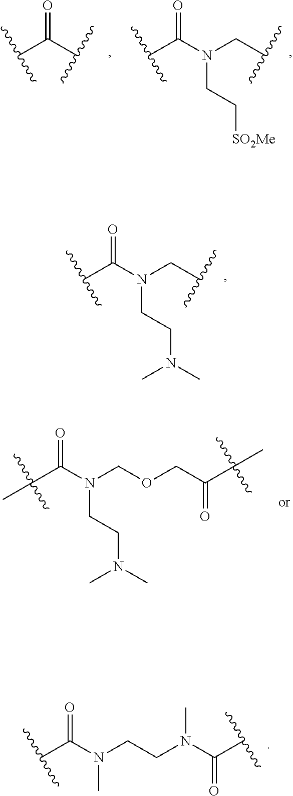

In some embodiments, E is a single bond, —NH—CH₂-, —NH—CH₂-O-CH₂—CO—,

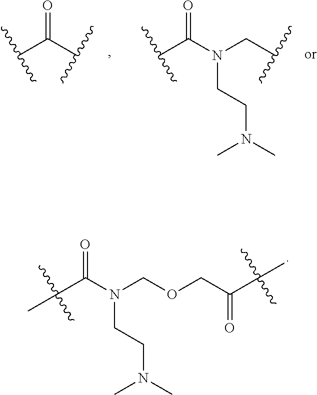

In some embodiments, E is —NH—CH₂-,

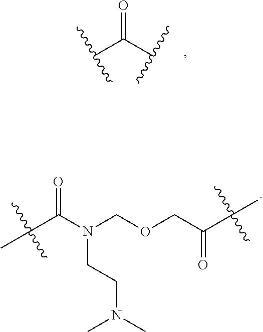

In some embodiments, E is —NH—CH₂— or

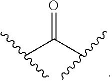 [SMILES: *C(*)=O]

In some embodiments, E is —NH—CH₂-O-CH₂—CO-.

In some embodiments,

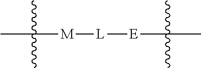

is selected from the following structures:

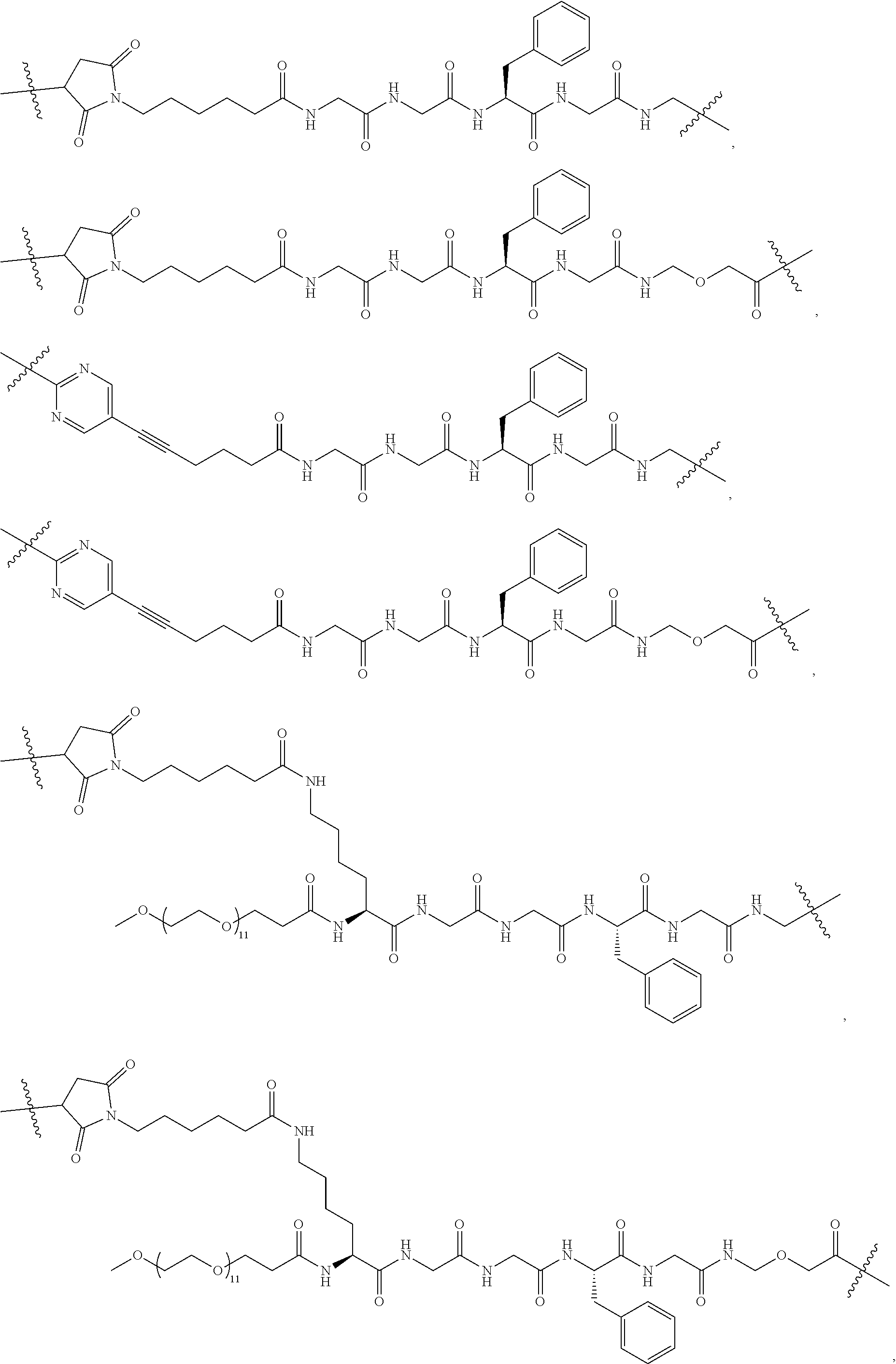

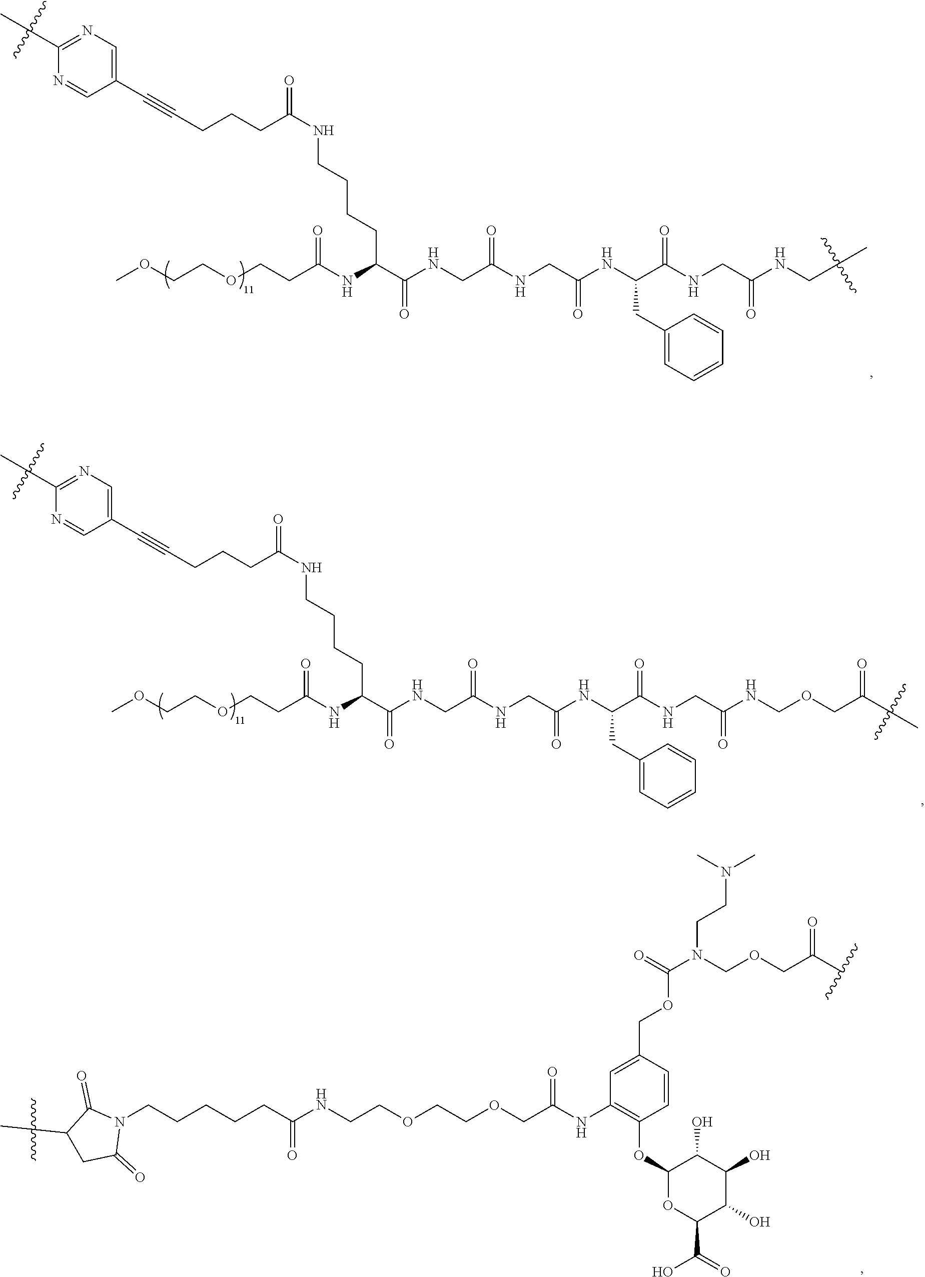

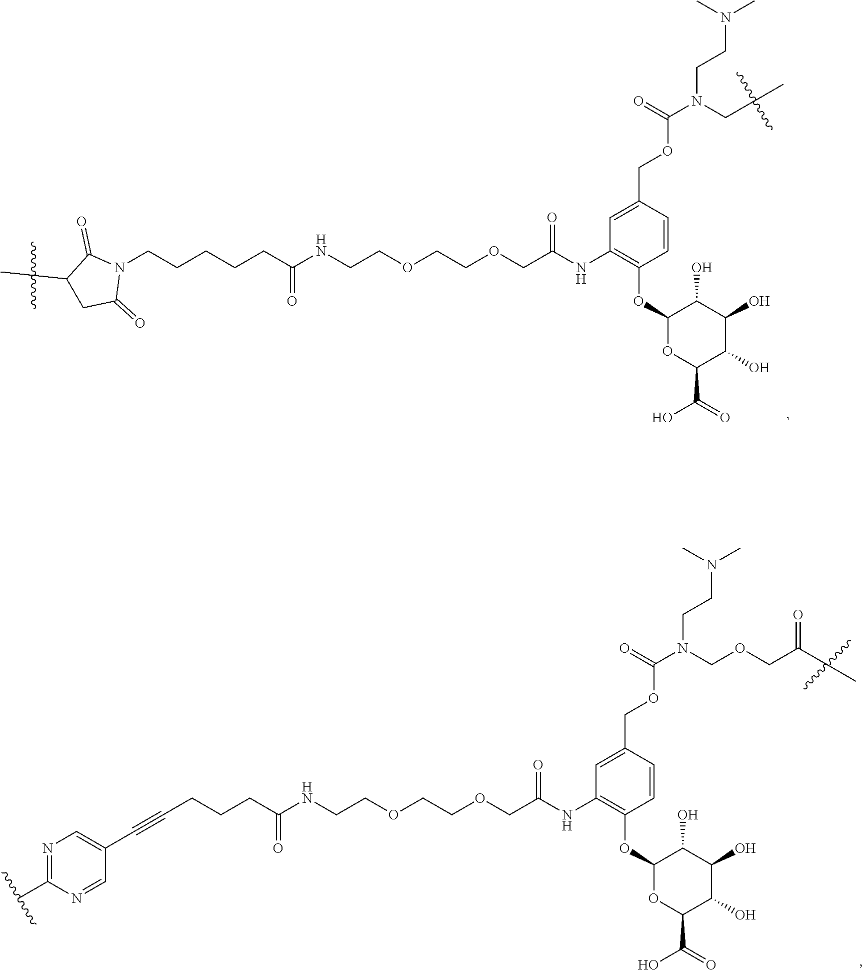 [SMILES: *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

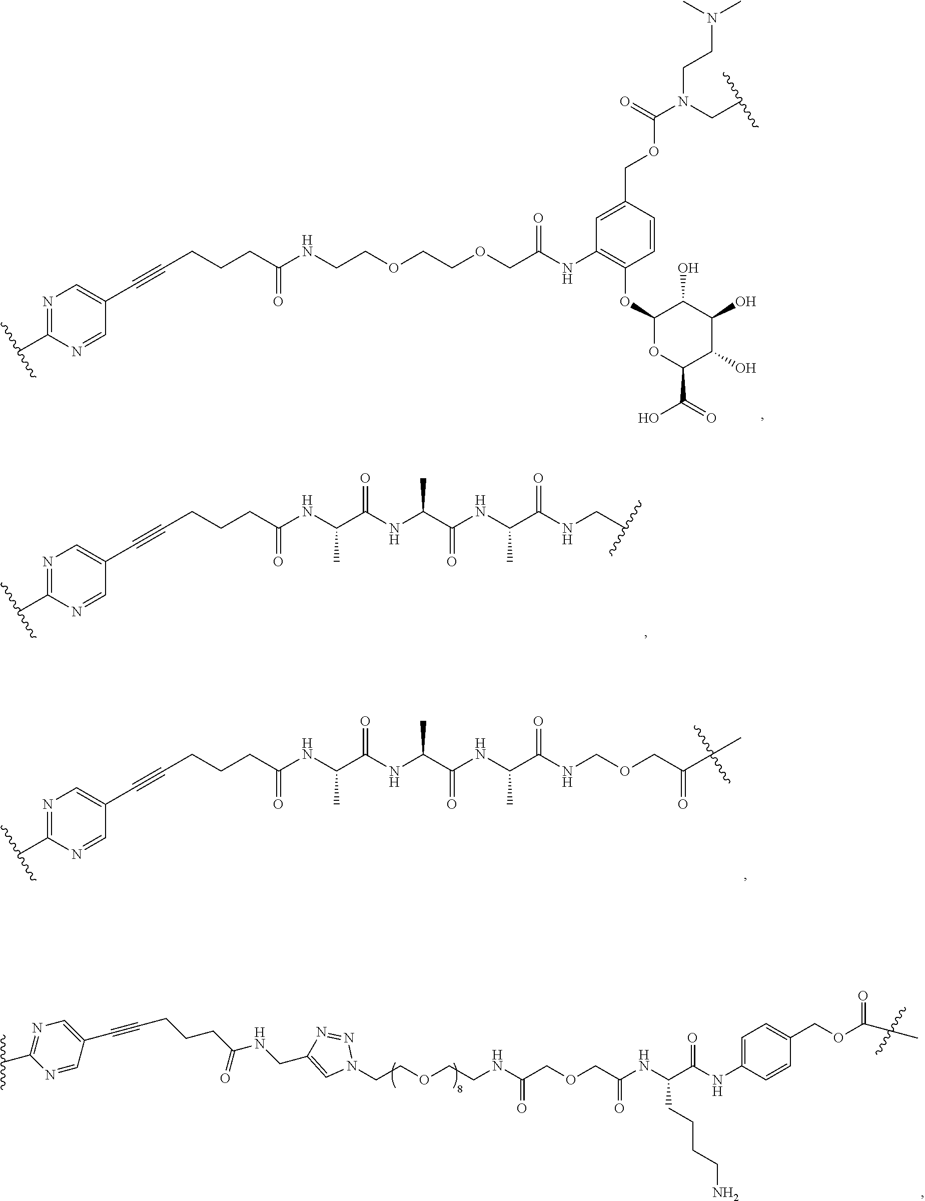

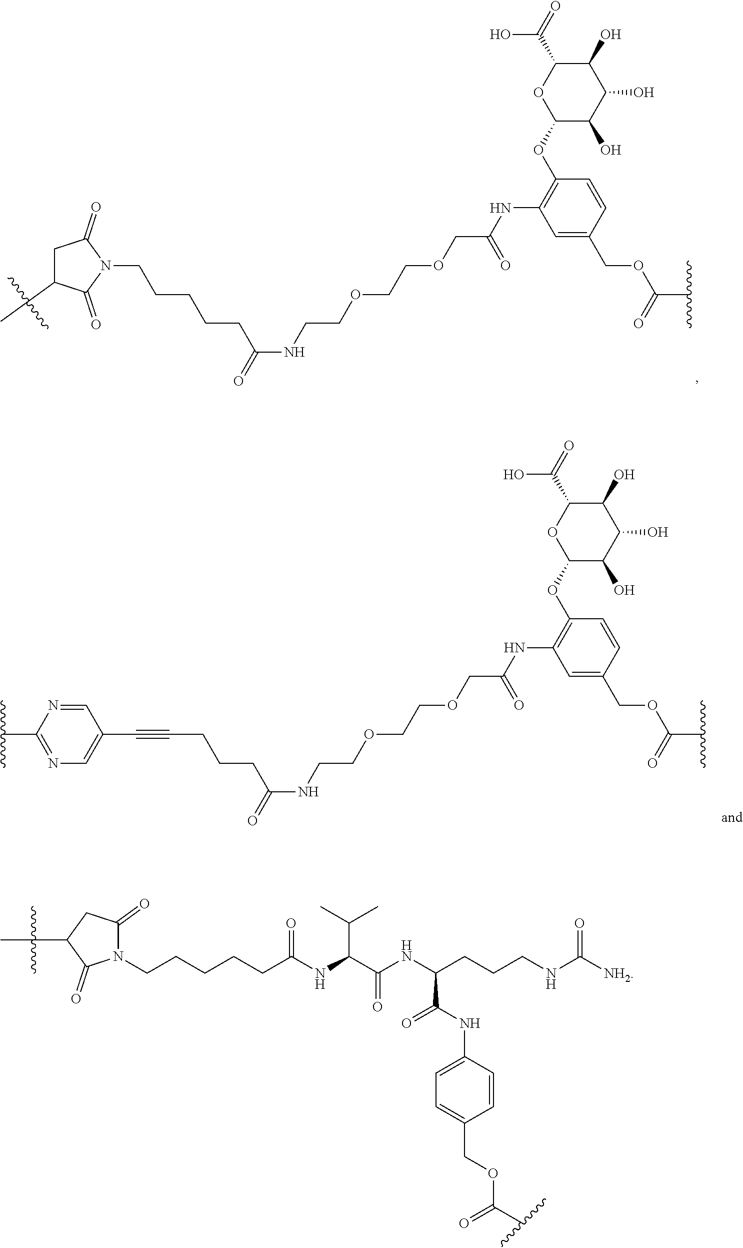 [SMILES: *C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments,

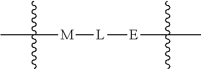

is selected from the following structures:

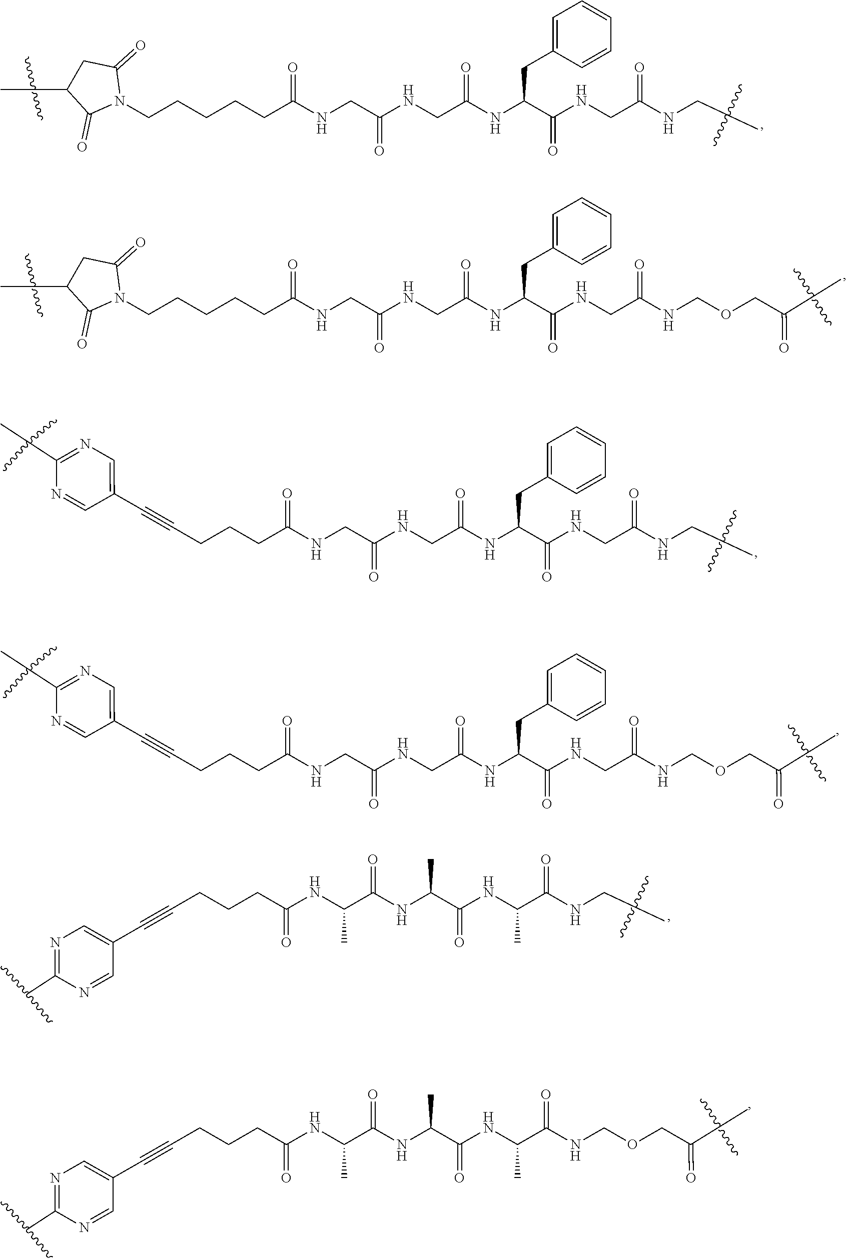 [SMILES: *CNC(=O)CNC(=O)[C@H](Cc1ccccc1)NC(=O)CNC(=O)CNC(=O)CCCCCN1C(=O)CC(*)C1=O ; *C(=O)COCNC(=O)CNC(=O)[C@H](Cc1ccccc1)NC(=O)CNC(=O)CNC(=O)CCCCCN1C(=O)CC(*)C1=O ; *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

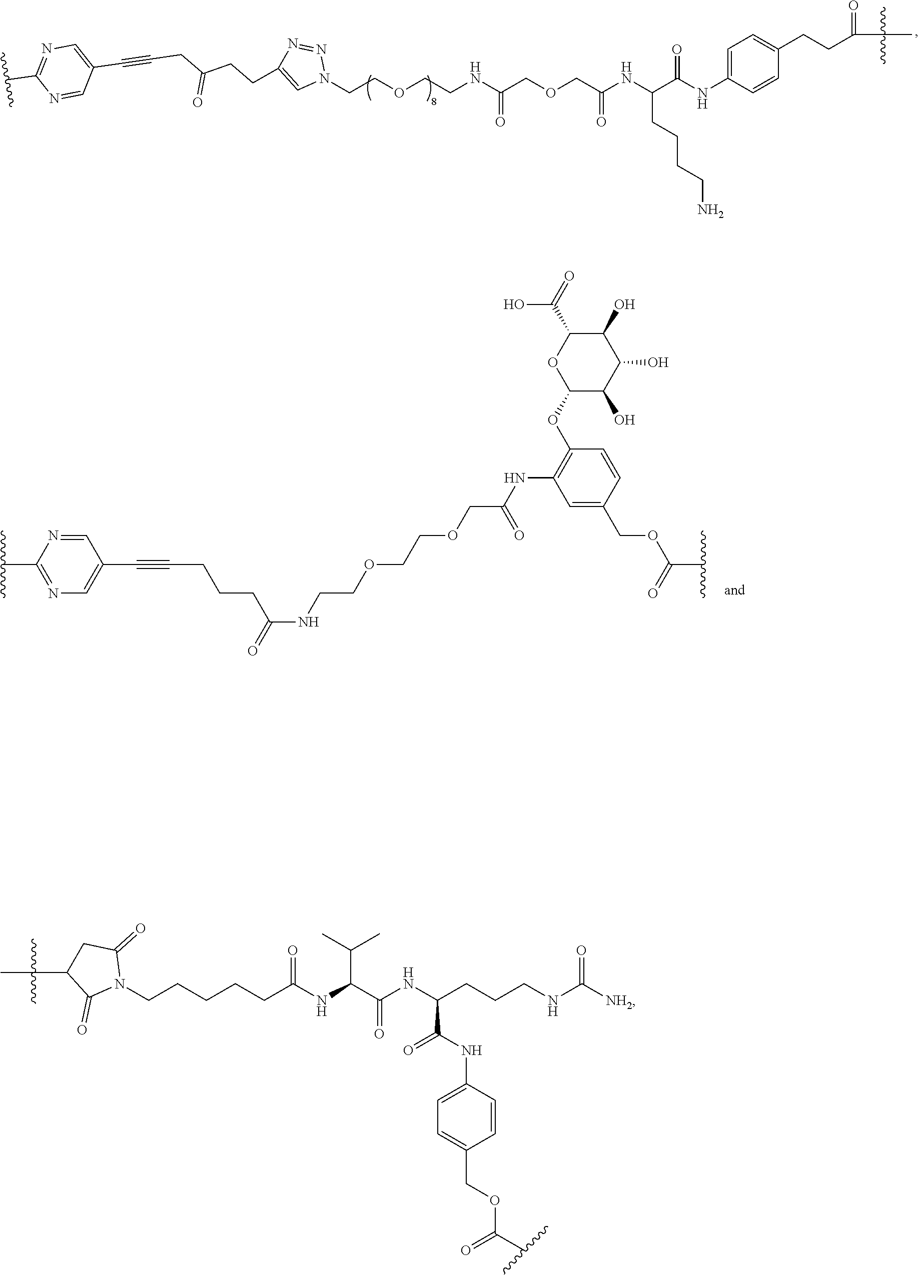

In some embodiments,

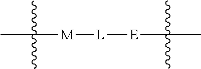

is selected from the following structures:

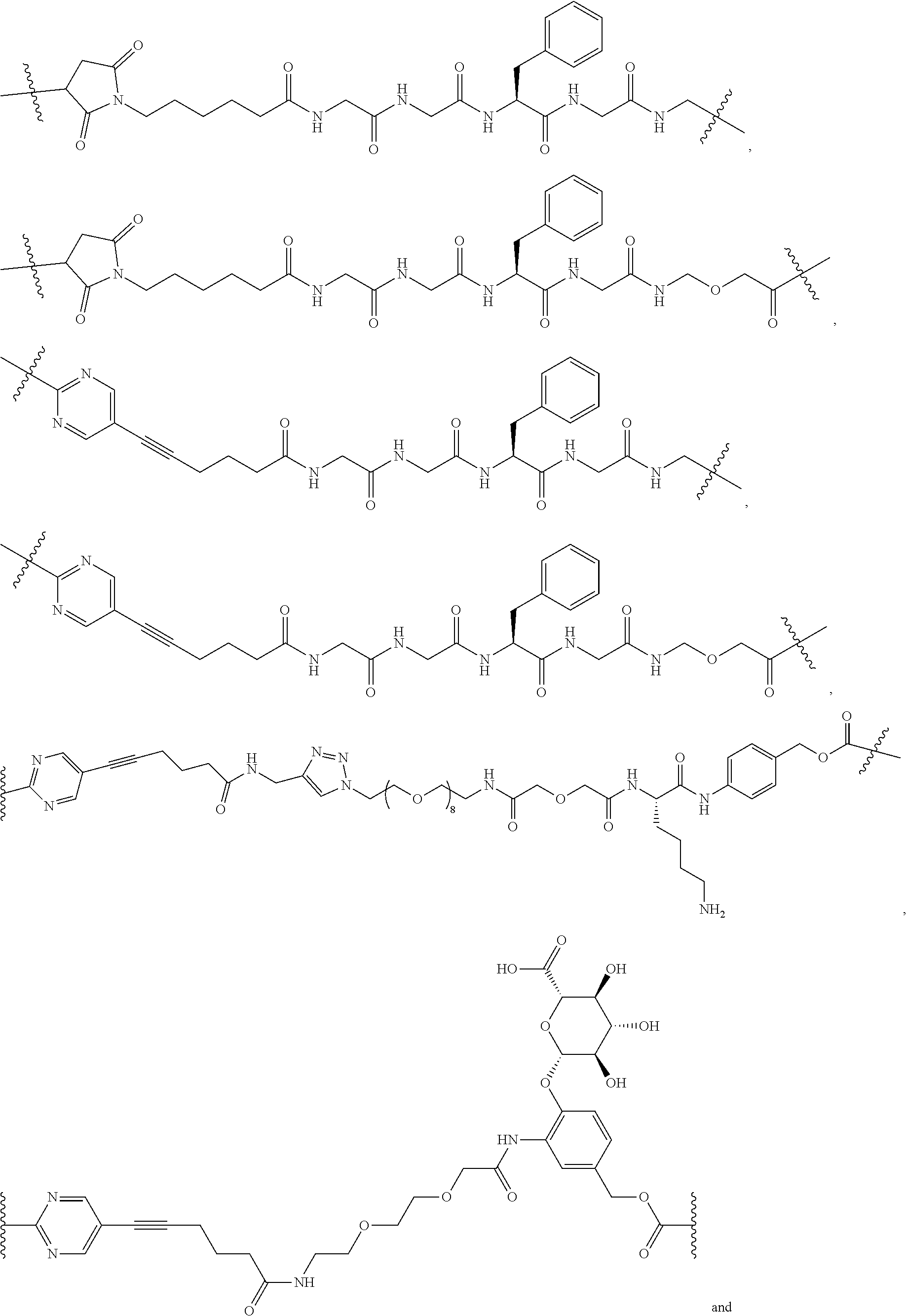

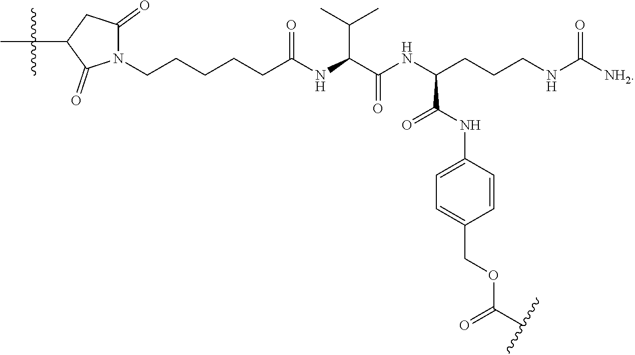 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments, M is selected from the following structures:

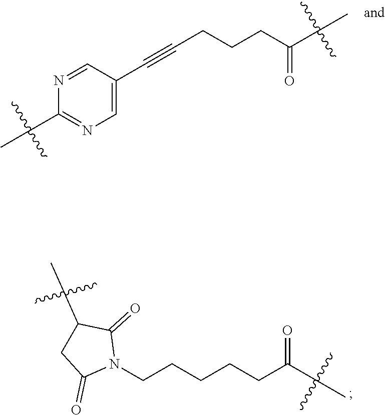 [SMILES: *C(=O)CCCC#Cc1cnc(*)nc1 ; *C(=O)CCCCCN1C(=O)CC(*)C1=O]

L is selected from the following structures:

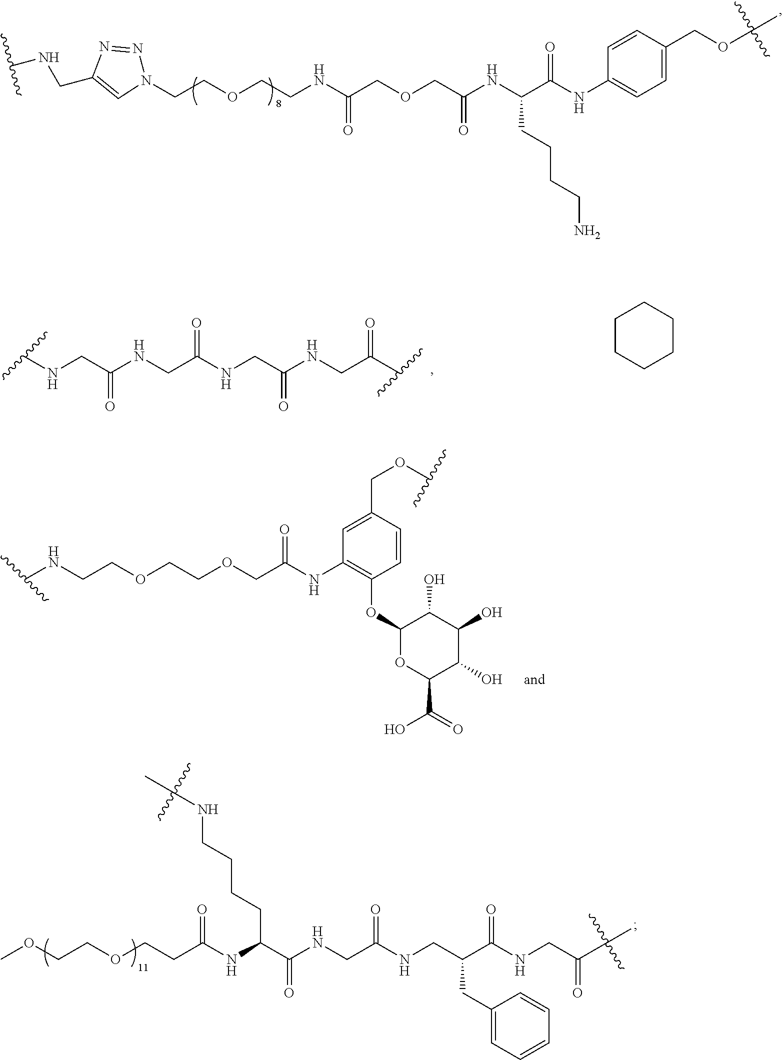

E is —NH—CH₂,

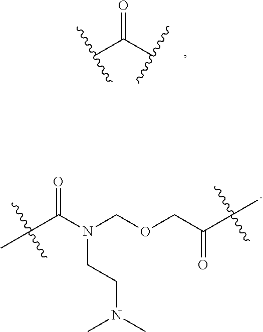

In some embodiments,

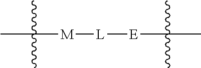

is selected from the following structures:

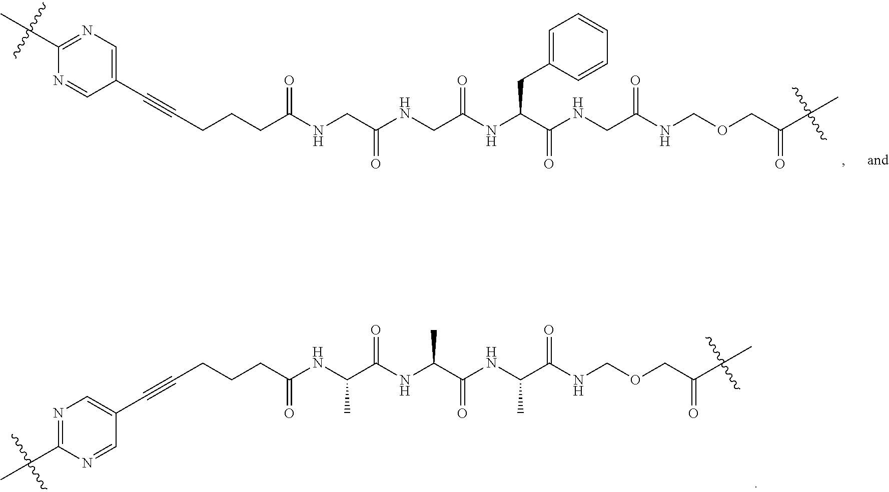 [SMILES: *C(=O)COCNC(=O)CNC(=O)[C@H](Cc1ccccc1)NC(=O)CNC(=O)CNC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

In some embodiments, the cytotoxic drug is selected from a tubulin inhibitor, a DNA intercalator, a DNA topoisomerase inhibitor and a RNA polymerase inhibitor. In some embodiments, the tubulin inhibitor is an auristatin compound or a maytansinoid compound. In some embodiments, the DNA intercalator is pyrrolobenzodiazepine (PBD). In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is a-amanitin or a pharmaceutically acceptable salt, ester or analog thereof.

The cytotoxic drug disclosed in the present application usually contains a variety of functional groups, for example hydroxyl(—OH), carboxyl(—COOH), sulfhydryl(—SH), primary amine (-NH₂), secondary amine (-NR_{A}H) or a tertiary amine group (-NR_{B}R_{C}), and R_{A}, R_{B} and R_{C} herein only represent non-hydrogen substitutes on N; and the cytotoxic drug can be linked to the linker in the conjugate through these functional groups.

In some embodiments, the cytotoxic drug (D) is linked to E in the antibody-drug conjugate through —OH, —SH, primary amine group, secondary amine group or tertiary amine group thereon.

In some embodiments, the cytotoxic drug is selected from the following formulas I and II:

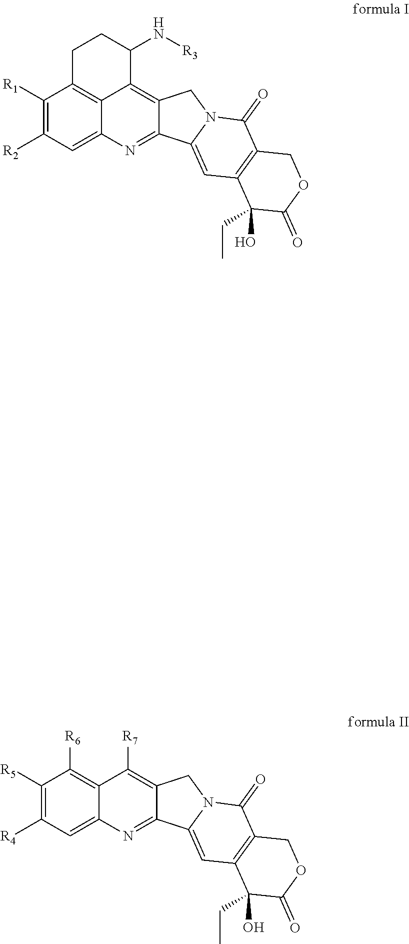

wherein
R₁ and R₂ are each independently selected from C₁₋₆ alkyl and halogen;
R₃ is selected from H and -CO-CH₂OH;
R₄ and R₅ are each independently selected from H, halogen and hydroxyl; or R₄ and R₅ are linked to associated carbon atoms to form 5-6 membered oxygen- containing heterocyclic ring;
R₆ is selected from hydrogen and -C₁₋₄ alkylene-NRₐR_{b};
R₇ is selected from hydrogen, C₁₋₆ alkyl and -C₁₋₄alkylene-NRₐR_{b}; and wherein Rₐ and R_{b} are each independently selected from H, C₁₋₆ alkyl, -SO₂-C₁₋₄ alkyl and -CO-C₁₋₆ alkyl at each occurrence.

In some embodiments, the cytotoxic drug is selected from the following compounds:

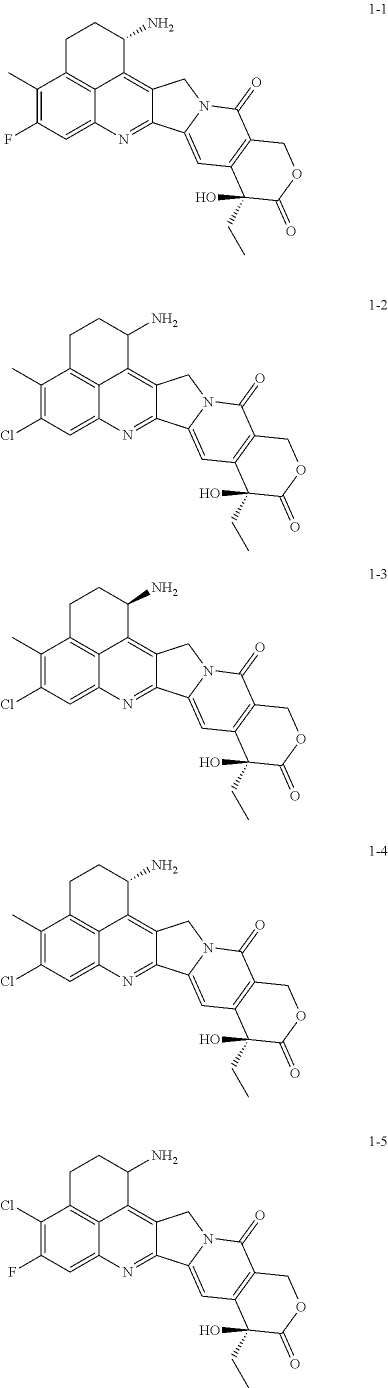 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

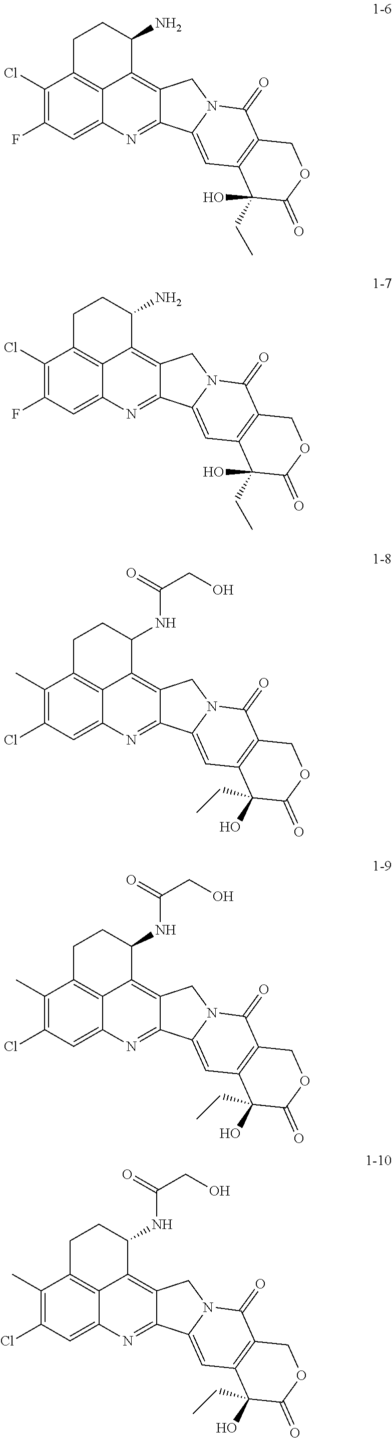 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

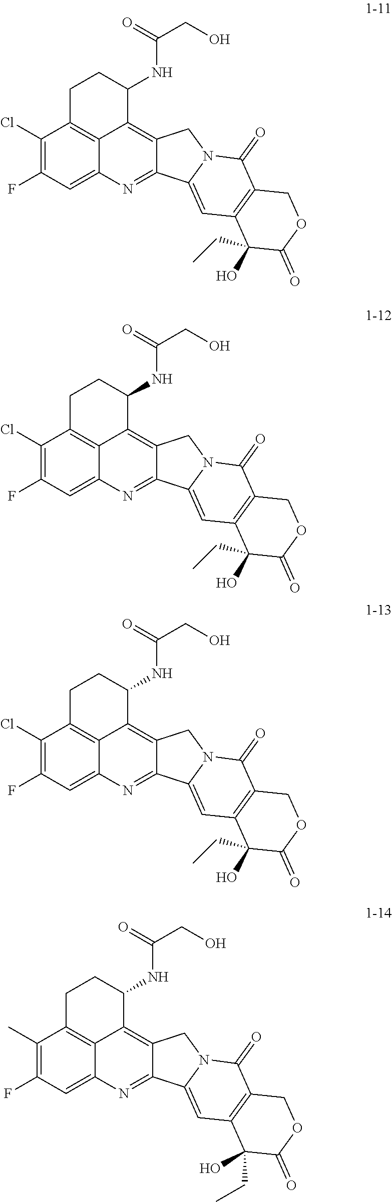 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

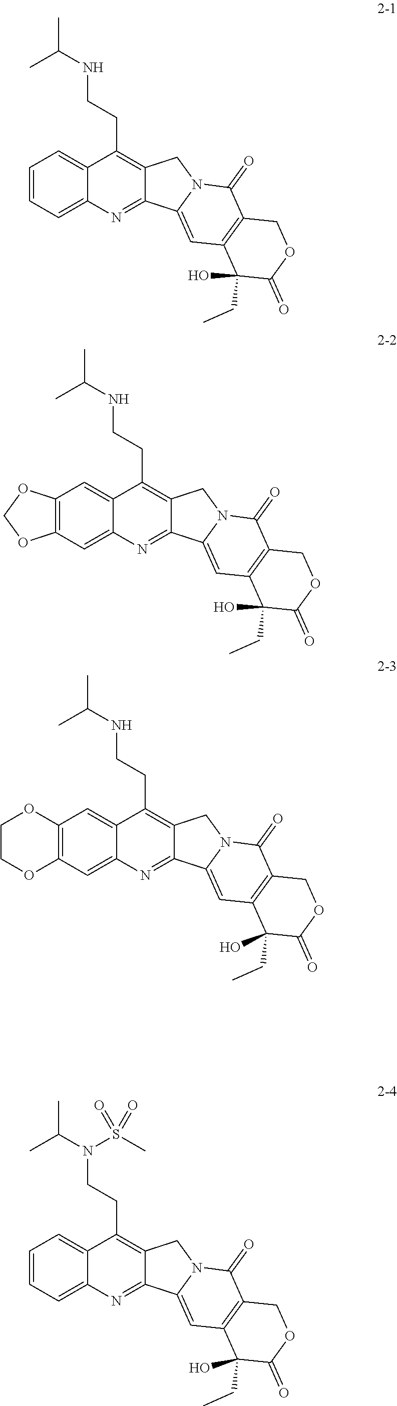 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O]

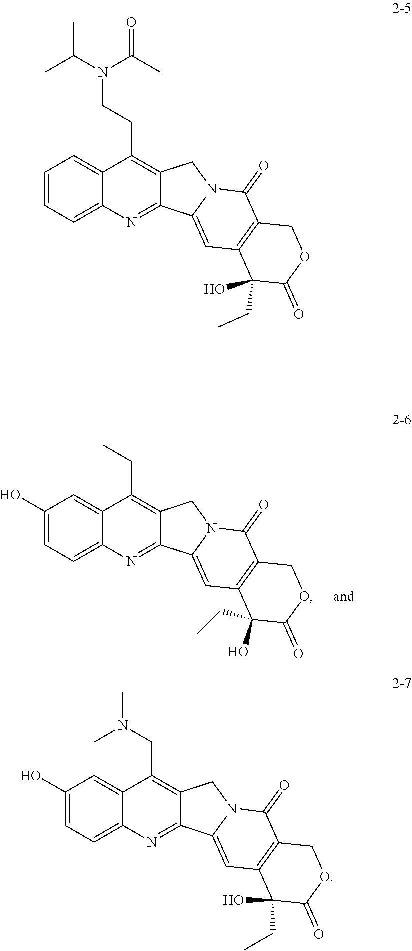 [SMILES: CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

In some embodiments, the cytotoxic drug is selected from the following compounds:

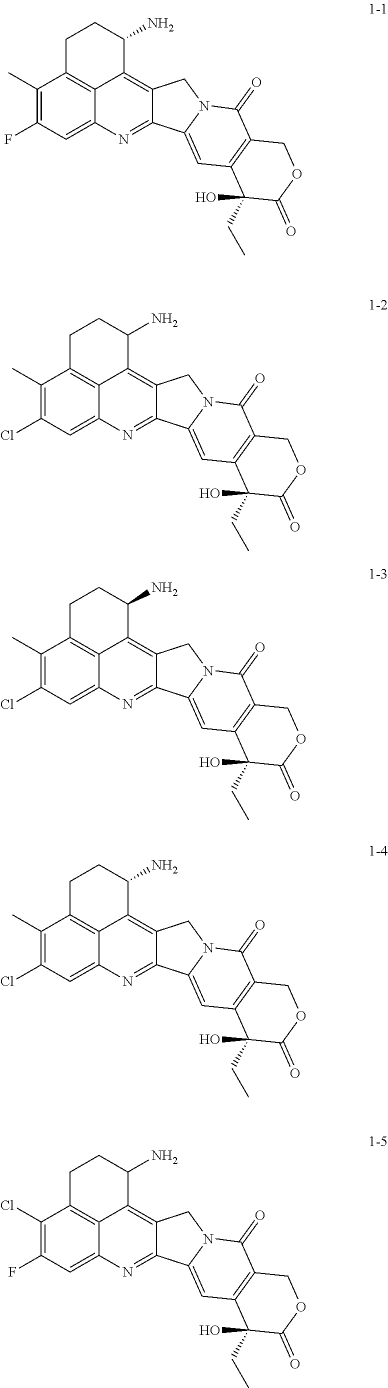 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

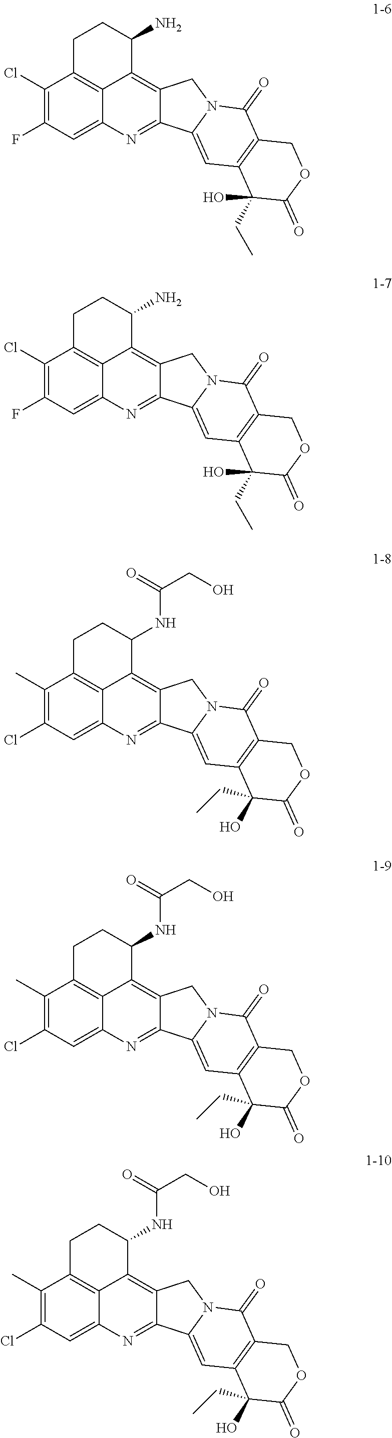 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

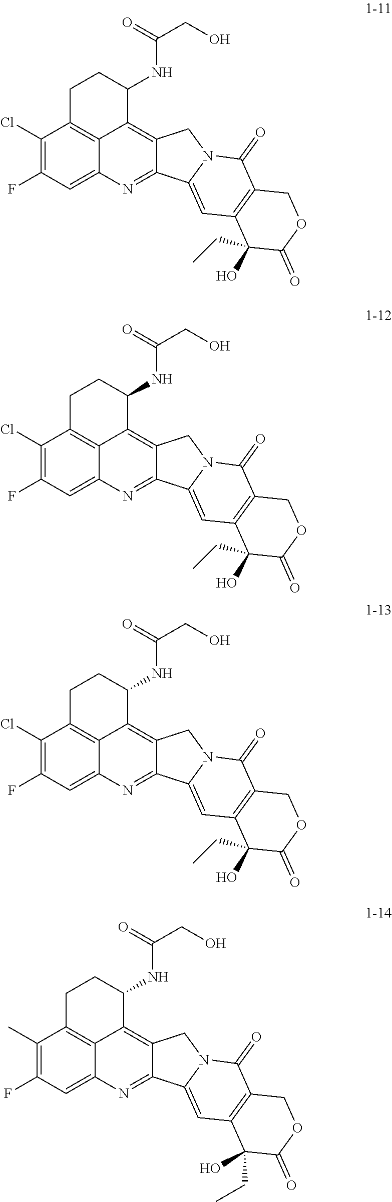 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

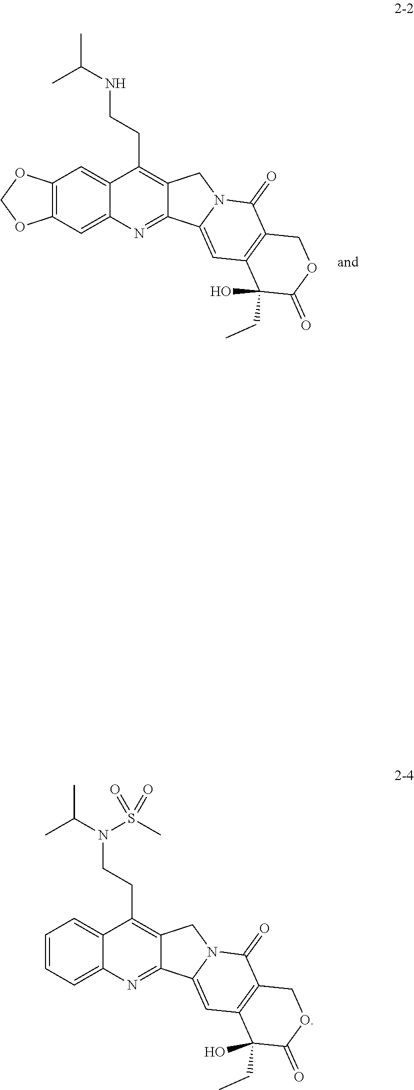 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1ccccc1c2CCN(C(C)C)S(C)(=O)=O]

In some embodiments, D is selected from the following structures:

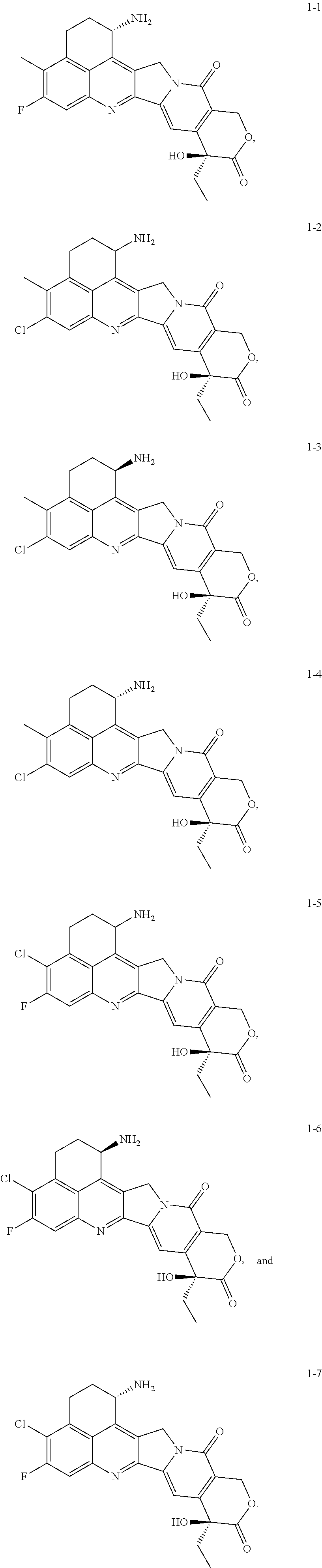

In some embodiments, the cytotoxic drug (D) is linked to E by the loss of one H from —OH, —NH₂, or secondary amine groups on the cytotoxic drug.

In some embodiments, -D is selected from the following structures:

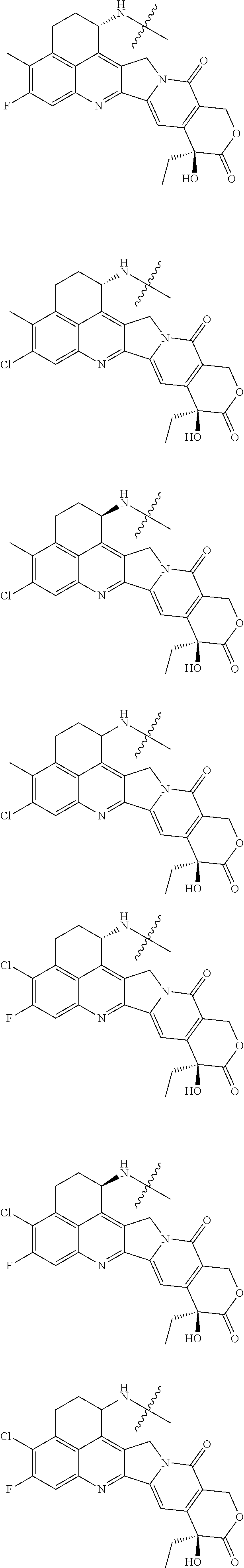

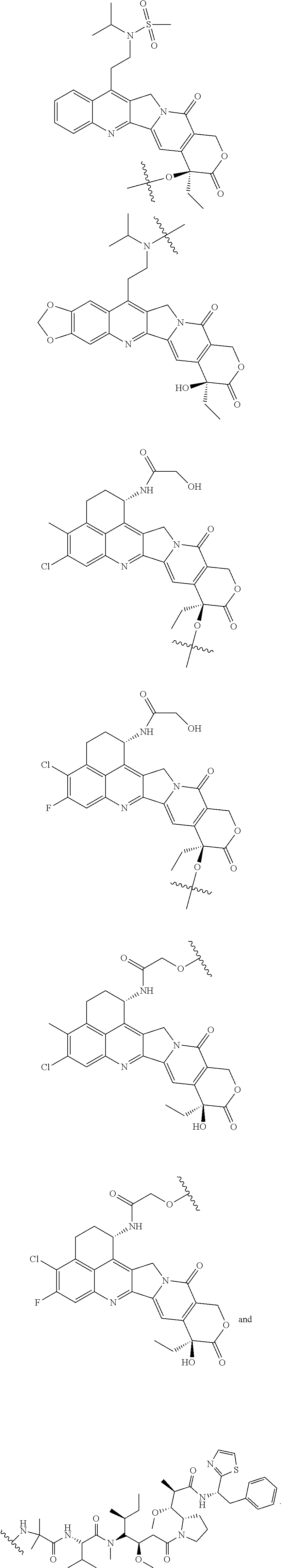

In some embodiments, the cytotoxic drug is

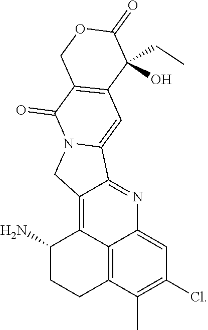 [SMILES: CC[C@@]1(O)C(=O)OCc2c1cc1n(c2=O)Cc2c-1nc1cc(Cl)c(C)c3c1c2[C@@H](N)CC3]

In some embodiments, the cytotoxic drug is

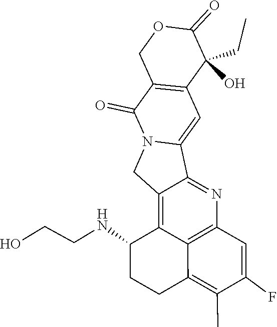 [SMILES: CC[C@@]1(O)C(=O)OCc2c1cc1n(c2=O)Cc2c-1nc1cc(F)c(C)c3c1c2[C@@H](NCCO)CC3]

In some embodiments, (-M-L-E-D) is selected from

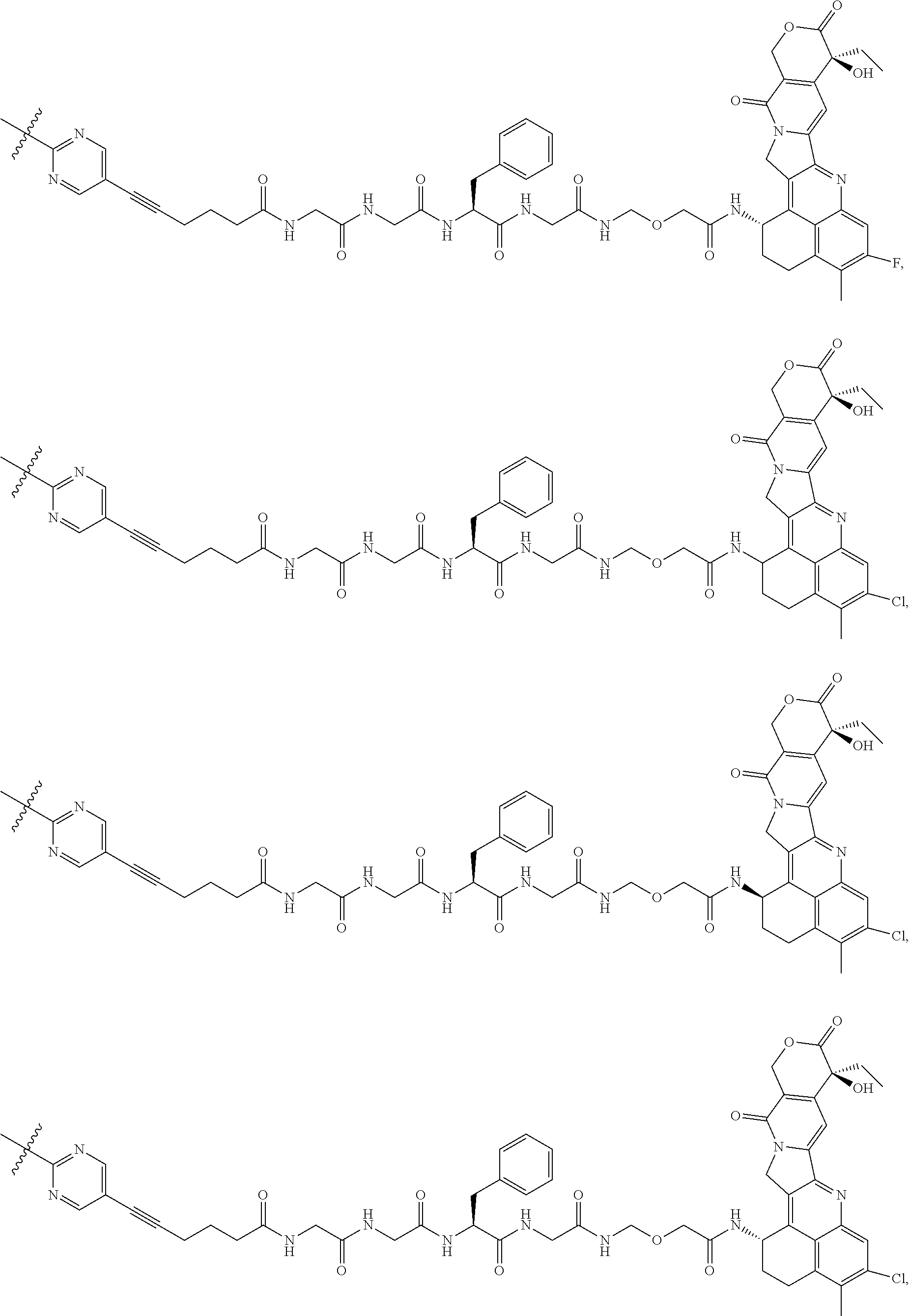

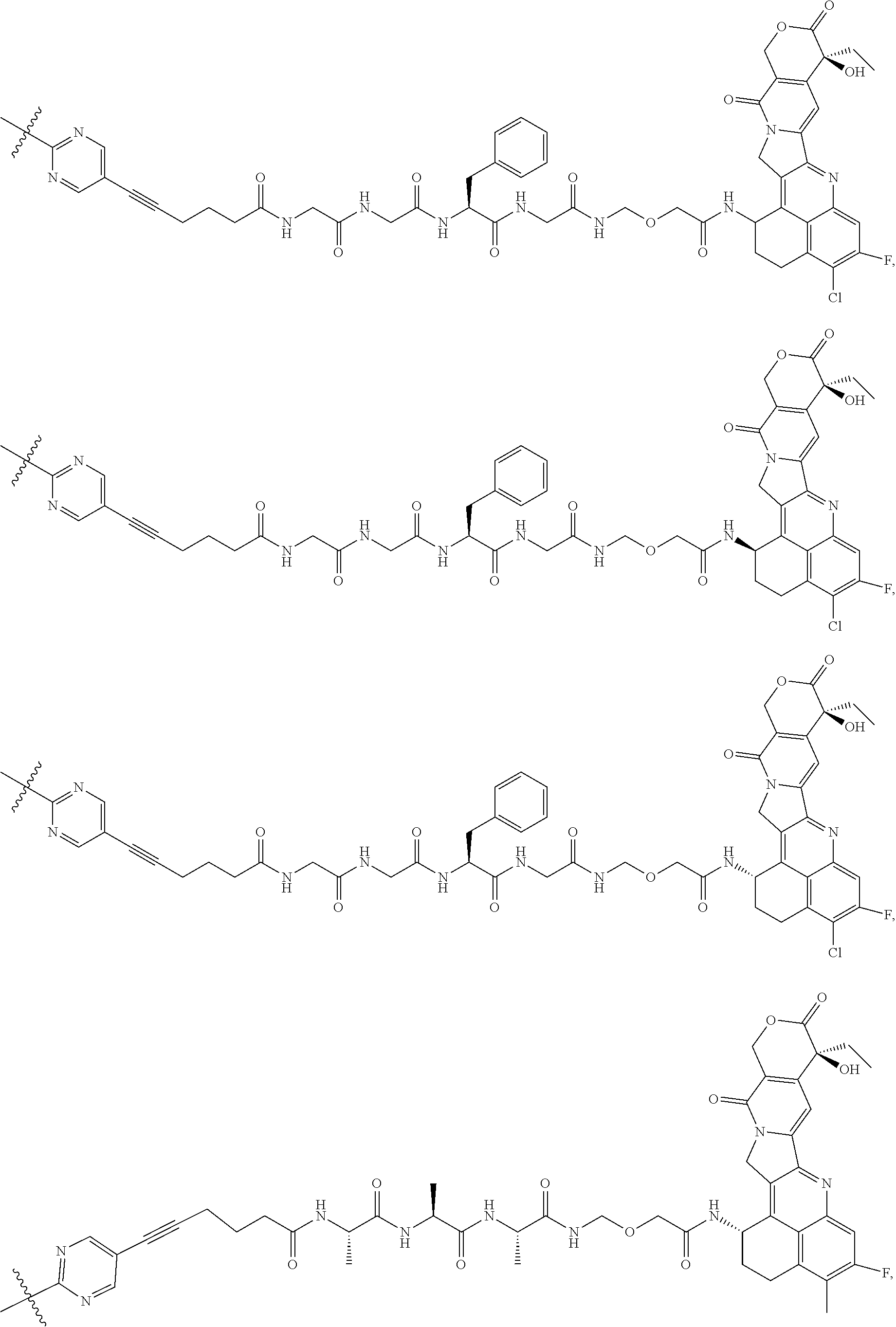

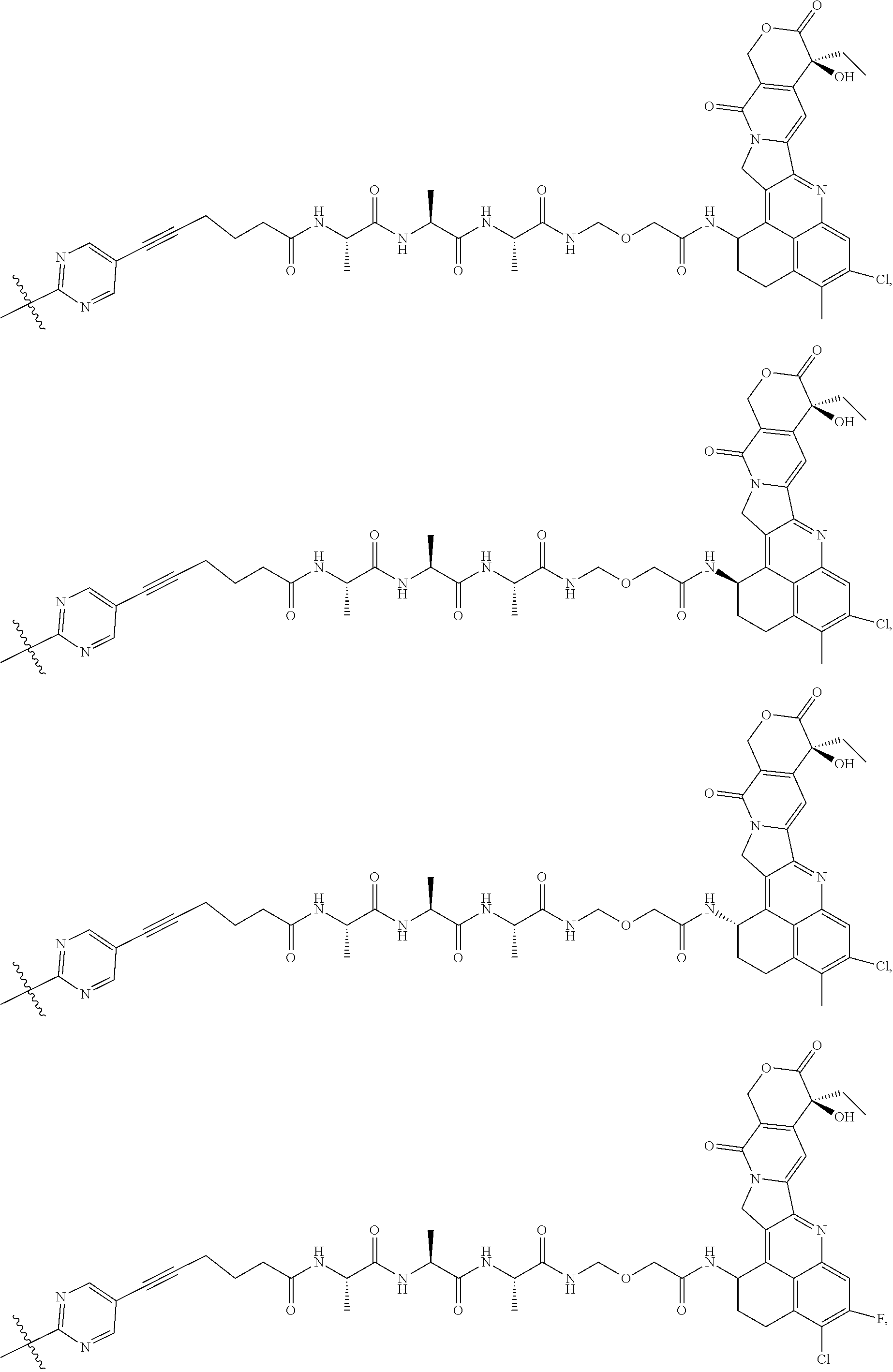

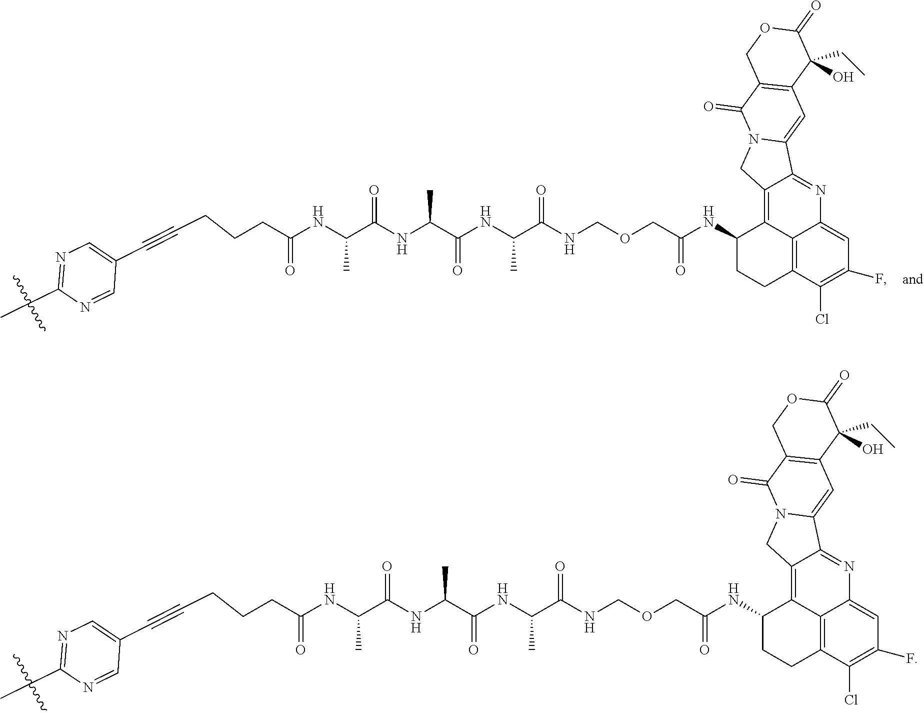 [SMILES: *c1ncc(C#CCCCC(=O)N[C@@H](C)C(=O)N[C@@H](C)C(=O)N[C@@H](C)C(=O)NCOCC(=O)N[C@@H]2CCc3c(Cl)c(F)cc4nc5c(c2c34)Cn2c-5cc3c(c2=O)COC(=O)[C@]3(O)CC)cn1 ; "and" ; *c1ncc(C#CCCCC(=O)N[C@@H](C)C(=O)N[C@@H](C)C(=O)N[C@@H](C)C(=O)NCOCC(=O)N[C@H]2CCc3c(Cl)c(F)cc4nc5c(c2c34)Cn2c-5cc3c(c2=O)COC(=O)[C@]3(O)CC)cn1]

In some embodiments, the antibody-drug conjugate is selected from ADC A-01 to ADC A-04, ADC A-08, ADC A-10, ADC A-12, ADC A-13, ADC A-16, ADC A-18, ADC A-20, ADC A-22, ADC A-24, ADC B-02, and ADC C-01 shown as follows:

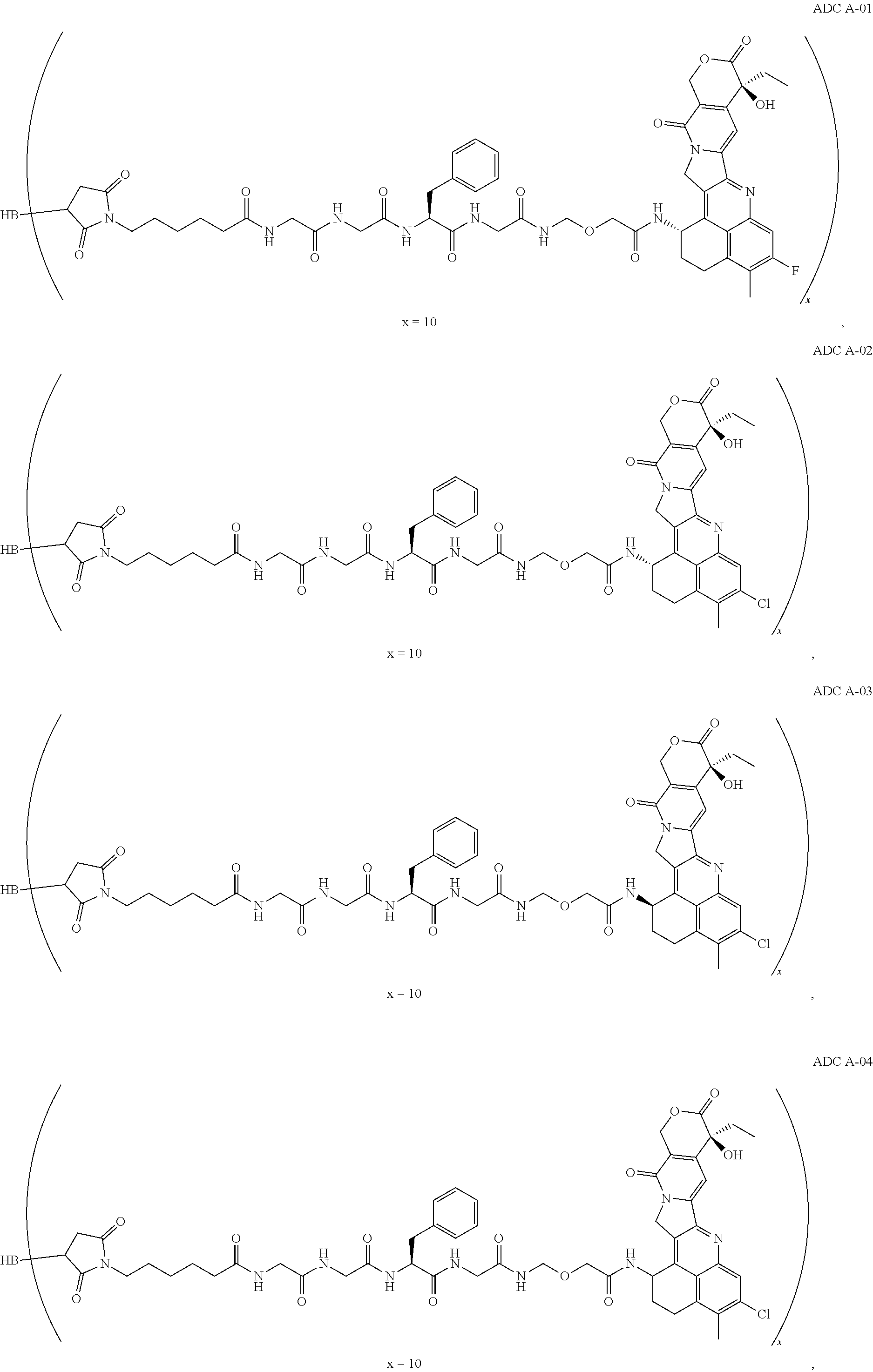

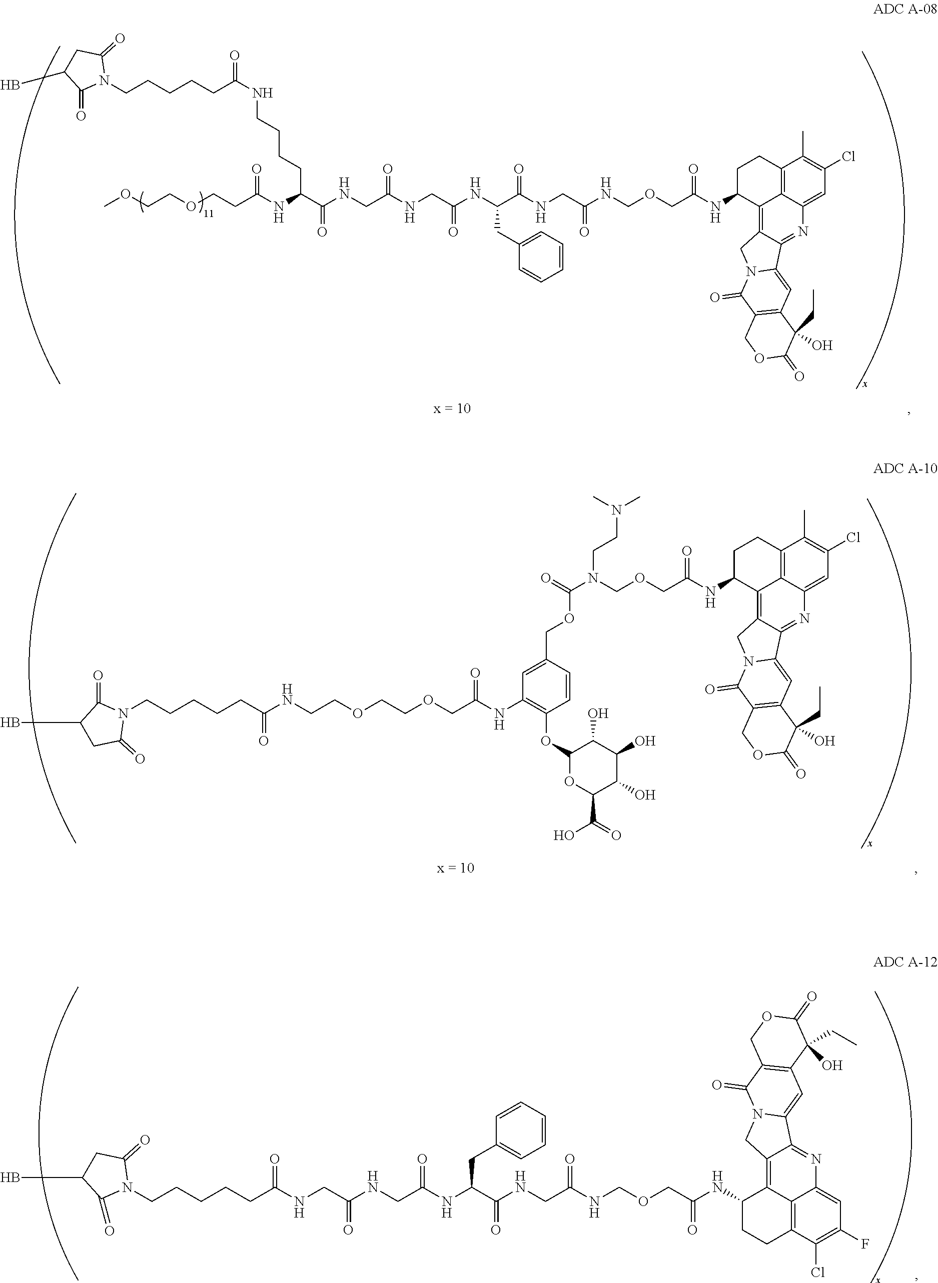

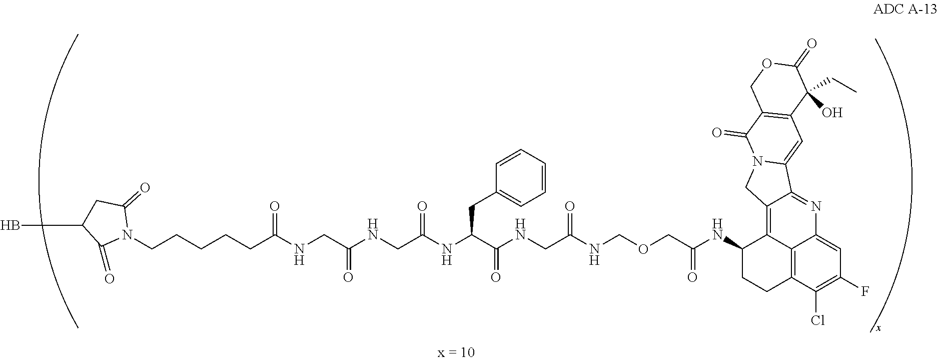

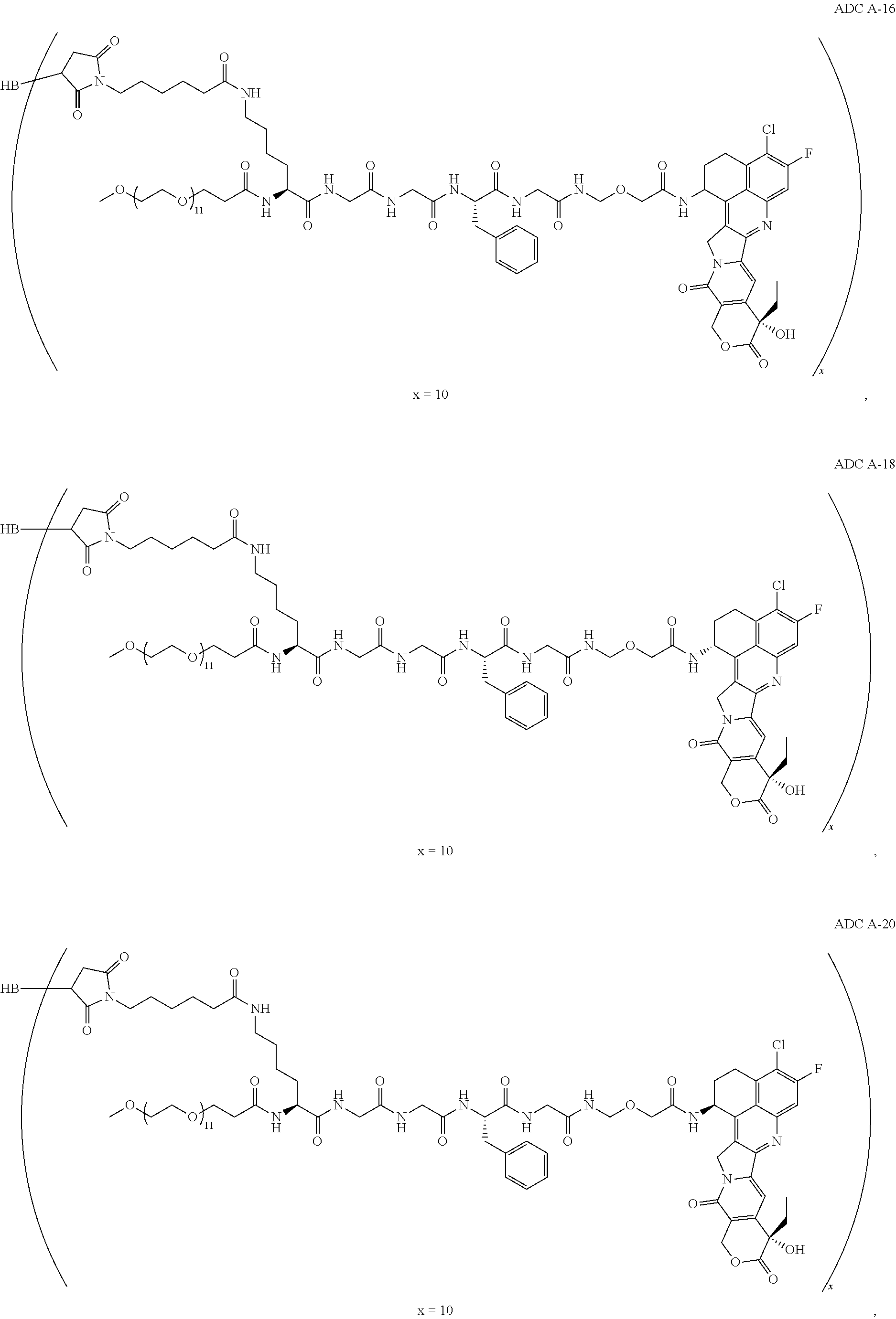

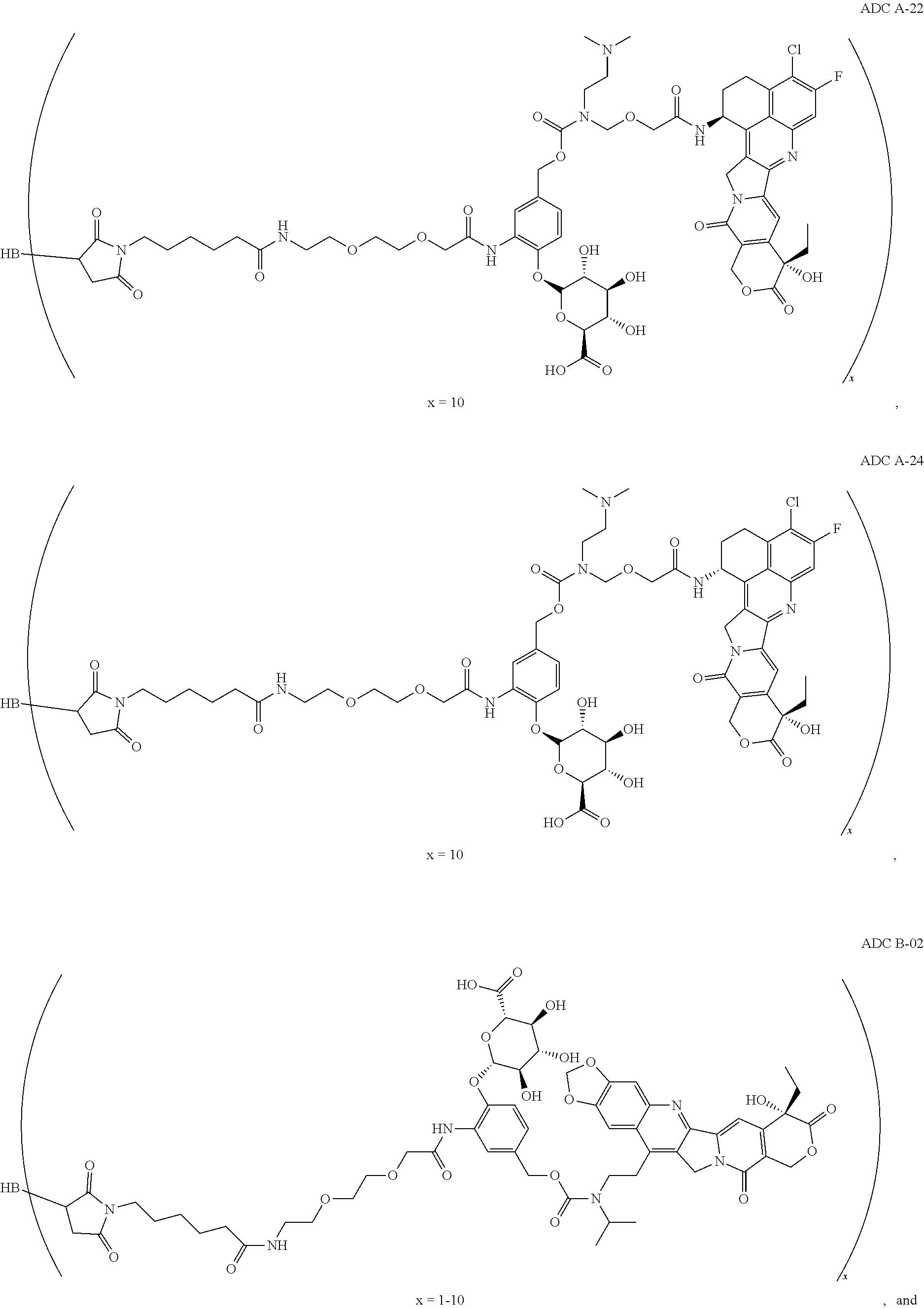

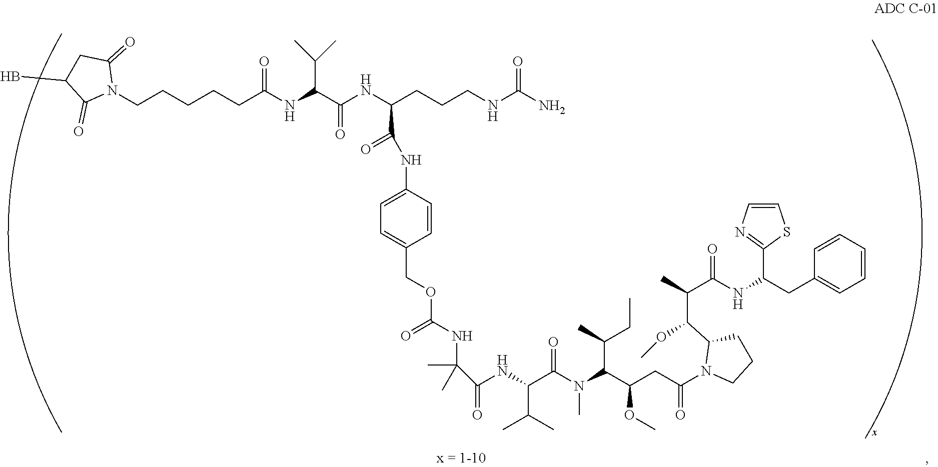

wherein HB-(- is HA-(Y-, wherein HB-(- comprises a V_{H} having the amino acid sequence as set forth in SEQ ID NO: 1 and a V_{L} having the amino acid sequence as set forth in SEQ ID NO: 2, for example, an antibody or antigen-binding fragment thereof comprising a V_{H} having the amino acid sequence as set forth in SEQ ID NO: 1 and a CH having the amino acid sequence as set forth in SEQ ID NO: 3, a V_{L} having the amino acid sequence as set forth in SEQ ID NO: 2 and a CL having the amino acid sequence as set forth in SEQ ID NO: 4; and -(Y-represents a linkage from an amino acid residue of the antibody or antigen-binding fragment thereof to each succinimidyl group in formulas ADC A-01 to ADC A-04, ADC A-08, ADC A-10, ADC A-12, ADC A-13, ADC A-16, ADC A-18, ADC A-20, ADC A-22, ADC A-24, ADC B-02 and ADC C-01.

In some embodiments, HA-(Y— comprises a heavy chain having the amino acid sequence as set forth in SEQ ID NO: 20 and a light chain having the amino acid sequence as set forth in SEQ ID NO: 21.

In some embodiments, HA-(Y- comprises a heavy chain having the amino acid sequence as set forth in SEQ ID NO: 22 and a light chain having the amino acid sequence as set forth in SEQ ID NO: 21.

In some embodiments, HA-(Y- comprises a heavy chain having the amino acid sequence as set forth in SEQ ID NO: 23 and a light chain having the amino acid sequence as set forth in SEQ ID NO: 21.

In some embodiments, HA-(Y- comprises a heavy chain having the amino acid sequence as set forth in SEQ ID NO: 24 and a light chain having the amino acid sequence as set forth in SEQ ID NO: 21.

In some embodiments, the antibody-drug conjugate is selected from ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06 shown as follows:

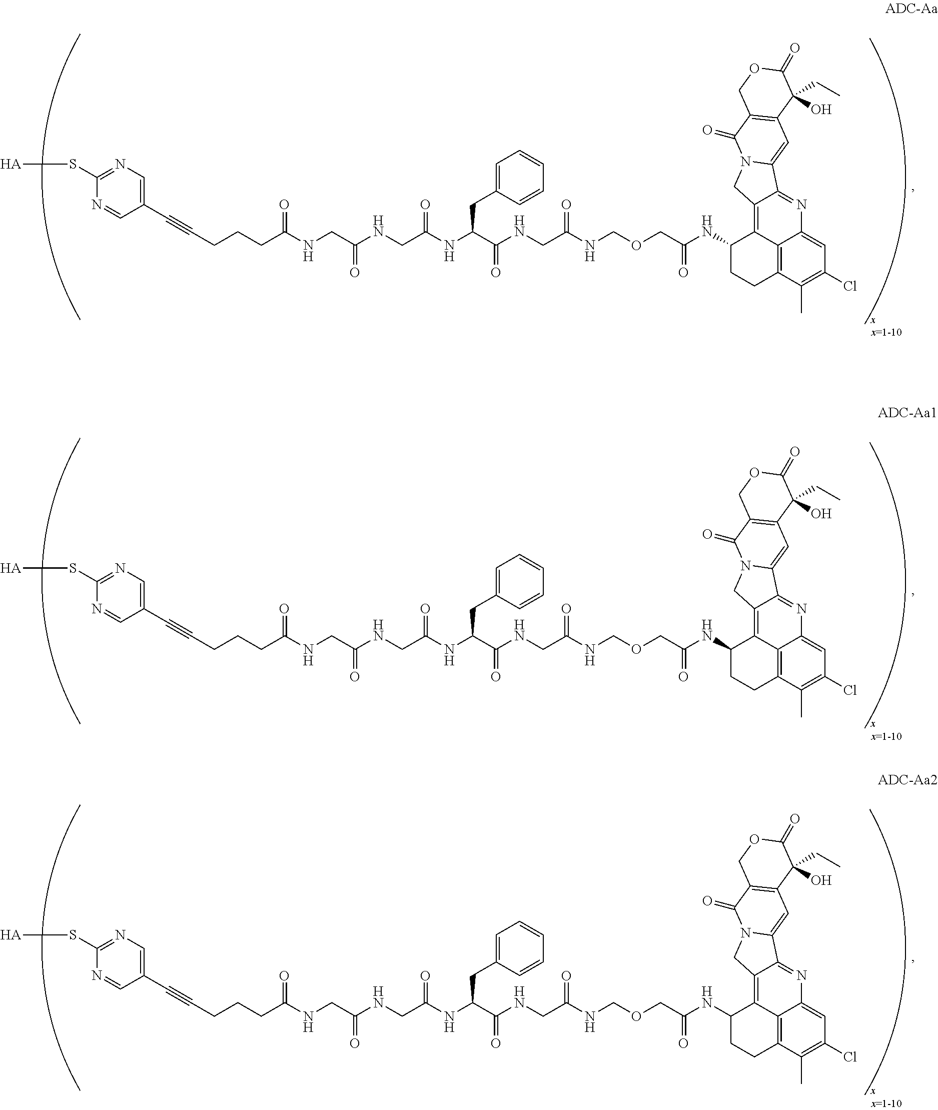

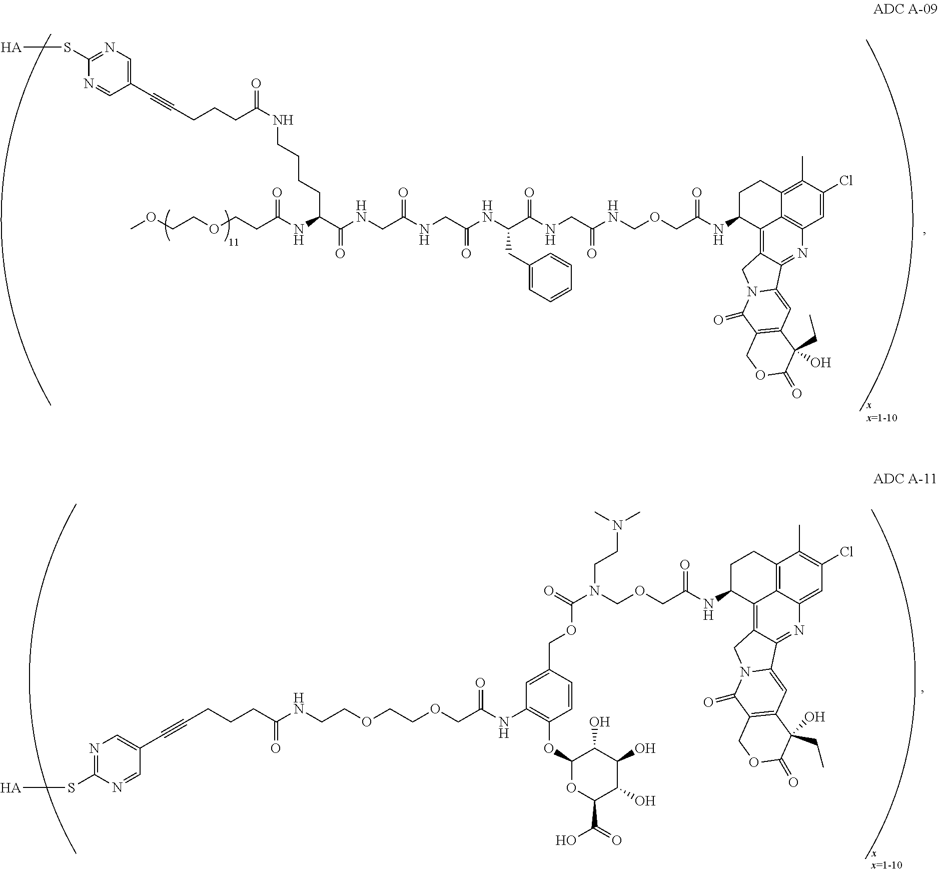

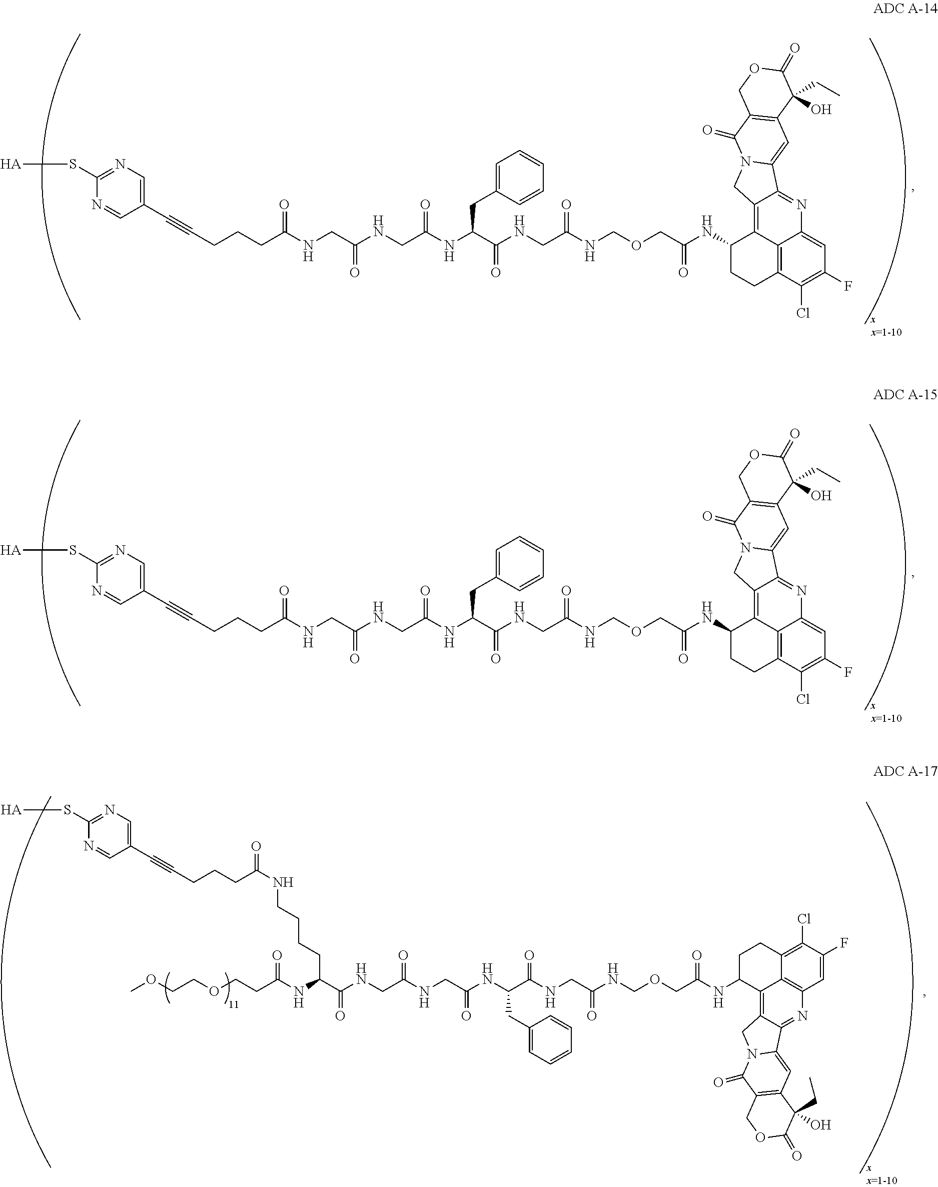

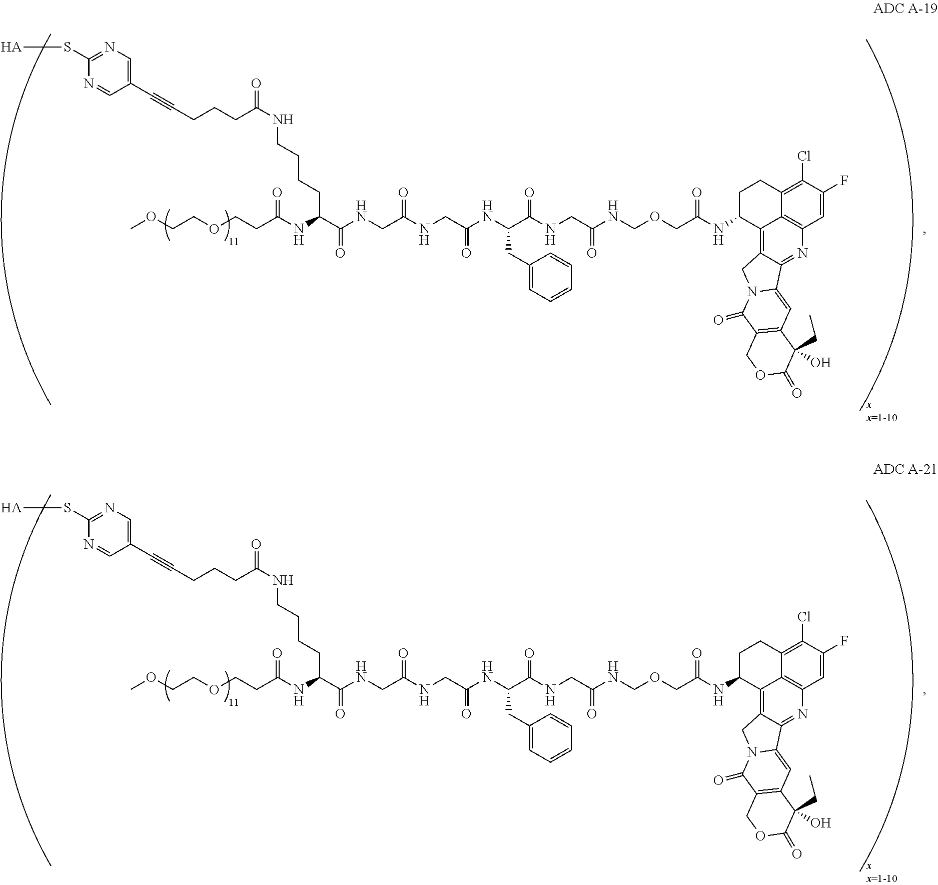

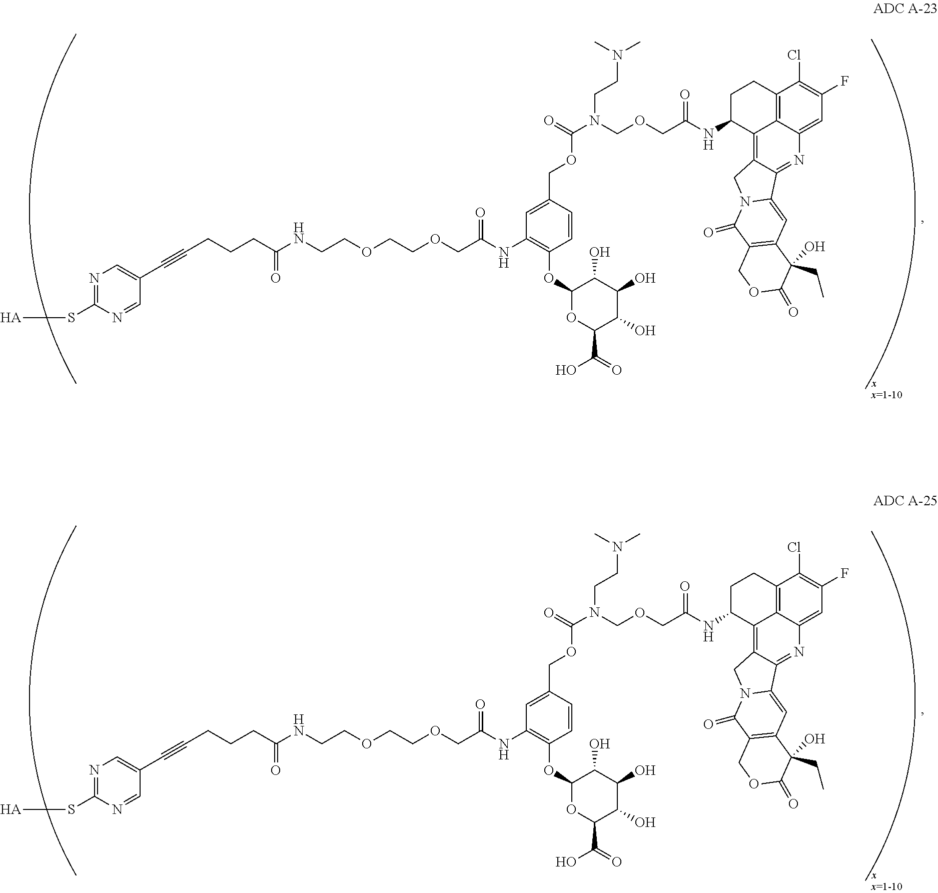

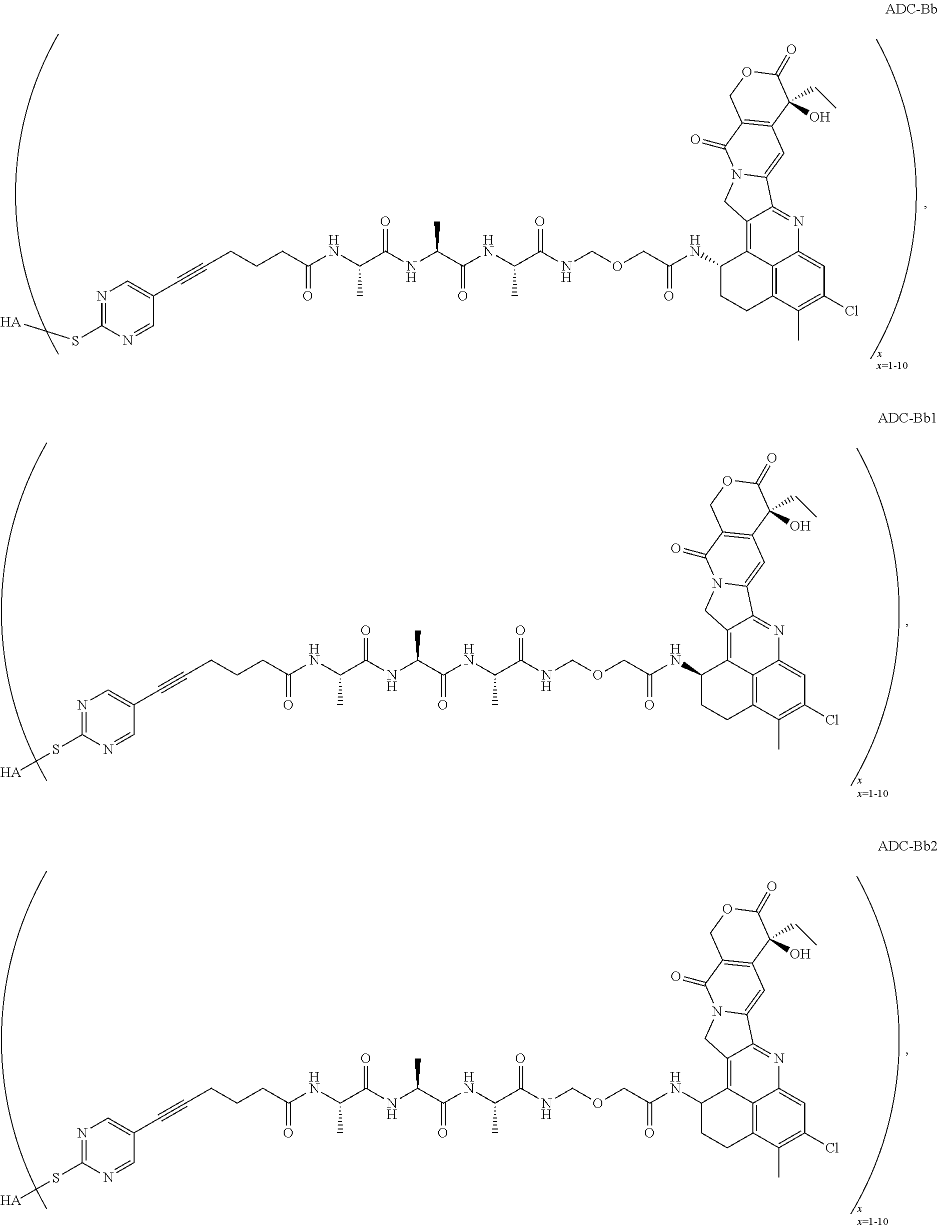

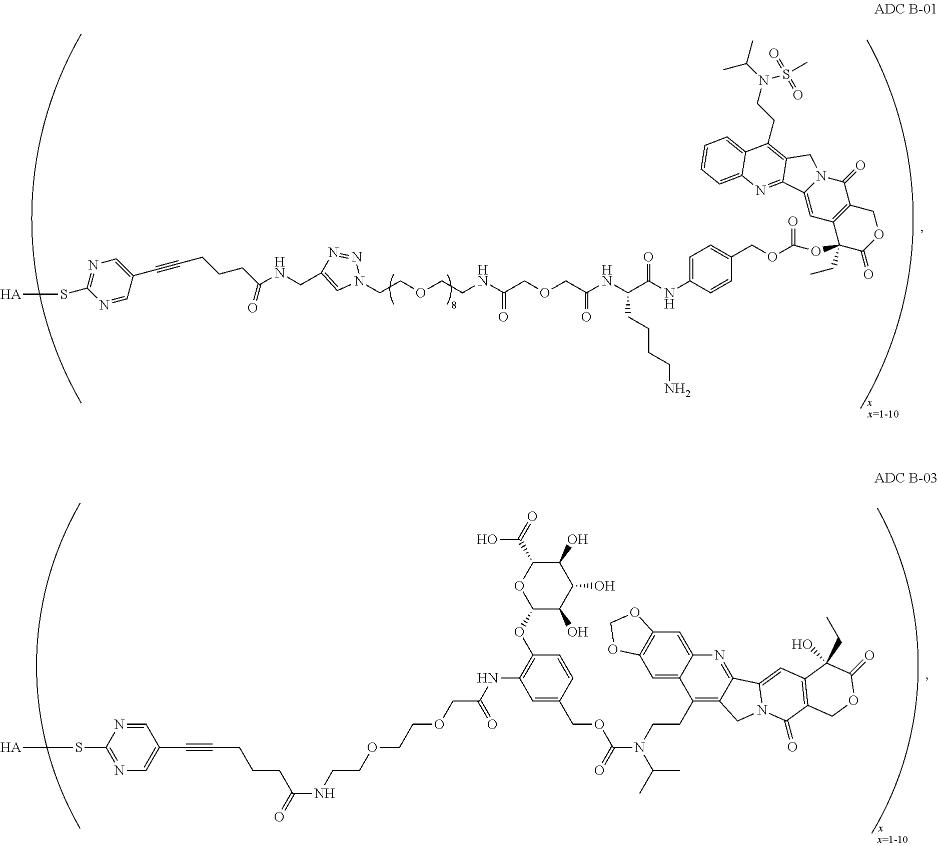

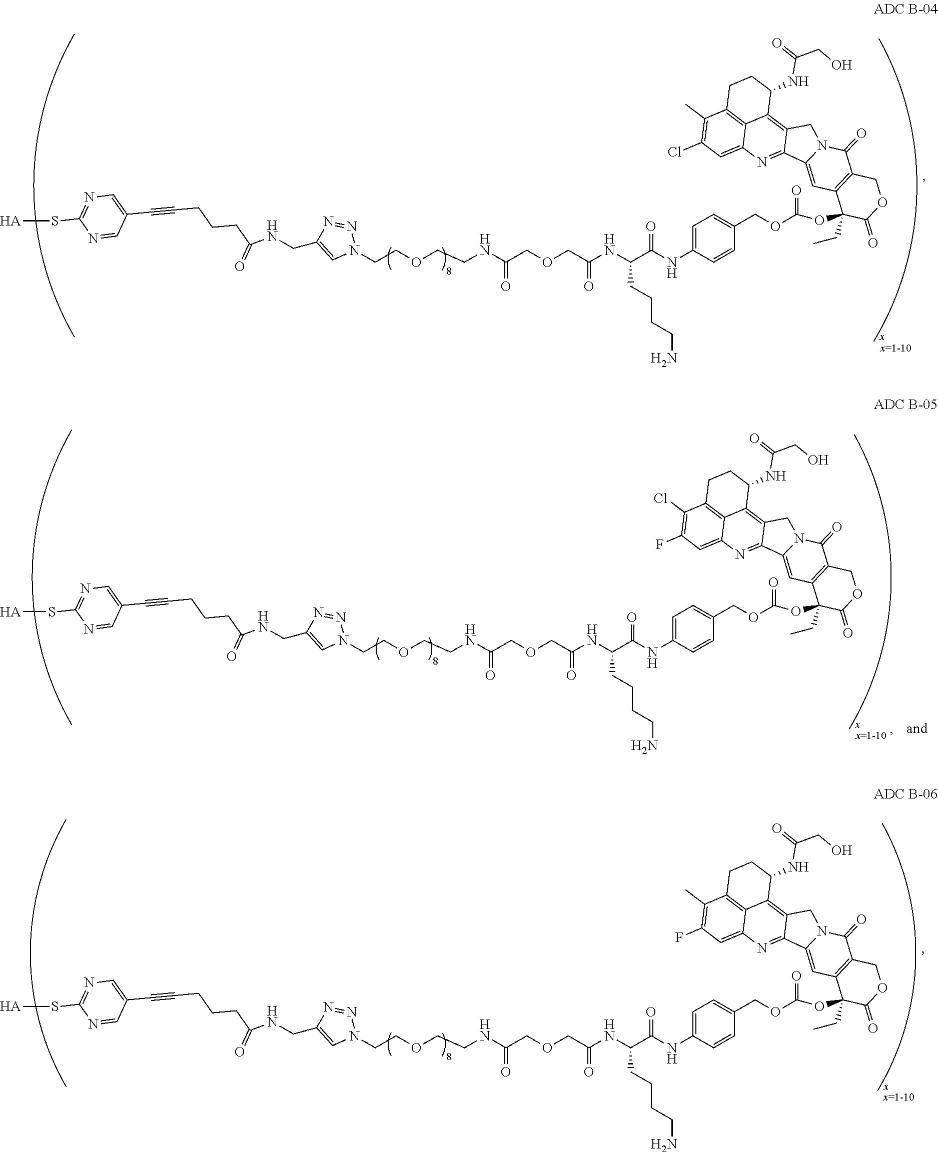

wherein
HA-(S— comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4; and
each -(S-represents a linkage from a sulfhydryl in the antibody to each pyrimidinyl group in formulas ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06.

In one embodiment of the antibody-drug conjugate HA-(S— comprises any one of the antibodies or antigen-binding fragment thereof disclosed herein; each -(S-represents a linkage from a sulfhydryl in the antibody to each pyrimidinyl group in formulas ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06; and x=1-10. In one embodiment of the antibody-drug conjugate, HA-(S— comprises any one of the antibodies or antigen-binding fragment thereof disclosed herein; each -(S-represents a linkage from a sulfhydryl in the antibody to each pyrimidinyl group in formulas ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06; and x=4-8. In one embodiment of the antibody-drug conjugate, HA-(S— comprises any one of the antibodies or antigen-binding fragment thereof disclosed herein; each -(S-represents a linkage from a sulfhydryl in the antibody to each pyrimidinyl group in formulas ADC-Aa, ADC-Aa1, ADC-Aa2, ADC A-09, ADC A-11, ADC A-14, ADC A-15, ADC A-17, ADC A-19, ADC A-21, ADC A-23, ADC A-25, ADC-Bb, ADC-Bb1, ADC-Bb2, ADC-B01, and ADC B-03 to ADC B-06; and x=7-8.

In some embodiments, the antibody-drug conjugate is selected from formulas ADC-Aa and ADC-Bb:

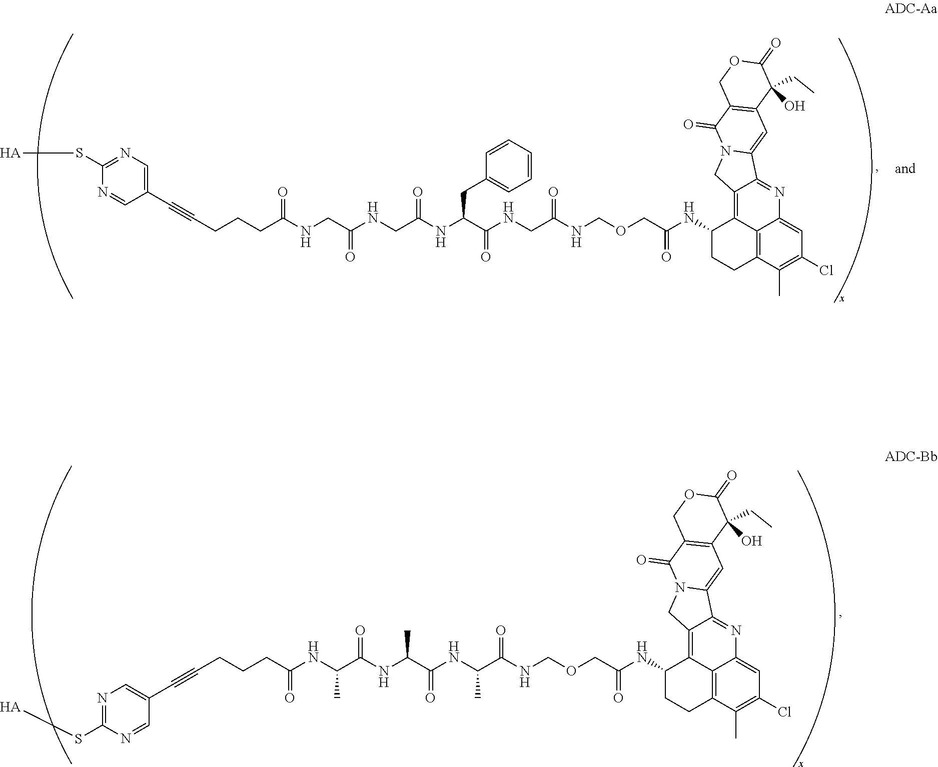

wherein the HA-(S— in the antibody-drug conjugate comprises a V_{H} having the amino acid sequence as set forth in SEQ ID NO: 1 and V_{L} having the amino acid sequence as set forth in SEQ ID NO: 2, for example HA-(S— comprises a V_{H} having the amino acid sequence as set forth in SEQ ID NO: 1 and CH having the amino acid sequence as set forth in SEQ ID NO: 3, VL having the amino acid sequence as set forth in SEQ ID NO: 2 and CL having the amino acid sequence as set forth in SEQ ID NO: 4; each -(S-represents a linkage from a sulfhydryl in the antibody to each pyrimidinyl group in formulas ADC-Aa and ADC-Bb; and x=1-16, 1-8, 4-8, 4 or 8.

In the foregoing embodiment for antibody drug conjugates, for example, for antibody drug conjugates of formulas ADC-Aa and ADC-Bb, in one embodiment, x is 1-16. In one embodiment, x is 1-8. In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formulas ADC-Aa and ADC-Bb.

In one embodiment, x is 3-9. In one embodiment, x is 4-8. In one embodiment, x is 3, 4, 7 or 8. In one embodiment, x is 3. In one embodiment, x is 4. In one embodiment, x is 7. In one embodiment, x is 8.

In some embodiments, the antibody-drug conjugate is selected from ADC-Aa and ADC-Bb:

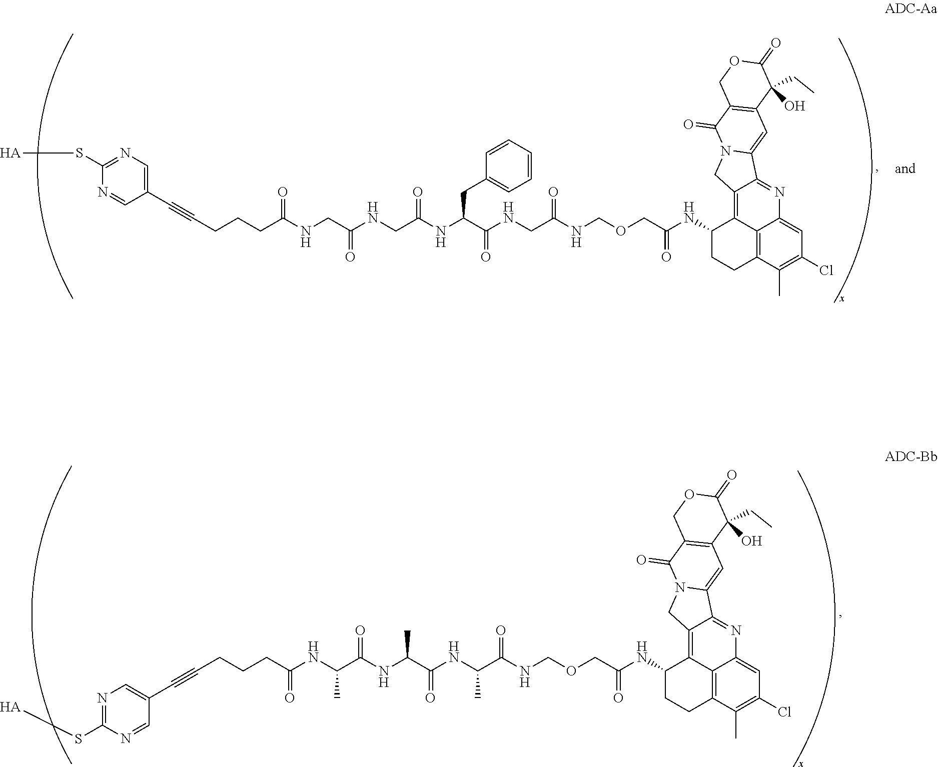

wherein HA-(S— comprises an antibody or antigen-binding fragment;
each -(S-represents a linkage from a sulfhydryl of the antibody or antigen-binding fragment to each pyrimidinyl group in formulas ADC-Aa and ADC-Bb;
x is 2-8, 3-4, or 7-8; and
the antibody or antigen binding fragment comprises:
(1) the following heavy chain variable regions (VH) and/or light chain variable regions (VL), wherein CDRs are defined according to a Chothia numbering system:
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (1) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (2) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants of (3) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (4) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement.

In the foregoing embodiment for antibody drug conjugates, for example, for antibody drug conjugates of formulas ADC-Aa and ADC-Bb, in one embodiment, x is 2-8. In one embodiment, x is 3-4. In one embodiment, x is 7-8. In one embodiment, x is an integer.

In certain embodiments, each -(S-represents a linkage from a sulfhydryl of a cysteine residue in the antibody to each pyrimidinyl group in formulas ADC-Aa and ADC-Bb.

In a further embodiment, the HA-(S— in the antibody-drug conjugate is selected from an antibody or antigen binding fragment comprising a V_{H} having the amino acid sequence as set forth in SEQ ID NO: 1 or a variant thereof, and/or, a V_{L} having the amino acid sequence as set forth in SEQ ID NO: 2 or a variant thereof;
wherein the variants have the amino acid sequence identity of at least 70%, at least 80%, at least 85%, at least 90%, at least 91%, at least 92%, at least 93%, at least 94%, at least 95%, at least 96%, at least 97%, at least 98%, at least 99% or 100% as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived; and preferably, the replacement is a conservative replacement.

In a further embodiment, the HA-(S- in the antibody-drug conjugate is an antibody or antigen binding fragment comprising a V_{H} having the amino acid sequence as set forth in SEQ ID NO: 1, and/or, a V_{L} having the amino acid sequence as set forth in SEQ ID NO: 2.

In a further embodiment, the HA-(S- in the antibody-drug conjugate is an antibody or antigen binding fragment comprising a heavy chain comprising a V_{H} having the amino acid sequence set forth in SEQ ID NO: 1.

In a further embodiment, the HA-(S- in the antibody-drug conjugate is an antibody or antigen binding fragment comprising a heavy chain comprising a V_{H} having the amino acid sequence set forth in SEQ ID NO: 1, wherein the N-terminal glutamine of the antibody variable region has undergone cyclization to pyroglutamate.

In a further embodiment, the HA-(S- in the antibody-drug conjugate is an antibody or antigen binding fragment comprising a heavy chain comprising a V_{H} having the amino acid sequence set forth in SEQ ID NO: 1, wherein the N-terminal glutamine of the antibody variable region has undergone cyclization to pyroglutamic acid.

In a further embodiment, the HA-(S- in the antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 or a variant thereof and/or a V_{L} having the amino acid sequence set forth in SEQ ID NO: 2 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived.

In a further embodiment, the HA-(S- in the antibody-drug conjugate is an antibody or antigen binding fragment obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 and/or a V_{L} having the amino acid sequence set forth in SEQ ID NO: 2.

In a further embodiment, the HA-(S- in the antibody-drug conjugate is an antibody or antigen binding fragment conjugated through one or more-S-from the one or more sulfhydryls in the antibody or antigen-binding fragment to the one or more M. In a further embodiment, the HA-(S- in the antibody-drug conjugate is an antibody or antigen binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through one or more-S-from the one or more sulfhydryls in the antibody or antigen-binding fragment to the one or more M.

In a further embodiment, the HA-(S- in the antibody-drug conjugate is an antibody or antigen binding fragment conjugated through x number of—S-from x number of sulfhydryl(s) in the antibody or antigen-binding fragment to x number of the one or more M. In a further embodiment, the HA-(S- in the antibody-drug conjugate is an antibody or antigen binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through x number of—S-from x number of sulfhydryl(s) in the antibody or antigen-binding fragment to x number of the one or more M.

In a further embodiment, the HA-(S- in the antibody-drug conjugates provided herein represents an antibody or antigen-binding fragment thereof conjugated through-S-from a sulfhydryl in the antibody or antigen-binding fragment to M. In a further embodiment, the HA-(S—in the antibody-drug conjugates provided herein represents an antibody or antigen-binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through-S-from a sulfhydryl in the antibody or antigen-binding fragment to M.

In a further embodiment, the HA-(S- in the antibody-drug conjugates provided herein represents an antibody or antigen-binding fragment thereof conjugated through-S-from x number of sulfhydryl(s) in the antibody or antigen-binding fragment to x number of M. In a further embodiment, the HA-(S- in the antibody-drug conjugates provided herein represents an antibody or antigen-binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through-S-from x number of sulfhydryl(s) in the antibody or antigen-binding fragment to x number of M.

In a further embodiment, the antibody-drug conjugate is selected from ADC-Aa and ADC-Bb:

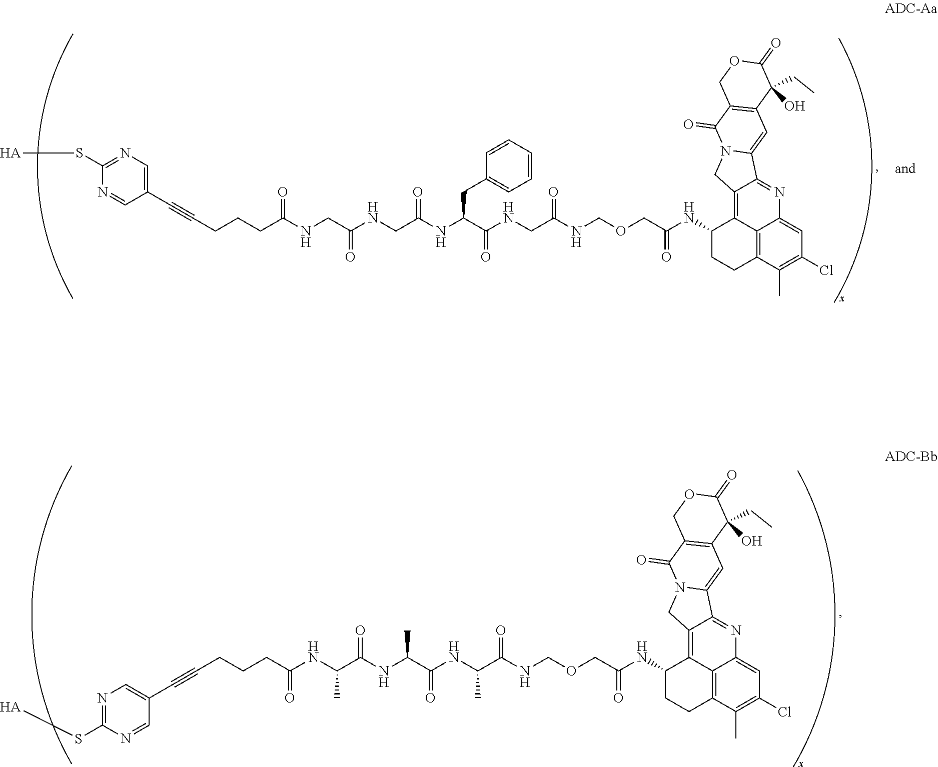

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formulas ADC-Aa and ADC-Bb; and
x=1-10, 1-8, 4-8, 4 or 8.

In the foregoing embodiment for antibody drug conjugates, for example, for antibody drug conjugates of formulas ADC-Aa and ADC-Bb, in one embodiment, x is 1-10. In one embodiment, x is 1-8. In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formulas ADC-Aa and ADC-Bb.

In some embodiments, the antibody-drug conjugate is selected from ADC-Aa, ADC-Aa1, ADC-Aa2, ADC-Bb, ADC-Bb1, and ADC-Bb2 shown as follows:

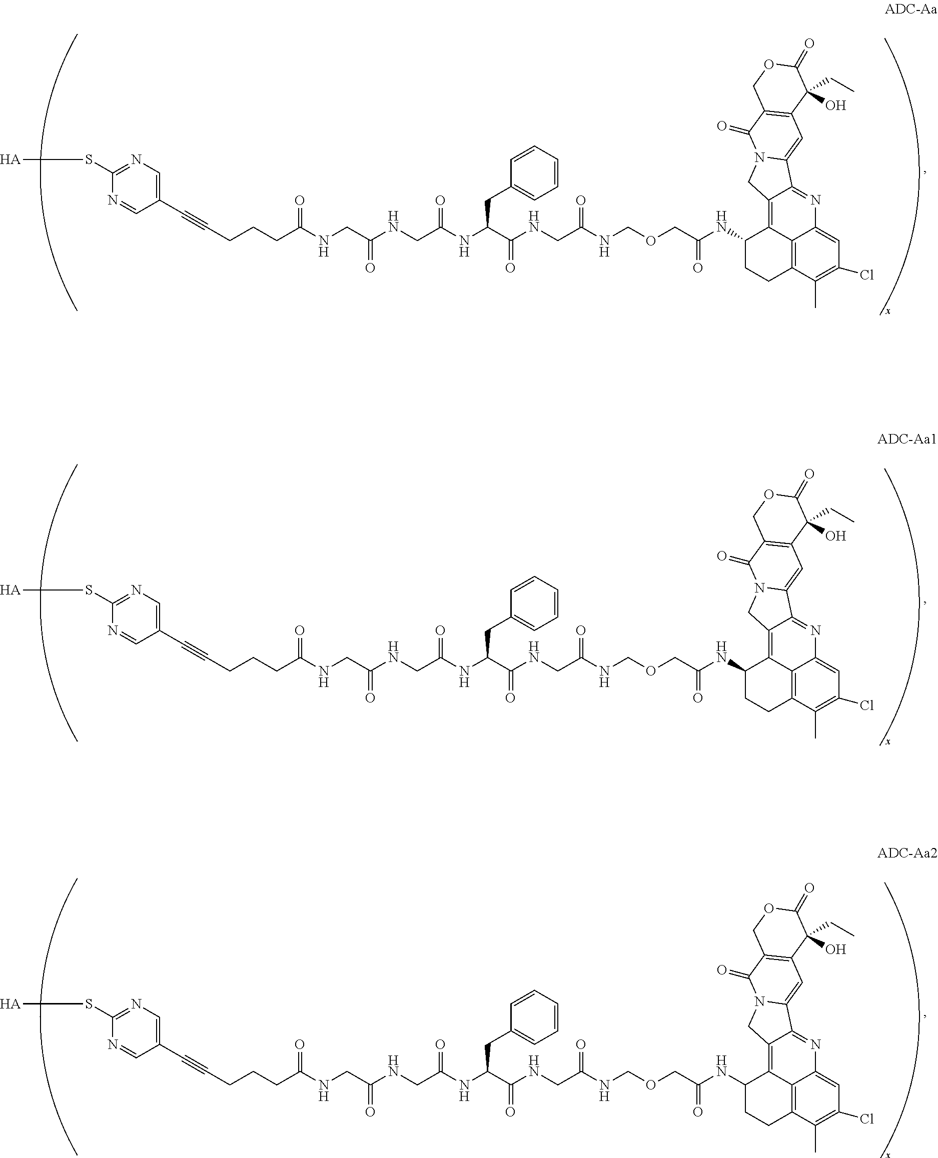

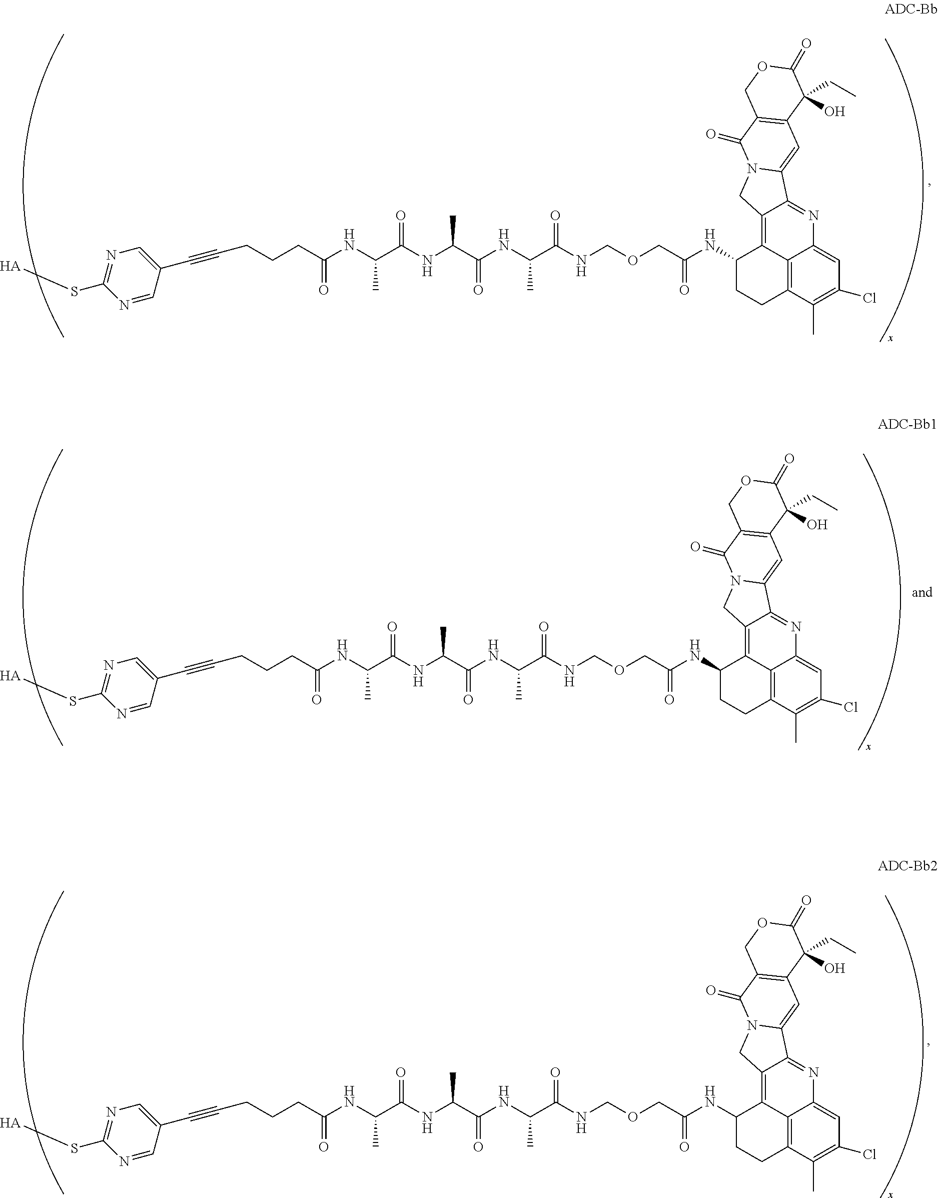

wherein HA-(S- comprises any one of the antibodies disclosed herein;
each -(S-represents a linkage from a sulfhydryl in the antibody to each pyrimidinyl group in formulas ADC-Aa, ADC-Aa1, ADC-Aa2, ADC-Bb, ADC-Bb1, and ADC-Bb2;
and x=1-10, 1-8, 4-8, 4 or 8.

In the foregoing embodiment for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-10. In one embodiment, x is 1-8. In one embodiment, x is 4-8. In one embodiment, x is 4. In one embodiment, x is 8. In one embodiment, x is an integer.

In some embodiments, the antibody-drug conjugate is selected from ADC-Aa, ADC-Aa1, ADC-Aa2, ADC-Bb, ADC-Bb1, and ADC-Bb2, wherein HA-(S- comprises a V_{H} having the amino acid sequence as set forth in SEQ ID NO: 1, and a V_{L} having the amino acid sequence as set forth in SEQ ID NO: 2; each -(S-represents a linkage from a sulfhydryl in the antibody to each pyrimidinyl group; and x=1-16, 1-10 or 1-8. In certain embodiments, each -(S-represents a linkage from a sulfhydryl of a cysteine residue in the antibody to each pyrimidinyl group in formulas ADC-Aa, ADC-Aa1, ADC-Aa2, ADC-Bb, ADC-Bb1, and ADC-Bb2.

In the foregoing embodiment for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-16. In one embodiment, x is 1-10. In one embodiment, x is 1-8.

In some embodiments, the antibody-drug conjugate is selected from ADC-Aa, ADC-Aa1, ADC-Aa2, ADC-Bb, ADC-Bb1, and ADC-Bb2, wherein HA-(S- comprises a heavy chain (HC) having the amino acid sequence as set forth in SEQ ID NO: 20 and a light chain (LC) having the amino acid sequence as set forth in SEQ ID NO: 21; each -(S-represents a linkage from a sulfhydryl in the antibody to each pyrimidinyl group in formulas ADC-Aa, ADC-Aa1, ADC-Aa2, ADC-Bb, ADC-Bb1, and ADC-Bb2; and x=1-10, 1-8, 4-8, 4 or 8. In certain embodiments, each -(S-represents a linkage from a sulfhydryl of a cysteine residue in the antibody to each pyrimidinyl group in formulas ADC-Aa, ADC-Aa1, ADC-Aa2, ADC-Bb, ADC-Bb1, and ADC-Bb2.

In the foregoing embodiment for antibody drug conjugates, for example, for antibody drug conjugates of formulas ADC-Aa, ADC-Aa1, ADC-Aa2, ADC-Bb, ADC-Bb1, and ADC-Bb2, in one embodiment, x is 1-16. In one embodiment, x is 1-10. In one embodiment, x is 1-8. In one embodiment, x is 4-8. In one embodiment, x is 4. In one embodiment, x is 8. In one embodiment, x is an integer.

In a further embodiment, the antibody-drug conjugate is ADC-Aa:

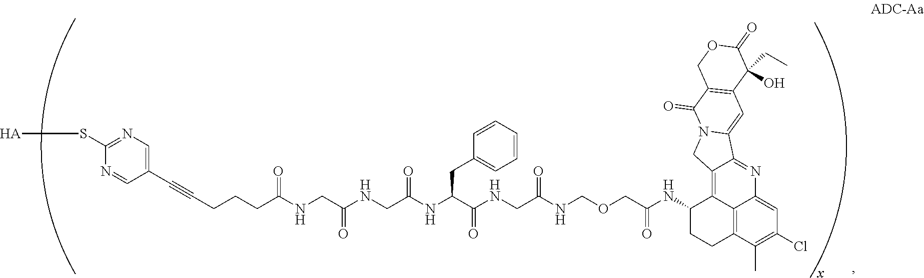

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Aa;
and x=1-10, 1-8, 4-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

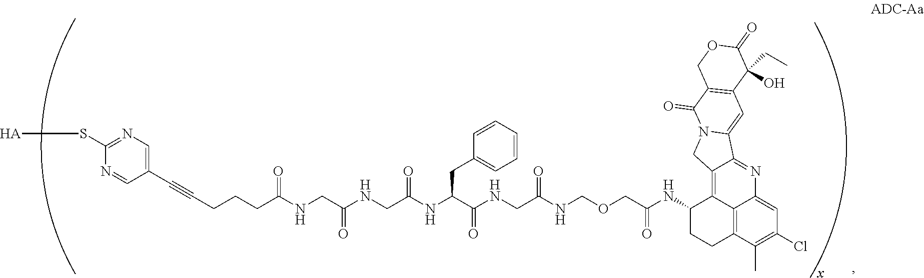

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Aa;
and x=3-4 or 7-9.

In the foregoing embodiment for antibody drug conjugate, for example, for antibody drug conjugate of formula ADC-Aa, in one embodiment, x is 3-4. In one embodiment, x is 7-9. In one embodiment, x is an integer.

In a further embodiment, the antibody-drug conjugate is ADC-Aa:

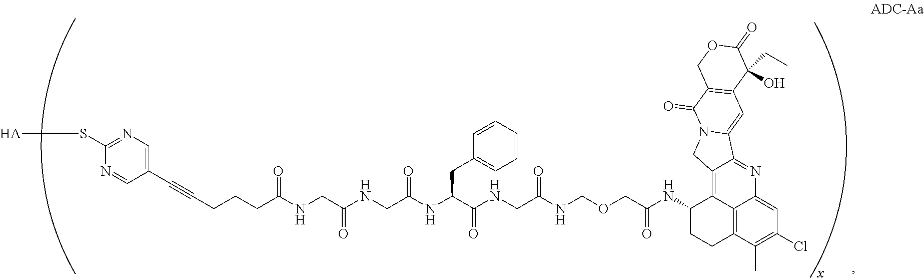

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Aa;
and x=7-9 or 7-8.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

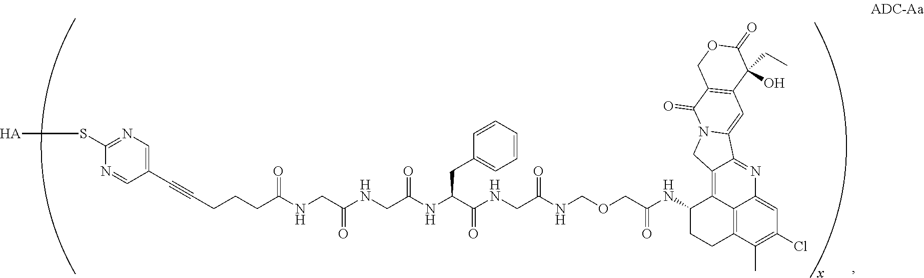

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Aa;
and x is 8-9 or 7-8.

In a further embodiment, the antibody-drug conjugate is ADC-Aa:

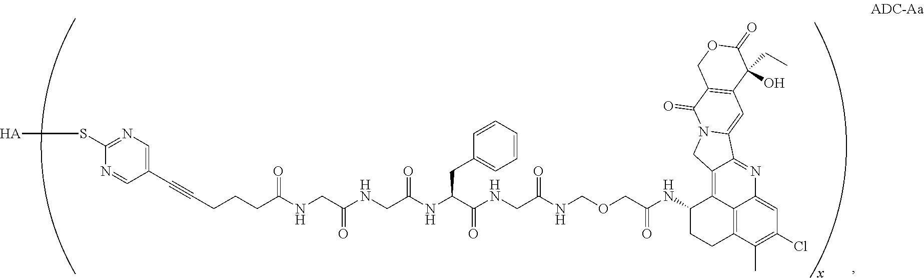

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Aa;
and x is 8.

In certain embodiments, each -(S-represents a linkage from a sulfhydryl of a cysteine residue in the antibody to each pyrimidinyl group in formula ADC-Aa.

In the foregoing embodiments for antibody drug conjugate, for example, for antibody drug conjugate of formula ADC-Aa, in one embodiment, x is 3-4. In one embodiment, x is 7-9. In one embodiment, x is 7-8. In one embodiment, x is an integer.

In a further embodiment, the antibody-drug conjugate is ADC-Bb:

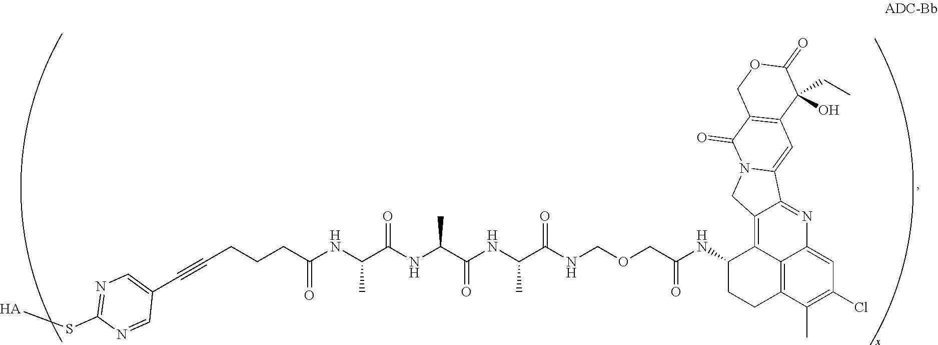

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x=1-10, 1-8, 4-8, 4 or 8.

In the foregoing embodiment for antibody drug conjugate, for example, for antibody drug conjugate of formula ADC-Aa, in one embodiment, x is 1-10. In one embodiment, x is 1-8.

In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

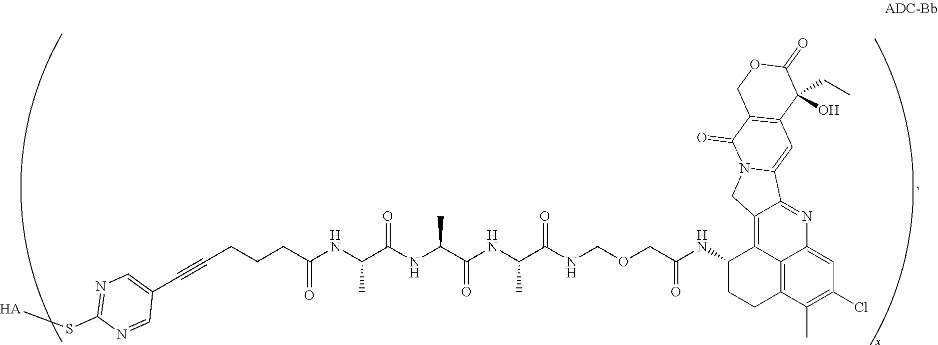

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb; and
x is 3-4, 7-8, or 7-9.

In the foregoing embodiment for antibody drug conjugate, for example, for antibody drug conjugate of formula ADC-Bb, in one embodiment, x is 3-4. In one embodiment, x is 7-9. In one embodiment, x is 7-8. In one embodiment, x is an integer.

In a further embodiment, the antibody-drug conjugate is ADC-Bb:

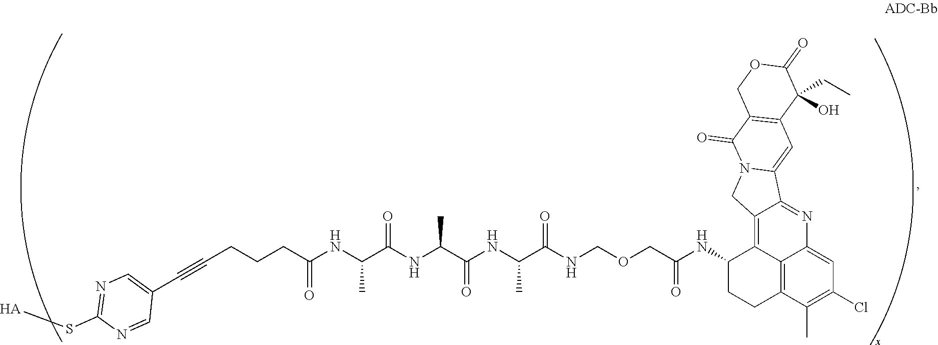

wherein HA-S(- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x is 7-9.

In a further embodiment, the antibody-drug conjugate is ADC-Bb:

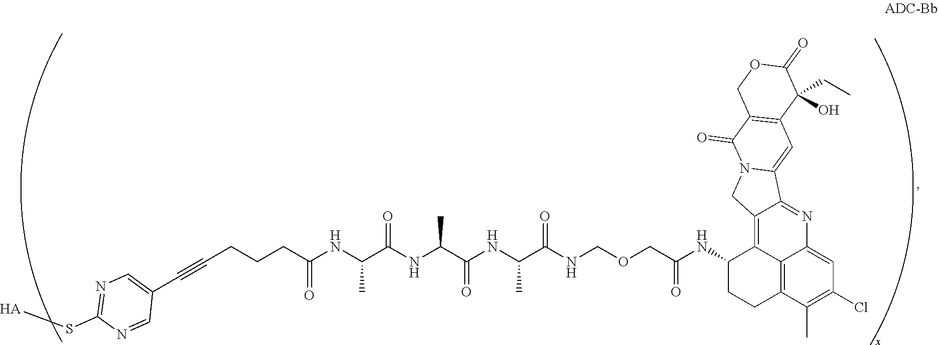

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x is 8-9.

In a further embodiment, the antibody-drug conjugate is ADC-Bb:

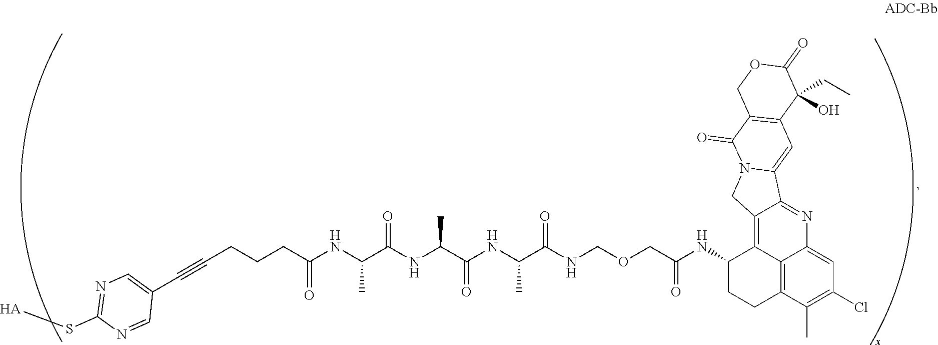

wherein HA-S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x=8.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

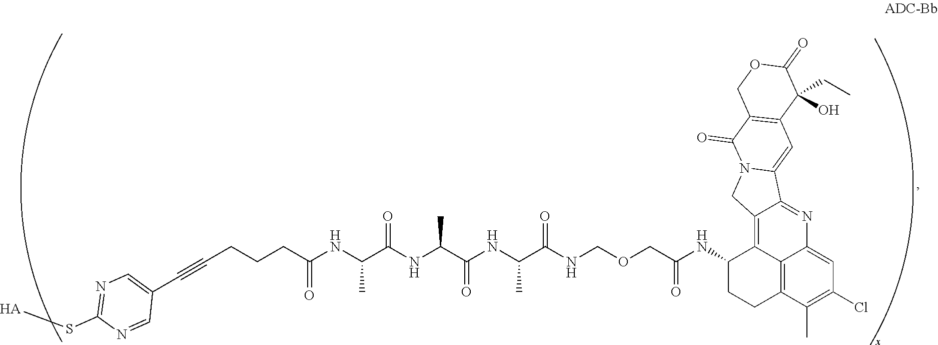

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x is 3-4.

In a further embodiment, the antibody-drug conjugate is ADC-Bb:

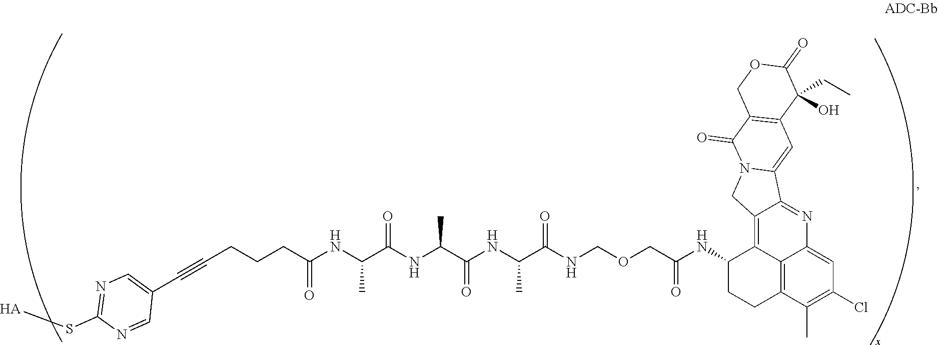

wherein HA-(S- comprises a V_{H} having the amino acid sequence as set forth in SEQ ID NO: 1 and V_{L} having the amino acid sequence as set forth in SEQ ID NO: 2, for example an antibody or antigen-binding fragment thereof comprising a V_{H} having the amino acid sequence as set forth in SEQ ID NO: 1 and CH having the amino acid sequence as set forth in SEQ ID NO: 3, V_{L} having the amino acid sequence as set forth in SEQ ID NO: 2 and CL having the amino acid sequence as set forth in SEQ ID NO: 4;
each -(S-represents a linkage from a sulfhydryl in the antibody to each pyrimidinyl group in formula ADC-Bb;
and x is 4.

In a further embodiment, the antibody-drug conjugate is ADC-Aa or ADC-Bb:

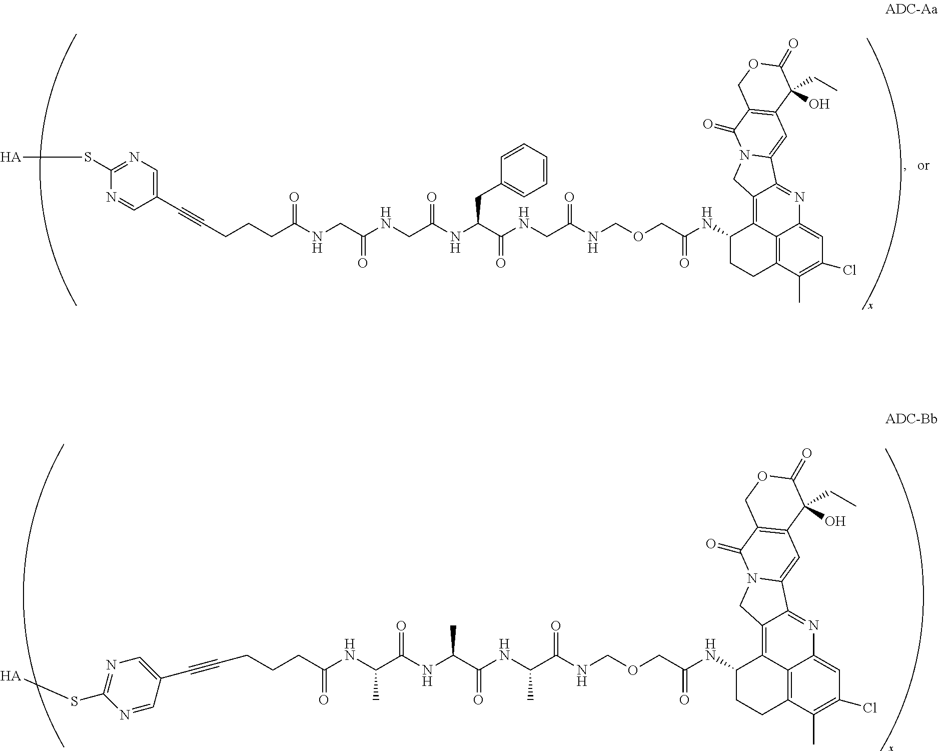

wherein HA-(S- comprises a heavy chain (HC) having the amino acid sequence as set forth in SEQ ID NO: 20 or 22 and a light chain (LC) having the amino acid sequence as set forth in SEQ ID NO: 21;
each -(S-represents a linkage from a sulfhydryl in the antibody to each pyrimidinyl group in formulas ADC-Aa and ADC-Bb;
and x=1-10, 1-8, 4-8, 4 or 8.

In the foregoing embodiment for antibody drug conjugates, for example, for antibody drug conjugates of formulas ADC-Aa and ADC-Bb, in one embodiment, x is 1-10. In one embodiment, x is 1-8. In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formulas ADC-Aa and ADC-Bb.

In a further embodiment, the antibody-drug conjugate is ADC-Aa or ADC-Bb:

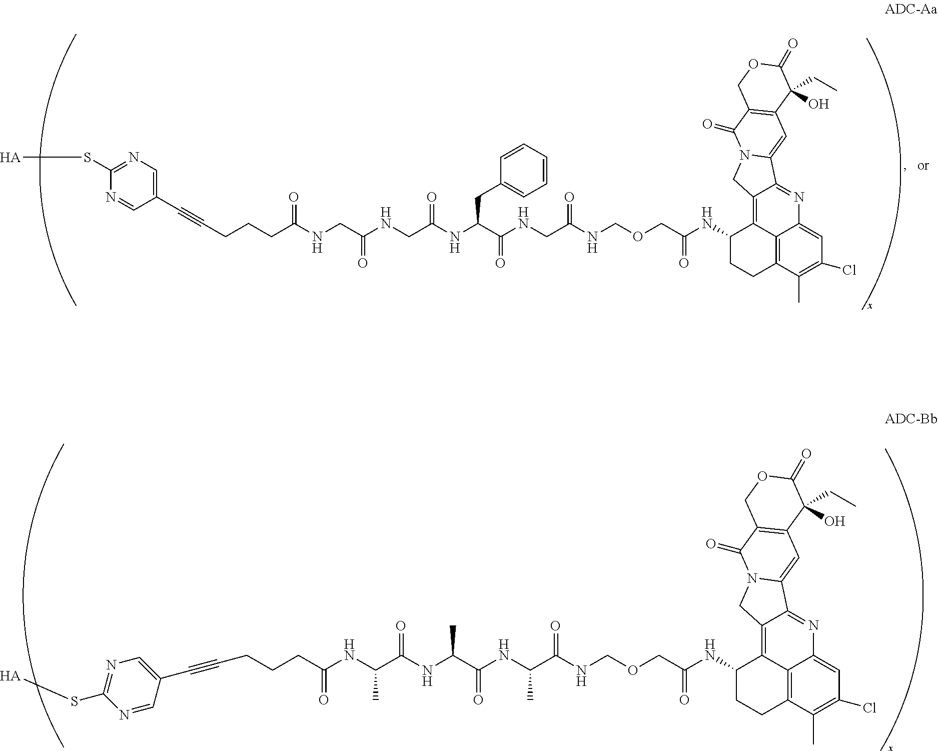

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formulas ADC-Aa and ADC-Bb;
and x is 3-4, 7-8, or 7-9.

In certain embodiments, each -(S-represents a linkage from a sulfhydryl of a cysteine residue in the antibody to each pyrimidinyl group in formulas ADC-Aa and ADC-Bb.

In the foregoing embodiment for antibody drug conjugates, for example, for antibody drug conjugates of formulas ADC-Aa and ADC-Bb, in one embodiment, x is 3-4. In one embodiment, x is 7-9. In one embodiment, x is 7-8. In one embodiment, x is an integer.

In a further embodiment, the antibody-drug conjugate is ADC-Aa:

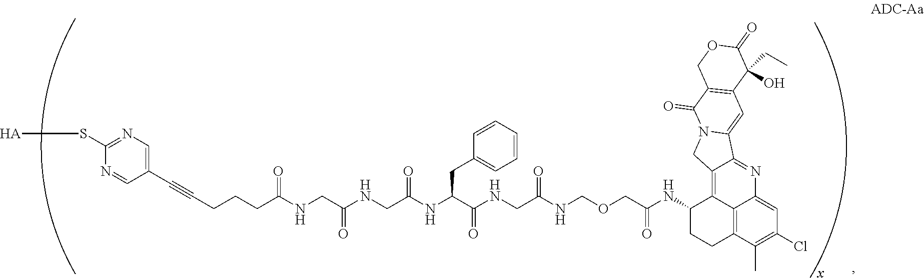

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Aa;
and x=1-10.

In one embodiment, x=4-8. In one embodiment, x=4. In one embodiment, x=8. In one embodiment, x=7-8. In one embodiment, x=7. In one embodiment, x is an integer.

In a further embodiment, the antibody-drug conjugate is ADC-Aa:

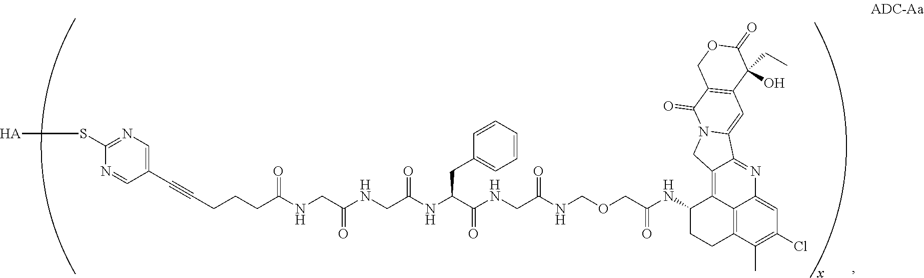

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Aa;
and x=7-9.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

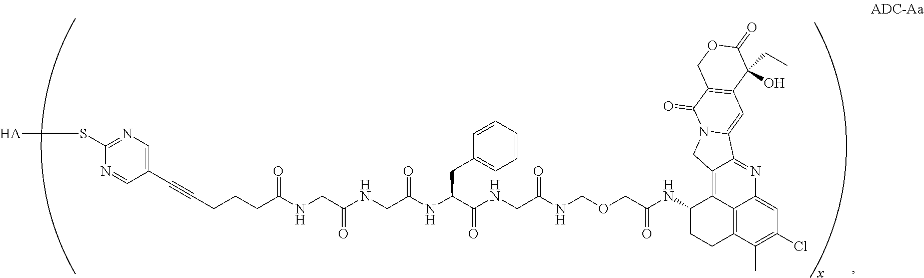

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x=7-9.

In a further embodiment, the antibody-drug conjugate is ADC-Aa:

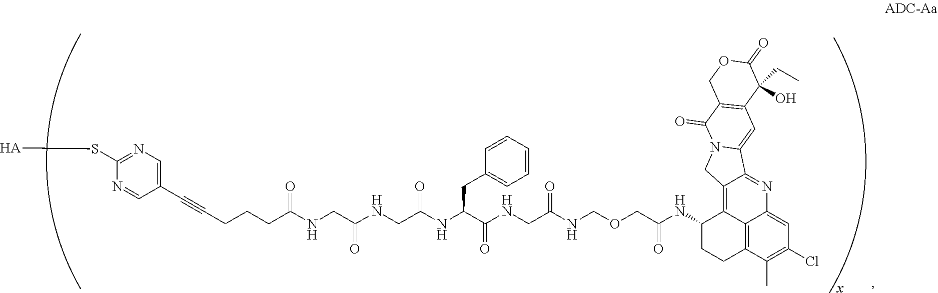

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Aa;
and x=7-8.

In a further embodiment, the antibody-drug conjugate is ADC-Aa:

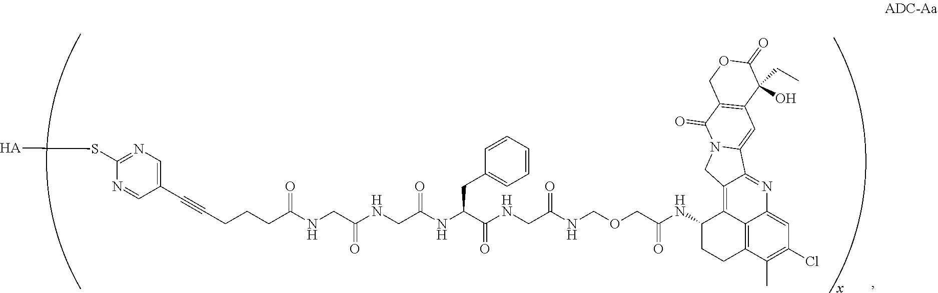

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Aa;
and x=8.

In a further embodiment, the antibody-drug conjugate is ADC-Aa:

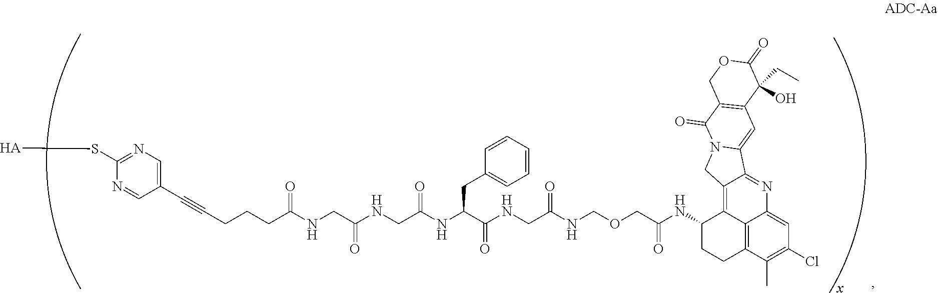

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x=8.

In a further embodiment, the antibody-drug conjugate is ADC-Aa:

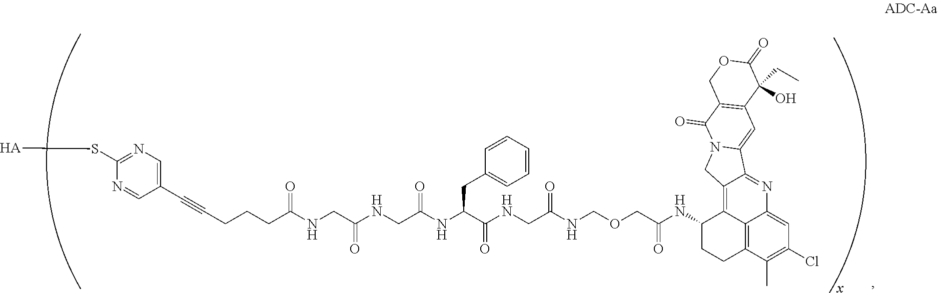

wherein HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl in the antibody to each pyrimidinyl group in formula ADC-Aa;
and x=1-10, 1-8, 4-8, 4 or 8.

In certain embodiments, each -(S-represents a linkage from a sulfhydryl of a cysteine residue in the antibody to each pyrimidinyl group in formulas ADC-Aa.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 4. In one embodiment, x is 8. In one embodiment, x is an integer.

In a further embodiment, the antibody-drug conjugate is ADC-Aa:

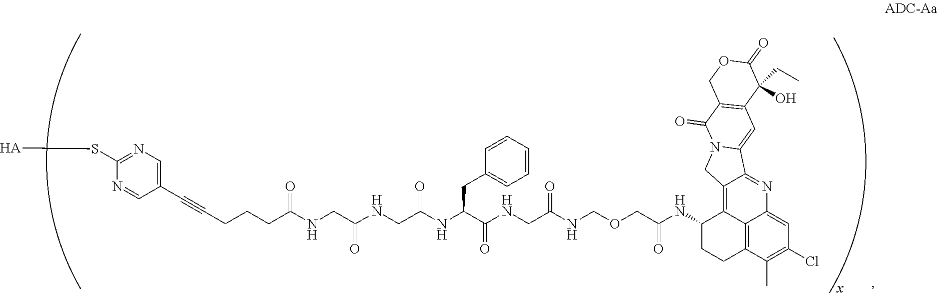

wherein HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl in the antibody to each pyrimidinyl group in formula ADC-Aa;
and x=7-8.

In a further embodiment, the antibody-drug conjugate is ADC-Aa:

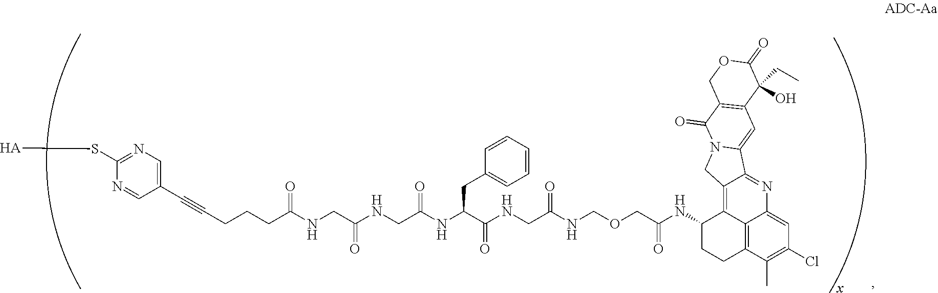

wherein HA-(S- comprises a heavy chain (HC) having the amino acid sequence as set forth in SEQ ID NO: 22 and a light chain (LC) having the amino acid sequence as set forth in SEQ ID NO: 21;
each -(S-represents a linkage from a sulfhydryl of the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

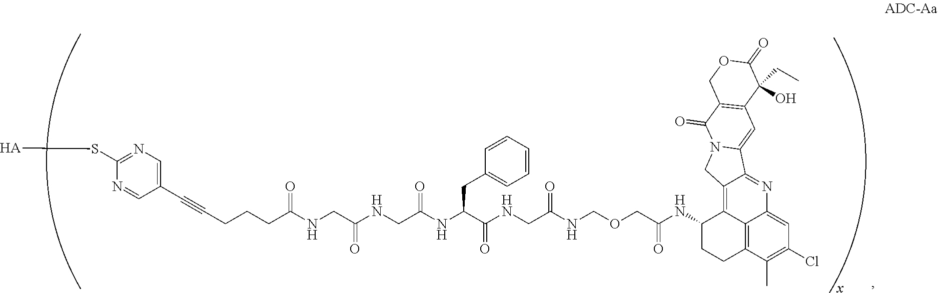

wherein HA-(S- comprises a heavy chain (HC) having the amino acid sequence as set forth in SEQ ID NO: 23 and a light chain (LC) having the amino acid sequence as set forth in SEQ ID NO: 21;
each -(S-represents a linkage from a sulfhydryl of the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

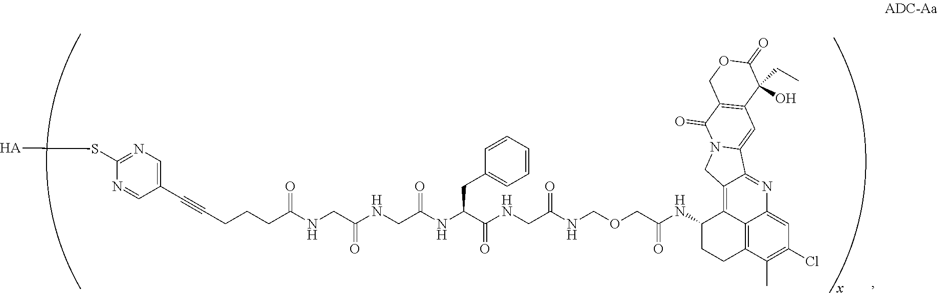

wherein HA-(S- comprises a heavy chain (HC) having the amino acid sequence as set forth in SEQ ID NO: 24 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Aa:

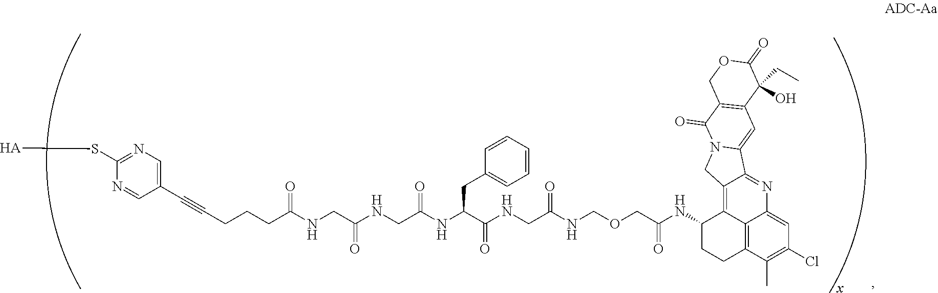

wherein HA-(S- comprises two heavy chains and two light chains, each heavy chain (HC) consists of the amino acid sequence as set forth in SEQ ID NO: 20 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In a further embodiment, the antibody-drug conjugate is ADC-Aa:

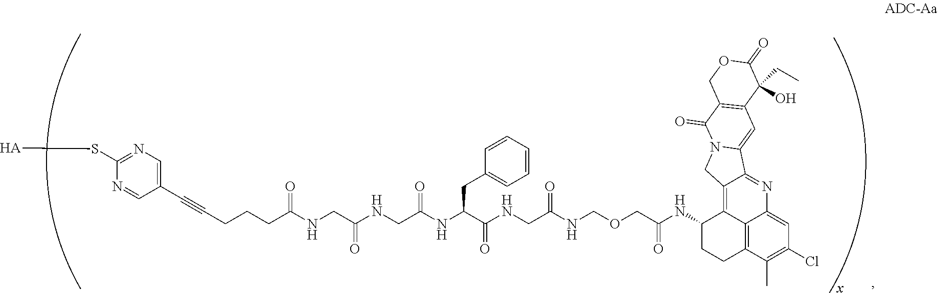

wherein HA-(S- consists of two heavy chains and two light chains, each heavy chain (HC) consists of the amino acid sequence as set forth in SEQ ID NO: 20 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In a further embodiment, the antibody-drug conjugate is ADC-Aa:

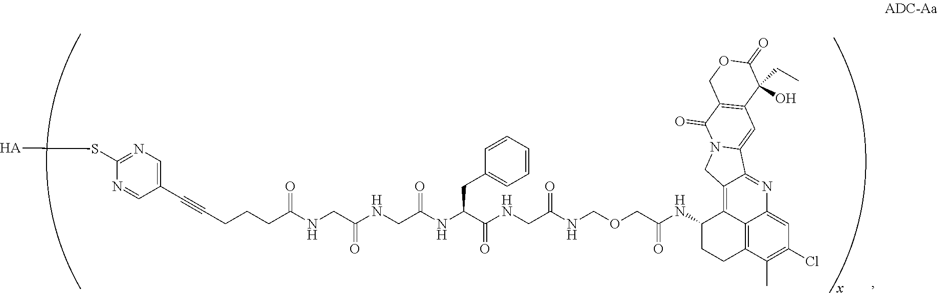

wherein HA-(S- comprises two heavy chains and two light chains, each heavy chain (HC) comprises of the amino acid sequence as set forth in SEQ ID NO: 22 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In a further embodiment, the antibody-drug conjugate is ADC-Aa:

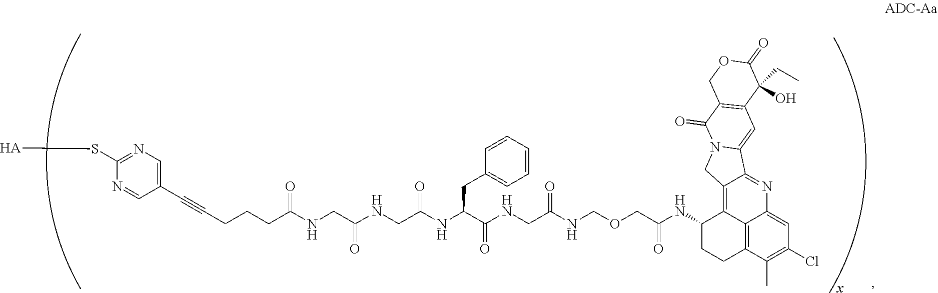

wherein HA-(S- consists of two heavy chains and two light chains, each heavy chain (HC) consists of the amino acid sequence as set forth in SEQ ID NO: 22 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In a further embodiment, the antibody-drug conjugate is ADC-Aa:

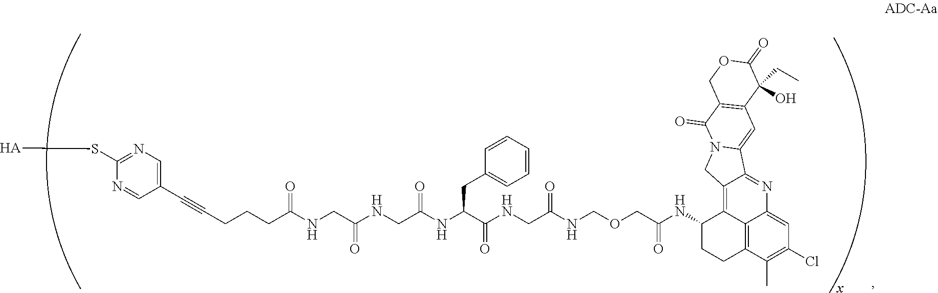

wherein HA-(S- comprises two heavy chains and two light chains, each heavy chain (HC) consists of the amino acid sequence as set forth in SEQ ID NO: 23 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In a further embodiment, the antibody-drug conjugate is ADC-Aa:

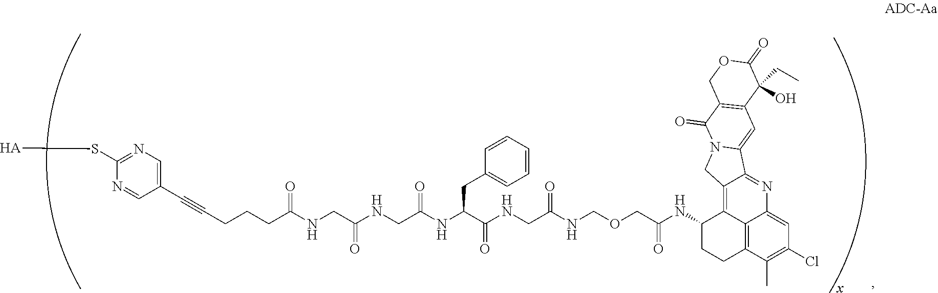

wherein HA-(S- comprises two heavy chains and two light chains, each heavy chain (HC) consists of the amino acid sequence as set forth in SEQ ID NO: 23 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In a further embodiment, the antibody-drug conjugate is ADC-Aa:

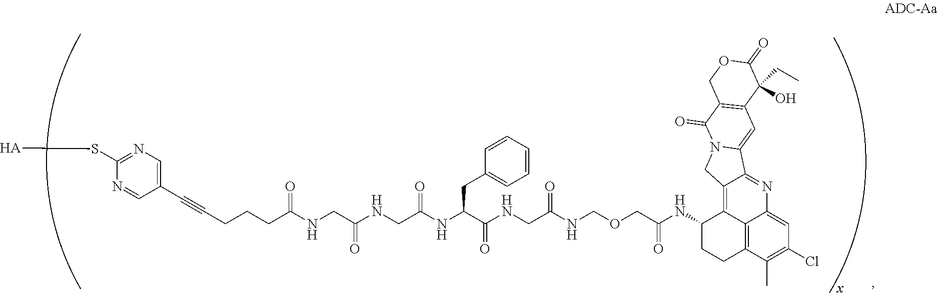

wherein HA-(S- comprises two heavy chains and two light chains, each heavy chain (HC) consists of the amino acid sequence as set forth in SEQ ID NO: 24 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In a further embodiment, the antibody-drug conjugate is ADC-Aa:

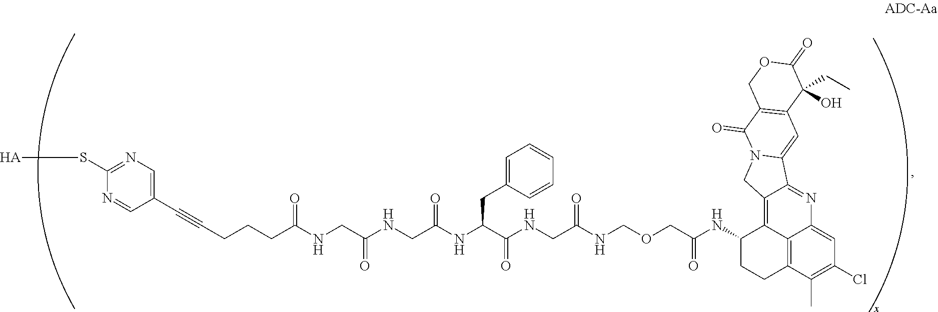

wherein HA-(S- comprises two heavy chains and two light chains, each heavy chain (HC) consists of the amino acid sequence as set forth in SEQ ID NO: 24 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Aa;
and x=1-10, 1-8, 4-8, 7-8, 4 or 8.

In the foregoing embodiment for antibody drug conjugate, for example, for antibody drug conjugate of formula ADC-Aa, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In a further embodiment, the antibody-drug conjugate is ADC-Bb:

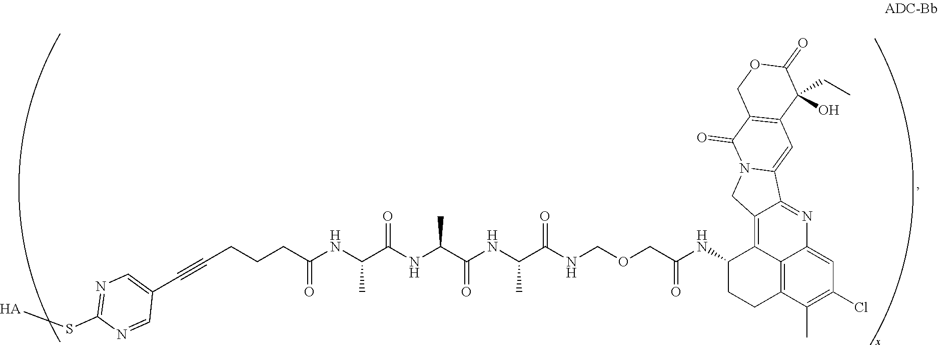

wherein HA-(S- comprises a heavy chain (HC) having the amino acid sequence as set forth in SEQ ID NO: 20 or 22 and a light chain (LC) having the amino acid sequence as set forth in SEQ ID NO: 21;
each -(S-represents a linkage from a sulfhydryl in the antibody to each pyrimidinyl group in formula ADC-Bb;
and x=1-10, 1-8, 4-8, 4 or 8.

In the foregoing embodiment for antibody drug conjugate, for example, for antibody drug conjugate of formula ADC-Bb, in one embodiment, x is 1-10. In one embodiment, x is 1-8. In one embodiment, x is 4-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

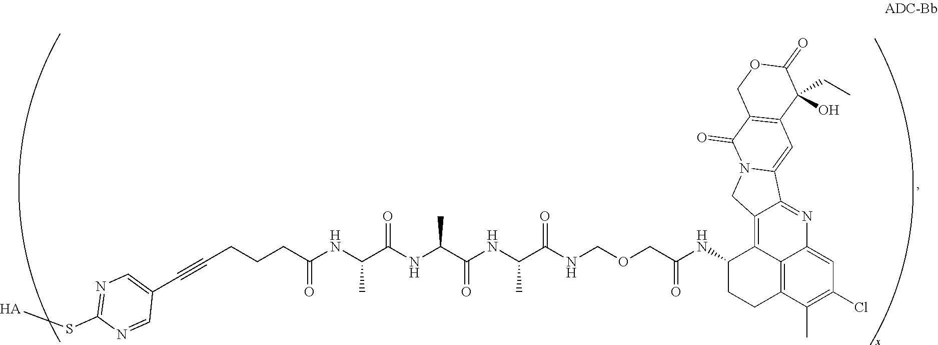

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Bb; and
x is 7-9.

In a further embodiment, the antibody-drug conjugate is ADC-Bb:

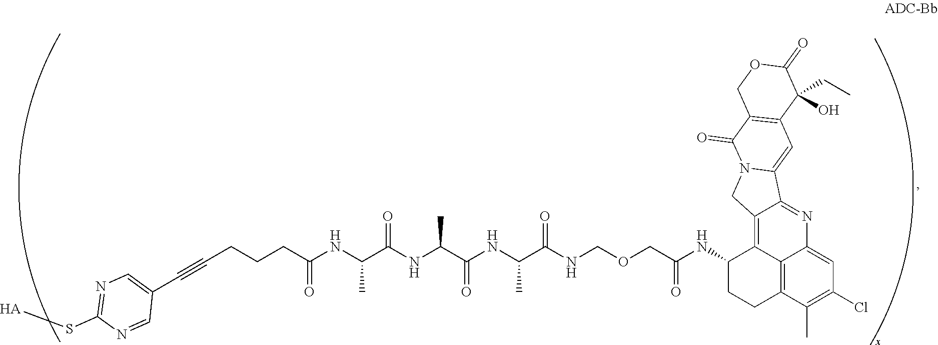

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 7-9.

In a further embodiment, the antibody-drug conjugate is ADC-Bb:

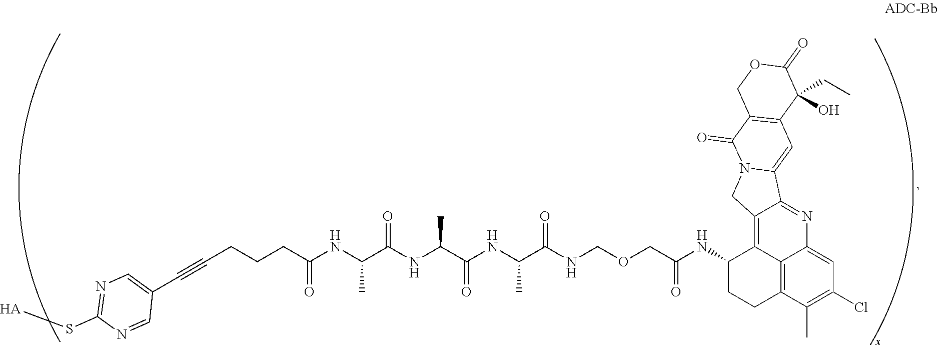

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Bb;
and x is 7-8.

In a further embodiment, the antibody-drug conjugate is ADC-Bb:

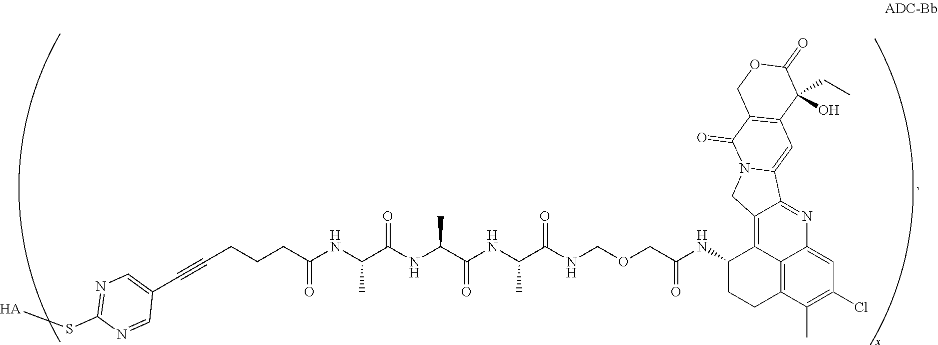

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl in the antibody to each pyrimidinyl group in formula ADC-Bb;
and x is 8.

In a further embodiment, the antibody-drug conjugate is ADC-Bb:

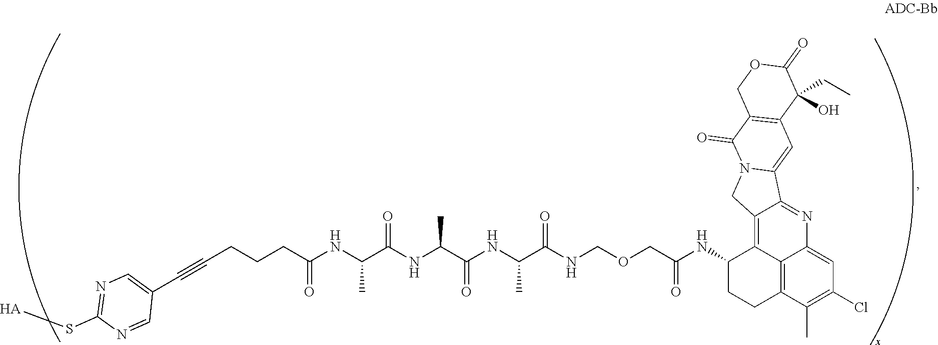

wherein HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 8.

In a further embodiment, the antibody-drug conjugate is ADC-Bb:

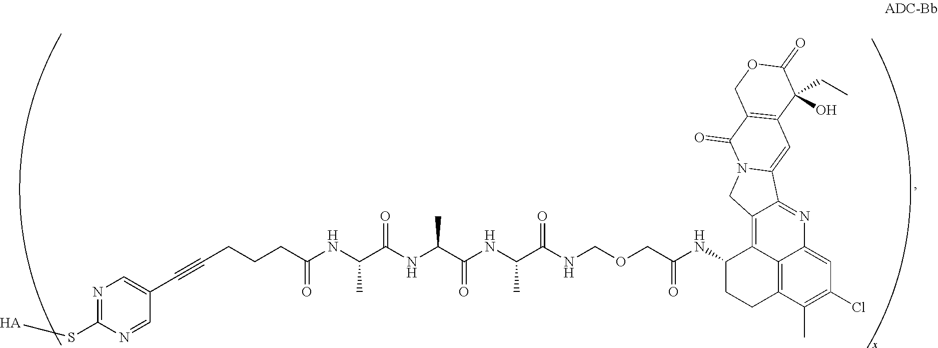

wherein HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 1-10, 1-8, 4-8, 4 or 8.

In the foregoing embodiment for antibody drug conjugate, for example, for antibody drug conjugate of formula ADC-Bb, in one embodiment, x is 1-10. In one embodiment, x is 1-8. In one embodiment, x is 4-8. In one embodiment, x is 4. In one embodiment, x is 8. In one embodiment, x is an integer.

In a further embodiment, the antibody-drug conjugate is ADC-Bb:
ADC-Bb

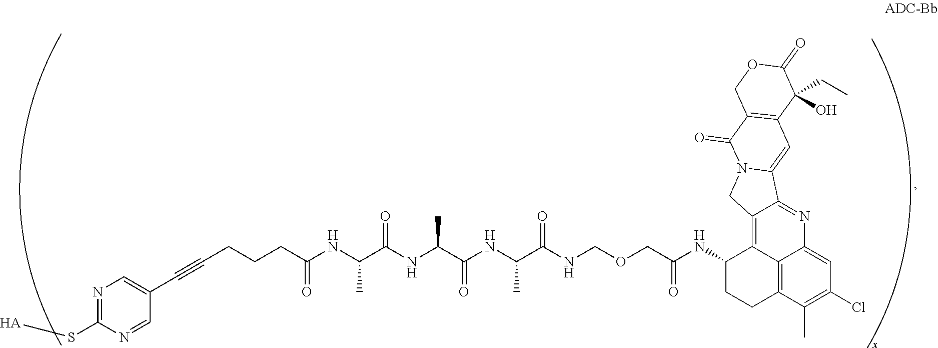

wherein HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl in the antibody to each pyrimidinyl group in formula ADC-Bb;
and x is 7-8.

In a further embodiment, the antibody-drug conjugate is ADC-Bb:

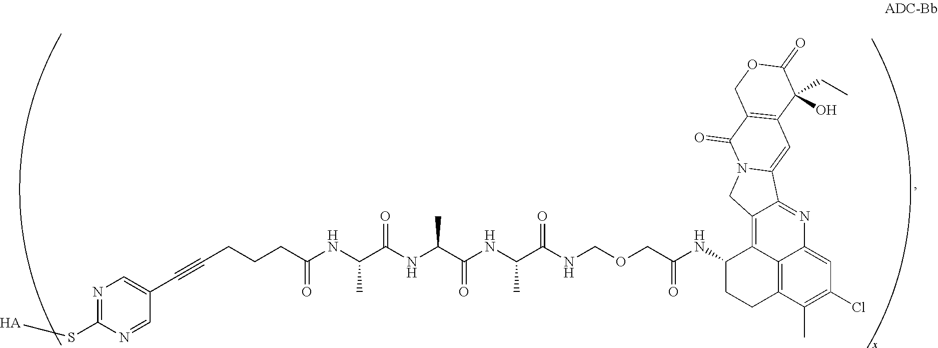

wherein HA-(S- comprises a heavy chain (HC) having the amino acid sequence as set forth in SEQ ID NO: 22 and a light chain (LC) having the amino acid sequence as set forth in SEQ ID NO: 21;
each -(S-represents a linkage from a sulfhydryl in the antibody to each pyrimidinyl group in formula ADC-Bb;
and x=1-10, 1-8, 4-8, 7-8, 4 or 8.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

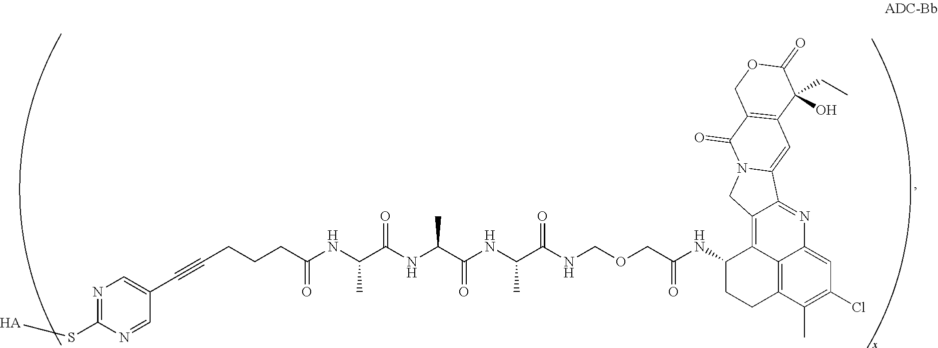

wherein HA-(S- comprises a heavy chain (HC) having the amino acid sequence as set forth in SEQ ID NO: 23 and a light chain (LC) having the amino acid sequence as set forth in SEQ ID NO: 21;
each -(S-represents a linkage from a sulfhydryl in the antibody to each pyrimidinyl group in formula ADC-Bb;
and x=1-10, 1-8, 4-8, 7-8, 4 or 8.

In the foregoing embodiment for antibody drug conjugate, for example, for antibody drug conjugate of formula ADC-Bb, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

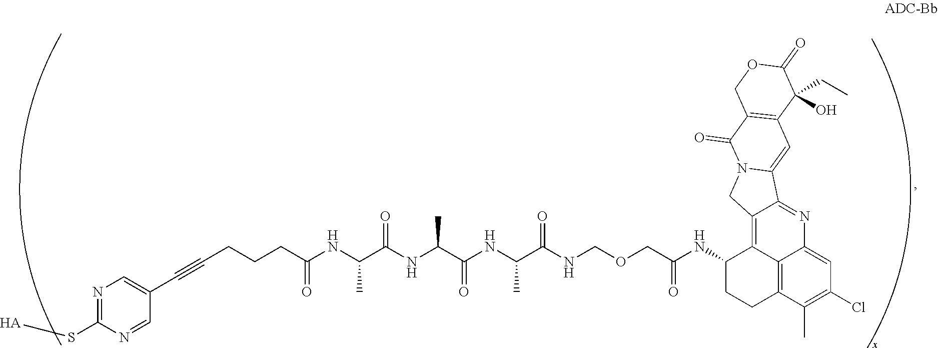

wherein HA-(S- comprises a heavy chain (HC) having the amino acid sequence as set forth in SEQ ID NO: 24 and a light chain (LC) having the amino acid sequence as set forth in SEQ ID NO: 21;
each -(S-represents a linkage from a sulfhydryl of the antibody to each pyrimidinyl group in formula ADC-Bb;
and x=1-10, 1-8, 4-8, 7-8, 4 or 8.

In the foregoing embodiment for antibody drug conjugate, for example, for antibody drug conjugate of formula ADC-Bb, in one embodiment, x is 1-10. In one embodiment, x is 1-8.

In one embodiment, x is 4-8. In one embodiment, x is 7-8. In one embodiment, x is 4. In one embodiment, x is 8. In one embodiment, x is an integer.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

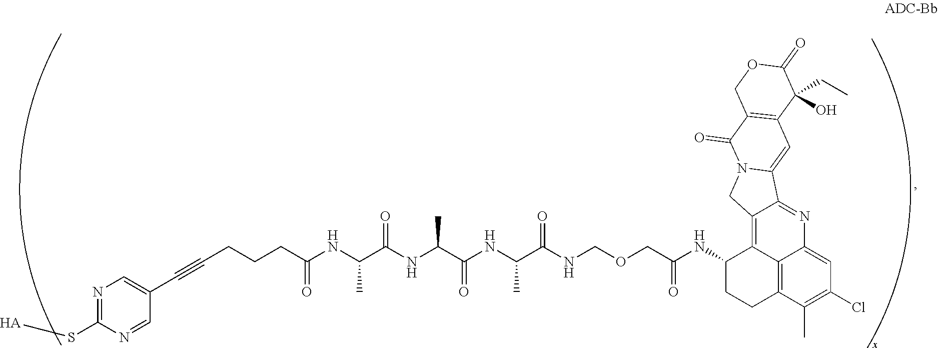

wherein HA-(S- comprises two heavy chains and two light chains, each heavy chain (HC) consists of the amino acid sequence as set forth in SEQ ID NO: 20 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In a further embodiment, the antibody-drug conjugate is ADC-Bb:

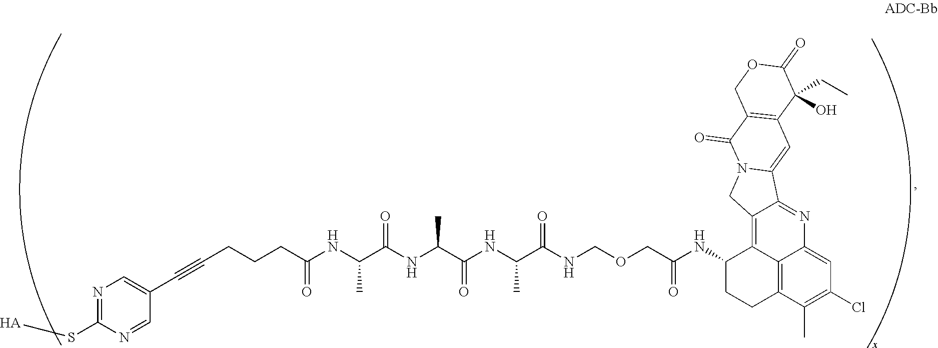

wherein HA-(S- consists of two heavy chains and two light chains, each heavy chain (HC) consists of the amino acid sequence as set forth in SEQ ID NO: 20 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In a further embodiment, the antibody-drug conjugate is ADC-Bb:

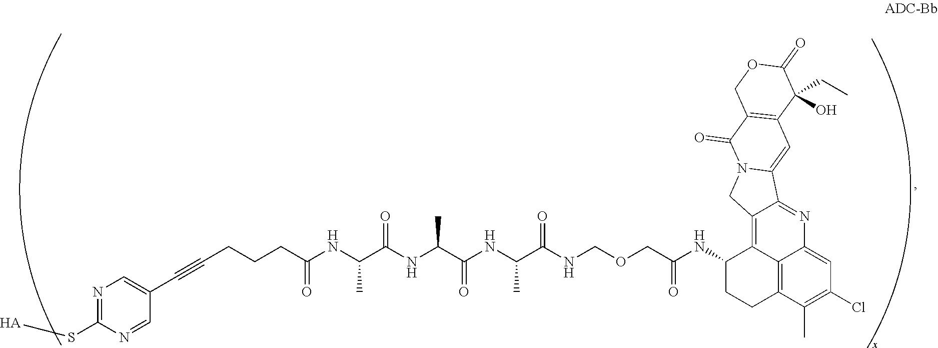

wherein HA-(S- comprises two heavy chains and two light chains, each heavy chain (HC) consists of the amino acid sequence as set forth in SEQ ID NO: 22 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In a further embodiment, the antibody-drug conjugate is ADC-Bb:

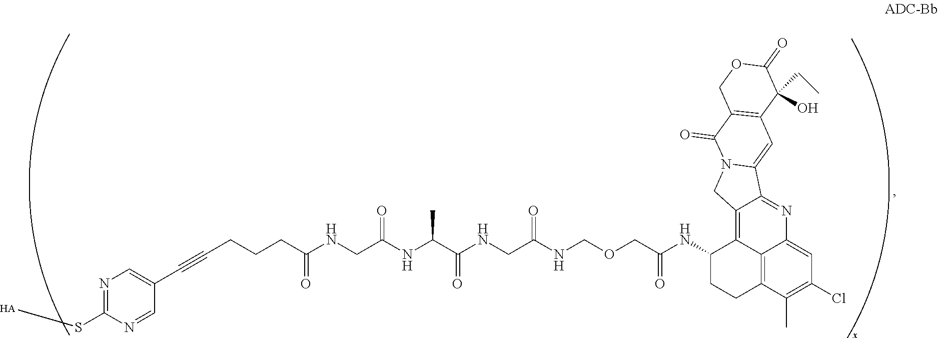

wherein HA-(S- consists of two heavy chains and two light chains, each heavy chain (HC) consists of the amino acid sequence as set forth in SEQ ID NO: 22 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In the foregoing embodiment for antibody drug conjugate, for example, for antibody drug conjugate of formula ADC-Bb, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In a further embodiment, the antibody-drug conjugate is ADC-Bb:

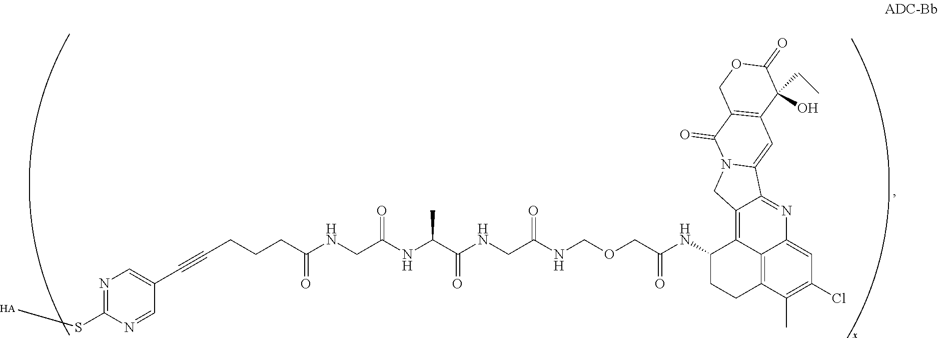

wherein HA-(S- comprises two heavy chains and two light chains, each heavy chain (HC) consists of the amino acid sequence as set forth in SEQ ID NO: 23 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In a further embodiment, the antibody-drug conjugate is ADC-Bb:

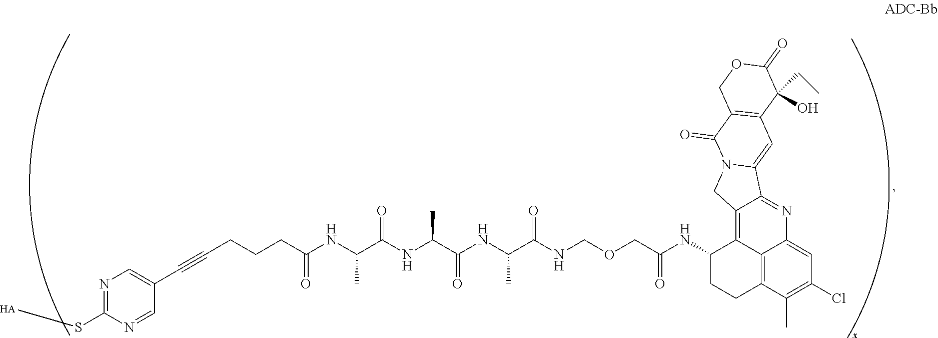

wherein HA-(S- consists of two heavy chains and two light chains, each heavy chain (HC) consists of the amino acid sequence as set forth in SEQ ID NO: 23 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In the foregoing embodiment for antibody drug conjugate, for example, for antibody drug conjugate of formula ADC-Bb, in one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In a further embodiment, the antibody-drug conjugate is ADC-Bb:

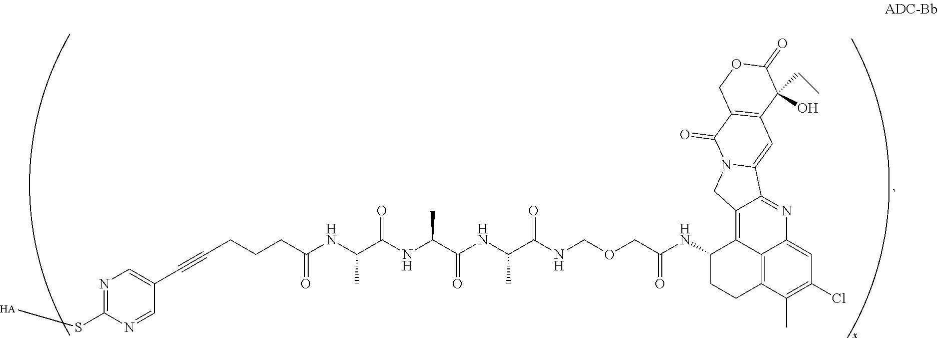

wherein HA-(S- comprises two heavy chains and two light chains, each heavy chain (HC) consists of the amino acid sequence as set forth in SEQ ID NO: 24 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In a further embodiment, the antibody-drug conjugate is ADC-Bb:

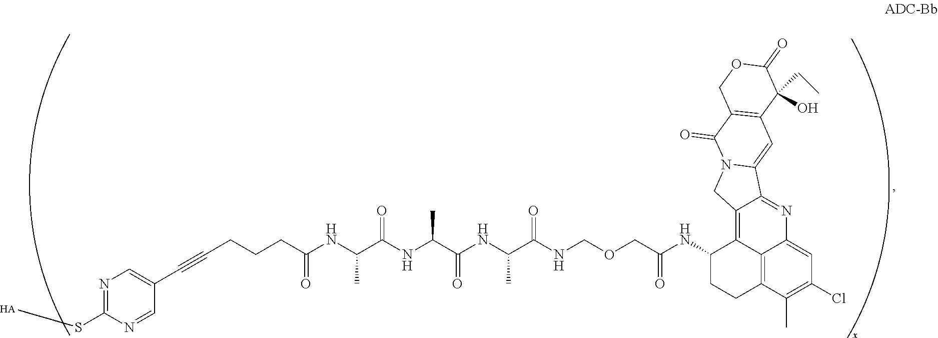

wherein HA-(S- consists of two heavy chains and two light chains, each heavy chain (HC) consists of the amino acid sequence as set forth in SEQ ID NO: 24 and a each light chain (LC) consists of the amino acid sequence as set forth in SEQ ID NO: 21;
each -(S-represents a linkage from a sulfhydryl of a cysteine residue in the antibody to each pyrimidinyl group in formula ADC-Bb;
and x=1-10, 1-8, 4-8, 7-8, 4 or 8.

In the foregoing embodiment for antibody drug conjugate, for example, for antibody drug conjugate of formula ADC-Bb, in one embodiment, x is 1-10. In one embodiment, x is 1-8.

In one embodiment, x is 4-8. In one embodiment, x is 7-8. In one embodiment, x is 4. In one embodiment, x is 8. In one embodiment, x is an integer.

In a further embodiment, the antibody-drug conjugate is selected from ADC-Aa and ADC-Bb:

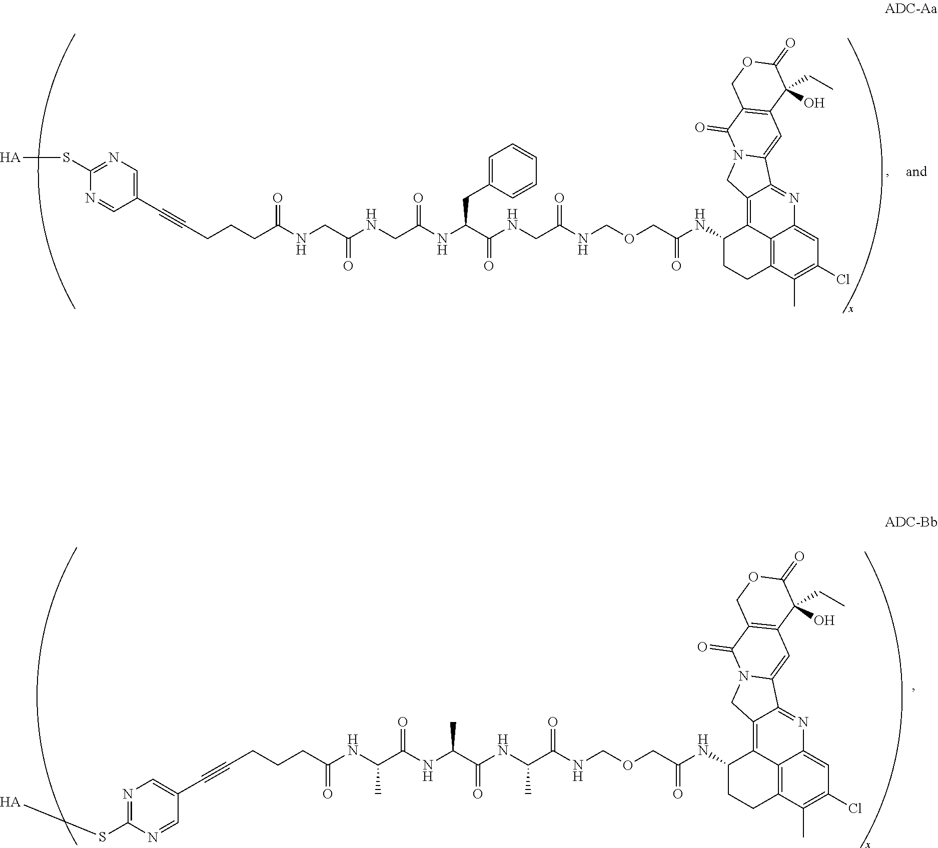

wherein HA-(S- comprises a heavy chain constant region (CH) having the amino acid sequence as set forth in SEQ ID NO: 3 and a light chain constant region (CL) having the amino acid sequence as set forth in SEQ ID NO: 4;
each -(S-represents a linkage from a sulfhydryl of a cysteine residue in the antibody to each pyrimidinyl group in formulas ADC-Aa and ADC-Bb;
and x is 4-9.

In a further embodiment, the antibody-drug conjugate is ADC-Aa:

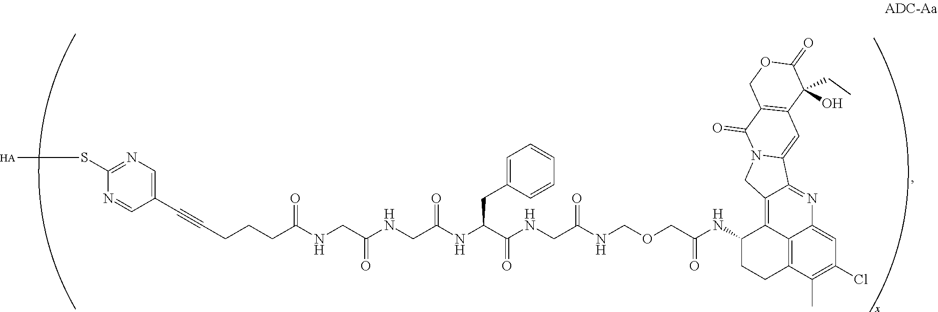

wherein HA-(S- comprises a heavy chain constant region (CH) having the amino acid sequence as set forth in SEQ ID NO: 3 and a light chain constant region (CL) having the amino acid sequence as set forth in SEQ ID NO: 4;
each -(S-represents a linkage from a sulfhydryl of a cysteine residue in the antibody to each pyrimidinyl group in formula ADC-Aa;
and x is 4-9.

In a further embodiment, the antibody-drug conjugate is ADC-Aa:

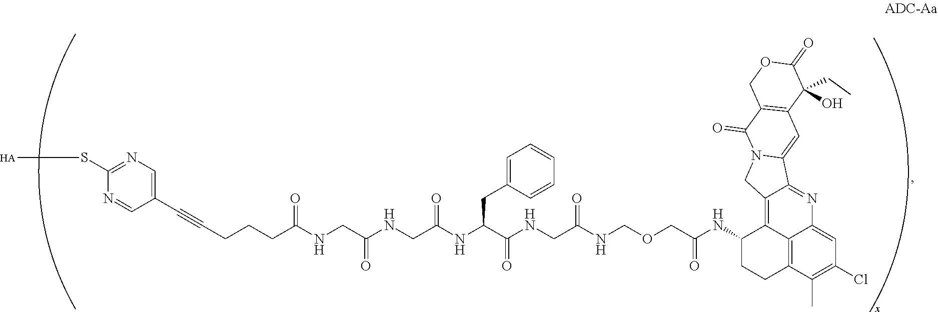

wherein HA-(S- comprises a heavy chain constant region (CH) having the amino acid sequence as set forth in SEQ ID NO: 3, and a light chain constant region (CL) having the amino acid sequence as set forth in SEQ ID NO: 4;
each -(S-represents a linkage from a sulfhydryl of a cysteine residue in the antibody to each pyrimidinyl group in formula ADC-Aa;
and x is 8-9.

In a further embodiment, the antibody-drug conjugate is ADC-Aa:

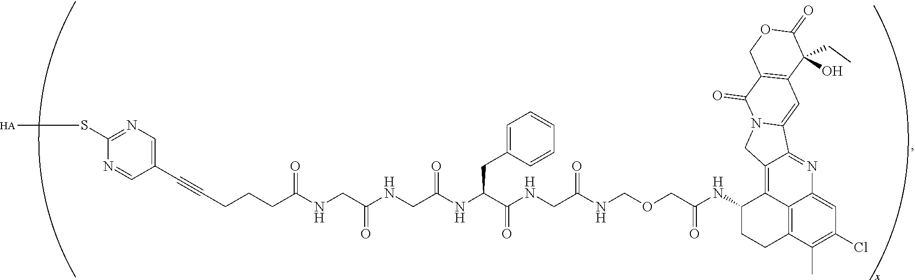

wherein HA-S- comprises a heavy chain constant region (CH) having the amino acid sequence as set forth in SEQ ID NO: 3, and a light chain constant region (CL) having the amino acid sequence as set forth in SEQ ID NO: 4;
each -(S-represents a linkage from a sulfhydryl in the antibody to each pyrimidinyl group in formula ADC-Aa;
and x is 8.

In certain embodiments, each -(S-represents a linkage from a sulfhydryl of a cysteine residue in the antibody to each pyrimidinyl group in formula ADC-Aa.

In a further embodiment, the antibody-drug conjugate is ADC-Bb:

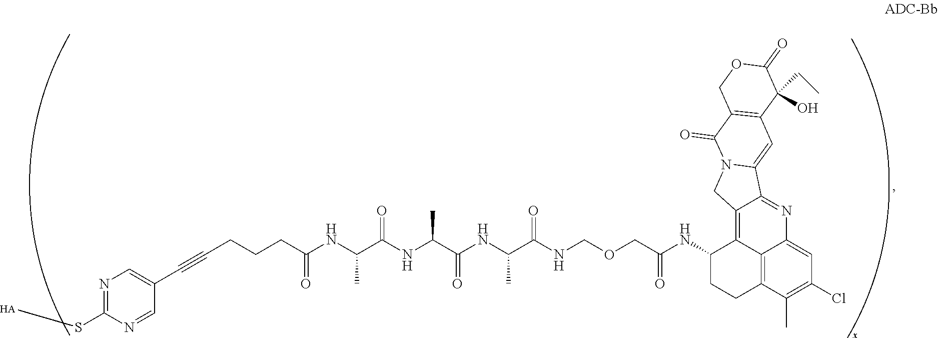

wherein HA-(S- comprises a heavy chain constant region (CH) having the amino acid sequence as set forth in SEQ ID NO: 3 and a light chain constant region (CL) having the amino acid sequence as set forth in SEQ ID NO: 4;
each -(S-represents a linkage from a sulfhydryl in the antibody to each pyrimidinyl group in formula ADC-Bb;
and x is 3-4 or 7-9.

In the foregoing embodiment for antibody drug conjugates, for example, for antibody drug conjugates of formulas ADC-Aa and ADC-Bb, in one embodiment, x is 3-4. In one embodiment, x is 7-9. In one embodiment, x is an integer.

In certain embodiments, each -(S-represents a linkage from a sulfhydryl of a cysteine residue in the antibody to each pyrimidinyl group in formula ADC-Bb.

In a further embodiment, the antibody-drug conjugate is ADC-Bb:

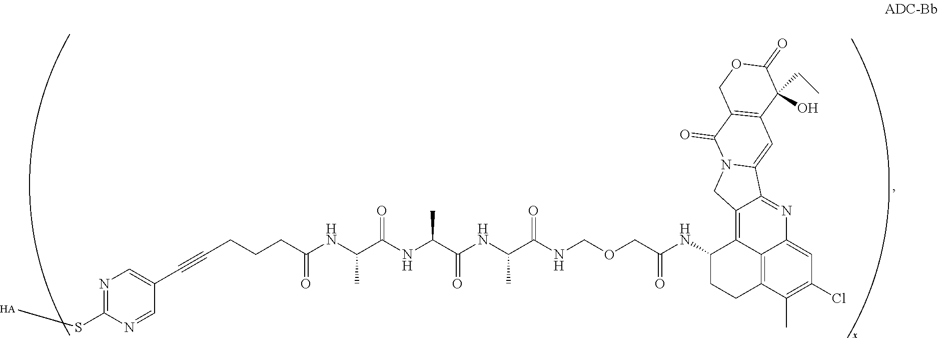

wherein HA-(S- comprises a heavy chain constant region (CH) having the amino acid sequence as set forth in SEQ ID NO: 3, and a light chain constant region (CL) having the amino acid sequence as set forth in SEQ ID NO: 4;
each -(S-represents a linkage from a sulfhydryl of a cysteine residue in the antibody to each pyrimidinyl group in formula ADC-Bb; and x is 8-9.

In a further embodiment, the antibody-drug conjugate is ADC-Bb:

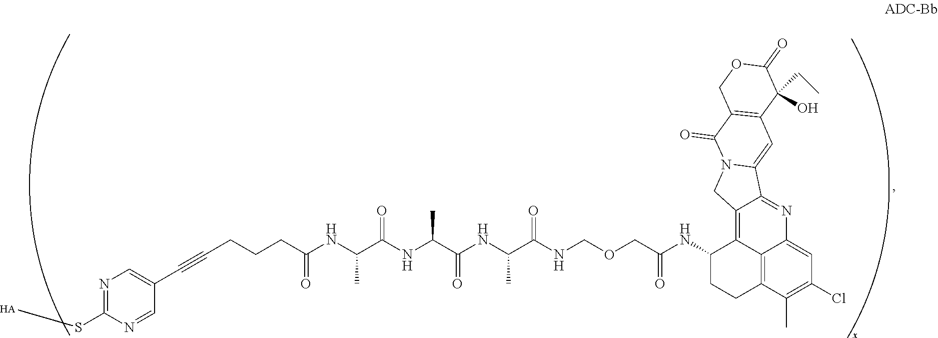

wherein HA-(S- comprises a heavy chain constant region (CH) having the amino acid sequence as set forth in SEQ ID NO: 3, and a light chain constant region (CL) having the amino acid sequence as set forth in SEQ ID NO: 4;
each -(S-represents a linkage from a sulfhydryl of a cysteine residue in the antibody to each pyrimidinyl group in formula ADC-Bb;
and x is 8.

In some embodiments, the antibody-drug conjugate is selected from ADC-1, ADC-2, ADC-3, ADC-4 and ADC-5 as follows:

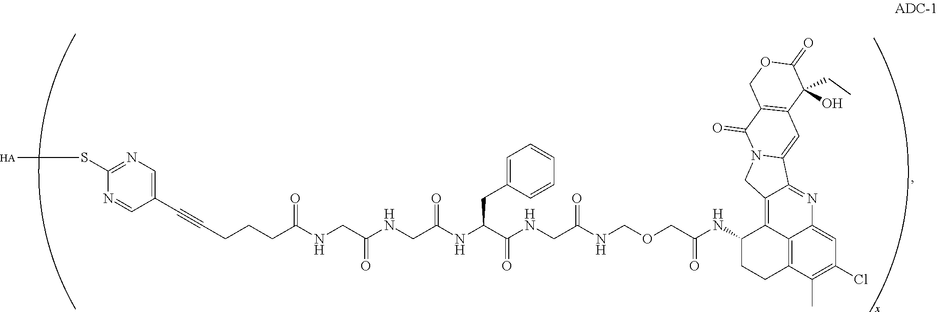

wherein x=8, HA-(S- comprises antibody Ab-hzDPmut, and
each -(S-represents a linkage from a sulfhydryl of a cysteine residue in the antibody to each pyrimidinyl group in formula ADC-1;

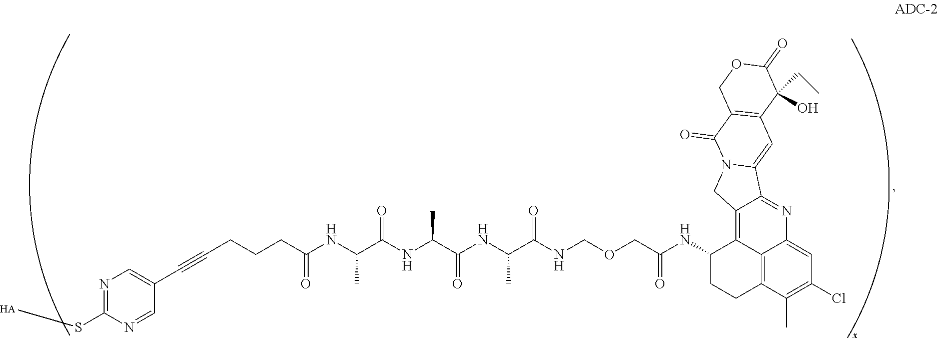

wherein x=8, HA-(S- comprises antibody Ab-hzDPmut, and
each -(S-represents a linkage from a sulfhydryl of a cysteine residue in the antibody to each pyrimidinyl group in formula ADC-2;

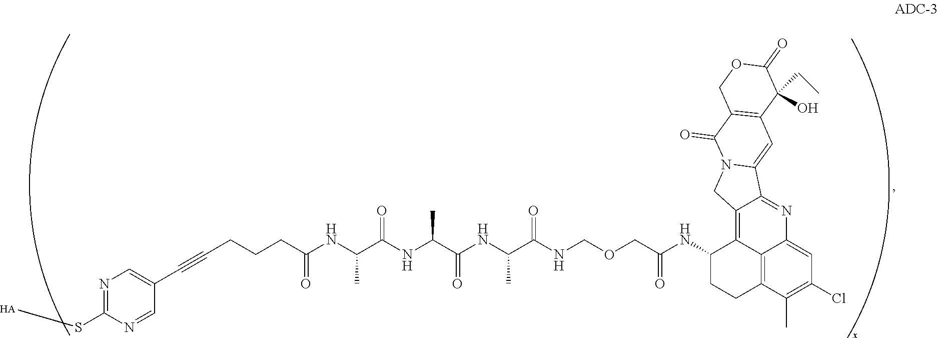

wherein x=4, HA-(S- comprises antibody Ab-hzDPmut, and
each -(S-represents a linkage from a sulfhydryl of a cysteine residue in the antibody to each pyrimidinyl group in formula ADC-3;

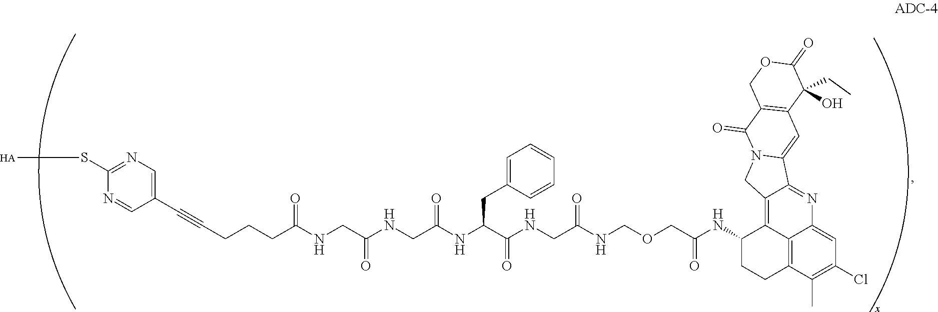

wherein x=8, HA-(S- comprises antibody tusamitamab, and
each -(S-represents a linkage from a sulfhydryl of a cysteine residue in the antibody to each pyrimidinyl group in formula ADC-4; and

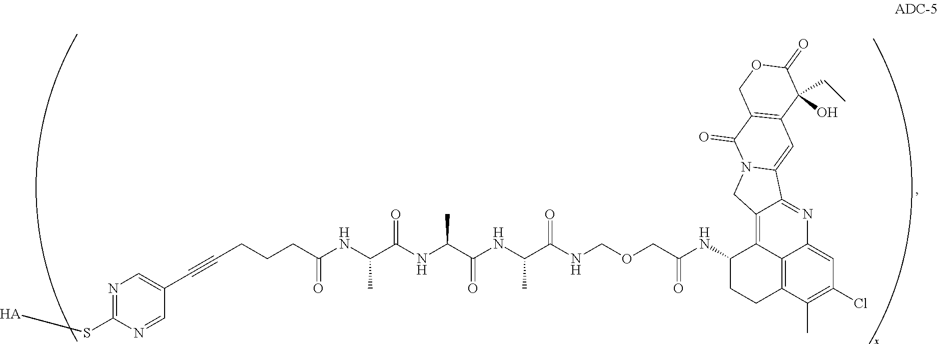

wherein x=8, HA-(S- comprises antibody tusamitamab, and
each -(S-represents a linkage from a sulfhydryl of a cysteine residue in the antibody to each pyrimidinyl group in formula ADC-5.

In one embodiment, provided herein is an antibody-drug conjugate of formula ADC-Aa′ or ADC-Bb′:

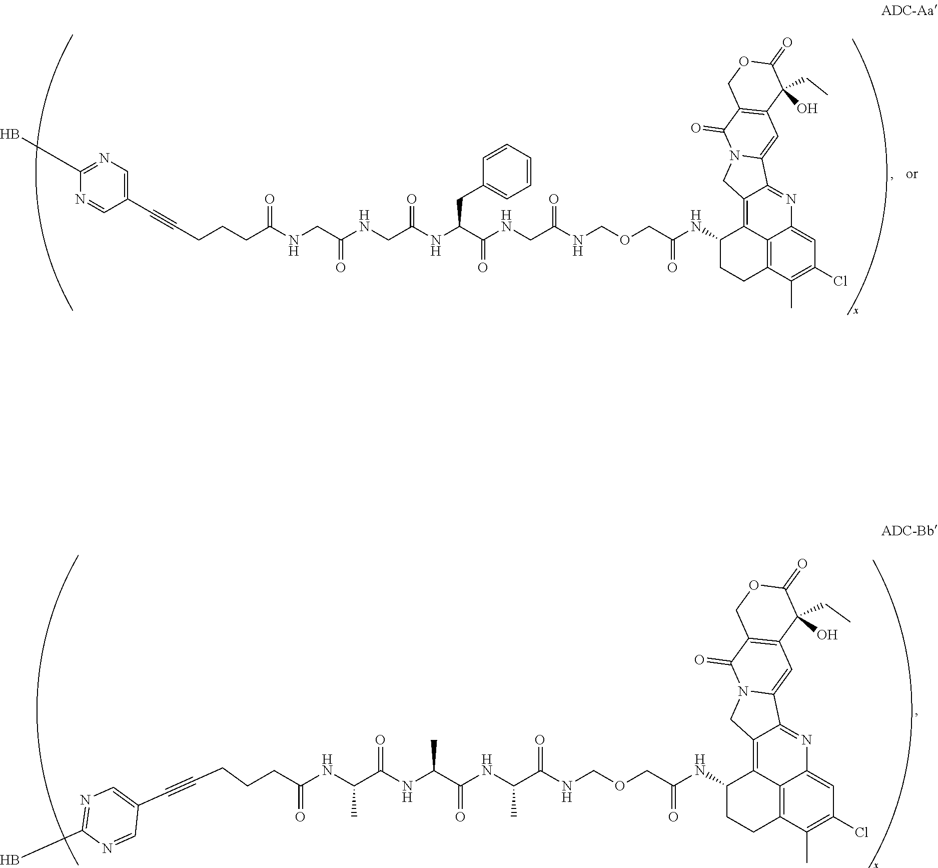

wherein HB-(- comprises any one of the antibodies disclosed herein, and x is an integer from 1-20.

In one embodiment, provided herein is an antibody-drug conjugate of formula ADC-Aa′ or ADC-Bb′:

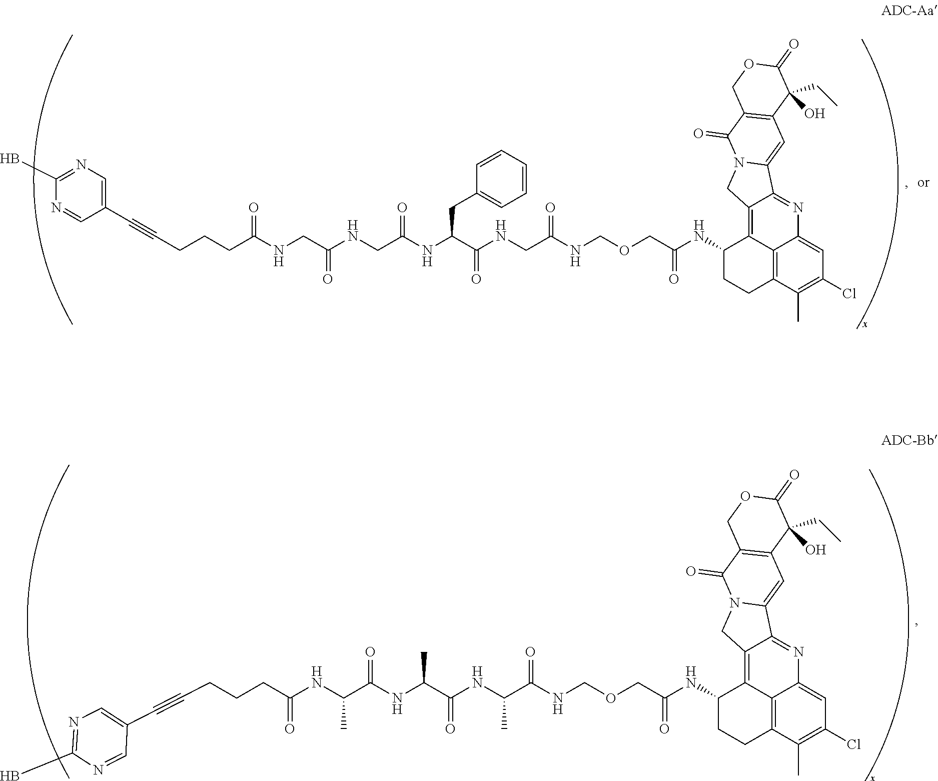

wherein HB-(- comprises a V_{H} having the amino acid sequence as set forth in SEQ ID NO: 1, and a V_{L} having the amino acid sequence as set forth in SEQ ID NO: 2, and x=1-20, 1-16, 1-10, 1-8, 4-8, 4 or 8.

In the foregoing embodiment for antibody drug conjugates, for example, for antibody drug conjugates of formulas ADC-Aa′ and ADC-Bb′, in one embodiment, x is 1-20. In one embodiment, x is 1-16. In one embodiment, x is 1-10. In one embodiment, x is 1-8. In one embodiment, x is 4-8. In one embodiment, x is 7-8. In one embodiment, x is 4. In one embodiment, x is 8. In one embodiment, x is an integer.

In one embodiment, provided herein is an antibody-drug conjugate of formula ADC-Aa′ or ADC-Bb′:

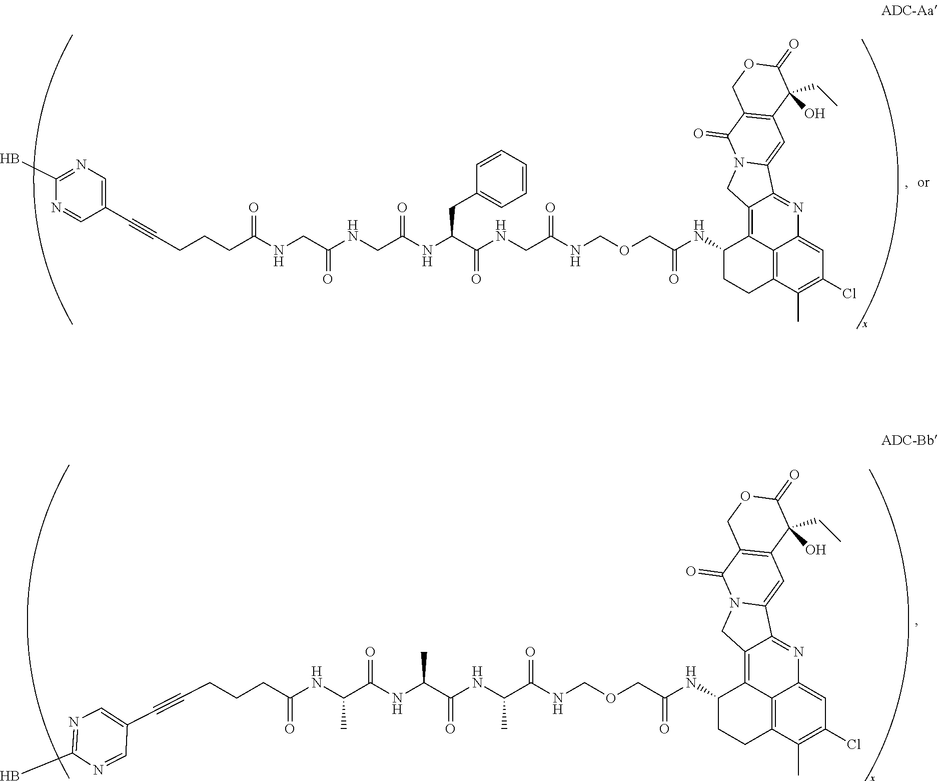

wherein HB-(- comprises an antibody or antigen-binding fragment comprising:
(1) the following heavy chain variable regions (VH) and/or light chain variable regions (VL), wherein CDRs are defined according to a Chothia numbering system:
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (1) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (2) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants of (3) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (4) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement; and

$$x = 1 ‐ 20 , 1 ‐ 16 , 1 ‐ 10 , 1 ‐ 8 , 4 ‐ 8 , 4 or 8.$$

In the foregoing embodiment for antibody drug conjugates, for example, for antibody drug conjugates of formulas ADC-Aa′ and ADC-Bb′, in one embodiment, x is 1-20. In one embodiment, x is 1-16. In one embodiment, x is 1-10. In one embodiment, x is 1-8. In one embodiment, x is 4-8. In one embodiment, x is 4. In one embodiment, x is 8. In one embodiment, x is an integer.

In a further embodiment, the HB-(- in the antibody-drug conjugate is selected from an antibody or antigen binding fragment comprising a V_{H} having the amino acid sequence as set forth in SEQ ID NO: 1 or a variant thereof, and/or, a V_{L} having the amino acid sequence as set forth in SEQ ID NO: 2 or a variant thereof;
wherein the variants have the amino acid sequence identity of at least 70%, at least 80%, at least 85%, at least 90%, at least 91%, at least 92%, at least 93%, at least 94%, at least 95%, at least 96%, at least 97%, at least 98%, at least 99% or 100% as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived; and preferably, the replacement is a conservative replacement.

In a further embodiment, the HB-(- in the antibody-drug conjugate is an antibody or antigen binding fragment comprising a V_{H} having the amino acid sequence as set forth in SEQ ID NO: 1, and/or, a V_{L} having the amino acid sequence as set forth in SEQ ID NO: 2.

In a further embodiment, the HB-(- in the antibody-drug conjugate is an antibody or antigen binding fragment comprising a heavy chain comprising a V_{H} having the amino acid sequence set forth in SEQ ID NO: 1.

In a further embodiment, the HB-(- in the antibody-drug conjugate is an antibody or antigen binding fragment comprising a heavy chain comprising a V_{H} having the amino acid sequence set forth in SEQ ID NO: 1, wherein the N-terminal glutamine of the antibody variable region has undergone cyclization to pyroglutamate.

In a further embodiment, the HB-(- in the antibody-drug conjugate is an antibody or antigen binding fragment comprising a heavy chain comprising a V_{H} having the amino acid sequence set forth in SEQ ID NO: 1, wherein the N-terminal glutamine of the antibody variable region has undergone cyclization to pyroglutamic acid.

In a further embodiment, the HB-(- in the antibody-drug conjugate is an antibody or antigen binding fragment obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 or a variant thereof and/or a V_{L} having the amino acid sequence set forth in SEQ ID NO: 2 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids (for example, replacement, deletion or addition of 1, 2, 3, 4 or 5 amino acids) as compared to the sequence from which the variants are derived.

In a further embodiment, the HB-(- in the antibody-drug conjugate is an antibody or antigen binding fragment obtainable by expressing the nucleic acid molecules encoding the heavy chains and the light chains of the antibody or antigen-binding fragment thereof in a host cell, wherein the antibody or antigen-binding fragment thereof comprises a V_{H} having the amino acid sequence set forth in SEQ ID NO: 1 and/or a V_{L} having the amino acid sequence set forth in SEQ ID NO: 2.

In a further embodiment, the HB-(- in the antibody-drug conjugate is an antibody or antigen binding fragment conjugated through one or more-S-from the one or more sulfhydryls in the antibody or antigen-binding fragment to the one or more M. In a further embodiment, the HB-(- in the antibody-drug conjugate is an antibody or antigen binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through one or more-S-from the one or more sulfhydryls in the antibody or antigen-binding fragment to the one or more M.

In a further embodiment, the HB-(- in the antibody-drug conjugate is an antibody or antigen binding fragment conjugated through x number of—S-from x number of sulfhydryl(s) in the antibody or antigen-binding fragment to x number of the one or more M. In a further embodiment, the HB-(- in the antibody-drug conjugate is an antibody or antigen binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through x number of—S-from x number of sulfhydryl(s) in the antibody or antigen-binding fragment to x number of the one or more M.

In a further embodiment, the HB-(- in the antibody-drug conjugates provided herein represents an antibody or antigen-binding fragment thereof conjugated through-S-from a sulfhydryl in the antibody or antigen-binding fragment to M. In a further embodiment, the HB-(-in the antibody-drug conjugates provided herein represents an antibody or antigen-binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through-S-from a sulfhydryl in the antibody or antigen-binding fragment to M.

In a further embodiment, the HB-(- in the antibody-drug conjugates provided herein represents an antibody or antigen-binding fragment thereof conjugated through-S-from x number of sulfhydryl(s) in the antibody or antigen-binding fragment to x number of M. In a further embodiment, the HB-(- in the antibody-drug conjugates provided herein represents an antibody or antigen-binding fragment thereof comprising a V_{H} having the amino acid sequence as set forth in SEQ ID NO: 1, and a V_{L} having the amino acid sequence as set forth in SEQ ID NO: 2, said antibody or antigen binding fragment thereof is conjugated through-S-from x number of sulfhydryl(s) in the antibody or antigen-binding fragment to x number of M.

In one embodiment, provided herein is an antibody-drug conjugate of formula ADC-Aa′ or ADC-Bb′:

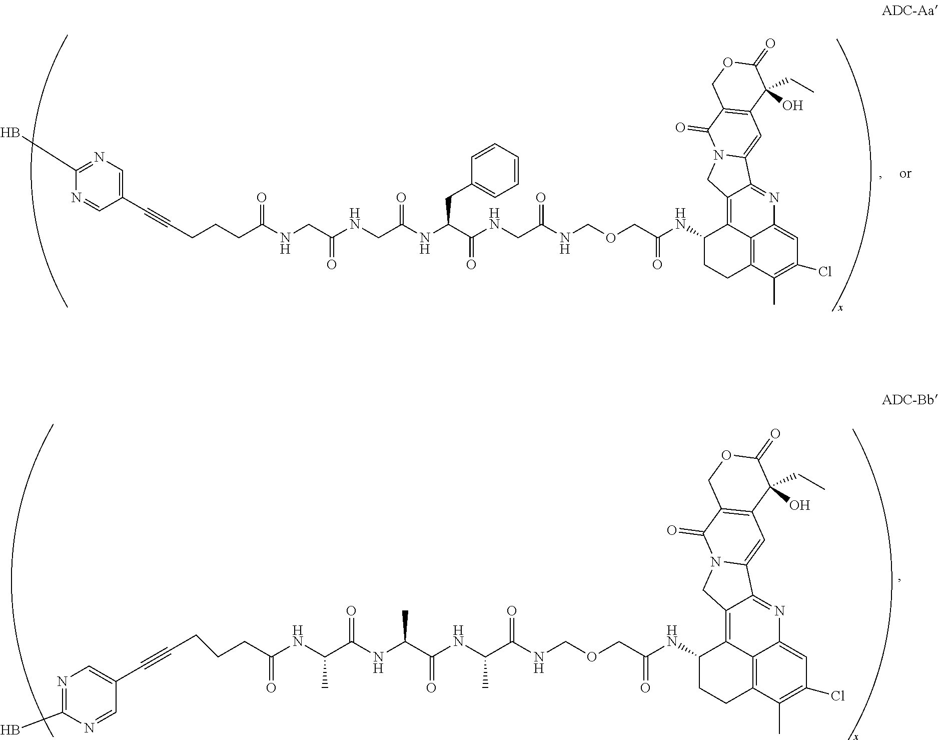

wherein HB-(- is a conjugated anti-CEACAM5 monoclonal antibody comprising a heavy chain (HC) having the amino acid sequence as set forth in SEQ ID NO: 20 or 22 and a light chain (LC) having the amino acid sequence as set forth in SEQ ID NO: 21, and x is an integer from 1-8, 4-8, 4 or 8.

In the foregoing embodiment for antibody drug conjugates, for example, for antibody drug conjugates of formulas ADC-Aa′ and ADC-Bb′, in one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Aa′:

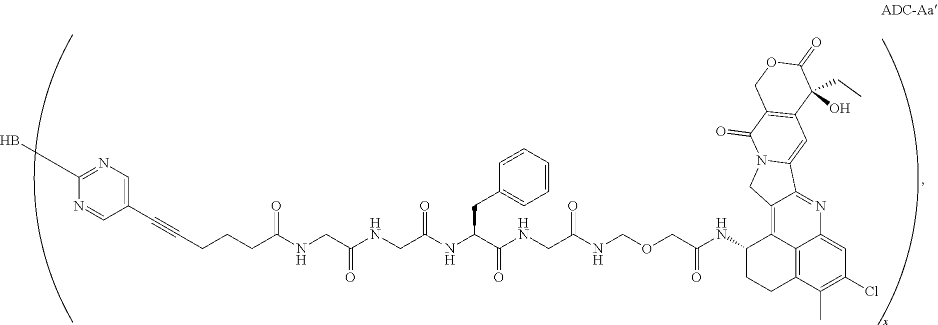

wherein HB-(- is a conjugated anti-CEACAM5 monoclonal antibody comprising a heavy chain (HC) having the amino acid sequence as set forth in SEQ ID NO: 20 or 22 and a light chain (LC) having the amino acid sequence as set forth in SEQ ID NO: 21, and x is an integer from 1-8, 4-8, 4 or 8.

In the foregoing embodiment for antibody drug conjugate, for example, for antibody drug conjugate of formula ADC-Aa′, in one embodiment, x is 1-8. In one embodiment, x is 4-8. In one embodiment, x is 4. In one embodiment, x is 8. In one embodiment, x is an integer.

In one embodiment, provided herein is an antibody-drug conjugate of formula ADC-Bb′:

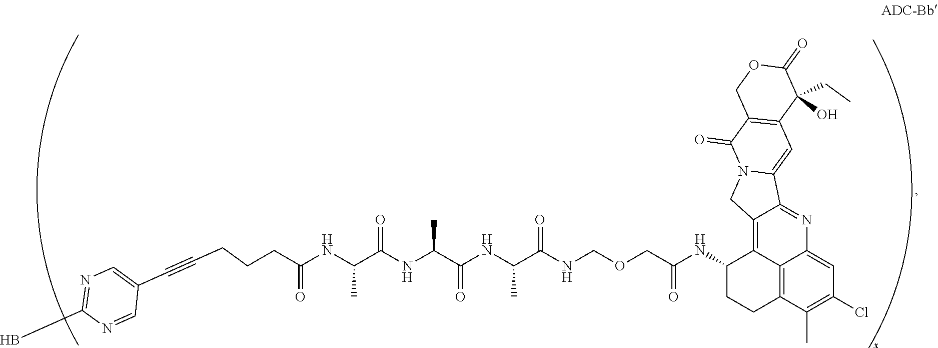

wherein HB-(- is a conjugated anti-CEACAM5 monoclonal antibody comprising a heavy chain (HC) having the amino acid sequence as set forth in SEQ ID NO: 20 or 22 and a light chain (LC) having the amino acid sequence as set forth in SEQ ID NO: 21, and x is an integer from 1-8, 4-8, 4 or 8.

In the foregoing embodiment for antibody drug conjugate, for example, for antibody drug conjugate of formula ADC-Bb′, in one embodiment, x is 1-8. In one embodiment, x is 4-8. In one embodiment, x is 4. In one embodiment, x is 8. In one embodiment, x is an integer.

In a further embodiment, the antibody-drug conjugate is ADC-Aa′ or ADC-Bb′:

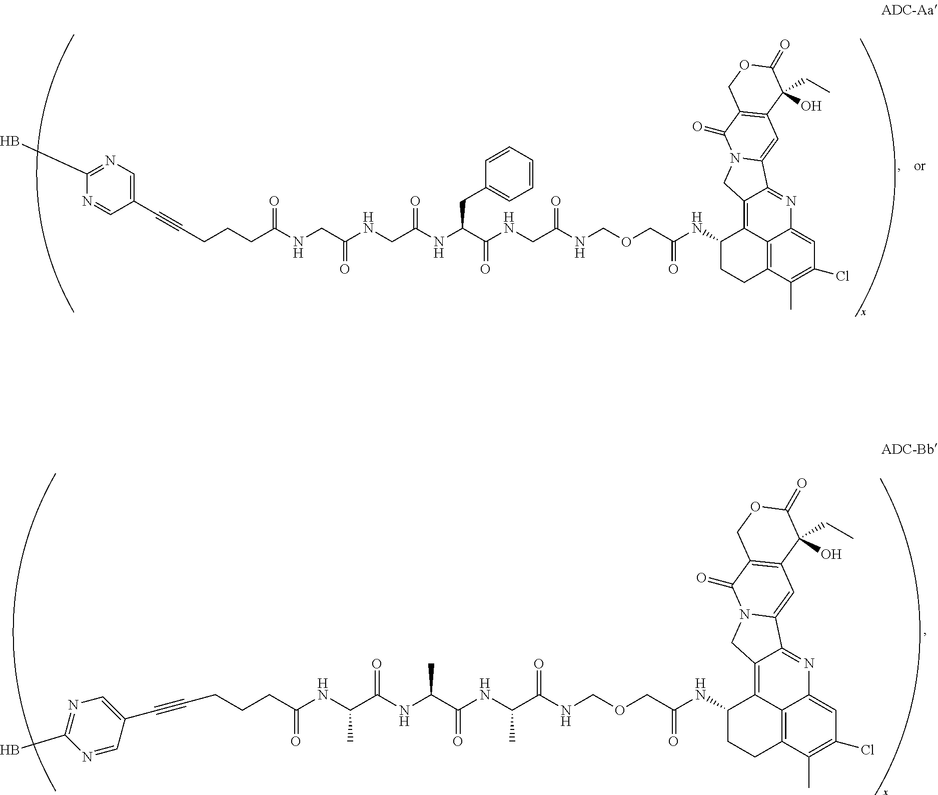

wherein HB-(- comprises a heavy chain (HC) having the amino acid sequence as set forth in SEQ ID NO: 22, 23 or 24 and a light chain (LC) having the amino acid sequence as set forth in SEQ ID NO: 21;
and x=1-20, 1-16, 1-8, 4-8, 4 or 8.

In the foregoing embodiment for antibody drug conjugates, for example, for antibody drug conjugates of formulas ADC-Aa′ and ADC-Bb, in one embodiment, x is 1-20. In one embodiment, x is 1-16. In one embodiment, x is 1-8. In one embodiment, x is 4-8. In one embodiment, x is 4. In one embodiment, x is 8. In one embodiment, x is an integer.

In one embodiment, HB-(- is linked via one or more sulfhydryl group(s) of cysteine residue(s), one or more amino group(s) of lysine residue(s), one or more hydroxy group(s) of threonine residue(s) or one or more hydroxy group(s) of serine residue(s) in the antibody to form the conjugate.

In one embodiment, HB-(- is linked via one or more sulfhydryl group(s) of cysteine residue(s) in the antibody to form the conjugate.

In one embodiment, HB-(- is linked via one or more amino group(s) of lysine residue(s) in the antibody to form the conjugate.

In one embodiment, HB-(- is linked via one or more hydroxy group(s) of threonine residue(s) in the antibody to form the conjugate.

In one embodiment, HB-(- is linked via one or more hydroxy group(s) of serine residue(s) in the antibody to form the conjugate.

In one embodiment, HB-(- is linked via x number of sulfhydryl group(s) of cysteine residue(s), x number of amino group(s) of lysine residue(s), x number of hydroxy group(s) of threonine residue(s) or x number of hydroxy group(s) of serine residue(s) in the antibody to form the conjugate.

In one embodiment, HB-(- is linked via x number of sulfhydryl group(s) of cysteine residue(s) in the antibody.

In one embodiment, HB-(- is linked via x number of amino group(s) of lysine residue(s) in the antibody.

In one embodiment, HB-(- is linked via x number of hydroxy group(s) of threonine residue(s) in the antibody.

In one embodiment, HB-(- is linked via x number of hydroxy group(s) of serine residue(s) in the antibody.

In some embodiments, the antibody-drug conjugate (ADC) has an x of 1 to 10, for example, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

In some embodiments, the antibody-drug conjugate (ADC) has an x of 1, 2, 3, 4, 5, 6, 7, 8, 9, or 10.

In some embodiments, the antibody disclosed herein is genetically engineered to comprise one or more cysteine or lysine residue or non-canonical amino acid substitutions of amino acids at defined locations within the antibody.

In some embodiments, the antibody disclosed herein is genetically engineered to comprise one or more cysteine residues or non-canonical amino acid substitutions of amino acids at defined locations within the antibody. These cysteine residues or non-canonical amino acid residues may then be conjugated to drug-linker via the sulfhydryl group of the cysteine residues or the reactive group of the non-canonical amino acid. Thus, the antibody-drug conjugate of the present invention may comprise one or more substitutions of an amino acid in the heavy chain or light chain of the antibody with a cysteine residue or non-canonical amino acid residue, which is then conjugated to a drug-linker disclosed herein. In particular embodiments, the amino acid positions that may be substituted are selected from positions 152, 153, 171, 172, 173, and 375 of the heavy chain constant domain (numbering according to the Eu numbering scheme) and positions 165 and 168 of the light chain constant domain (numbering beginning with amino acid 1 at the N-terminus).

In some embodiments, as explained herein, a composition of the ADC has a DAR value (drug-to-antibody ratio) of 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 9, for example, 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0 or 7.5 to 9.0.

In some embodiments, as explained herein, a composition of the ADC has a DAR value (drug-to-antibody ratio) of 3 to 9, for example: 3.0, 3.01, 3.02, 3.03, 3.04, 3.05, 3.06, 3.07, 3.08, 3.09, 3.1, 3.11, 3.12, 3.13, 3.14, 3.15, 3.16, 3.17, 3.18, 3.19, 3.2, 3.21, 3.22, 3.23, 3.24, 3.25, 3.26, 3.27, 3.28, 3.29, 3.3, 3.31, 3.32, 3.33, 3.34, 3.35, 3.36, 3.37, 3.38, 3.39, 3.4, 3.41, 3.42, 3.43, 3.44, 3.45, 3.46, 3.47, 3.48, 3.49, 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, 3.75, 3.76, 3.77, 3.78, 3.79, 3.8, 3.81, 3.82, 3.83, 3.84, 3.85, 3.86, 3.87, 3.88, 3.89, 3.9, 3.91, 3.92, 3.93, 3.94, 3.95, 3.96, 3.97, 3.98, 3.99, 4.0, 4.01, 4.02, 4.03, 4.04, 4.05, 4.06, 4.07, 4.08, 4.09, 4.1, 4.11, 4.12, 4.13, 4.14, 4.15, 4.16, 4.17, 4.18, 4.19, 4.2, 4.21, 4.22, 4.23, 4.24, 4.25, 4.26, 4.27, 4.28, 4.29, 4.3, 4.31, 4.32, 4.33, 4.34, 4.35, 4.36, 4.37, 4.38, 4.39, 4.4, 4.41, 4.42, 4.43, 4.44, 4.45, 4.46, 4.47, 4.48, 4.49, 4.5, 4.51, 4.52, 4.53, 4.54, 4.55, 4.56, 4.57, 4.58, 4.59, 4.6, 4.61, 4.62, 4.63, 4.64, 4.65, 4.66, 4.67, 4.68, 4.69, 4.7, 4.71, 4.72, 4.73, 4.74, 4.75, 4.76, 4.77, 4.78, 4.79, 4.8, 4.81, 4.82, 4.83, 4.84, 4.85, 4.86, 4.87, 4.88, 4.89, 4.9, 4.91, 4.92, 4.93, 4.94, 4.95, 4.96, 4.97, 4.98, 4.99, 5.0, 5.01, 5.02, 5.03, 5.04, 5.05, 5.06, 5.07, 5.08, 5.09, 5.1, 5.11, 5.12, 5.13, 5.14, 5.15, 5.16, 5.17, 5.18, 5.19, 5.2, 5.21, 5.22, 5.23, 5.24, 5.25, 5.26, 5.27, 5.28, 5.29, 5.3, 5.31, 5.32, 5.33, 5.34, 5.35, 5.36, 5.37, 5.38, 5.39, 5.4, 5.41, 5.42, 5.43, 5.44, 5.45, 5.46, 5.47, 5.48, 5.49, 5.5, 5.51, 5.52, 5.53, 5.54, 5.55, 5.56, 5.57, 5.58, 5.59, 5.6, 5.61, 5.62, 5.63, 5.64, 5.65, 5.66, 5.67, 5.68, 5.69, 5.7, 5.71, 5.72, 5.73, 5.74, 5.75, 5.76, 5.77, 5.78, 5.79, 5.8, 5.81, 5.82, 5.83, 5.84, 5.85, 5.86, 5.87, 5.88, 5.89, 5.9, 5.91, 5.92, 5.93, 5.94, 5.95, 5.96, 5.97, 5.98, 5.99, 6.0, 6.01, 6.02, 6.03, 6.04, 6.05, 6.06, 6.07, 6.08, 6.09, 6.1, 6.11, 6.12, 6.13, 6.14, 6.15, 6.16, 6.17, 6.18, 6.19, 6.2, 6.21, 6.22, 6.23, 6.24, 6.25, 6.26, 6.27, 6.28, 6.29, 6.3, 6.31, 6.32, 6.33, 6.34, 6.35, 6.36, 6.37, 6.38, 6.39, 6.4, 6.41, 6.42, 6.43, 6.44, 6.45, 6.46, 6.47, 6.48, 6.49, 6.5, 6.51, 6.52, 6.53, 6.54, 6.55, 6.56, 6.57, 6.58, 6.59, 6.6, 6.61, 6.62, 6.63, 6.64, 6.65, 6.66, 6.67, 6.68, 6.69, 6.7, 6.71, 6.72, 6.73, 6.74, 6.75, 6.76, 6.77, 6.78, 6.79, 6.8, 6.81, 6.82, 6.83, 6.84, 6.85, 6.86, 6.87, 6.88, 6.89, 6.9, 6.91, 6.92, 6.93, 6.94, 6.95, 6.96, 6.97, 6.98, 6.99, 7.0, 7.01, 7.02, 7.03, 7.04, 7.05, 7.06, 7.07, 7.08, 7.09, 7.1, 7.11, 7.12, 7.13, 7.14, 7.15, 7.16, 7.17, 7.18, 7.19, 7.2, 7.21, 7.22, 7.23, 7.24, 7.25, 7.26, 7.27, 7.28, 7.29, 7.3, 7.31, 7.32, 7.33, 7.34, 7.35, 7.36, 7.37, 7.38, 7.39, 7.4, 7.41, 7.42, 7.43, 7.44, 7.45, 7.46, 7.47, 7.48, 7.49, 7.5, 7.51, 7.52, 7.53, 7.54, 7.55, 7.56, 7.57, 7.58, 7.59, 7.6, 7.61, 7.62, 7.63, 7.64, 7.65, 7.66, 7.67, 7.68, 7.69, 7.7, 7.71, 7.72, 7.73, 7.74, 7.75, 7.76, 7.77, 7.78, 7.79, 7.8, 7.81, 7.82, 7.83, 7.84, 7.85, 7.86, 7.87, 7.88, 7.89, 7.9, 7.91, 7.92, 7.93, 7.94, 7.95, 7.96, 7.97, 7.98, 7.99, 8.0, 8.01, 8.02, 8.03, 8.04, 8.05, 8.06, 8.07, 8.08, 8.09, 8.1, 8.11, 8.12, 8.13, 8.14, 8.15, 8.16, 8.17, 8.18, 8.19, 8.2, 8.21, 8.22, 8.23, 8.24, 8.25, 8.26, 8.27, 8.28, 8.29, 8.3, 8.31, 8.32, 8.33, 8.34, 8.35, 8.36, 8.37, 8.38, 8.39, 8.4, 8.41, 8.42, 8.43, 8.44, 8.45, 8.46, 8.47, 8.48, 8.49, 8.5, 8.51, 8.52, 8.53, 8.54, 8.55, 8.56, 8.57, 8.58, 8.59, 8.6, 8.61, 8.62, 8.63, 8.64, 8.65, 8.66, 8.67, 8.68, 8.69, 8.7, 8.71, 8.72, 8.73, 8.74, 8.75, 8.76, 8.77, 8.78, 8.79, 8.8, 8.81, 8.82, 8.83, 8.84, 8.85, 8.86, 8.87, 8.88, 8.89, 8.9, 8.91, 8.92, 8.93, 8.94, 8.95, 8.96, 8.97, 8.98, 8.99, 9.0.

### Drug-Linker Conjugation to Amino Acid Residues

The present invention further provides an ADC comprising an antibody that binds CEACAM5 conjugated via a cysteine, lysine, serine or threonine or a non-canonical amino acid substitution of an amino acid within the antibody residue to a drug-linker provided herein.

The present invention further provides an ADC comprising an antibody that binds CEACAM5 conjugated via a cysteine or a non-canonical amino acid substitution of an amino acid within the antibody residue to a drug-linker provided herein.

Those skilled in the art should understand that the antibody-drug conjugate described in the present application can be prepared in a modularized manner. For example, the “drug-linker” in a pre- conjugated form (which can be understood as M′-L-E-D, wherein M′ is a structural form before covalent linkage of M to the antibody or antigen binding fragment thereof) is obtained, and then the ‘drug-linker’ in the pre- conjugated form is covalently linked to the antibody or antigen binding fragment thereof to obtain the antibody-drug conjugate according to the present application. Correspondingly, M′ in the pre- conjugated form of ‘drug-linker’ is linked to one or more sulfhydryl(—SH), amino (-NH₂) or carboxyl(—COOH) on the antibody or antigen binding fragment thereof through a substitution reaction (for example, removal —f-SO₂Me or -Br and the like structures thereon) or through an addition reaction and other manners.

In another aspect, the present invention provides a drug-linker having a structure shown as the formula M′-L-E-D, where

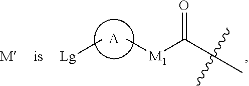

and Lg is a leaving group for a nucleophilic substitution reaction (for example, halogen, methanesulfonyl, fluorophenol, or

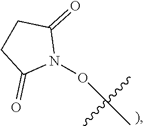 [SMILES: *ON1C(=O)CCC1=O]

or hydroxyl(—OH), sulfhydryl(—SH) or amino (-NH₂); or, Lg forms an unsaturated double bond with an adjacent atom on ring A; ring A is a 5-6 membered aliphatic heterocyclic ring, or a 5-20 membered aromatic ring system, and the aliphatic heterocyclic ring and the aromatic ring system are optionally substituted by one or more groups selected from oxo (═O), halogen, cyano, amino, carboxyl, sulfhydryl and C₁₋₆ alkyl; and M₁ is selected from a single bond, C₁₋₂₀ alkylene, C₂₋₂₀ alkenylene and C₂₋₂₀ alkynylene; and
L, E and D structures are defined according to any one of above antibody-drug conjugate.

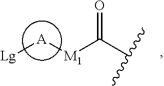

In some embodiments, M′is Lg is methanesulfonyl, or Lg and adjacent atoms on the ring A form carbon-carbon double bonds; the ring A is a 5-membered alicyclic heterocyclic ring, a 6-membered heteroaromatic ring, or a polycyclic ring formed by linking more than one 6-membered heteroaromatic ring and a benzene ring through a single bond, and the alicyclic heterocyclic ring is optionally substituted by one or more groups selected from oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M′ i:

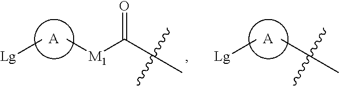

is selected from

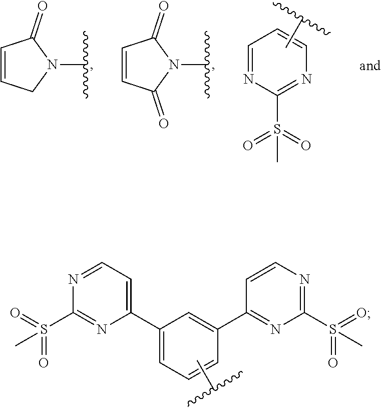

and M₁ is selected from a single bond, C₅₋₈ alkylene, C₅₋₈ alkenylene and C₅₋₈ alkynylene.

In some embodiments, M′ is selected from

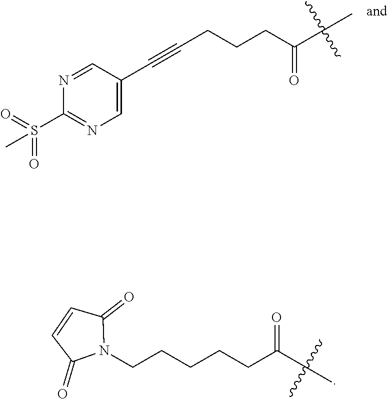

In some embodiments, the “drug-linker” in pre- conjugated form is selected from A-01 to A-03, A-1, A04 to A-25, A-2, B-01 to B-06, and C-01 shown as follows:

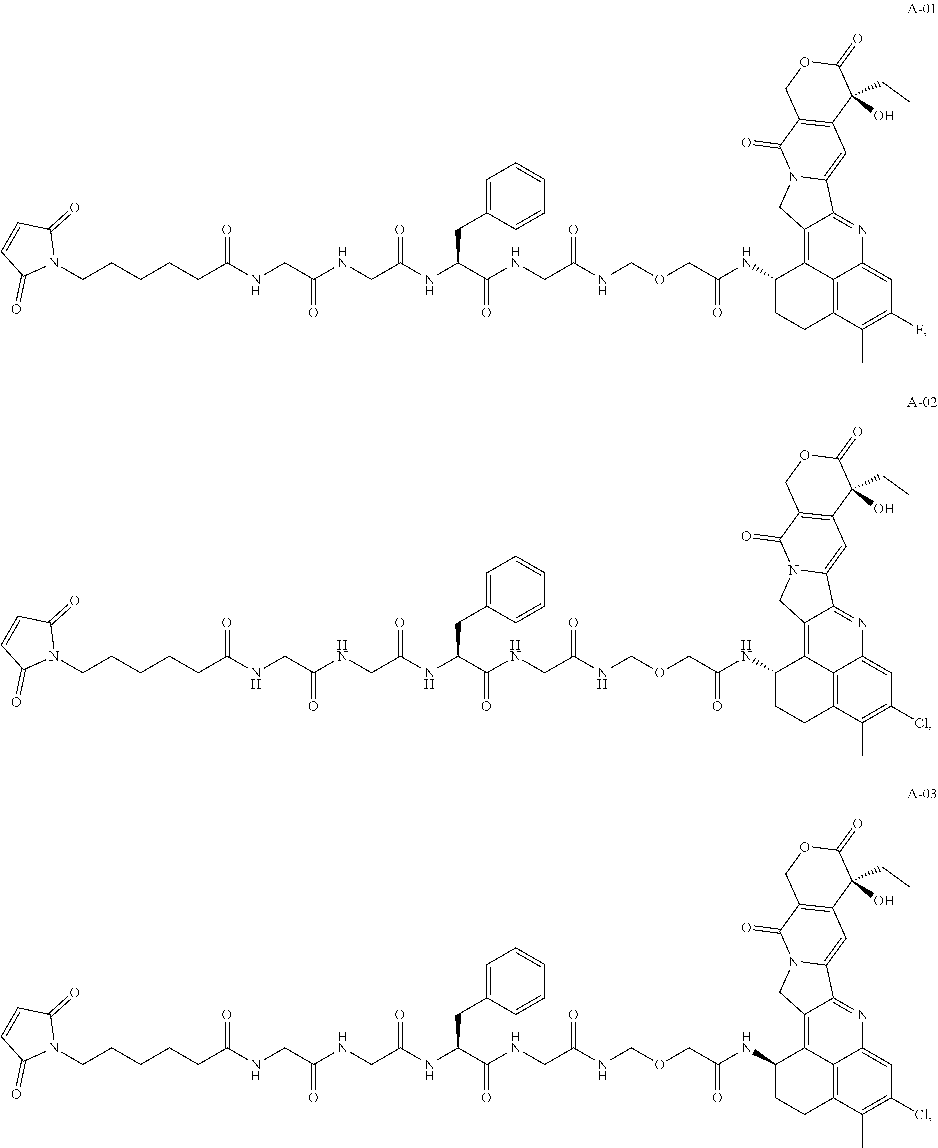 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(Cl)c(C)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

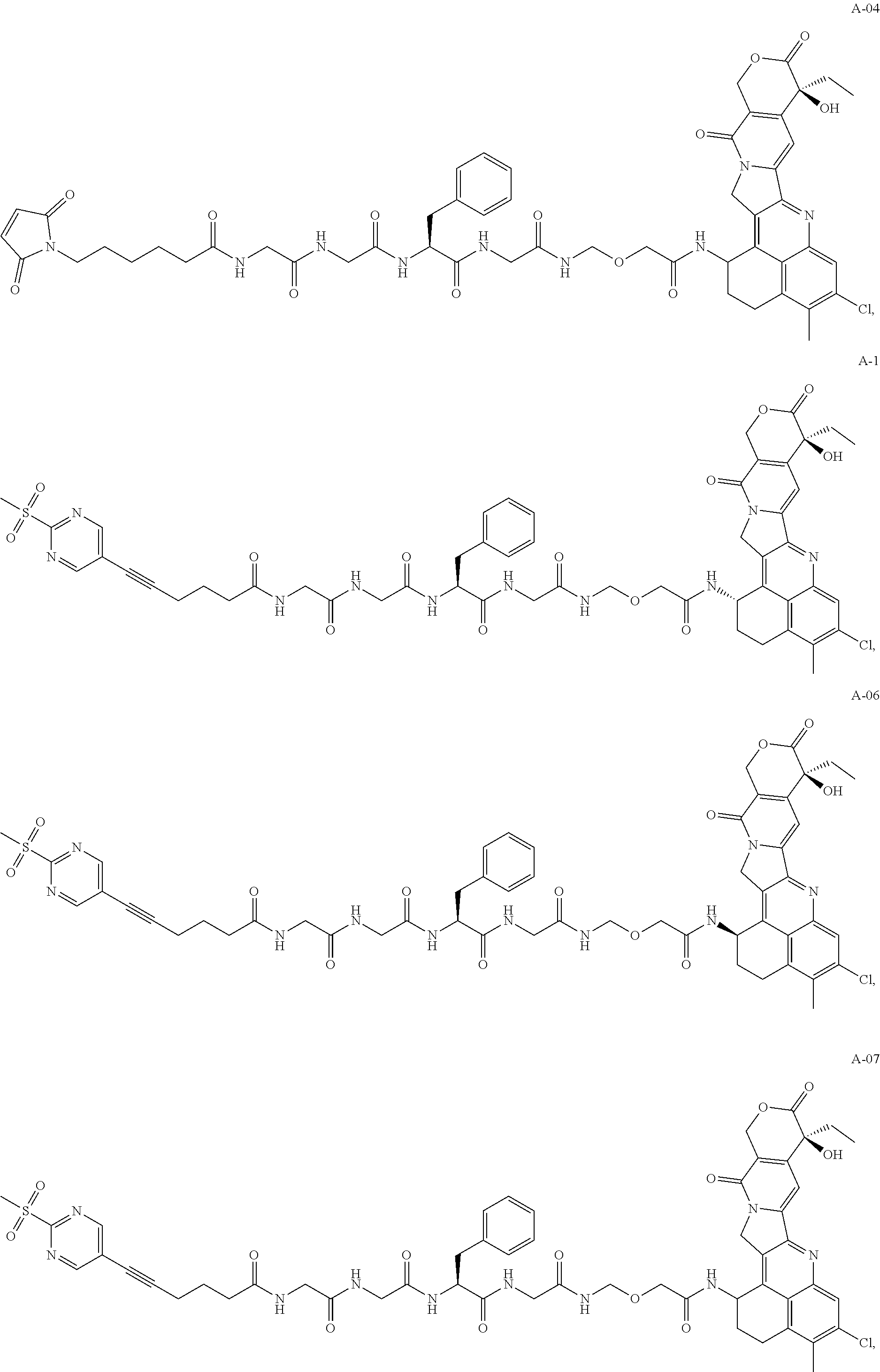

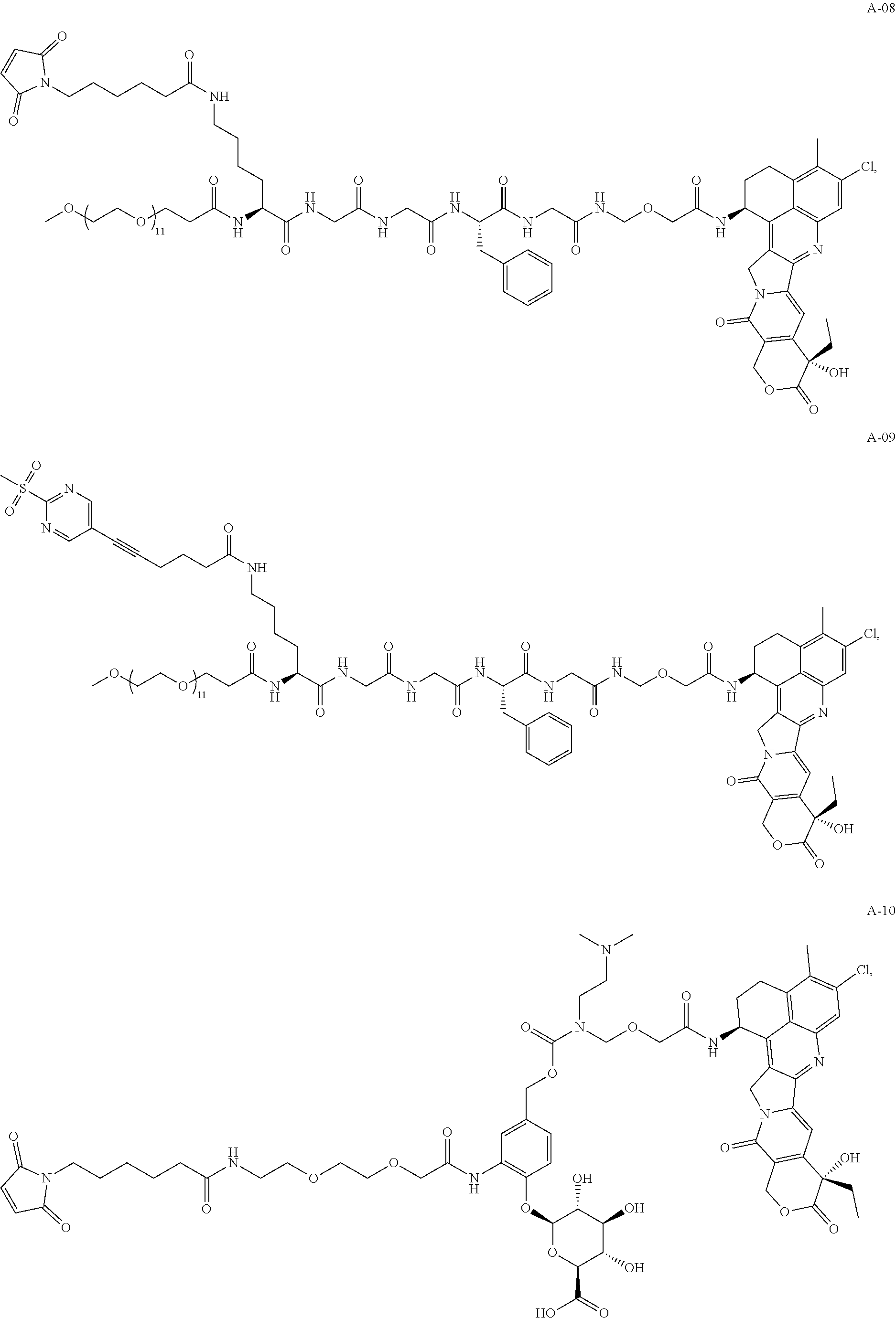

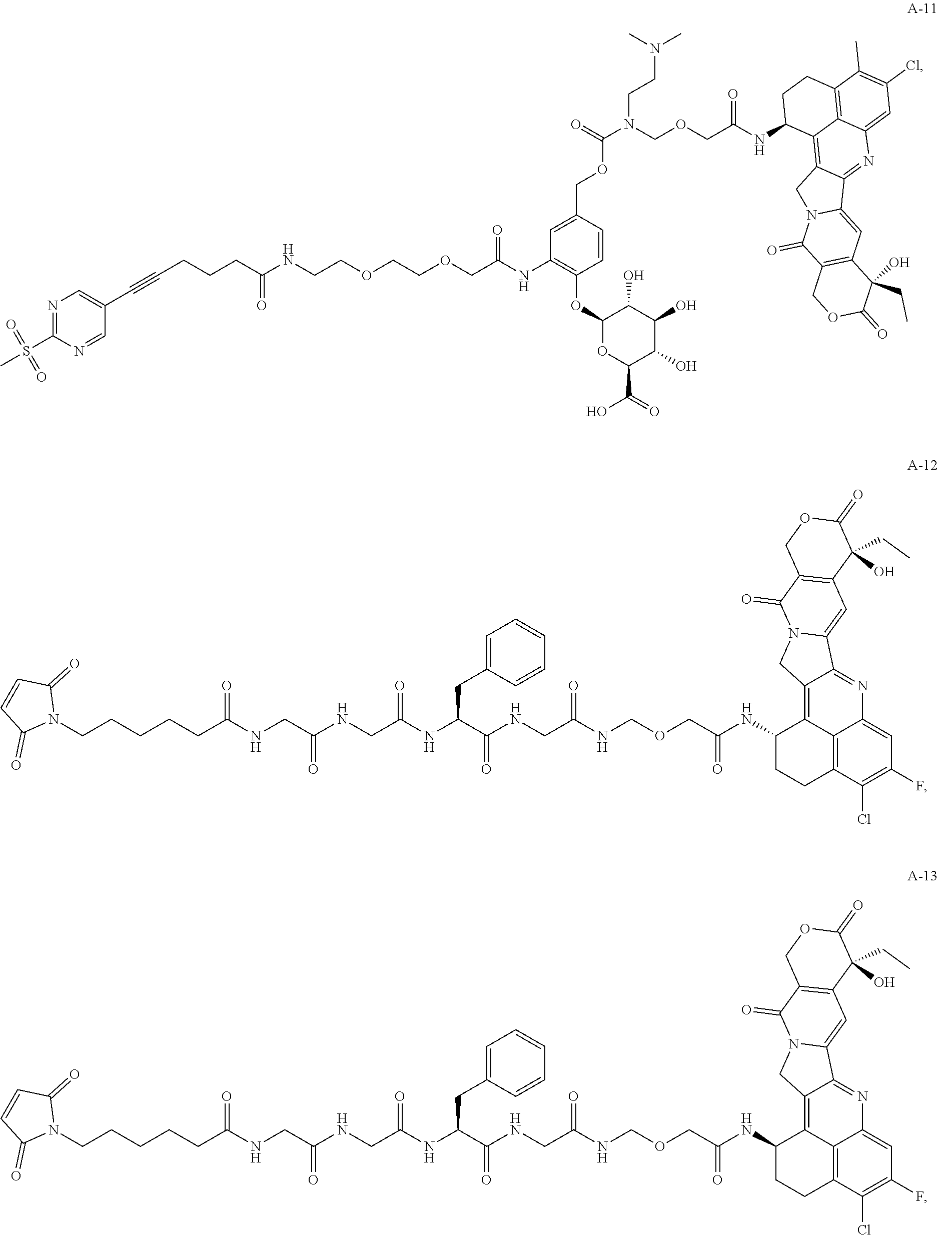 [SMILES: CC[C@@]1(O)C(=O)OCc2c1cc1n(c2=O)Cc2c-1nc1cc(Cl)c(C)c3c1c2[C@@H](NC(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(S(C)(=O)=O)nc2)c1)CC3 ; CC[C@@]1(O)C(=O)OCc2c1cc1n(c2=O)Cc2c-1nc1cc(F)c(Cl)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(Cl)c3c1c2[C@H](NC(=O)COCNC(=O)CNC(=O)[C@H](Cc1ccccc1)NC(=O)CNC(=O)CNC(=O)CCCCCN1C(=O)C=CC1=O)CC3]

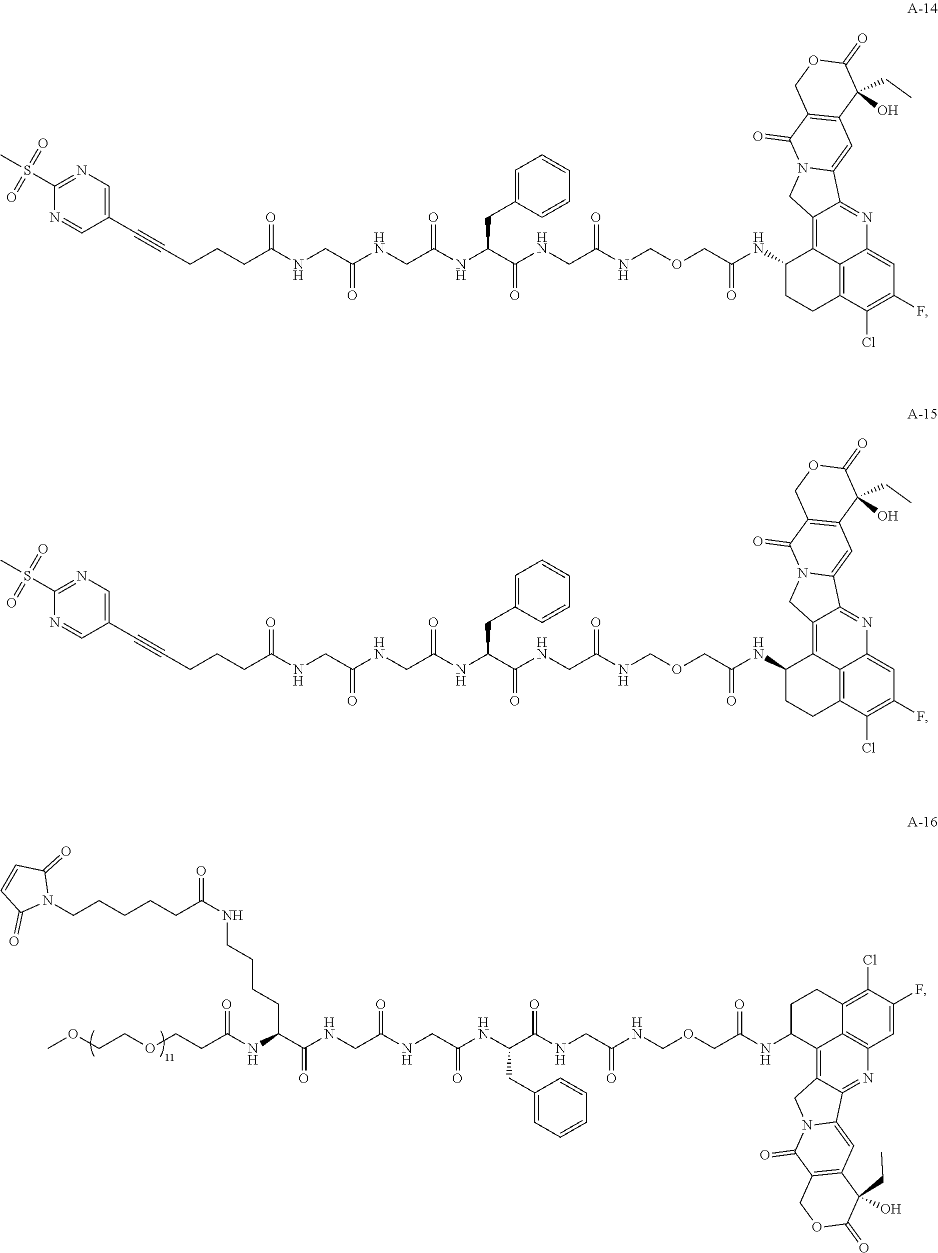

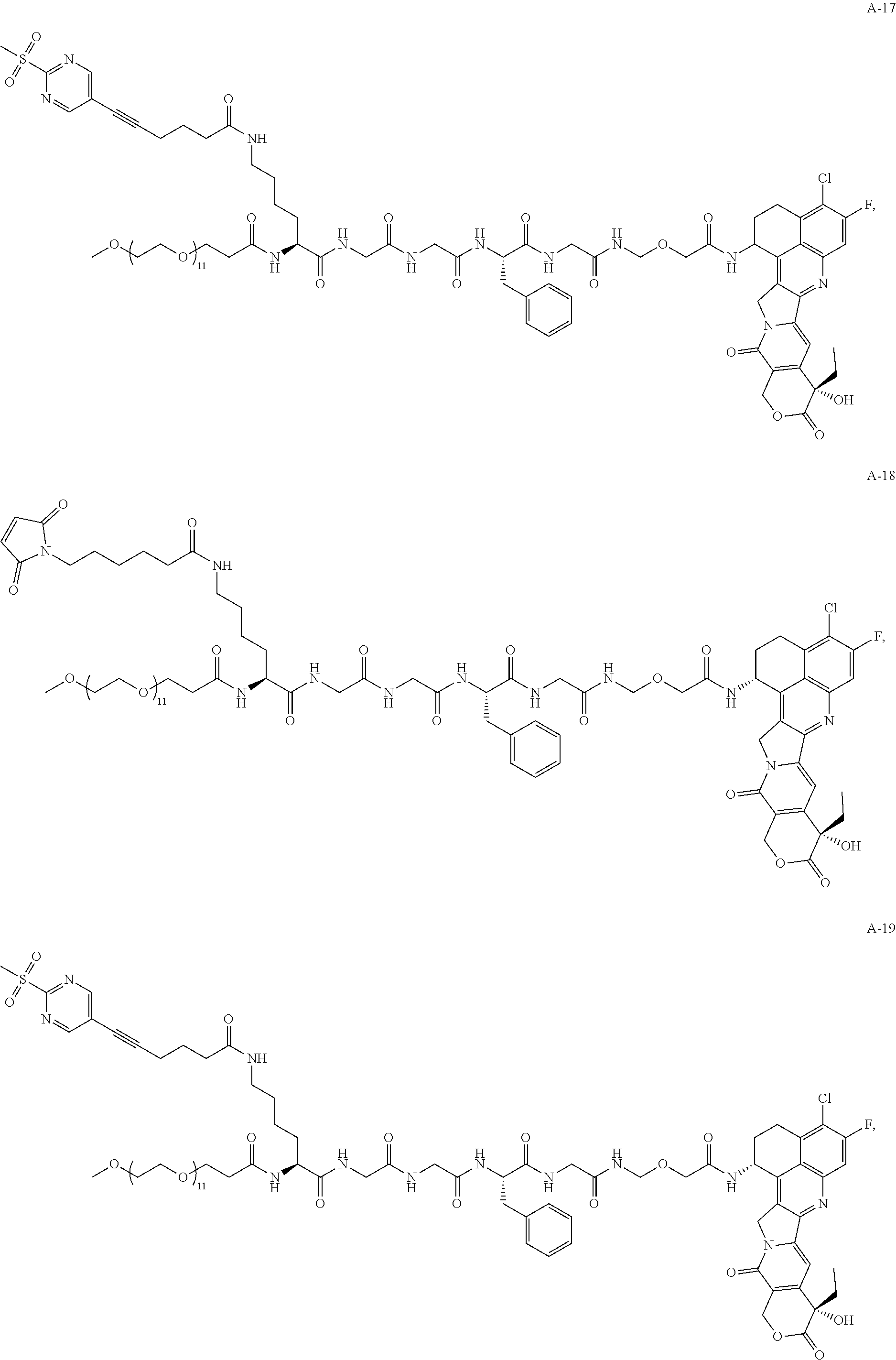

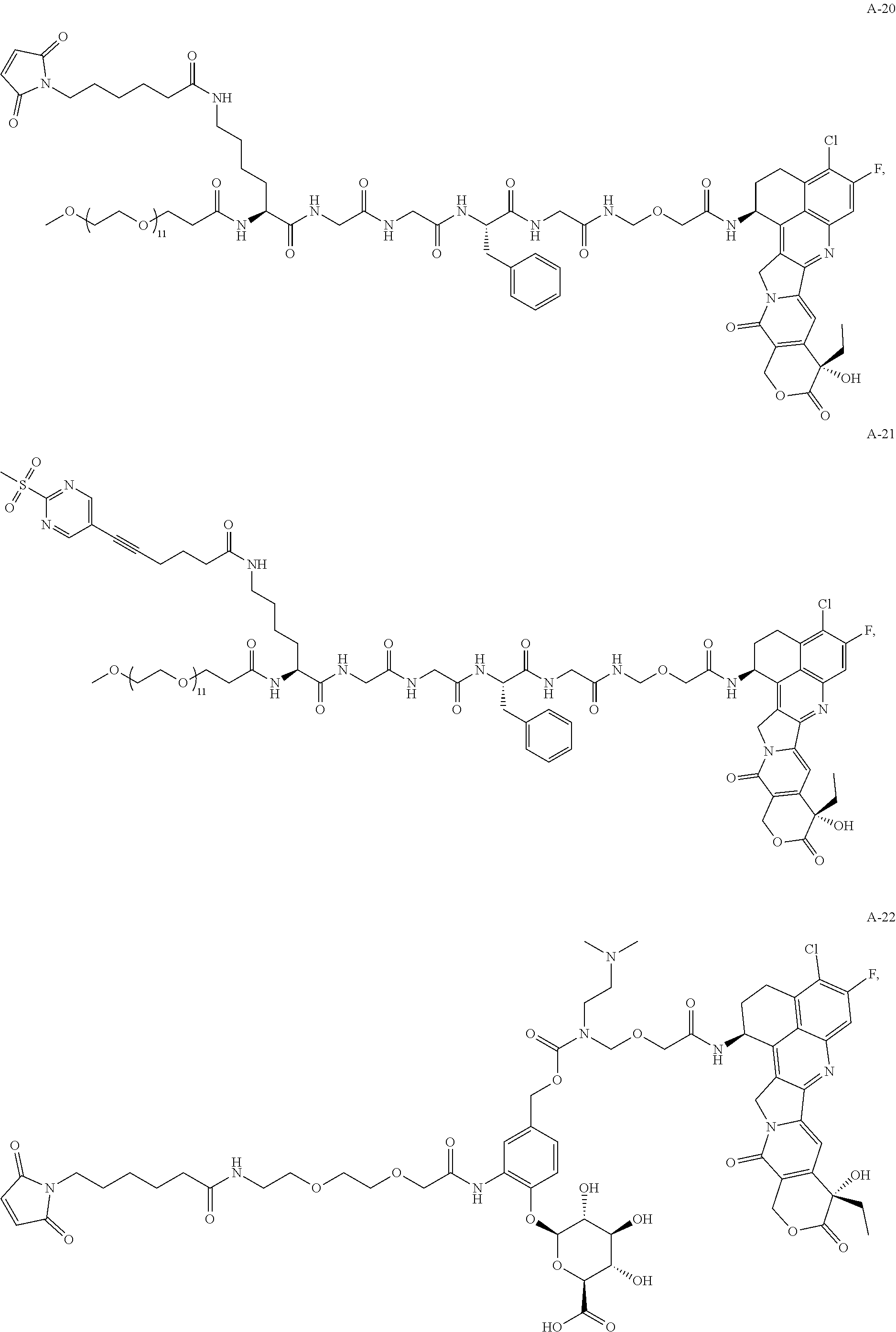

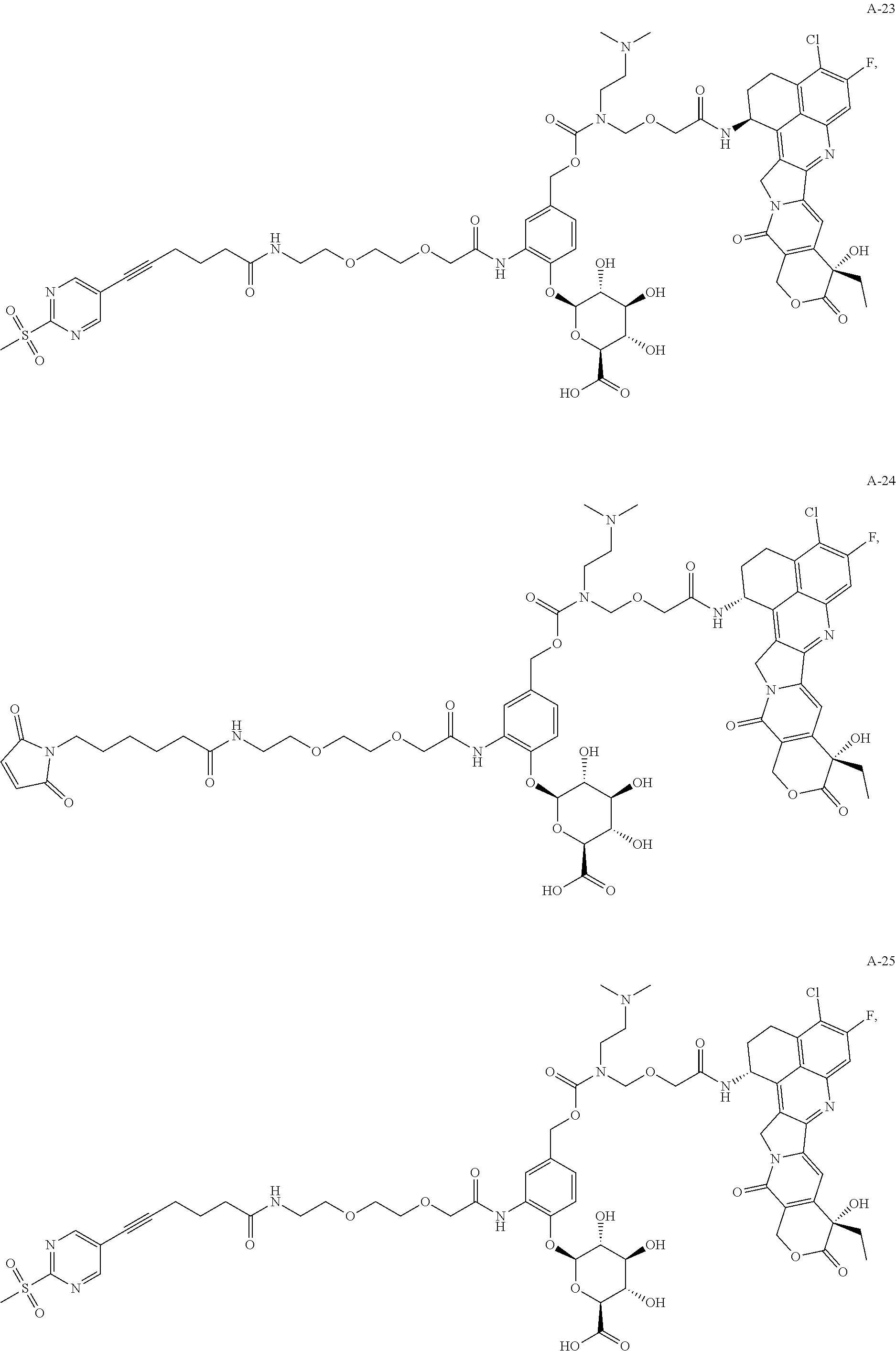

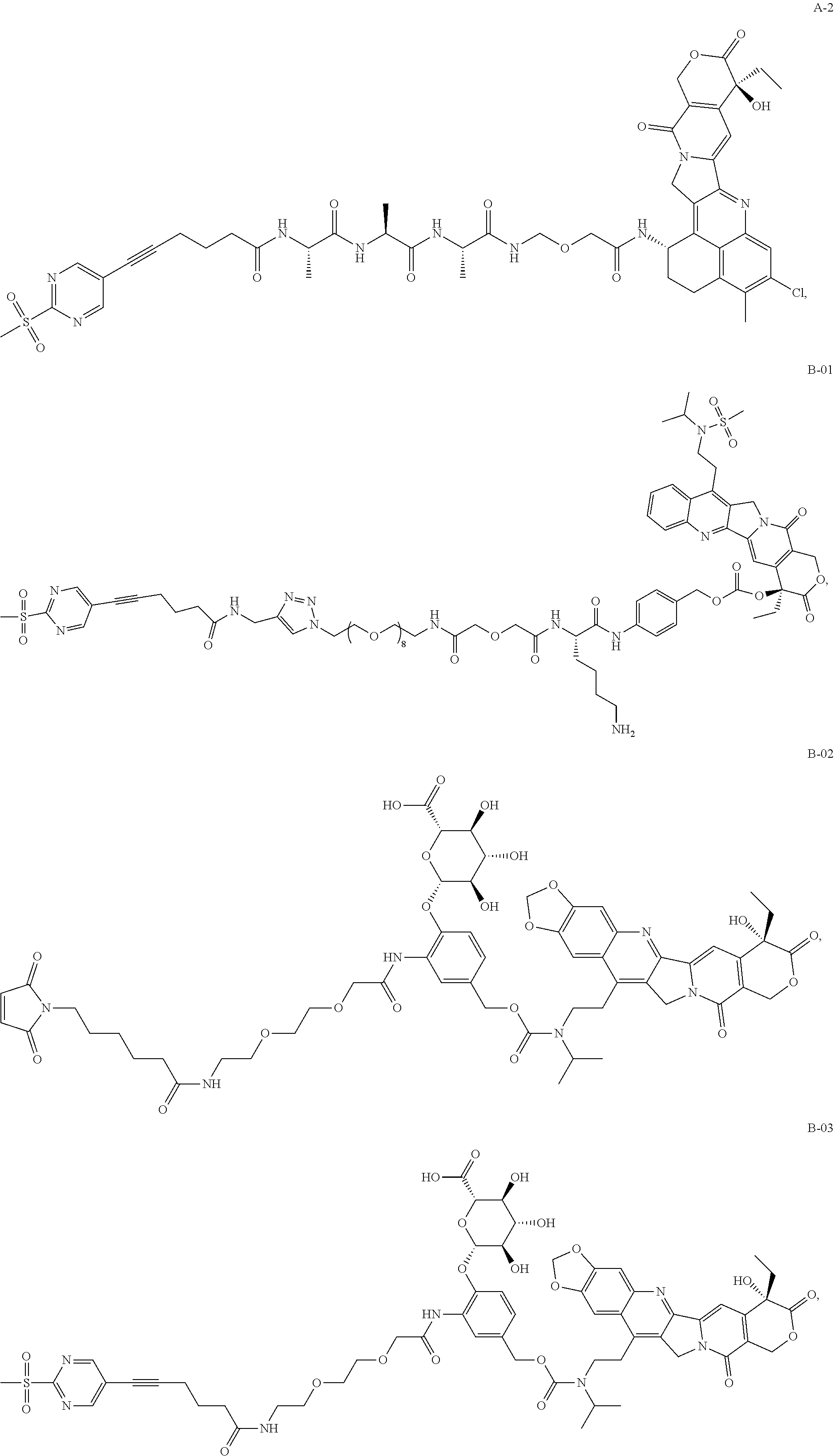

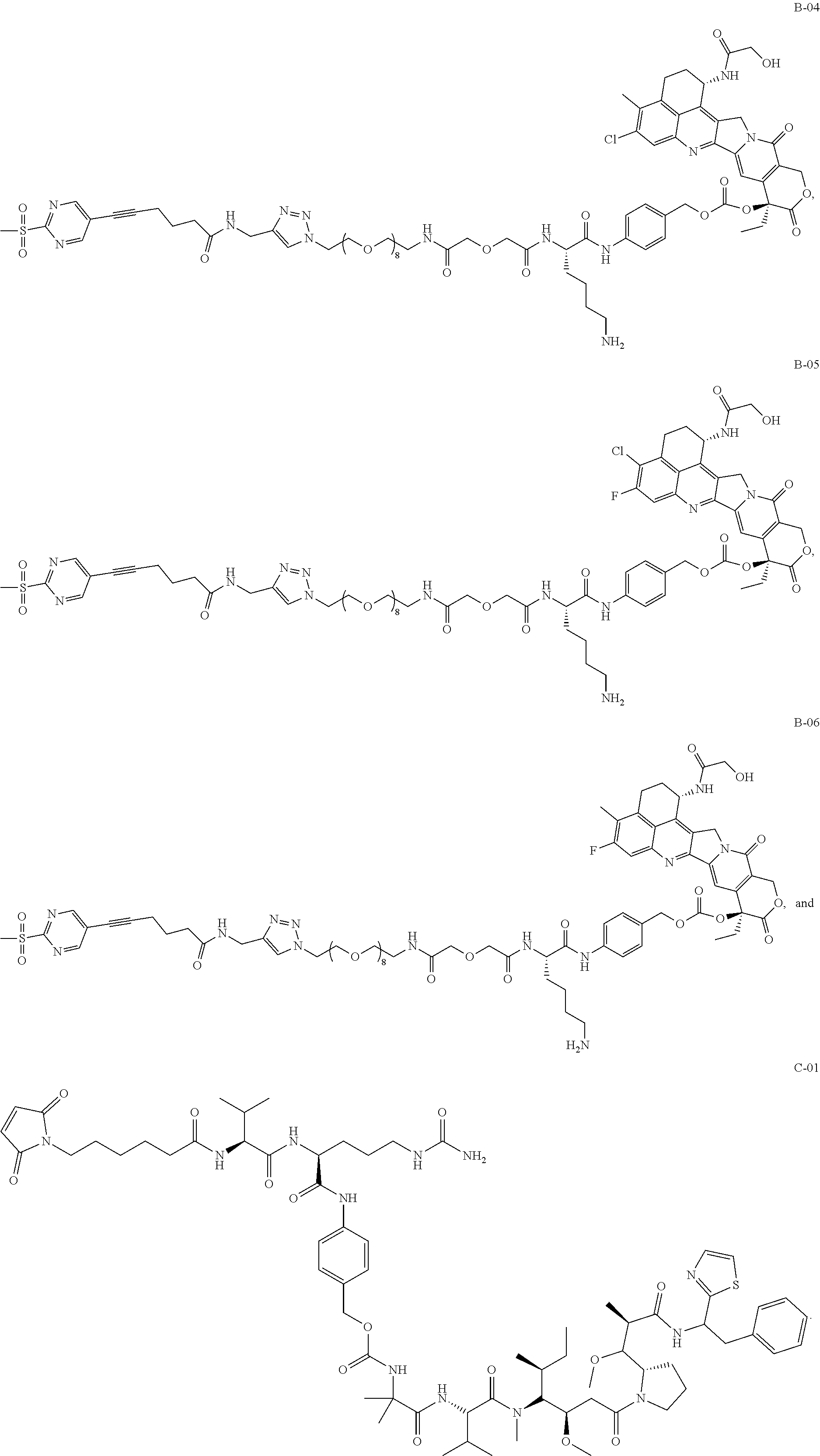

In a further embodiment of the ADC, the antibody is an antibody or antigen binding fragment selected from the group consisting of:
(1) the following heavy chain variable regions (VH) and/or light chain variable regions (VL), wherein CDRs are defined according to a Chothia numbering system: a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (1) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (2) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants of (3) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement;
or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants of (4) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids (for example, replacement, deletion or addition of 1, 2 or 3 amino acids) compared with the sequence from which the variants are derived; preferably, the replacement is a conservative replacement.

In some embodiments, the antibody or antigen-binding fragment thereof comprises (1) a heavy chain variable region (VH) comprising the following 3 CDRs: CDR-H1 having the amino acid sequence as set forth in SEQ ID NO: 14, CDR-H2 having the amino acid sequence as set forth in SEQ ID NO: 18, and CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises (2) a heavy chain variable region (VH) comprising the following 3 CDRs: CDR-H1 having the amino acid sequence as set forth in SEQ ID NO: 15, CDR-H2 having the amino acid sequence as set forth in SEQ ID NO: 19, CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises (3) a heavy chain variable region (VH) comprising the following 3 CDRs: CDR-H1 having the amino acid sequence as set forth in SEQ ID NO: 10, CDR-H2 having the amino acid sequence as set forth in SEQ ID NO: 17, CDR-H3 having the amino acid sequence as set forth in SEQ ID NO: 11; and a light chain variable region (VL) comprising the following 3 CDRs: CDR-L1 having the amino acid sequence as set forth in SEQ ID NO: 12, CDR-L2 having the amino acid sequence as set forth in SEQ ID NO: 13, and CDR-L3 having the amino acid sequence as set forth in SEQ ID NO: 9.

In some embodiments, the antibody or antigen-binding fragment thereof comprises (4) a heavy chain variable region (VH) comprising the following 3 CDRs: CDR-H1 having the amino acid sequence as set forth in SEQ ID NO: 5, CDR-H2 having the amino acid sequence as set forth in SEQ ID NO: 16, and CDR-H3 having the amino acid sequence as set forth in SEQ ID NO: 6; and a light chain variable region (VL) comprising the following 3 CDRs: CDR-L1 having the amino acid sequence as set forth in SEQ ID NO: 7, CDR-L2 having the amino acid sequence as set forth in SEQ ID NO: 8, and CDR-L3 having the amino acid sequence as set forth in SEQ ID NO: 9.

In some embodiments, the antibody or antigen-binding fragment thereof comprises a V_{H} having the amino acid sequence as set forth in SEQ ID NO: 1, and a V_{L} having the amino acid sequence as set forth in SEQ ID NO: 2.

In a further embodiment of the ADC, the antibody or antigen binding fragment comprises a V_{H} having the amino acid sequence as set forth in SEQ ID NO: 1 or a variant thereof, and/or, a V_{L} having the amino acid sequence as set forth in SEQ ID NO: 2 or a variant thereof.

In a further embodiment of the ADC, the antibody or antigen binding fragment comprises a heavy chain (HC) having the amino acid sequence as set forth in SEQ ID NO: 20 and a light chain (LC) having the amino acid sequence as set forth in SEQ ID NO: 21.

In a further embodiment of the ADC, the antibody or antigen binding fragment comprises a heavy chain (HC) consisting of the amino acid sequence as set forth in SEQ ID NO: and a light chain (LC) consisting of the amino acid sequence as set forth in SEQ ID NO: 21.

In a further embodiment of the ADC, the antibody or antigen binding fragment comprises a heavy chain (HC) having the amino acid sequence as set forth in SEQ ID NO: 22, 23 or 24 and a light chain (LC) having the amino acid sequence as set forth in SEQ ID NO: 21.

In a further embodiment of the ADC, the antibody or antigen binding fragment comprises a heavy chain (HC) consisting of the amino acid sequence as set forth in SEQ ID NO: 22, 23 or 24 and a light chain (LC) consisting of the amino acid sequence as set forth in SEQ ID NO: 21.

In a further embodiment of the ADC, the antibody or antigen binding fragment comprises a CH having the amino acid sequence as set forth in SEQ ID NO: 3, and a CL having the amino acid sequence as set forth in SEQ ID NO: 4.

### Antibody Preparation

The antibodies described herein can be prepared by various methods known in the art, for example, by genetic engineering and recombination techniques. For example, DNA molecules encoding the heavy and light chain genes of the antibodies of the present invention are obtained by chemical synthesis or PCR amplification. The resulting DNA molecule is inserted into an expression vector, which is then transfected into a host cell. Then, the transfected host cells are cultured under specific conditions, and express the antibody of the present invention.

### Conjugation

In another aspect, the present application provides a method for conjugating the drug-linkers described herein to the antibodies described herein to make the antibody-drug conjugates (ADCs) described herein.

In certain embodiments, the antibody described herein is conjugated to a drug-linker described herein via conjugation to a lysine in the antibody.

In certain embodiments, the antibody described herein is conjugated to a drug-linker described herein via conjugation to a cysteine residue in the antibody. In certain embodiments, the cysteine residues are from reduced intrachain disulfide bonds in the antibody. In certain embodiments, the cysteine residues are from reduced interchain disulfide bonds in the antibody.

In some embodiments, the antibody is conjugated to a drug-linker via conjugation to reduced interchain disulfide bonds in the antibody. For example, an IgG1 antibody consists of four polypeptide chains, two heavy chains comprising VH, CH1 and Fc (e.g., hinge, CH₂ and CH₃) domains, and two light chains comprising V_{L} and CL domains, connected by interchain cysteine disulfide (—S—S-) bonds (e.g., two heavy chain-light chain interchain disulfide bonds and two hinge heavy chain-heavy chain interchain disulfide bonds). In certain embodiments, when these disulfide bonds are broken under reducing conditions, eight (8) reactive cysteine sulfhydryl moieties are produced. In certain embodiments, each of the eight reactive cysteine sulfhydryl moieties is a point of attachment for a linker-drug, such that a maximum of eight (x=8) drug-linkers may be attached to the reduced antibody. In certain embodiments, any one of the four disulfide bonds is broken under reducing conditions, two (2) reactive cysteine sulfhydryl moieties are produced. In further embodiments, each of the two reactive cysteine sulfhydryl moieties is a point of attachment for a linker-drug, such that two (x=2) linker-drugs may be attached to the reduced antibody. In certain embodiments, any two of the four disulfide bonds are broken under reducing conditions, four (4) reactive cysteine sulfhydryl moieties are produced. In further embodiments, each of the four reactive cysteine sulfhydryl moieties is a point of attachment for a linker-drug, such that four (x=4) linker-drugs may be attached to the reduced antibody. In certain embodiments, any three of the four disulfide bonds are broken under reducing conditions, six (6) reactive cysteine sulfhydryl moieties are produced. In further embodiments, each of the six reactive cysteine sulfhydryl moieties is a point of attachment for a linker-drug, such that six (x=6) linker-drugs may be attached to the reduced antibody.

In some embodiments, the interchain disulfide bond is between two cysteine residues, which are broken under reducing conditions, resulting in two reactive cysteine sulfhydryl moieties. In further embodiments, the interchain disulfide bridge in the antibody is between a heavy chain and a light chain, such as between C220 of a heavy chain according to EU numbering and C214 of a kappa light chain according to the EU and/or Kabat numbering, or between C220 of a heavy chain according to the EU numbering and C214 of a lambda light chain according to the EU and/or Kabat numbering. Additionally, or alternatively, the interchain disulfide bridge in the antibody is between two heavy chains, such as between C226 and/or C229 of a first heavy chain and C226 and/or C229 of a second heavy chain according to the EU numbering. In some embodiments, the cysteine residues are in the hinge region of the antibody. In some embodiments, the cysteine residue is at any one or more of positions 220, 226, or 229 in the heavy chain according to EU numbering (also referred to herein as C220, C226 or C229, respectively). In some embodiments, the cysteine residue is at position 214 in the light chain according to EU and/or Kabat numbering (also referred to herein as C214, such as position 214 in the kappa light chain according to EU and Kabat numbering or position 214 in the lambda light chain according to the EU and Kabat numbering). In one embodiment, the cysteine residues are at each of positions 220, 226, and 229 in the heavy chain according to the EU numbering and position 214 in the light chain, according to EU or Kabat numbering. In one embodiment, the cysteine residues are at each of positions 220, 226, and 229 in the heavy chain according to the EU numbering and position 214 in the kappa light chain, according to EU and Kabat numbering. In one embodiment, the cysteine residues are at each of positions 220, 226, and 229 in the heavy chain according to the EU numbering and position 214 in the lambda light chain, according to EU and Kabat numbering. In one embodiment, the cysteine residues are at any one or more of the following positions:
(i) any one, or any two, or any three of positions 220, 226, and 229 in a first heavy chain according to the EU numbering;
(ii) any one, or any two, or any three of positions 220, 226, and 229 in a second heavy chain according to the EU numbering;
(iii) position 214 in a first light chain according to the Kabat numbering and/or EU numbering; and/or
(iv) position 214 in a second light chain according to the Kabat numbering and/or EU numbering.

As used herein, C220, C226, and C229 refer to amino acid residues (cysteine, Cys, C) of an immunoglobulin identified according to the EU numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGHGnber.html.

As used herein, the cysteine residue at position 214 of kappa light chain refer to amino acid residues (cysteine, Cys, C) of an immunoglobulin identified according to the Eu or Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGKCnber.html.

As used herein, the cysteine residue at position 214 of lambda light chain refer to amino acid residues (cysteine, Cys, C) of an immunoglobulin identified according to the Eu or

Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGLCnber.html.

In certain embodiments, the antibodies described herein comprise four interchain disulfide bonds in the hinge region which may be reduced, thereby breaking the bond, and revealing a reactive sulfhydryl moiety that may be conjugated with a maleimide moiety on a drug-linker, such as the maleimide moiety on drug-linkers described herein.

In certain embodiments, the antibodies described herein comprise lysine residues wherein the reactive amine side chains of lysine residues may be conjugated with a drug-linker, such as a maleimide moiety on drug-linkers described herein.

In one embodiment, the present disclosure provides a method of making an ADC described herein, comprising the steps of:
a) providing a solution comprising the antibody;
b) contacting the solution of a) with a reducing agent;
c) contacting the solution of b) with a solution comprising a drug-linker or a salt thereof, as described herein, to make the ADC.

In one embodiment, the reducing agent is tris (2 carboxyethyl) phosphine (TCEP).

### Compositions

In another aspect, the present application provides a composition of antibody-drug conjugates (ADCs) as described herein. Such composition may comprise a plurality of ADCs as described herein, wherein each ADC comprises a drug-linker as described herein, wherein x is independently 1, 2, 3, 4, 5, 6, 7, 8, 9 or 10. In other words, each antibody molecule in the composition may be conjugated to 1, 2, 3, 4, 5, 6, 7, 8, 9 or 10 drug-linkers. Therefore, a composition may be characterized by a “drug-to-antibody” ratio (DAR) ranging from about 1 to about 10. Methods to determine DAR are well known to the skilled person and include methods using Reverse Phase Chromatography, or HPLC-MS.

For example, in any embodiment, an ADC composition described herein has a DAR of about 1 to about 10 or any subrange there between, for example: about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, about 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, an ADC composition described herein has a DAR of about 3 to 9, for example, about 3.0 to 3.5, about 3.0 to 4.0, about 3.0 to 4.5, about 3.0 to 5.0, about 3.0 to 5.5, about 3.0 to 6.0, about 3.5 to 4.0, about 3.5 to 4.5, about 3.5 to 5.0, about 3.5 to 5.5, about 3.5 to 6.0, about 3.5 to 6.5, about 3.5 to 7.0, about 3.5 to 7.5, about 3.5 to 8.0, about 4.0 to 4.5, about 4.0 to 5.0, about 4.0 to 5.5, about 4.0 to 6.0, about 4.0 to 6.5, about 4.0 to 7.0, about 4.0 to 7.5, about 4.0 to 8.0, about 4.5 to 5.0, about 4.5 to 5.5, about 4.5 to 6.0, about 4.5 to 6.5, about 4.5 to 7.0, about 4.5 to 7.5, about 4.5 to 8.0, about 5.0 to 5.5, about 5.0 to 6.0, about 5.0 to 6.5, about 5.0 to 7.0, about 5.0 to 7.5, about 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.5, about 7.0 to 7.5, about 7.0 to 9.0 or about 7.5 to 9.0.

### Pharmaceutical Compositions

In another aspect, the present application provides a pharmaceutical composition, which comprises the antibody-drug conjugate (ADC) described in any one of the foregoing embodiments and optionally the drug-linker described in any one of the foregoing embodiments, and one or more pharmaceutically acceptable auxiliaries.

In one embodiment, the pharmaceutical compositions provided herein comprise an antibody-drug conjugate (ADC) and one or more pharmaceutically acceptable auxiliaries, wherein the ADC is:

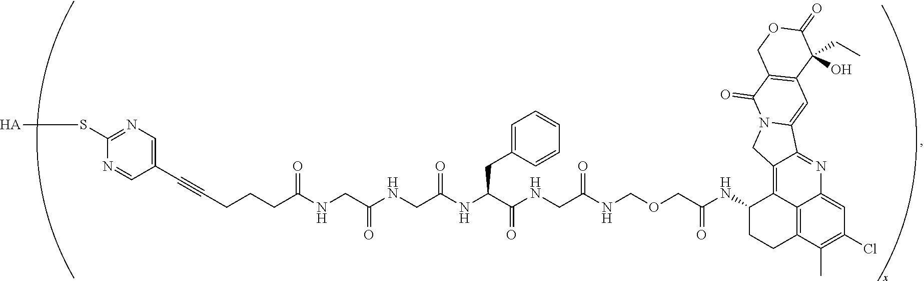

wherein HA-(S- comprises a heavy chain (HC) having the amino acid sequence as set forth in SEQ ID NO: 20 or 22 and a light chain (LC) having the amino acid sequence as set forth in SEQ ID NO: 21; and x is 4-9.

In one embodiment, the pharmaceutical compositions provided herein comprise an antibody-drug conjugate (ADC) and one or more pharmaceutically acceptable auxiliaries, wherein the ADC is ADC-Aa:

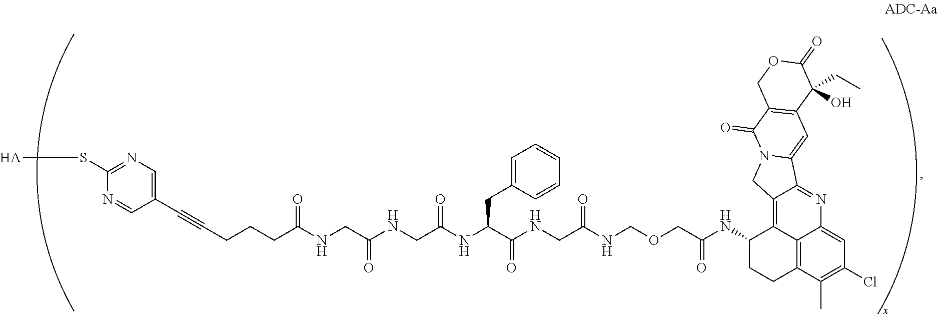

wherein HA-(S- comprises a heavy chain (HC) having the amino acid sequence as set forth in SEQ ID NO: 20 or 22 and a light chain (LC) having the amino acid sequence as set forth in SEQ ID NO: 21; and x is 4 or 8.

In the foregoing embodiment for the pharmaceutical compositions, for example, for the pharmaceutical compositions comprising the antibody drug conjugate of formula ADC-Aa, in one embodiment of the antibody-drug conjugate, x is 4. In one embodiment, x is 8. In one embodiment, x is an integer.

In one embodiment, the pharmaceutical compositions provided herein comprise an antibody-drug conjugate (ADC) and one or more pharmaceutically acceptable auxiliaries, wherein the ADC is ADC-Bb:

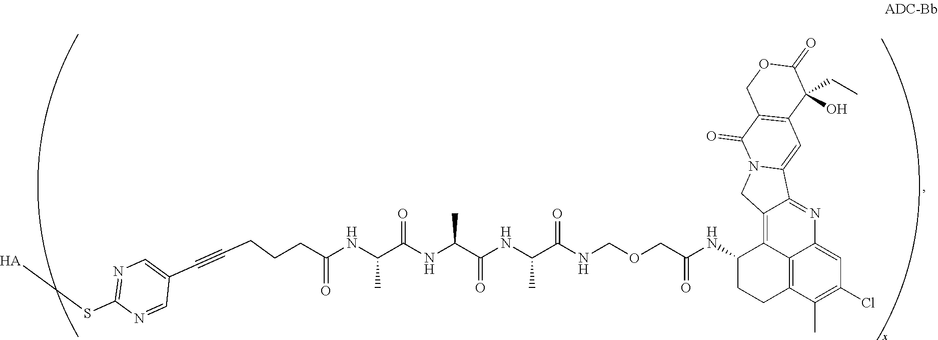

wherein HA-(S- comprises a heavy chain (HC) having the amino acid sequence as set forth in SEQ ID NO: 20 or 22 and a light chain (LC) having the amino acid sequence as set forth in SEQ ID NO: 21; and x is 4-9.

In one embodiment, the pharmaceutical compositions provided herein comprise an antibody-drug conjugate (ADC) and one or more pharmaceutically acceptable auxiliaries, wherein the ADC is:

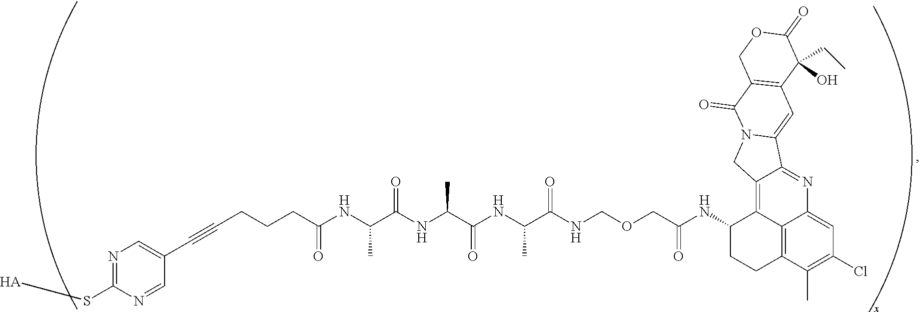

wherein HA-(S- comprises a heavy chain (HC) having the amino acid sequence as set forth in SEQ ID NO: 20 or 22 and a light chain (LC) having the amino acid sequence as set forth in SEQ ID NO: 21; and x is 4 or 8.

In the foregoing embodiment for the pharmaceutical compositions, for example, for the pharmaceutical compositions comprising the antibody drug conjugate of formula ADC-Bb, in one embodiment of the antibody-drug conjugate, x is 4. In one embodiment, x is 8. In one embodiment, x is an integer.

Pharmaceutically acceptable auxiliaries include, for example, a pharmaceutically acceptable carrier and/or excipient. Pharmaceutically acceptable auxiliaries further includes salts and solvates.

The ADC described herein is typically formulated with a pharmaceutically acceptable parenteral vehicle to form a unit injectable form for parenteral application, such as bolus injection, intravenous injection, intratumoral injection, and the like. Optionally, the antibody-drug conjugate having the desired purity is mixed with a pharmaceutically acceptable diluents, carriers, excipients or stabilizers in the form of lyophilizate or solution (Remington's Pharmaceutical Sciences (1980) 16th edition, Osol, A. Ed.). The antibody-drug conjugate described herein, or the pharmaceutical composition comprising the antibody-drug conjugate, can be administered via any route appropriate for the individual to be treated.

The ADC and pharmaceutical compositions described herein can be formulated into any dosage form known in the medical field, for example, tablets, pills, suspensions, emulsions, solutions, gels, capsules, powders, granules, elixirs, lozenges, suppositories, injections (including injections, sterile powders for injections and concentrated solutions for injections), inhalants, sprays, etc. The preferred dosage form depends on the intended mode of administration and therapeutic use. Pharmaceutical compositions of the invention should be sterile and stable under the conditions of manufacture and storage. A preferred dosage form is injection. Such injections can be sterile injectable solutions. For example, sterile injectable solutions can be prepared by: a necessary dose of the antibody of the present invention is incorporated in an appropriate solvent, and optionally, other desired ingredients (including but not limited to, pH adjusters, surfactants, adjuvants, ionic strength enhancers, etc. osmotic agent, preservative, diluent, or any combination thereof), followed by filter sterilization. In addition, sterile injectable solutions can be prepared as sterile lyophilized powder (e.g., by vacuum drying or freeze-drying) for ease of storage and use. Such sterile lyophilized powder can be dispersed in a suitable carrier, such as sterile pyrogen-free water, before use.

Furthermore, the ADCs described herein may be presented in pharmaceutical compositions in unit dosage form for ease of administration by any suitable method known in the art, including but not limited to, oral, oral, sublingual, ophthalmic, topical, parenteral, rectal, intrathecal, intracytoplasmic reticulum, inguinal, intravesical, topical (e.g., powder, ointment, or drops), or nasal routes. However, for many therapeutic uses, the preferred route/mode of administration is parenteral (e.g., intravenous, subcutaneous, intraperitoneal, intramuscular). The skilled artisan will understand that the route and/or manner of administration will vary depending on the intended purpose. In some preferred embodiments, the ADCs and pharmaceutical compositions described herein are administered by intravenous infusion or injection.

In certain embodiments, the pharmaceutical composition may also comprise additional pharmaceutically active agents. In certain embodiments, the additional pharmaceutically active agent is a drug having antineoplastic activity. In certain embodiments, the additional pharmaceutically active agent is selected from the group consisting of EGFR inhibitors, HER2 inhibitors, HER3 inhibitors, HER4 inhibitors, IGFR-1 inhibitors, mTOR inhibitors, PI3 kinase inhibitors, PTK7 inhibitors, c-met or VEGF inhibitors, chemotherapy drugs, or any combination thereof. In certain embodiments, the ADCs described herein and the additional pharmaceutically active agent are provided as separate components or as admixed components. Accordingly, the antibody or antigen-binding fragment thereof of the invention and the additional pharmaceutically active agent may be administered simultaneously, separately or sequentially.

### Methods of Use

The antibody-drug conjugate described herein, drug-linker described herein, compositions described herein, or pharmaceutical compositions thereof can be used for treating various diseases or conditions, such as cancers with high expression of CEACAM5, including solid tumors, such as, rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer).

In one embodiment, the cancer is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor or ovarian serous cystadenocarcinoma.

In one embodiment, the cancer is colorectal cancer, gastric cancer or GEJ adenocarcinoma (adenocarcinomas of the esophagogastric junction), non-small cell lung cancer, pancreatic ductal adenocarcinoma, or esophageal carcinoma.

Therefore, the present application provides use of the antibody-drug conjugate (ADC), the drug-linker, compositions described herein, or the pharmaceutical composition containing the same as described in any one of foregoing embodiments, in the manufacture of a medicament for the treatment of a cancer with high expression of CEACAM5.

At the same time, the present application also provides a method for treating a cancer with high expression of CEACAM5, which comprises a step of administering to a subject in need thereof a therapeutically effective amount of the antibody-drug conjugate (ADC), the drug-linker, compositions described herein, or the pharmaceutical composition comprising the same as described in any one of foregoing embodiments.

The present application also provides the antibody-drug conjugate (ADC), the drug-linker, compositions described herein, or the pharmaceutical composition containing the same as described in any one of foregoing embodiments, for use in the treatment of a cancer with high expression of CEACAM5.

In certain embodiments, the antibody-drug conjugate (ADC), drug-linker, or

pharmaceutical composition is sufficient (e.g., in a subject) to:
(1) Inhibit the proliferation of cells (such as tumor cells);
(2) inhibit tumor growth;
(3) Induce and/or increase antibody-dependent cytotoxic activity;
(4) Inhibit CEACAM5-mediated signal transduction;
(5) Prevention and/or treatment of CEACAM5-mediated diseases/disorders; or
(6) Any combination of the above (1)-(5).

In certain embodiments, the CEACAM5-mediated disease/disorder is a tumor, e.g., a CEACAM5-expressing tumor. In certain embodiments, the tumor is selected from rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, gastric cancer or GEJ adenocarcinoma (adenocarcinomas of the esophagogastric junction), bladder epithelial carcinoma, breast ductal carcinoma, ovarian endometriosis carcinoma, prostate transitional cell carcinoma, cervical squamous cell carcinoma, colorectal cancer, and non-small cell lung cancer (including advanced non-squamous (NSq) non-small cell lung cancer), or any combination thereof.

In one embodiment, the cancer is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor or ovarian serous cystadenocarcinoma.

In certain embodiments, the cancer is a cancer of the rectum, colon, stomach, pancreas, esophagus, lung, head and neck, breast, bladder, uterine, prostate, skin, testicle, ovary, liver, thyroid, kidney, or brain. In certain embodiments, the cancer is a lymphoma, a melanoma or a leukemia.

In one embodiment, the cancer is colorectal cancer, gastric or GEJ adenocarcinoma (adenocarcinomas of the esophagogastric junction), non-small cell lung cancer, pancreatic ductal adenocarcinoma, or esophageal carcinoma.

### Examples

The following descriptions of specific embodiments will further illustrate the present invention, but this is not a limitation to the present invention. Those skilled in the art can make various modifications or improvements according to the teaching of the present invention without departing from the basic idea and scope of the present invention.

Information about the sequences involved in the present invention is described in the following table:

| SEQ | | |
|---|---|---|
| ID | | |
| NO: | Sequence Name | Sequence Information |
| 1 | Ab-hzDPmut VH | QVQLVQSGAEVVKPGASVKVSCKASGYTFTRYWMHW |
| | amino acid sequence | VRQAPGQGLEWMGRIEPNAGEPTYNEKFKNRVTMTVD |
| | | TSTSTVYMELSSLRSEDTAVYYCARSTTTVEAMDYWGQ |
| | | GTSVTVSS |
| 2 | Ab-hzDPmut VL | DIQLTQSPSSLSASVGDRVTITCRASESVDNYGFTFMHW |
| | amino acid sequence | YQQKPGKAPKLLIYRASNLESGIPSRFSGSGSGTDFTLTIS |
| | | SVQPEDFATYYCQQSNKNPTFGGGTKLEIK |
| 3 | Human mutant IgG1 | ASTKGPSVFPLAPSSKSTSGGTAALGCLVKDYFPEPVTVS |
| | amino acid sequence | WNSGALTSGVHTFPAVLQSSGLYSLSSVVTVPSSSLGTQT |
| | of a heavy chain | YICNVNHKPSNTKVDKKVEPKSCDKTHTCPPCPAPEAA |
| | constant region | GAPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVY |
| | | TLPPSRDELTKNQVSLTCLVKGFYPSDIAVEWESNGQPEN |
| | | NYKTTPPVLDSDGSFFLYSKLTVDKSRWQQGNVFSCSV |
| | | MHEALHNHYTQKSLSLSPGK |
| 4 | Human IgG1 CL | RTVAAPSVFIFPPSDEQLKSGTASVVCLLNNFYPREAKV |
| | amino acid sequence | QWKVDNALQSGNSQESVTEQDSKDSTYSLSSTLTLSKA |
| | of a light chain | DYEKHKVYACEVTHQGLSSPVTKSFNRGEC |
| | constant region | |
| 5 | Ab-hzDPmut Abm | GYTFTRYWMH |
| | CDRH1 | |
| 6 | Ab-hzDPmut | STTTVEAMDY |
| | Abm/Chothia/Kabat | |
| | CDRH3 | |
| 7 | Ab-hzDPmut | RASESVDNYGFTFMH |
| | Abm/Chothia/Kabat | |
| | CDRL1 | |
| 8 | Ab-hzDPmut | RASNLES |
| | Abm/Chothia/Kabat | |
| | CDRL2 | |
| 9 | Ab-hzDPmut | QQSNKNPT |
| | Abm/Chothia/Kabat/ | |
| | IMGT CDRL3 | |
| 10 | Ab-hzDPmut IMGT | GYTFTRYW |
| | CDRH1 | |
| 11 | Ab-hzDPmut IMGT | ARSTTTVEAMDY |
| | CDRH3 | |
| 12 | Ab-hzDPmut IMGT | ESVDNYGFTF |
| | CDRL1 | |
| 13 | Ab-hzDPmut IMGT | RAS |
| | CDRL2 | |
| 14 | Ab-hzDPmut Chothia | GYTFTRY |
| | CDRH1 | |
| 15 | Ab-hzDPmut kabat | RYWMH |
| | CDRH1 | |
| 16 | Ab-hzDPmut AbM | RIEPNAGEPT |
| | CDRH2 | |
| 17 | Ab-hzDPmut IMGT | IEPNAGEP |
| | CDRH2 | |
| 18 | Ab-hzDPmut Chothia | EPNAGE |
| | CDRH2 | |
| 19 | Ab-hzDPmut Kabat | RIEPNAGEPTYNEKFKN |
| | CDRH2 | |
| 20 | Ab-hzDPmut heavy | QVQLVQSGAEVVKPGASVKVSCKASGYTFTRYWMHW |
| | chain amino acid | VRQAPGQGLEWMGRIEPNAGEPTYNEKFKNRVTMTVD |
| | sequence | TSTSTVYMELSSLRSEDTAVYYCARSTTTVEAMDYWGQ |
| | | GTSVTVSSASTKGPSVFPLAPSSKSTSGGTAALGCLVKD |
| | | YFPEPVTVSWNSGALTSGVHTFPAVLQSSGLYSLSSVVT |
| | | VPSSSLGTQTYICNVNHKPSNTKVDKKVEPKSCDKTHTC |
| | | PPCPAPEAAGAPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| 21 | Ab-hzDPmut light | DIQLTQSPSSLSASVGDRVTITCRASESVDNYGFTFMHW |
| | chain amino acid | YQQKPGKAPKLLIYRASNLESGIPSRFSGSGSGTDFTLTIS |
| | sequence | SVQPEDFATYYCQQSNKNPTFGGGTKLEIKRTVAAPSVFI |
| | | FPPSDEQLKSGTASVVCLLNNFYPREAKVQWKVDNALQ |
| | | SGNSQESVTEQDSKDSTYSLSSTLTLSKADYEKHKVYAC |
| | | EVTHQGLSSPVTKSFNRGEC |
| 22 | Ab-hzDPmut heavy | XVQLVQSGAEVVKPGASVKVSCKASGYTFTRYWMHW |
| | chain amino acid | VRQAPGQGLEWMGRIEPNAGEPTYNEKFKNRVTMTVD |
| | sequence | TSTSTVYMELSSLRSEDTAVYYCARSTTTVEAMDYWGQ |
| | (Pyroglutamate or | GTSVTVSSASTKGPSVFPLAPSSKSTSGGTAALGCLVKD |
| | Pyroglutamic acid at | YFPEPVTVSWNSGALTSGVHTFPAVLQSSGLYSLSSVVT |
| | N-terminus; no C- | VPSSSLGTQTYICNVNHKPSNTKVDKKVEPKSCDKTHTC |
| | terminal lysine) | PPCPAPEAAGAPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| | | X is pyroglutamate or pyroglutamic |
| | | acid |
| 23 | Ab-hzDPmut heavy | QVQLVQSGAEVVKPGASVKVSCKASGYTFTRYWMHW |
| | chain amino acid | VRQAPGQGLEWMGRIEPNAGEPTYNEKFKNRVTMTVD |
| | sequence | TSTSTVYMELSSLRSEDTAVYYCARSTTTVEAMDYWGQ |
| | (no C-terminal | GTSVTVSSASTKGPSVFPLAPSSKSTSGGTAALGCLVKD |
| | lysine) | YFPEPVTVSWNSGALTSGVHTFPAVLQSSGLYSLSSVVT |
| | | VPSSSLGTQTYICNVNHKPSNTKVDKKVEPKSCDKTHTC |
| | | PPCPAPEAAGAPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| 24 | Ab-hzDPmut heavy | XVQLVQSGAEVVKPGASVKVSCKASGYTFTRYWMHW |
| | chain amino acid | VRQAPGQGLEWMGRIEPNAGEPTYNEKFKNRVTMTVD |
| | sequence | TSTSTVYMELSSLRSEDTAVYYCARSTTTVEAMDYWGQ |
| | (Pyroglutamate or | GTSVTVSSASTKGPSVFPLAPSSKSTSGGTAALGCLVKD |
| | Pyroglutamic acid at | YFPEPVTVSWNSGALTSGVHTFPAVLQSSGLYSLSSVVT |
| | N-terminus) | VPSSSLGTQTYICNVNHKPSNTKVDKKVEPKSCDKTHTC |
| | | PPCPAPEAAGAPSVFLFPPKPKDTLMISRTPEVTCVVVDV |
| | | SHEDPEVKFNWYVDGVEVHNAKTKPREEQYNSTYRVV |
| | | SVLTVLHQDWLNGKEYKCKVSNKALPAPIEKTISKAKG |
| | | QPREPQVYTLPPSRDELTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| | | X is pyroglutamate or pyroglutamic |
| | | acid |
| 25 | Amino acid portion | GGFG |
| | of Linker | |
| 26 | Amino acid portion | GFLG |
| | of Linker | |
| 27 | Amino acid portion | GGVA |
| | of Linker | |
| 28 | Amino acid portion | GGGGG |
| | of Linker | |
| 29 | Amino acid portion | GGGGS |
| | of Linker | |
| 30 | Amino acid portion | GGGGSGGGGSGGGGSGGGGS |
| | of Linker | |
| 31 | Artificial sequence | CTGACT |
| 32 | Artificial sequence | CAGGTT |

Abbreviations herein have the following meanings:

| Abbreviation | Meaning |
|---|---|
| CDR | Complementarity determining region in immunoglobulin variable region |
| FR | Antibody framework region: amino acid residues except for CDR |
| | residues in antibody variable region |
| VH | Antibody heavy chain variable region |
| VL | Antibody light chain variable region |
| IgG | Immunoglobulin G |
| IMGT | The numbering system based on the international ImMunoGeneTics |
| | information system ® (IMGT) initiated by Lefranc et al. can be found in |
| | Lefranc et al., Dev. Comparat. Immunol. 27: 55-77, 2003. |
| Kabat | Immunoglobulin comparison and numbering system presented by Elvin |
| | A. Kabat (see, e.g., Kabat et al., Sequences of Proteins of Immunological |
| | Interest, 5th Ed. Public Health Service, National Institutes of Health, |
| | Bethesda, Md., 1991). |
| Chothia | Immunoglobulin numbering system presented by Chothia et al. is a classic |
| | rule for identifying CDR region boundaries based on a position of a |
| | structural loop region (see, e.g., Chothia & Lesk (1987) J. Mol. Biol. |
| | 196: 901-917; Chothia et al. (1989) Nature 342: 878-883). |
| AbM | The definition of CDR comes from Martin′s related research (Martin |
| | A C R, Cheetham J C, Rees A R (1989) Modelling antibody hypervariable |
| | loops: A combined algorithm. Proc Natl Acad Sci USA 86: 9268-9272). |
| mAb | Monoclonal antibody |
| EC₅₀ | Concentration that produces 50% efficacy or binding |
| IC₅₀ | Concentration that produces 50% inhibition |
| ELISA | Enzyme-linked immunosorbent assay |
| PCR | Polymerase chain reaction |
| HRP | Horseradish peroxidase |
| K_{D} | Equilibrium dissociation constant |
| Ka | Binding rate constant |
| Kd | Dissociation rate constant |
| ADCC | Antibody-dependent cell-mediated cytotoxicity |
| CDC | Complement-dependent cytotoxicity |
| FACS | Flow cytometer technology |
| PTM | Post-translational modification |
| CDR-H1 | Complementarity determining region 1 in immunoglobulin heavy chain |
| | variable region |
| CDR-H2 | Complementarity determining region 2 in immunoglobulin heavy chain |
| | variable region |
| CDR-H3 | Complementarity determining region 3 in immunoglobulin heavy chain |
| | variable region |
| CDR-L1 | Complementarity determining region 1 in immunoglobulin light chain |
| | variable region |
| CDR-L2 | Complementarity determining region 2 in immunoglobulin light chain |
| | variable region |
| CDR-L3 | Complementarity determining region 3 in immunoglobulin light chain |
| | variable region |

| Abbreviation | Meaning | Abbreviation | Meaning |
|---|---|---|---|
| HATU | N,N,N′,N′-tetramethyl-O- | HOBt | 1-hydroxybenzotriazole |
| | (7-azabenzotriazol-1- | | |
| | yl)uronium | | |
| | hexafluorophosphate | | |
| DIPEA | Diisopropylethylamine | EDCI | 1-(3-dimethylaminopropyl)- |
| | | | 3-ethylcarbodiimide |
| | | | hydrochloride |
| TFA | Trifluoroacetate | DMF | N,N-dimethylformamide |
| Pd/C | Palladium on Carbon | DMTMM | 4-(4,6-dimethoxytriazin-2- |
| | | | yl)-4-methylmorpholine |
| | | | hydrochloride |
| NBS | N-bromosuccinimide | IPA | Isopropanol |
| NaIO₄ | Sodium periodate | RuCl₃•H₂O | Ruthenium trichloride |
| | | | hydrate |
| XPhos Pd G3 | Methanesulfonato(2- | LiOH•H₂O | Lithium hydroxide hydrate |
| | dicyclohexylphosphino- | | |
| | 2′, 4′, 6′-tri-isopropyl-1,l′- | | |
| | biphenyl)(2′-amino-1,1′- | | |
| | biphenyl-2- | | |
| | yl)palladium(II) | | |
| K₃PO₄ | Potassium phosphate | m-CPBA | Metachloroperoxybenzoic |
| | | | acid |
| THF | Tetrahydrofuran | DMSO | Dimethyl sulfoxide |
| DCM | Dichloromethane | MeOH | Methanol |
| HPLC | High performance liquid | LC-MS | Liquid chromatography- |
| | chromatography | | mass chromatography |
| TCEP | (Tris(2- | Na₂HPO₄ | Disodium hydrogen |
| | carboxyethyl)phosphine | | phosphate |
| EDTA | Ethylenediaminetetra- | PB | Phosphate buffer |
| | acetic acid | | |
| DAR | Drug load ratio | PyroE | Pyroglutamate |
| SPDB | N-Succinimidyl-4-(2- | DM4 | N2′-Deacetyl-N2′-(4- |
| | pyridyldithio)butanoate | | mercapto-4-methyl-1- |
| | | | oxopentyl)-maytansine |
| DMA | N,N-Dimethylacetamide | SNPB | 2,5-Dioxo-1-pyrrolidinyl 4- |
| | | | [(5-nitro-2- |
| | | | pyridinyl)dithio]butanoate |
| PBS | Phosphate buffered saline | PPTS | Pyridinium p- |
| | | | Toluenesulfonate |
| PBST | Phosphate Buffered | BSA | Bovine Serum Albumin |
| | Saline with Tween 20 | | |

The structures of the compounds described in the following Examples were determined by nuclear magnetic resonance (¹H NMR) or mass spectrometry (MS).

The determination of nuclear magnetic resonance (¹H NMR) was carried out through a Bruker 400 MHz nuclear magnetic resonance instrument; a deuterated reagent was hexadeuteriodimethylsulfoxide (DMSO-d6); and an internal standard was tetramethylsilane (TMS).

Abbreviations in nuclear magnetic resonance (NMR) spectra used in Examples are shown below.
s: singlet, d: doublet, t: triplet, q: quartet, m: multiplet, br: broad, J: coupling constant, Hz: Hertz, and DMSO-d6: deuterated dimethylsulfoxide. 8 value was expressed in ppm.

The determination of mass spectrum (MS) was carried out using Agilent (ESI) mass spectrometer, with model of Agilent 6120B.

### I: Preparation of Drug Linkers in Pre- Conjugated Form

Ia: Preparation of N-((S)-10-benzyl-1-(((1S,9S)-5-chloro-9-ethyl-9-hydroxy-4-methyl-10,13-dioxo-2,3,9, 10,13, 15-hexahydro-1H, 12H-benzo[de]pyrano[3′,4′: 6, 7] indolizino[1,2-b]quinolin-1-yl)amino)-1,6,9,12, 15-pentaoxo-3-oxy-5,8, 11, 14-tetraazahexadecan-16-yl)-6-(2-(methylsulfonyl)pyrimidin-5-yl)hex-5-ynamide (A-1)

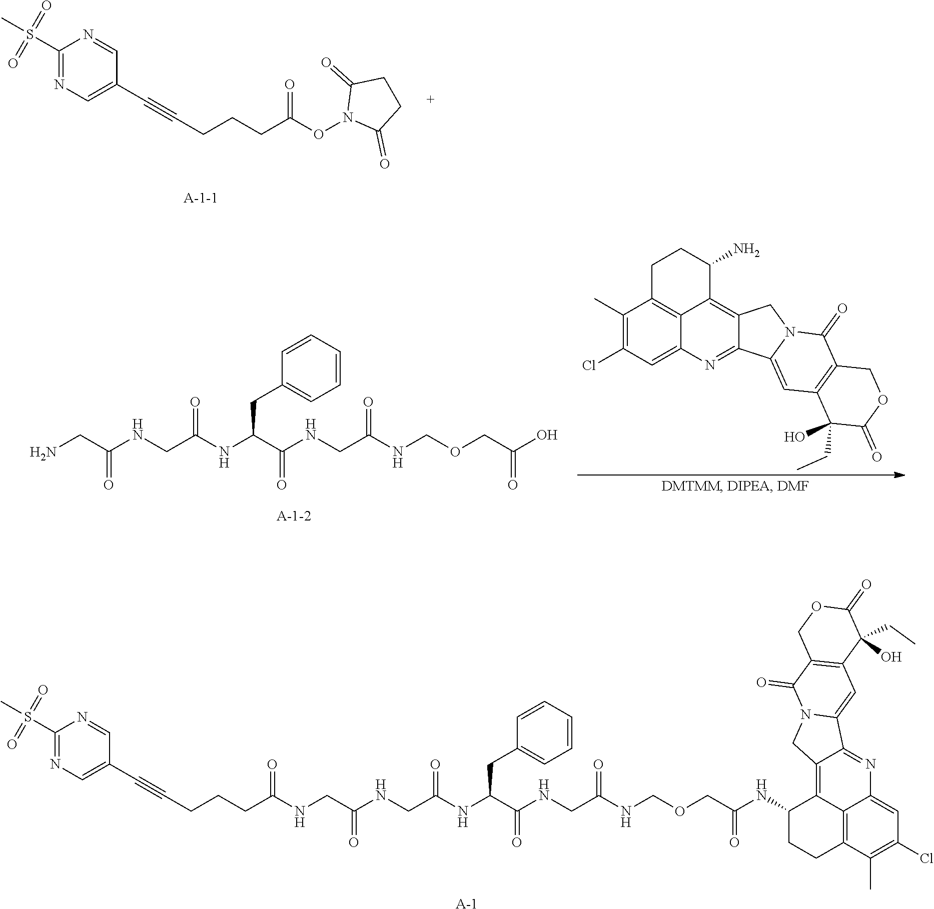 [SMILES: CS(=O)(=O)c1ncc(C#CCCCC(=O)ON2C(=O)CCC2=O)cn1 ; NCC(=O)NCC(=O)N[C@@H](Cc1ccccc1)C(=O)NCC(=O)NCOCC(=O)O ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(Cl)c(C)c3c1c2[C@@H](NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; "A-1"]

Under nitrogen protection, 2,5-dioxopyrrolidin-1-yl-6-(2-(methylsulfonyl)pyrimidin-5-yl)hex-5-ynoate (0.66 g, 1.80 mmol) and (R)-16-amino-10-benzyl-6,9,12, 15-tetraoxo-3-oxa-5,8,11, 14-tetraazahexadecanoic acid (0.75 g, 1.77 mmol) were added into DMF (19 mL). After the mixture was heated to a temperature of 35° C. for reaction for 16 h, (1S,9S)-1-amino-5-chloro-9-ethyl-9-hydroxy-4-methyl-1,2,3,9,12,15-hexahydro-10H, 13H-benzo[de]pyrano[3′,4′: 6, 7] indolizino[1,2-b]quinoline-10,13-dione (1.00 g, 1.77 mmol) was added into the system, the resulting mixture was cooled with ice water to 5-15° C., DMTMM (0.98 g, 3.53 mmol) was added, and then DIPEA (1.14 g, 8.84 mmol) was added dropwise. The resulting mixture was kept at 25° C. for reaction for 16 h. The reaction mixture was poured into a liquid mixture of DCM (600 mL), IPA (60 mL), and water (100 mL). The resulting mixture was stirred for 10 min to separate the DCM phase, which was washed with brine (100 mL). The organic phase was concentrated to obtain a crude product, which was purified by preparative high performance liquid chromatography and then lyophilized to provide 0.98 g of the title compound.

The separation and purification method is as follows: Chromatographic column: Waters SunFire Prep C18 OBD (5 μm*19 mm*150 mm)
Mobile phase A: acetonitrile; mobile phase B: water (0.05% formic acid)

| | Mobile | Mobile | |
|---|---|---|---|
| Time [min] | phase A [%] | phase B [%] | Flow rate [mL/min] |
| 0.00 | 30 | 70 | 24 |
| 2.00 | 30 | 70 | 24 |
| 18.00 | 80 | 20 | 24 |

The structural characterization data are as follows:
MS m/z (ESI): 1107.3 [M+H]⁺.

¹H NMR (400 MHZ, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J-7.2 Hz, 3H).

### 3] Ib: Preparation of N-((7S, 10S, 13S)-1-(((1S,9S)-5-chloro-9-ethyl-9-hydroxy-4-methyl-10,13-dioxo-2,3,9,10,13, 15-hexahydro-1H, 12H-benzo[de]pyrano[3′,4′: 6, 7] indolizino[1,2-b]quinolin-1-yl)amino)-7,10-dimethyl-1,6,9,12-tetraoxo-3-oxa-5,8,11-triazatetradecan-13-yl)-6-(2-(methylsulfonyl)pyrimidin-5-yl)hex-5-ynamide (A-2)

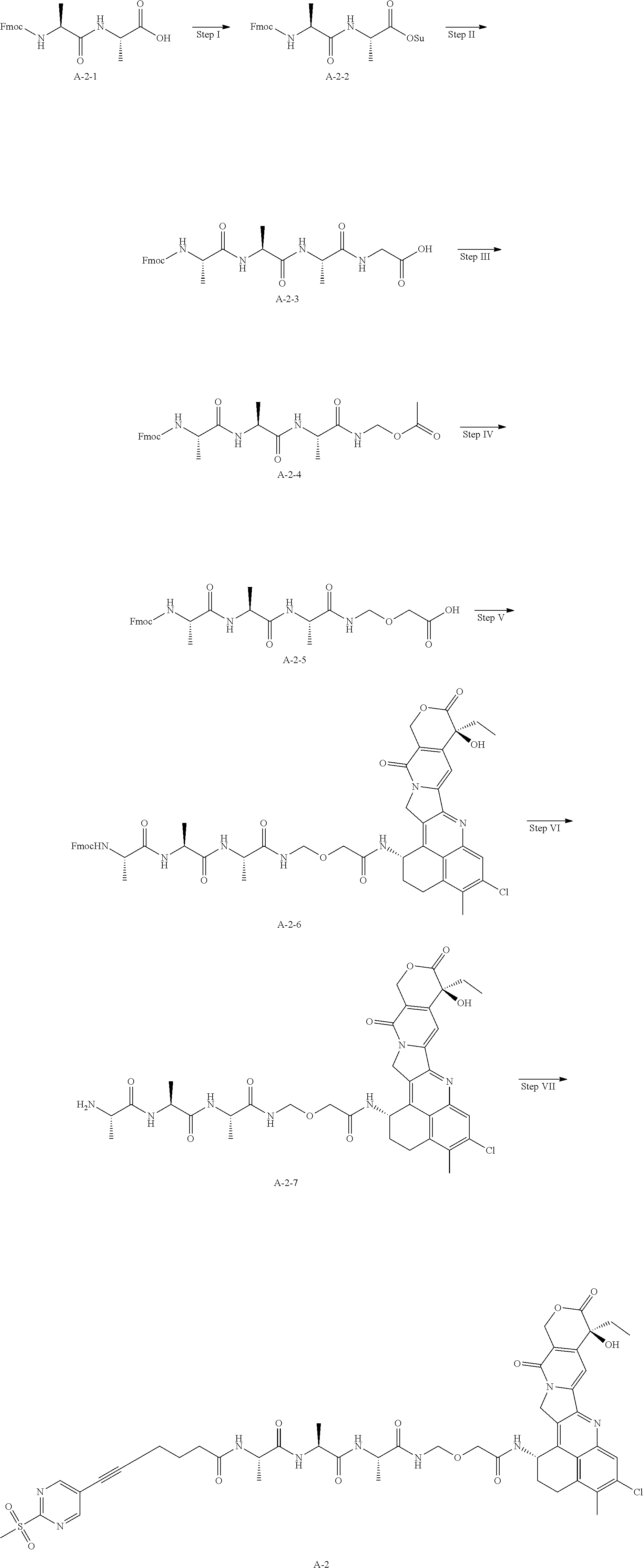

### Step 1: Preparation of 2,5-dioxopyrrolidin-1-yl(((9H-fluoren-9-yl) methoxy) carbonyl)-L-alanyl-L-alanine (A-2-2)

(((9H-fluoren-9-yl) methoxy) carbonyl)-L-alanyl-L-alanine (10.0 g, 26.2 mmol) was dissolved in dichloromethane (100 mL), DCC (8.09 g, 39.2 mmol, 7.93 mL) and 1-hydroxypyrrolidine-2,5-dione (4.51 g, 39.2 mmol) were added, and then the mixture was stirred at 25° C. for 12 h. The reaction mixture was directly filtered (to remove DCU), and the filter cake was rinsed with dichloromethane 3 times (10.0 mL×3). The filtrate was concentrated, added into tetrahydrofuran (100 mL), cooled to 0° C., and then stirred for an additional 1 h. The mixture was re-filtered (to remove DCU), and the filtrate was concentrated to provide the crude title compound (11.7 g), which was directly used in the next step without purification.

The structural characterization data are as follows: ESI-MS (m/z): 502.1 (M+Na).

### Step II: Preparation of (((9H-fluoren-9-yl) methoxy) carbonyl)-L-alanyl-L-alanyl-L-alanylglycine (A-2-3)

2,5-dioxopyrrolidin-1-yl((9H-fluoren-9-yl) methoxy) carbonyl)-L-alanyl-L-alanine (5.00 g, 10.4 mmol) and L-alanylglycine (1.52 g, 10.4 mmol) were added into a mixed solvent of water (25.0 mL) and tetrahydrofuran (50.0 mL). After an aqueous solution of sodium bicarbonate (1.75 g, 20.8 mmol, 811 μL) was added, the mixture was stirred at 25° C. for 2 h. The mixture was stirred at 25° C. for 2 h. The reaction mixture was adjusted with IN dilute hydrochloric acid to pH=2-3, and extracted with dichloromethane 3 times (60.0 mL×3). The organic phases were combined, dried over anhydrous sodium sulfate, filtered, and concentrated, to provide the crude title compound (3.18 g, 6.23 mmol), which was directly used in the next step without purification.

The structural characterization data are as follows:
ESI-MS (m/z): 511.1 (M+H)⁺.

### Step III: Preparation of (5S,8S, 11S)-1-(9H-fluoren-9-yl)-5,8-11-trimethyl-3,6,9,12-tetraoxo-2-oxa-4,7, 10, 13-tetraazatetradecan-14-yl acetate (A-2-4)

(((9H-fluoren-9-yl) methoxy) carbonyl)-L-alanyl-L-alanyl-L-alanylglycine (3.10 g, 6.07 mmol), copper acetate (882 mg, 4.86 mmol), and lead tetraacetate (6.46 g, 14.5 mmol) were dissolved in DMF (40.0 mL). After glacial acetic acid (1.60 g, 26.7 mmol, 1.53 mL) was added, the mixture was heated to a temperature of 60° C. and stirred for 1 h. The reaction mixture was directly concentrated to obtain a crude product. The crude product was purified through a silica gel column (dichloromethane/methanol=100/1-10/1), and re- concentrated to provide the crude title compound (709 mg, 1.28 mmol), which was directly used in the next step without purification.

The structural characterization data are as follows:

¹H NMR (400 MHZ, DMSO) δ 8.85 (m, 1H), 7.97 (m, 2H), 7.89 (d, J=7.6 Hz, 2H), 7.72 (m, 2H), 7.53 (m, 1H), 7.39-7.44 (m, 2H), 7.30-7.35 (m, 2H), 5.04-5.10 (m, 2H), 4.24-4.28 (m, 3H), 3.17 (d, J=5.2 Hz, 3H), 1.98 (s, 3H), 1.18-1.22 (m, 9H)

### Step IV: Preparation of (5S,8S, 11S)-1-(9H-fluoren-9-yl)-5,8,11-trimethyl-3,6,9,12-tetraoxo-2, 15-dioxa-4,7, 10, 13-tetraazaheptadecan-17-oic acid (A-2-5)

(5S,8S, 11S)-1-(9H-fluoren-9-yl)-5,8-11-trimethyl-3,6,9,12-tetraoxo-2-oxa-4,7,10,13-tetraazatetradecan-14-yl acetate (300 mg, 571.90 μmol) and 2-hydroxyacetic acid (52.2 mg, 686 umol, 41.7 μL) were dissolved in dichloromethane (3.00 mL). After PPTS (144 mg, 572 μmol) was added, the mixture was stirred at 25° C. for 2 h. Water (100 mL) was added into the reaction mixture, which was adjusted with IN dilute hydrochloric acid to a pH of 4-5, and then extracted with dichloromethane 3 times (80.0 mL×3). The organic phases were combined, dried over anhydrous sodium sulfate, filtered, and concentrated to obtain a crude product. which was purified by preparative high performance liquid chromatography and then lyophilized to provide the title compound (176 mg, 325.10 μmol).

The structural characterization data are as follows:

ESI-MS (m/z): 558.2 [M+H]⁺.

The purification method is as follows:
Chromatographic column: Waters xbridge C18 (150 mm*25 mm*10 μm)
Mobile phase A: acetonitrile; mobile phase B: water (0.05% methylammonium bicarbonate)

| | Mobile | Mobile | |
|---|---|---|---|
| Time [min] | phase A [%] | phase B [%] | Flow rate [mL/min] |
| 0.00 | 17 | 83 | 25 |
| 10.00 | 47 | 53 | 25 |

### Step V: Preparation of (9H-fluoren-9-yl)methyl((7S, 10S, 13S)-1-(((1S,9S)-5-chloro-9-ethyl-9-hydroxy-4-methyl-10,13-dioxo-2,3,9,10,13,15-hexahydro-1H, 12H-benzo[de]pyrano[3′,4′: 6, 7] indolizino[1,2-b]quinolin-1-yl)amino)-7, 10-dimethyl-1,6,9, 12-tetraoxo-3-oxa-5,8, 11-triazatetradecan-13-yl) carbamate (A-2-6)

The compound (5S, 8S, 11S)-1-(9H-fluoren-9-yl)-5,8,11-trimethyl-3,6,9,12-tetraoxo-2,15-dioxa-4,7, 10, 13-tetraazaheptadecan-17-oic acid (657 mg, 1.22 mmol) and (1S,9S)-1-amino-5-chloro-9-ethyl-9-hydroxy-4-methyl-1,2,3,9,12, 15-hexahydro-10H, 13H-benzo[de]pyrano[3′,4′: 6, 7] indolizino[1,2-b]quinoline-10,13-dione (500 mg, 1.11 mmol) were dissolved in N,N-dimethylformamide (10 mL), and then HATU (630.67 mg, 1.66 mmol) and N,N-diisopropylethylamine (428 mg, 3.32 mmol) were added. The mixture was stirred at room temperature for 1 h. After the reaction was complete, the reaction mixture was directly purified by high performance liquid chromatography and then lyophilized to provide 700 mg of the title compound.

The separation and purification method is as follows:
Chromatographic column: Waters SunFire Prep C18 OBD (5 μm*19 mm*150 mm)
Mobile phase A: acetonitrile; mobile phase B: water (0.05% formic acid)

| | Mobile | Mobile | |
|---|---|---|---|
| Time [min] | phase A [%] | phase B [%] | Flow rate [mL/min] |
| 0.00 | 30 | 70 | 28 |
| 2.00 | 30 | 70 | 28 |
| 18.00 | 90 | 10 | 28 |

### Step VI: Preparation of(S)-2-amino-N-((S)-1-(((S)-1-(((2-(((1S,9S)-5-chloro-9-ethyl-9-hydroxy-4-methyl-10,13-dioxo-2,3,9,10, 13,15-hexahydro-1H, 12H-benzo[de]pyrano[3′,4′: 6, 7] indolizino[1,2-b]quinolin-1-yl)amino)-2-oxoethoxy)methyl)amino)-1-oxoprop-2-yl)amino)-1-oxoprop-2-yl)propionamide (A-2-7)

(9H-fluoren-9-yl)methyl((7S, 10S, 13S)-1-(((1S,9S)-5-chloro-9-ethyl-9-hydroxy-4-methyl-10,13-dioxo-2,3,9, 10,13, 15-hexahydro-1H, 12H-benzo[de]pyrano[3′,4′: 6, 7] indolizino[1,2-b]quinolin-1-yl)amino)-7,10-dimethyl-1,6,9,12-tetraoxo-3-oxa-5,8,11-triazatetradecan-13-yl) carbamate (500 mg, 0.513 mmol) was dissolved in N,N-dimethylformamide (2 mL), diethylamine (75.05 mg, 1.03 mmol) was added, and the mixture was kept at room temperature for reaction for 1 h. After the reaction was complete, the reaction mixture was directly purified by high performance liquid chromatography and then lyophilized to provide 307 mg of the title compound.

The separation and purification method is as follows:
Chromatographic column: Waters SunFire Prep C18 OBD (5 μm*19 mm*150 mm)
Mobile phase A: acetonitrile; mobile phase B: water (0.05% formic acid)

| | Mobile | Mobile | |
|---|---|---|---|
| Time [min] | phase A [%] | phase B [%] | Flow rate [mL/min] |
| 0.00 | 20 | 80 | 24 |
| 2.00 | 20 | 80 | 24 |
| 18.00 | 80 | 20 | 24 |

### Step VII: Preparation of N-((7S, 10S, 13S)-1-(((1S,9S)-5-chloro-9-ethyl-9-hydroxy-4-methyl-10,13-dioxo-2,3,9,10, 13, 15-hexahydro-1H, 12H-benzo[de]pyrano[3′,4′: 6, 7] indolizino[1,2-b]quinolin-1-yl)amino)-7,10-dimethyl-1,6,9, 12-tetraoxo-3-oxa-5,8,11-triazatetradecan-13-yl)-6-(2-(methylsulfonyl)pyrimidin-5-yl)hex-5-ynamide (A-2)

(S)-2-amino-N-((S)-1-(((S)-1-(((2-(((1S,9S)-5-chloro-9-ethyl-9-hydroxy-4-methyl-10,13-dioxo-2,3,9,10,13,15-hexahydro-1H, 12H-benzo[de]pyrano[3′,4′: 6, 7] indolizino[1,2-b]quinolin-1-yl)amino)-2-oxoethoxy)methyl)amino)-1-oxoprop-2-yl)amino)-1-oxoprop-2-yl)propionamide (170 mg, 0.226 mmol) and 2,5-dioxopyrrolidin-1-yl-6-(2-(methylsulfonyl)pyrimidin-5-yl)hex-5-ynoate (90.83 mg, 0.249 mmol) were dissolved in N,N-dimethylformamide (10 mL), and N,N-diisopropylethylamine (29.21 mg, 0.226 mmol) was added. The reaction mixture was stirred at room temperature for 16 h. The reaction mixture was directly purified by high performance liquid chromatography and then lyophilized to provide 50.56 mg of the title compound.

The structural characterization data are as follows:

MS m/z (ESI): 1002.4 [M+H]⁺

Its separation and purification method is as follows:
Chromatographic column: Waters SunFire Prep C18 OBD (5 μm*19 mm*150 mm)
Mobile phase A: acetonitrile; mobile phase B: water (0.05% formic acid)

| | Mobile | Mobile | |
|---|---|---|---|
| Time [min] | phase A [%] | phase B [%] | Flow rate [mL/min] |
| 0.00 | 30 | 70 | 28 |
| 2.00 | 30 | 70 | 28 |
| 18.00 | 90 | 10 | 28 |

¹H NMR (400 MHZ, DMSO) δ 9.11 (s, 2H), 8.68 (t, J=6.4 Hz, 1H), 8.49 (d, J=8.8 Hz, 1H), 8.16 (s, 1H), 8.10 (d, J=7.2 Hz, 1H), 8.01 (d, J=7.2 Hz, 1H), 7.91 (d, J=6.8 Hz, 1H), 7.31 (s, 1H), 6.55 (s, 1H), 5.65-5.55 (m, 1H), 5.43 (s, 2H), 5.21 (s, 2H), 4.67-4.55 (m, 2H), 4.29-4.15 (m, 3H), 3.98 (s, 2H), 3.41 (s, 3H), 3.25-3.15 (m, 2H), 2.57-2.56 (m, 2H), 2.35-2.27 (m, 2H), 2.22-2.12 (m, 2H), 1.91-1.75 (m, 4H), 1.23-1.09 (m, 9H), 0.87 (t, J=7.2 Hz, 3H).

### II: Preparation of Antibodies

BALB/c mice were immunized with human and monkey CEACAM5-His proteins, and serum titers were monitored through ELISA and flow cytometry. Optimal mice were selected based on the titer results. Their spleen cells were fused, screened, and subcloned to detect binding activities of different monoclones to human\monkey proteins, cells, and antigens of the same family, and obtain an optimal clone Ab-C8. After a mouse sequence was obtained, the antibody sequence was humanized, and the antibody sequence was transformed by removing PTM sites. After a heavy chain constant region was transformed by removing ADCC, fully human antibody Ab-hzDPmut (heavy chain variable region, SEQ ID NO: 1; light chain variable region, SEQ ID NO: 2), an amino acid sequence (SEQ ID NO: 3) modified by removing ADCC activity from a human IgG1 heavy chain constant region, and a human kappa light chain constant region (SEQ ID NO: 4) were finally obtained to form a complete humanized antibody (see Table 1). The antibody was entrusted to Nanjing GenScript Biotechnology Co., Ltd for codon optimization and gene synthesis, and constructed into pTT5 plasmid. Heavy chain and light chain plasmids were simultaneously transfected into CHO-S cells, and the antibody expressed in the supernatant was purified with protein A, thus obtaining the corresponding antibody protein Ab-hzDPmut. The heavy chain amino acid sequence and the light chain amino acid sequence of the Ab-hzDPmut are as shown in SEQ ID NO: 20 and SEQ ID NO: 21 respectively.

hIgG1 is an anti-chicken lysozyme antibody, of which the heavy chain variable region was fused to a mutant human IgG1 heavy chain constant region (SEQ ID NO: 3), and the light chain variable region was fused to a wild-type human kappa light chain constant region (SEQ ID NO. NO: 4). The antibody hIgG1 was obtained by expression and purification using the above method.

A control antibody tusamitamab of CEACAM5 came from international patent publication no. WO 2022/101165. After codon optimization, nucleotide sequences of the antibody heavy chain variable region were synthesized and cloned into a pTT5 vector respectively, and expressed and purified using the above method, to obtain the antibody tusamitamab.

The sequence of another CEACAM5 control antibody so8G4 (also referred to herein as mAb1) was obtained from WO 2022048883 (Anderl Jan. Mar. 10, 2022), and the control antibody mAb1 was prepared by a similar method.

### III. Conjugation of a Compound Comprising a Cell Bioactive Molecule and a Linker to an Antibody

The antibodies Ab-hzDPmut, tusamitamab, and hIgG1 involved in the antibody-drug conjugates prepared in the following examples are the corresponding antibodies in the above part II.

### 1. Preparation of ADC-1 (DAR 8)

1.579 mL of antibody Ab-hzDPmut (16.94 mg/mL) was diluted with 79 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 113 μL, pH 7.60) was added. The resulting mixture was fully mixed, and left to stand at room temperature for 1.5 h. Then, a solution of A-1 (211.83 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-1 (i.e., Ab-hzDPmut-A-1). The DAR value determined by mass spectrometry was 8.0.

### 2. Preparation of ADC-2 (DAR 8)

1.579 mL of antibody Ab-hzDPmut (16.94 mg/mL) was diluted with 79 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 113 μL, pH 7.60) was added. The resulting mixture was fully mixed, and left to stand at room temperature for 1.5 h. Then, a solution of A-2 (209.67 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-2 (i.e., Ab-hzDPmut-A-2 (DAR8)). The DAR value determined by mass spectrometry was 8.0.

### 3. Preparation of ADC-3 (DAR 4)

0.87 mL of antibody Ab-hzDPmut (27.5 mg/mL) was diluted with 43.6 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 39.5 μL, pH 7.60) was added. The resulting mixture was fully mixed, and left to stand at room temperature for 1.5 h. Then, a solution of A-2 (86.63 μL, 10 mM) dissolved in dimethyl sulfoxide (5 times as much as the amount of the antibody) was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-3 (i.e., Ab-hzDPmut-A-2 (DAR4)). The DAR value determined by mass spectrometry was 4.04.

### 4. Preparation of ADC-4 (DAR 8)

0.8 mL of antibody tusamitamab (15 mg/mL) was diluted with 40 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 45.6 μL, pH 7.60) was added. The resulting mixture was fully mixed, and left to stand at room temperature for 1.5 h. Then, a solution of A-1 (86.4 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-4 (i.e., tusamitamab-A-1). The DAR value determined by mass spectrometry was 7.99.

### 5. Preparation of ADC-5 (DAR 8)

0.338 mL of antibody tusamitamab (29.6 mg/mL) was diluted with 17 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 37.9 μL, pH 7.60) was added. The resulting mixture was fully mixed, and left to stand at room temperature for 1.5 h. Then, a solution of A-2 (70.4 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-5 (i.e., tusamitamab-A-2). The DAR value determined by mass spectrometry was 7.86.

### 6. Preparation of ADC-6 (tusamitamab-SPDB-DM4) (DAR 4)

22.73 mL of antibody tusamitamab (11 mg/mL) was adjusted with IM Na₂HPO₄ solution to a pH of 7.4. Then, DMA (227 μL) and SNPB (726.5 μL, 15 mM) were sequentially added, and the mixture was left to stand at room temperature for 3 h. Then, PBS (22 mL), DMA (227 μL) and DM4 (1.056 mL, 15 mM) solutions were sequentially added into the reaction mixture. The mixture was fully mixed, and left to stand at room temperature for 18 h. After completion, the buffer solution was replaced with a PBS buffer solution at a pH of 7.4 through a NAP™ gel column (Cytiva), to provide the antibody-drug conjugate ADC-6 (i.e., tusamitamab-SPDB-DM4). The DAR value determined by mass spectrometry was 3.75.

### 7. Preparation of ADC-7 (DAR 8)

3.26 mL of antibody hIgG1 (18.41 mg/mL) was diluted with 163 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 228.96 μL, pH 7.60) was added. The resulting mixture was fully mixed, and left to stand at room temperature for 1.5 h. Then, a solution of A-1 (429.16 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-7 (i.e., hIgG1-A-1). The DAR value determined by mass spectrometry was 7.98.

### 8. Preparation of ADC-8 (DAR 8)

3.26 mL of antibody hIgG1 (18.41 mg/mL) was diluted with 163 μL of 20 mM PB+0.1M EDTA (pH 7.60), then the mixture was adjusted with 1M Na₂HPO₄ solution to a pH of 7.60, and a solution of 10 mM TCEP (tris(2-carboxyethyl) phosphine, 228.96 μL, pH 7.60) was added. The resulting mixture was fully mixed, and left to stand at room temperature for 1.5 h. Then, a solution of A-2 (424.79 μL, 10 mM, 10 times as much as the amount of the antibody) dissolved in dimethyl sulfoxide was added. The mixture was fully mixed, and left to stand at room temperature for 2 h. After completion, the buffer solution was replaced with 20 mM histidine buffer solution at a pH of 6.0 through a NAP™ gel column (Cytiva), to provide the antibody-drug conjugate ADC-8 (i.e., hIgG1-A-2). The DAR value determined by mass spectrometry was 8.02.

### IV. Detection of Antibody-Drug Conjugate Activity

### 1. Detection of Kinetic Affinity of an Anti-Human CEACAM5 Antibody and a Conjugate Thereof

A ForteBio (Pall life sciences) instrument was used to detect the kinetic affinity of the anti-human CEACAM5 antibody and the conjugate thereof to human CEACAM5-ECD-his, monkey CEACAM5-ECD-his, mouse CEACAM5-ECD His, and Native CEACAM5 proteins. The Native CEACAM5 is CEACAM5 proteins shed from the surface of tumor cells of a tumor patient. These proteins can be shed into tumor tissues or enter plasma or other tissues. The specific method is as follows: the to-be-tested antibody and ADC were diluted with PBST (0.02% Tween-20) to 5 μg/ml, and each antigen protein was diluted gradiently to 200 nM, 100 nM, 50 nM, 25 nM, 12.50 nM, 6.25 nM, 3.125 nM, and 0 nM. Then, the to-be-tested antibody was captured with a Protein A Sensor (Pall life sciences) in a PBST (0.02% Tween-20) solution for 60 s respectively, then bound to 4 proteins for 60 s respectively, and was then dissociated for 180 s. The test result was opened in Data Analysis 11.0 software, and analyzed by global fitting in 1:1 mode, to obtain an affinity constant. The result is as shown in Tables 1-1 and 1-2: the antibody Ab-hzDPmut and a conjugate thereof bind to human and monkey CEACAM5-ECD-His, but do not bind to mouse CEACAM5-ECD-his. The Ab-hzDPmut and the conjugate thereof do not bind to the native CEACAM5, while the tusamitamab, a conjugate thereof and mAb1 bind to the native CEACAM5 proteins. The native CEACAM5 proteins were at a high concentration in plasma of the tumor patient. If the antibody or conjugate can bind to the native CEACAM5 in the plasma, the binding of the antibody to target tumor cells may be affected. Therefore, compared with the tusamitamab and the conjugate thereof, the anti-human CEACAM5 antibody and the conjugate thereof in the present application do not bind to free native CEACAM5 present in the plasma, to avoid affecting the binding of the antibody or the conjugate to the target tumor cells.

TABLE 1-1
Detection results of kinetic affinity of anti-human
CEACAM5 antibody and conjugate thereof to human,
monkey, and mouse CEACAM5-ECD-his proteins

| | Human | Monkey | Mouse |
|---|---|---|---|
| | CEACAM5- | CEACAM5- | CEACAM5- |
| | ECD-His | ECD-His | ECD-His |
| Abs | KD (M) | KD (M) | KD (M) |
| Ab-hzDPmut | 2.62E−08 | 5.24E−10 | No binding |
| ADC-1 | 5.93E−08 | 3.05E−10 | No binding |
| ADC-2 | 2.86E−08 | 1.27E−09 | No binding |

TABLE 1-2
Detection results of kinetic affinity of anti-
human CEACAM5 antibody and conjugate thereof
to Native CEACAM5 dynamic affinity assay results

| | Native CEACAM5 |
|---|---|
| Abs | KD (M) |
| Ab-hzDPmut | No binding |
| ADC-1 | No binding |
| ADC-2 | No binding |
| mAb1 | 2.77E−08 |
| Tusamitamab | <1.0E−12 |
| ADC-6 | 7.53E−09 |

### 2. Cellular Affinity Detection of Anti-Human CEACAM5 Antibody and Conjugate Thereof

Evaluation of the cellular affinity activity of the anti-human CEACAM5 antibody and the conjugate thereof: the evaluation was implemented by overexpression of FACS cells using human gastric cancer cells MKN45 (Nanjing Cobier), human colorectal adenocarcinoma cells LS174T (Nanjing Cobier), and CHOS-machin CEACAM5. The cells were diluted with PBS 2% BSA to a density of 2×10⁶ cells/ml, and 50 μl of the mixture was spread on a 96-well PCR tip plate (100,000 cells/well). A chimeric antibody was transformed at gradiently diluted fragmentation sites, and fully mixed with the cells in the well plate. The mixture was incubated at 4° C. for 1 h, and washed with PBS+2% BSA 3 times. 50 μl/well of PE Goat Anti-Human IgG second antibody (Biolegend) was added at 4° C., and the cells were resuspended. The cells were incubated at 4° C. for 30 min, washed with PBS+2% BSA 3 times, and centrifuged. 200 μl/well of the PBS+2% BSA was added to resuspend the cells. The binding affinity of the antibody to cells was evaluated by FACS (Beckman, Cytoflex).

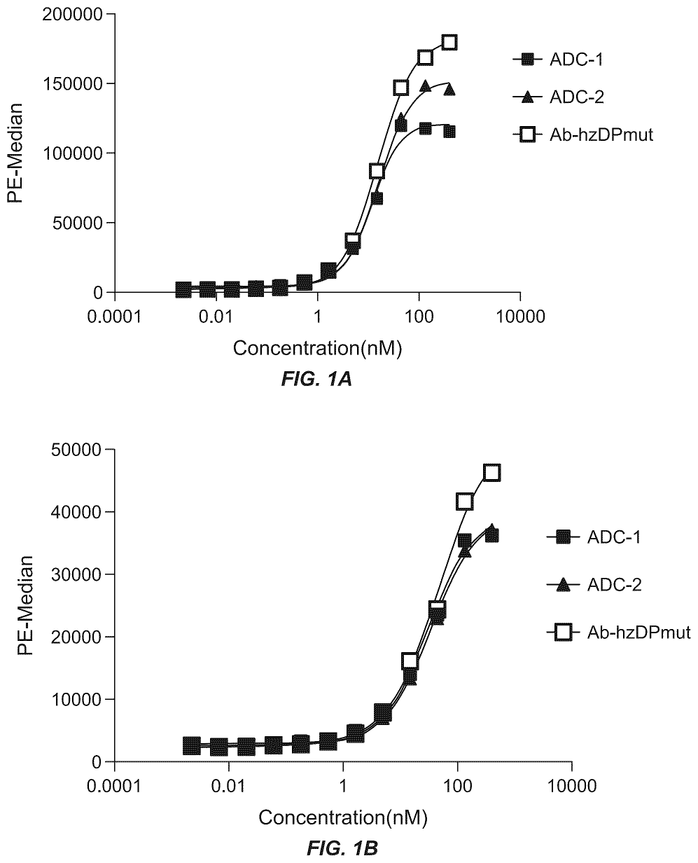

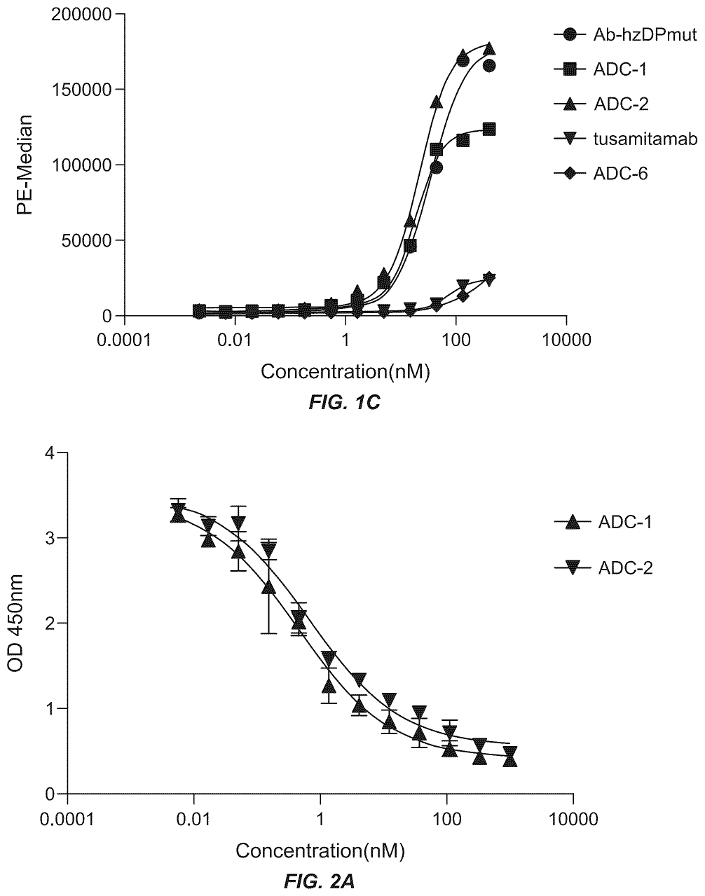

Data processing: The Median PE value was outputted, and then inputted into data analysis software, to compute EC₅₀ by nonlinear fitting. The result is as shown in Tables 2-1 and 2-2, and FIGS. 1A, 1B, and 1C. The above result shows that the antibody Ab-hzDPmut and the conjugate thereof in the present invention have good cellular affinity. In addition, the antibody Ab-hzDPmut and the conjugate thereof have stronger binding activity to monkey antigens than the tusamitamab and the conjugate thereof.

TABLE 2-1
Cellular affinity of anti-human CEACAM5 antibody and ADC

| | MKN45 | LS174T |
|---|---|---|
| Sample | EC₅₀ (nM) | EC₅₀ (nM) |
| ADC-1 | 11.44 | 31 |
| ADC-2 | 16.15 | 35.89 |
| Ab-hzDPmut | 15.71 | 48.13 |

TABLE 2-2
Affinity of anti-human CEACAM5 antibody
and ADC to CHOS-cCEACAM5 cells

| | CHOS-cCEACAM5 |
|---|---|
| Sample | EC₅₀ (nM) |
| ADC-1 | 18.54 |
| ADC-2 | 21.58 |
| ADC-6 | 522.1 |
| Ab-hzDPmut | 35.02 |
| Tusamitamab | 72.61 |

### 3. Detection of Endocytic Activity of Anti-Human CEACAM5 Antibody and Conjugate Thereof

To evaluate the endocytic activity of the anti-CEACAM5 antibody and the conjugate thereof in the human gastric cancer cells MKN45, the cells were adjusted with a complete medium to a density of 1×10⁵ cells/ml. 100 μl of the cell suspension (cell count: 1×104 cells/well) was added to each well of a 96-well plate, and cultured in a thermostatic incubator with CO₂ at 37° C. for 24 h. The culture medium was discarded, and 50 μl of a fresh complete medium was added. The to-be-tested antibody and the negative control antibody hIgG1 were diluted with the complete medium, starting with a concentration of 4.8 μg/ml, by 3-fold gradient dilution to 8 concentration points. A PHrodo reagent (Thermo) was diluted with the complete medium to a concentration of 12 μg/ml; the to-be-tested antibody was mixed with the PHrodo reagent at a ratio of 1:1 (30 μl: 30 μl), and incubated away from light at room temperature for 30 min; 50 μl of the mixture of the to-be-tested antibody and the PHrodo reagent was added into the cells, which were incubated with 5% CO₂ at 37° C. for 24 h; and the cells were detected on a flow cytometer (Thermo, model Attune NxT).

Data processing: The Median YLI value was output, and then inputted into data analysis software, to compute EC50 by nonlinear fitting. The result is as shown in Table 3. The above result shows that the antibody Ab-hzDPmut and the conjugate thereof in the present invention have good endocytic activity.

TABLE 3
Endocytic activity of anti-human CEACAM5 antibody
and ADC in gastric cancer cells MKN45

| Sample | MKN45 EC₅₀ (nM) |
|---|---|
| ADC-1 | 1.517 |
| ADC-2 | 1.238 |
| ADC-4 | 2.098 |
| ADC-5 | 1.635 |
| Ab-hzDPmut | 1.147 |

### 4. In Vitro Cell Killing Assay of Anti-Human CEACAM5 Antibody-Drug Conjugate

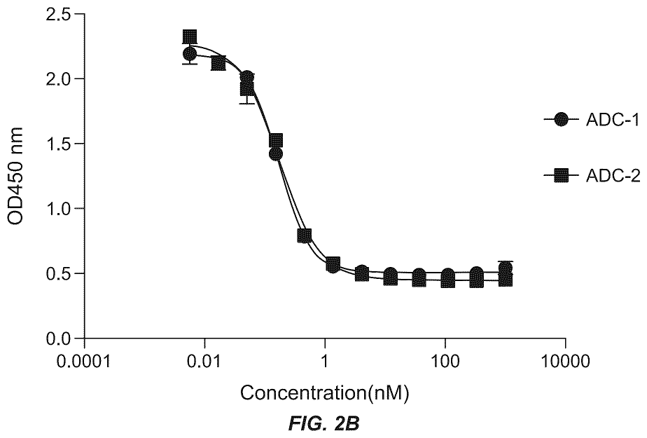

The cells MKN45 and SK-CO-1 after adherent growth were digested with a Trypsin-EDTA (0.25%) (Thermo) solution, counted, and adjusted with the complete medium to a cell density of 1×10⁴ cells/ml. 100 μl of the cell suspension was added into each well of a 96-well plate (the cell count was 1000/well respectively), and the 96-well plate was placed in a thermostatic incubator with 5% CO₂ at 37° C. for incubation for 24 h. The to-be-tested ADC was diluted with the complete medium, starting with a final concentration of 3333.3 nM, by 3-fold gradient dilution total to 12 concentration points. 100 μl of the diluted ADC was added into a 96-well plate, and incubated with 5% CO₂ at 37° C. for 4-7 days. 20 μl of a CCK8 reagent was added into each well of the 96-well plate for incubation at 37° C. for 2-3 h. Signal values at OD450 nm were detected using an ELISA, and then input into data analysis software to compute IC₅₀ by nonlinear curve fitting. The result is as shown in Table 4 and FIGS. 2A and 2B. ADC-1 and ADC-2 have obvious tumor cell killing activity.

TABLE 4
Tumor cell killing activity of ADC drug

| Killing activity | ADC-1 | ADC-2 |
|---|---|---|
| MKN45 EC₅₀ (nM) | 0.4565 | 0.6592 |
| SK-CO-1 EC₅₀ (nM) | 0.1729 | 0.1766 |

### 5. In Vivo Efficacy Testing of Different Antibody-Drug Conjugates in a MKN45 Model

The human gastric cancer cell strain MKN45 used in this example is a CEACAM5-positive cell strain, which is used to construct a subcutaneous transplant tumor model of a human gastric cancer. The cells MKN45 were cultured in 1640 culture medium with 10% fetal calf serum with 5% CO₂ at 37° C. The cells MKN45 in an exponential growth phase were collected, resuspended in PBS to a suitable concentration, and inoculated subcutaneously into female NOD-SCID mice to construct a human gastric cancer xenograft tumor model. When the tumors grew to an average volume of approximately 150 mm³, the animals were randomly divided into 0.9% NaCl injection group, ADC-7 group (negative control), ADC-1 group, and ADC-4 group based on the tumor volume. After grouping, the corresponding drugs were injected into the tail veins once every week for a total of 2 times. After injection, the tumor volumes and body weights of the mice were observed and measured regularly. The result is shown in Table 5.

TABLE 5
Human gastric cancer MKN45 model

| | | | Day 21 | | |
|---|---|---|---|---|---|
| | | | Tumor | | |
| | | Dose | volume | TGI | P value* |
| Grouping | Group No. | (mg/kg) | (mm³) | (%) | (vs. group 1) |
| Group 1 | 0.9% NaCl | — | 886.30 | — | — |
| | injection | | | | |
| Group 2 | ADC-7 | 3 | 1061.52 | −23.02 | 0.15 |
| Group 3 | ADC-4 | 3 | 595.26 | 37.86 | 0.0003 |
| Group 4 | ADC-1 | 1 | 581.27 | 40.82 | 0.0001 |
| Group 5 | ADC-1 | 3 | 118.75 | 111.39 | <0.0001 |

*P value less than 0.05 means significant difference.

As can be seen from Table 5, compared with the 0.9% NaCl injection control group, the ADC-4 group has weaker inhibition of tumor growth; and ADC-1 groups at low- and medium-dose significantly inhibit tumor growth in a dose-dependent manner. At the equal dose of 3 mg/kg, the anti-tumor efficacy of the ADC-1 group is significantly better than that of the ADC-4 group (P<0.05). The above results show that with conjugating to the same toxin-linker, the efficacy of Ab-hzDPmut is better than that of tusamitamab. Further, during the experiment, there was no obvious change in the body weights of mice in each dose group of ADC-1, showing that the ADC of the present application has good safety.

### 6. In Vivo Efficacy Testing of Different Antibody-Drug Conjugates in a MKN45 Model

The subcutaneous xenograft tumor mouse model of human gastric cancer MKN45 was constructed using the same method as Experiment 5. When the tumors grew to an average volume of approximately 150 mm³, the animals were randomly grouped based on the tumor volume. After grouping, 0.9% NaCl injection, ADC-1 to ADC-4, ADC-7, or ADC-8 was administered respectively at the tail veins once every week based on the dose shown in Table 6. The mice were administered for a total of 2 times, and observed until day 28. After injection, the tumor volumes and body weights of the mice were observed and measured regularly. The result is shown in Table 6.

TABLE 6
Human gastric cancer MKN45 model

| | | | Day 28 | | |
|---|---|---|---|---|---|
| | | | Tumor | | |
| | | Dose | volume | TGI | P value* |
| Grouping | Group No. | (mg/kg) | (mm³) | (%) | (vs. group 1) |
| Group 1 | 0.9% NaCl | — | 1677.44 | — | — |
| | injection | | | | |
| Group 2 | ADC-7 | 3 | 1749.16 | −4.58 | >0.9999 |
| Group 3 | ADC-8 | 3 | 1740.08 | −4.04 | >0.9999 |
| Group 4 | ADC-1 | 3 | 231.7 | 94.83 | <0.0001 |
| Group 5 | ADC-2 | 3 | 114.3 | 124.85 | <0.0001 |
| Group 6 | ADC-3 | 6 | 590.66 | 71.36 | <0.0001 |
| Group 7 | ADC-4 | 3 | 1314.59 | 23.68 | 0.0897 |

*P value less than 0.05 means significant difference.

As can be seen from Table 6, compared with the 0.9% NaCl injection control group, the ADC-1-3 groups significantly inhibit tumor growth; and at the equal dose of 3 mg/kg, the anti-tumor efficacy of ADC-1 and ADC-2 is significantly better than that of the ADC-4 group (P<0.05), showing that under the same toxin-linker strategy, the efficacy of the Ab-hzDPmut conjugate is better than that of the tusamitamab conjugate; and the anti-tumor efficacy of ADC-2 is further better than that of ADC-3, showing that when the conjugates ADC-1 to 3 all can significantly inhibit tumor growth, the efficacy of the DAR8 conjugate is further better than that of the DAR4 conjugate. During the experiment, there was no obvious change in the body weights of mice in each group of ADC-1 to 3, showing that the ADC of the present application has good safety.

### 7. In Vivo Efficacy Testing of Different Drug Conjugates in a MKN45 Model

The subcutaneous xenograft tumor mouse model of human gastric cancer MKN45 was constructed using the same method as Experiment 5. When the tumors grew to an average volume of approximately 150 mm³, the animals were randomly grouped based on the tumor volume. After grouping, 0.9% NaCl injection, ADC-8, ADC-5, ADC-2, or ADC-6 was administered respectively at the tail veins once every week based on the dose shown in Table 7. The mice were administered for a total of 2 times, and observed until day 24. After injection, the tumor volumes and body weights of the mice were observed and measured regularly. The result is shown in Table 7.

TABLE 7
Human gastric cancer MKN45 model

| | | | Day 24 | | |
|---|---|---|---|---|---|
| | | | Tumor | | |
| | | Dose | volume | TGI | P value* |
| Grouping | Group No. | (mg/kg) | (mm³) | (%) | (vs. group 1) |
| Group 1 | 0.9% NaCl | — | 1334.36 | — | — |
| | injection | | | | |
| Group 2 | ADC-8 | 3 | 1106.52 | 19.13 | 0.21 |
| Group 3 | ADC-2 | 1 | 585.84 | 63.87 | <0.0001 |
| Group 4 | ADC-2 | 3 | 75.56 | 152.28 | <0.0001 |
| Group 5 | ADC-5 | 3 | 274.84 | 90.45 | <0.0001 |
| Group 6 | ADC-6 | 1 | 629.28 | 60.49 | <0.0001 |
| Group 7 | ADC-6 | 3 | 160.54 | 99.95 | <0.0001 |

*P value less than 0.05 means significant difference.

As can be seen from Table 7, compared with the 0.9% NaCl injection control group, the test drugs all significantly inhibit tumor growth at the experimental dose; at the dose of 3 mg/kg, the anti-tumor effect of ADC-2 is significantly better than that of ADC-5 (P<0.05), showing that when different antibodies Ab-hzDPmut and Tusamitamab are conjugated to the same toxin-linker, the efficacy of the Ab-hzDPmut conjugate is better than that of the Tusamitamab conjugate. Further, even if conjugated to different toxin-linkers, ADC-2 has obviously better anti-tumor effects than the highly toxic ADC-6, showing the excellent effects of the toxin-linker strategy of the present application. Further, during the experiment, there was no obvious change in the body weights of mice in each dose group of ADC-2, showing that the ADC of the present application has good safety.

### 8. Efficacy Testing of Antibody-Drug Conjugate at Different Doses in a LS-174T Model

The human colorectal cancer cell LS174T used in this example is CEACAM5-positive. LS174T cells were cultured in 1640 culture median with 10% fetal bovine serum with 5% CO₂ at 37° C. The cells in an exponential growth phase were collected, resuspended in PBS to a suitable concentration, and inoculated subcutaneously into female Balb/c nude mice to construct a human colorectal cancer xenograft tumor model. When the tumors grew to an average volume of approximately 150 mm³, the animals were randomly divided into 0.9% NaCl injection group, ADC-8 group (negative control), ADC-1 group, and ADC-2 group based on the tumor volume. After grouping, the corresponding drugs were injected into the tail veins once every week for a total of 2 times. After injection, the tumor volumes and body weights of the mice were observed and measured regularly. The result is shown in Table 8.

TABLE 8
Human colorectal cancer LS174T model

| | | | Day 14 | | |
|---|---|---|---|---|---|
| | | | Tumor | | |
| | | Dose | volume | TGI | P value* |
| Grouping | Group No. | (mg/kg) | (mm³) | (%) | (vs. group 1) |
| Group 1 | 0.9% NaCl | — | 2391.81 | — | — |
| | injection | | | | |
| Group 2 | ADC-8 | 3 | 2041.80 | 15.60 | 0.1871 |
| Group 3 | ADC-1 | 3 | 711.47 | 74.81 | <0.001 |
| Group 4 | ADC-2 | 3 | 853.64 | 68.39 | <0.001 |

*P value less than 0.05 means significant difference.

As can be seen from Table 8, compared with the 0.9% NaCl injection control group, both the ADC-1 and the ADC-2 significantly inhibit tumor growth at the experimental doses. During the experiment, there was no obvious change in the body weights of mice in the ADC-1 group and the ADC-2 group, showing that the ADC of the present application has good safety.

Although the specific embodiment of the present invention has been described in detail, those skilled in the art will understand that according to all the teachings disclosed, various modifications and replacements can be made to those details, and these changes are all within the protection scope of the present invention. The full scope of the invention is given by the appended claims and any equivalents thereof.

1. An antibody-drug conjugate, having a structure shown as a formula:
HA-(Y-M-L-E-D)ₓ′,
wherein HA-(Y-represents an antibody or antigen-binding fragment thereof that specifically binds to CEACAM5, wherein-(Y-represents a linkage from an amino acid residue of the antibody or antigen-binding fragment thereof to M;
M-L-E is a linker connecting -(Y-to D, wherein M is the portion of the linker connecting to Y, L is the central portion of the linker, and E is the portion of the linker connecting to D;
D is a cytotoxic drug; and
x′ is selected from 1-20.

2. The antibody- conjugate of claim 1, wherein the amino acid residue is a cysteine, lysine, serine or threonine.

3. The antibody-drug conjugate according to claim 1 or 2, wherein the antibody or antigen-binding fragment thereof comprises:
(1) the following heavy chain variable regions (VH) and/or light chain variable regions (VL), wherein CDRs are defined according to a Chothia numbering system:
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
(2) the following heavy chain variable region (VH) and/or light chain variable region (VL), wherein the CDRs are defined according to the Kabat numbering system:
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
(3) the following heavy chain variable regions (VH) and/or light chain variable regions (VL), wherein the CDRs are defined according to the IMGT numbering system:
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
or,
(4) the following heavy chain variable regions (VH) and/or light chain variable regions (VL), wherein the CDRs are defined according to the AbM numbering system:
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement.

4. The antibody-drug conjugate according to any one of claims 1 to 3, wherein the antibody or antigen-binding fragment thereof comprises a V_{H} having the amino acid sequence as set forth in SEQ ID NO: 1 or a variant thereof, and/or, a V_{L} having the amino acid sequence as set forth in SEQ ID NO: 2 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids as compared to the sequence from which the variants are derived.

5. The antibody-drug conjugate according to any one of claims 1 to 4, wherein the antibody or antigen-binging fragment thereof further comprises:
(a) a human immunoglobulin heavy chain constant region (CH) or a variant thereof, wherein the variant has replacement, deletion or addition of one, two, or more amino acids as compared to a wild-type sequence from which the variant is derived; and
(b) a human immunoglobulin light chain constant region (CL) or a variant, wherein the variant has replacement, deletion or addition of one, two, or more amino acids as compared to a wild-type sequence from which the variant is derived.

6. The antibody-drug conjugate according to any one of claims 1 to 5, wherein HA-(Y-comprises a heavy chain comprising a V_{H} having the amino acid sequence as set forth in SEQ ID NO: 1 and heavy chain constant region (CH) having the amino acid sequence as set forth in SEQ ID NO: 3, and a light chain comprising a V_{L} having the amino acid sequence as set forth in SEQ ID NO: 2 and light chain constant region (CL) having the amino acid sequence as set forth in SEQ ID NO: 4.

7. The antibody-drug conjugate according to any one of claims 1 to 5, wherein HA-(Y-comprises a heavy chain having the amino acid sequence as set forth in SEQ ID NO: 20 and a light chain having the amino acid sequence as set forth in SEQ ID NO: 21.

8. The antibody-drug conjugate according to any one of claims 1 to 7, wherein Mis 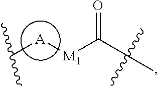
ring A is a 5-6 membered aliphatic heterocyclic ring, or 5-20 membered aromatic ring system, and the aliphatic heterocyclic ring and the aromatic ring system are optionally substituted with one or more members independently selected from the group consisting of oxo (═O), halogen, cyano, amino, carboxyl, mercapto and C₁₋₆ alkyl; and M₁ is selected from single bond, C₁₋₂₀ alkylene, C₂₋₂₀ alkenylene and C₂₋₂₀ alkynylene.

9. The antibody-drug conjugate according to any one of claims 1 to 7, wherein M is 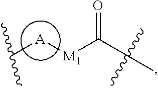
wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted with one or more members independently selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene.

10. The antibody-drug conjugate according to any one of claims 1 to 7, wherein M is 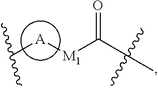
wherein ring A is 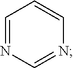
and M₁ is selected from a single bond, C₅₋₈ alkylene, C₅₋₈ alkenylene and C₅₋₈ alkynylene.

11. The antibody-drug conjugate according to any one of claims 1 to 7, wherein M is

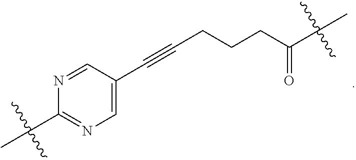 [SMILES: *C(=O)CCCC#Cc1cnc(*)nc1]

12. The antibody-drug conjugate according to any one of claims 1 to 11, wherein L is selected from a structure comprising one or more of C₁₋₆ alkylene, -N(R′)—, carbonyl, -O—, Val, Cit, Phe, Lys, Lys(COCH₂CH₂ (OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 25), Gly-Phe-Leu-Gly (SEQ ID NO: 26), Gly-Gly-Val-Ala (SEQ ID NO: 27), Gly-Gly-Gly-Gly-Gly (SEQ ID NO: 28), 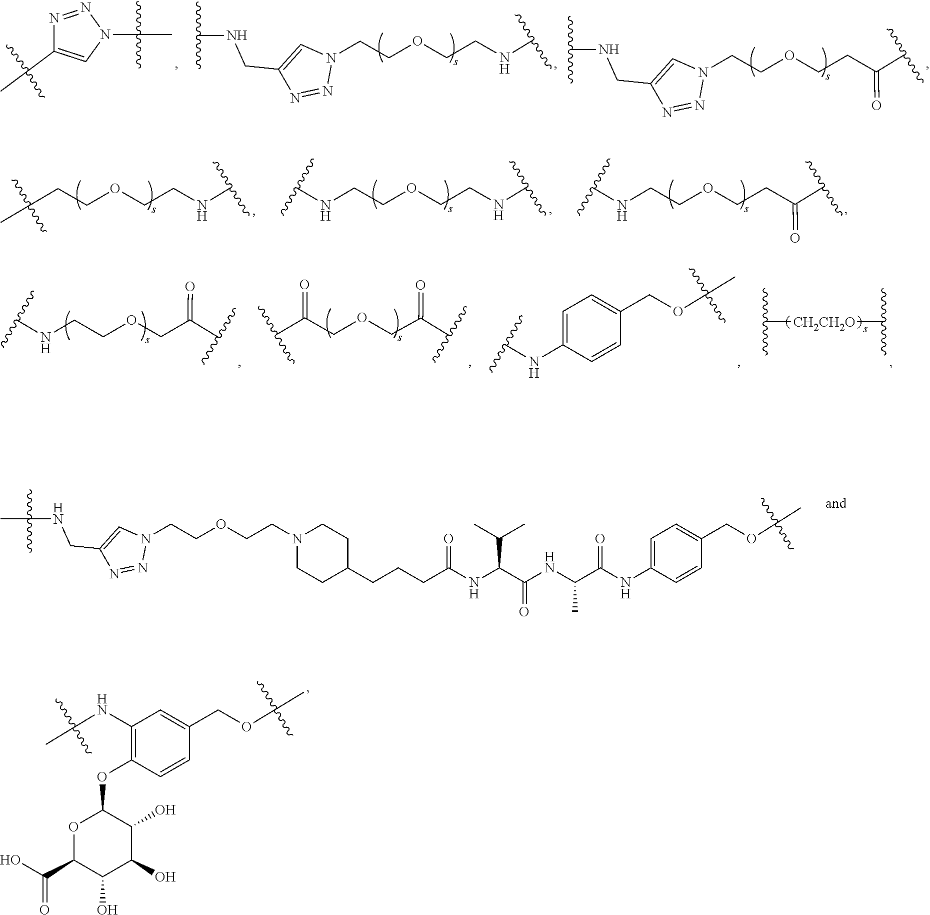
or a combination thereof, wherein R′ represents hydrogen, C₁₋₆ alkyl or alkyl containing —(CH₂CH₂O)ᵣ-; r is an integer selected from 1-10; and s is an integer selected from 1-20.

13. The antibody-drug conjugate according to any one of claims 1 to 11, wherein L is selected from the following structures:

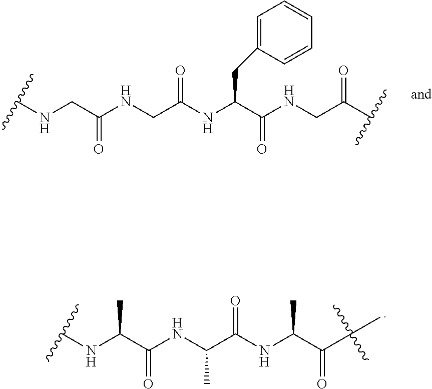 [SMILES: *NCC(=O)NCC(=O)N[C@@H](Cc1ccccc1)C(=O)NCC(*)=O ; *N[C@@H](C)C(=O)N[C@@H](C)C(=O)N[C@@H](C)C(*)=O]

14. The antibody-drug conjugate according to any one of claims 1 to 13, wherein E is a single bond, —NH—CH₂-, —NH—CH₂-O-CH₂—CO—,

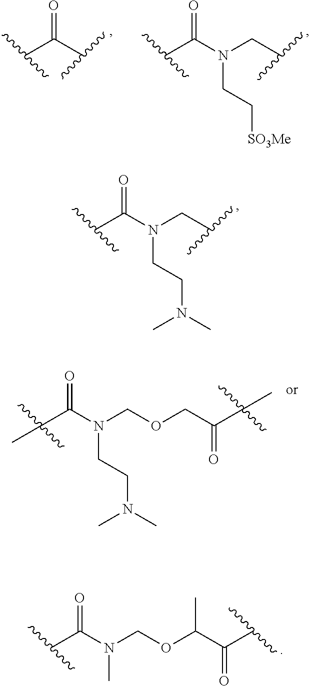

15. The antibody-drug conjugate according to any one of claims 1 to 13, wherein E is —NH—CH₂-O-CH₂—CO-.

16. The antibody-drug conjugate according to any one of claims 1 to 15, wherein 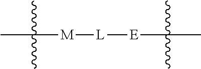
is selected from the following structures:

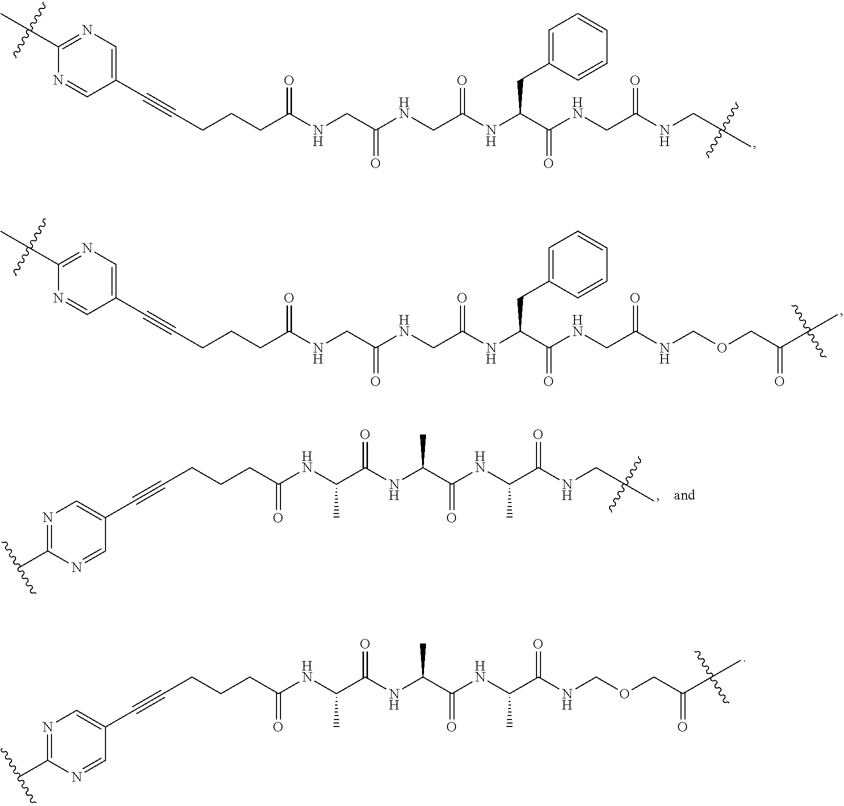 [SMILES: *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

17. The antibody-drug conjugate according to any one of claims 1 to 16, wherein the cytotoxic drug is selected from a tubulin inhibitor, a DNA intercalator, a DNA topoisomerase inhibitor and a RNA polymerase inhibitor or pharmaceutically acceptable salt, ester or analog thereof.

18. The antibody-drug conjugate according to claim 17, wherein the cytotoxic drug is selected from a compound shown as formula I and formula II: 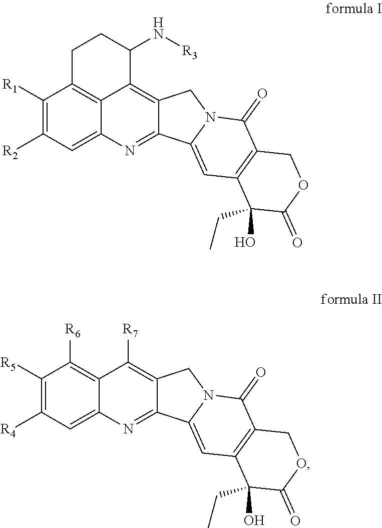
R₁ and R₂ are each independently selected from C₁₋₆ alkyl and halogen;
R₃ is selected from H and -CO-CH₂OH;
R₄ and R₅ are each independently selected from H, halogen and hydroxyl; or R₄ and R₅ are linked to associated carbon atoms to form 5-6 membered oxygen- containing heterocyclic ring;
R₆ is selected from hydrogen and -C₁₋₄ alkylene-NRₐR_{b};
R₇ is selected from hydrogen, C₁₋₆ alkyl and -C₁₋₄ alkylene-NRₐR_{b}; and
Rₐ and R_{b} are each independently selected from H, C₁₋₆ alkyl, -SO₂-C₁₋₆ alkyl and -CO-C₁₋₆ alkyl at each occurrence.

19. The antibody-drug conjugate according to claim 18, wherein the cytotoxic drug is selected from the following compounds:

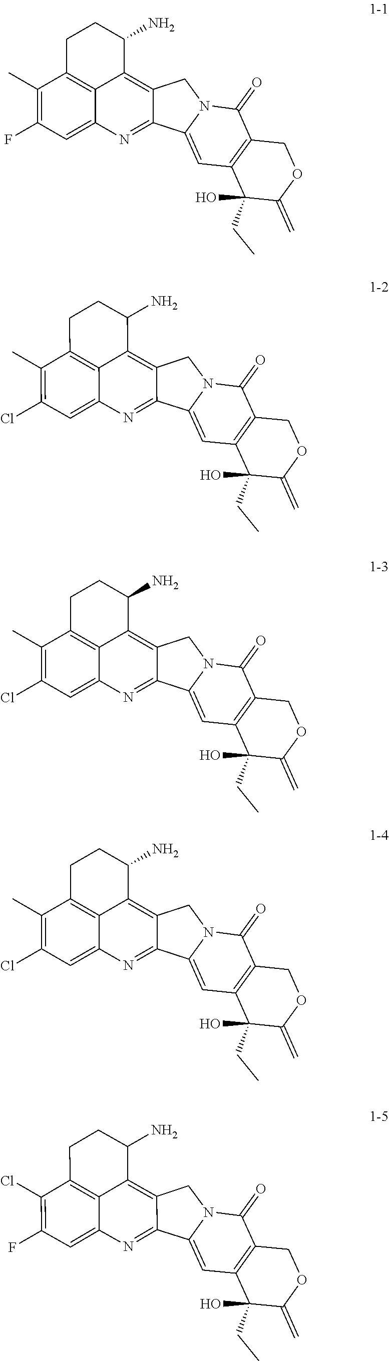 [SMILES: C=C1OCc2c(cc3n(c2=O)Cc2c-3nc3cc(F)c(C)c4c3c2[C@@H](N)CC4)[C@@]1(O)CC ; C=C1OCc2c(cc3n(c2=O)Cc2c-3nc3cc(Cl)c(C)c4c3c2C(N)CC4)[C@@]1(O)CC ; C=C1OCc2c(cc3n(c2=O)Cc2c-3nc3cc(Cl)c(C)c4c3c2[C@H](N)CC4)[C@@]1(O)CC ; C=C1OCc2c(cc3n(c2=O)Cc2c-3nc3cc(Cl)c(C)c4c3c2[C@@H](N)CC4)[C@@]1(O)CC ; C=C1OCc2c(cc3n(c2=O)Cc2c-3nc3cc(F)c(Cl)c4c3c2C(N)CC4)[C@@]1(O)CC]

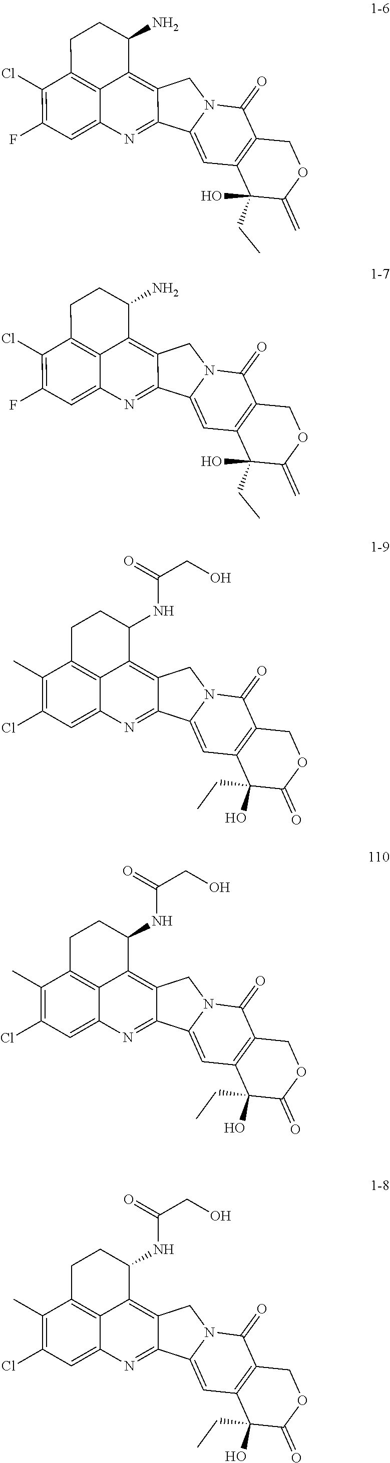 [SMILES: C=C1OCc2c(cc3n(c2=O)Cc2c-3nc3cc(F)c(Cl)c4c3c2[C@H](N)CC4)[C@@]1(O)CC ; C=C1OCc2c(cc3n(c2=O)Cc2c-3nc3cc(F)c(Cl)c4c3c2[C@@H](N)CC4)[C@@]1(O)CC ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

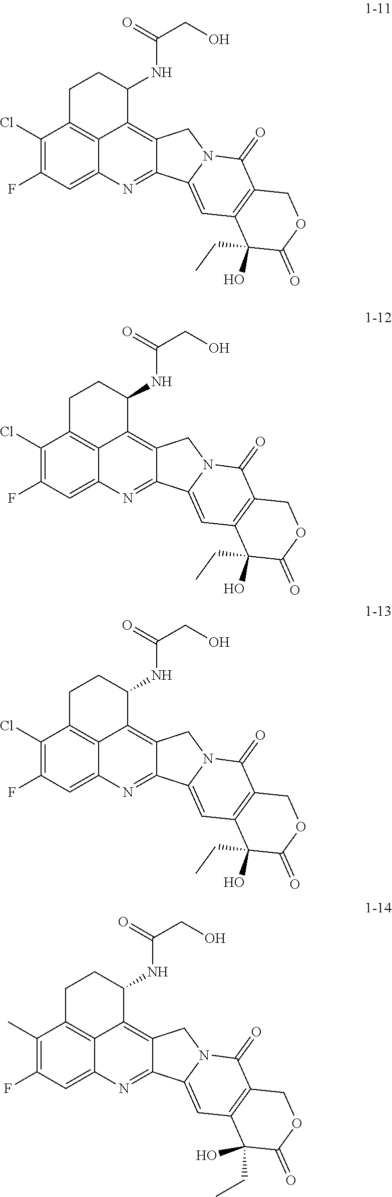 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

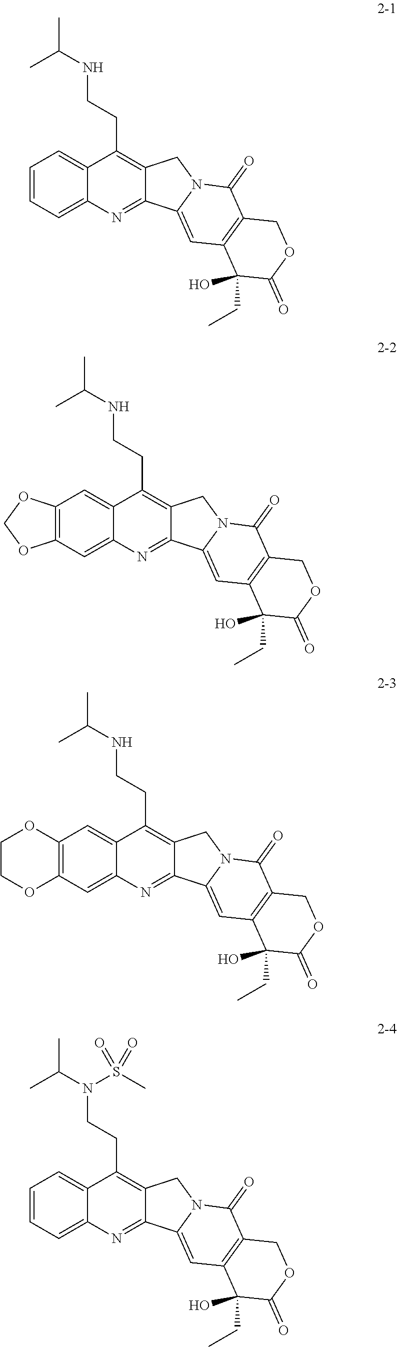 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O]

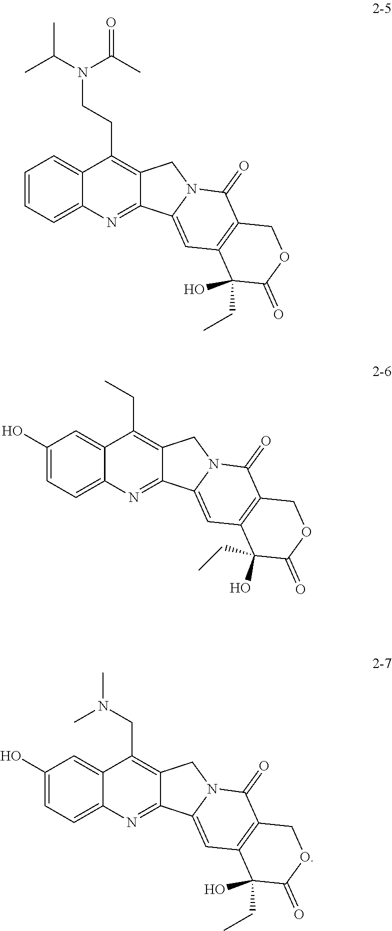 [SMILES: CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

20. The antibody-drug conjugate according to claim 19, wherein D is linked to E by the loss of one H from —OH, —NH₂, or secondary amine groups on the cytotoxic drug.

21. The antibody-drug conjugate according to any one of claims 1 to 20, wherein D is selected from

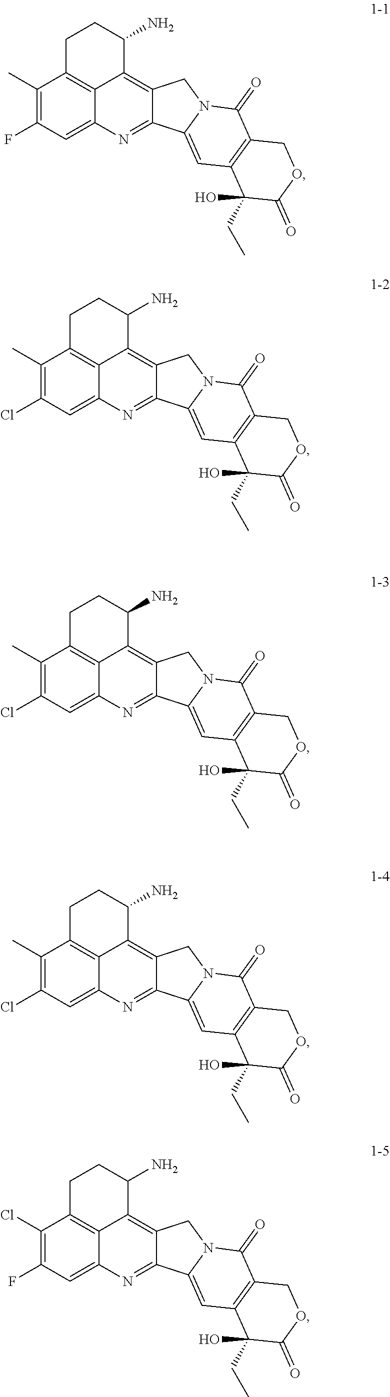 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

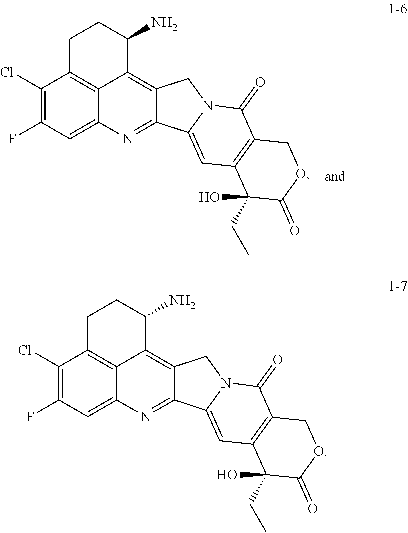 [SMILES: CC[C@@]1(O)C(=O)OCc2c1cc1n(c2=O)Cc2c-1nc1cc(F)c(Cl)c3c1c2[C@H](N)CC3 ; "and" ; CC[C@@]1(O)C(=O)OCc2c1cc1n(c2=O)Cc2c-1nc1cc(F)c(Cl)c3c1c2[C@@H](N)CC3]

22. The antibody-drug conjugate according to any one of claims 1 to 21, wherein (-M-L-E-D) is selected from

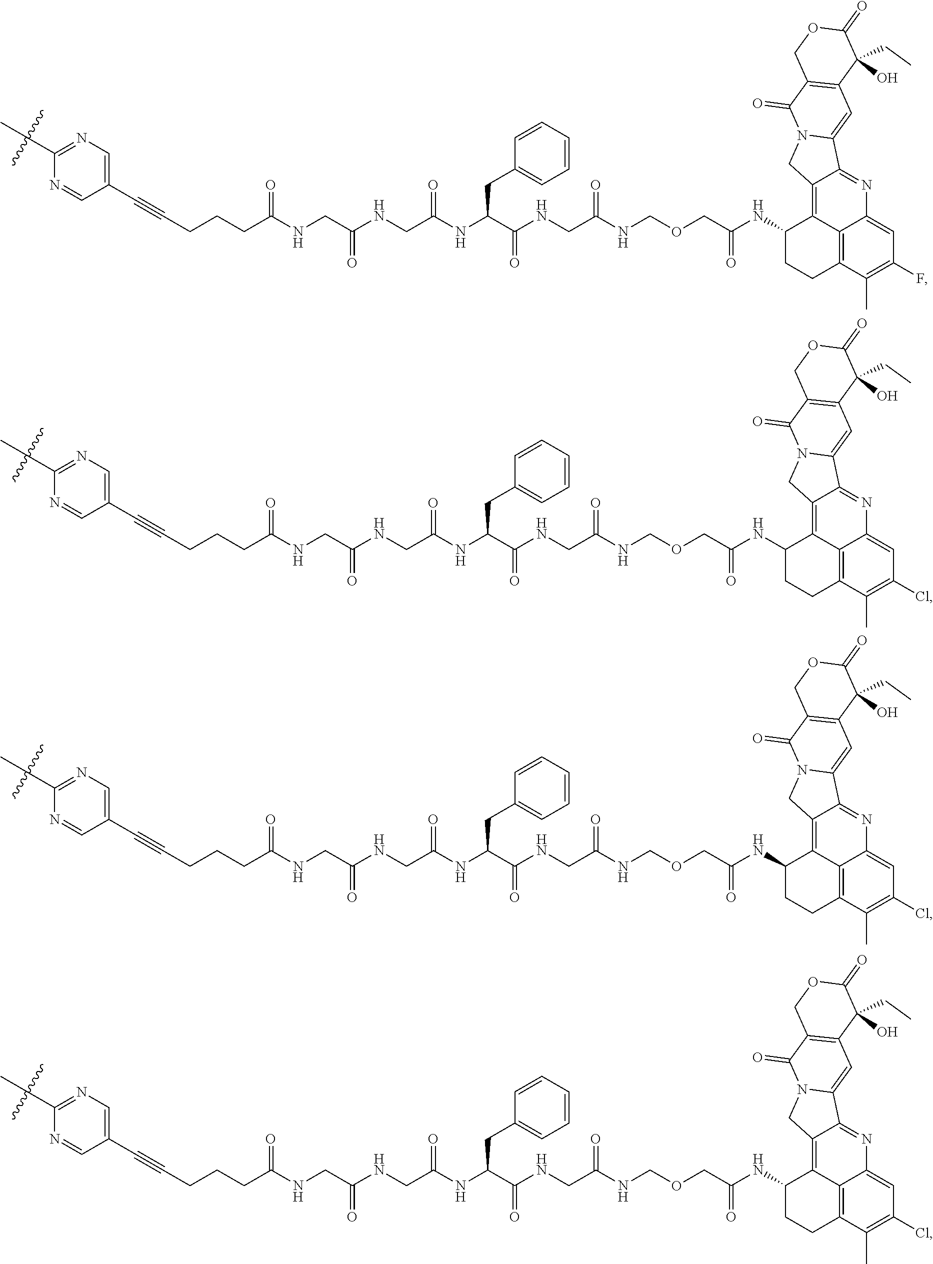

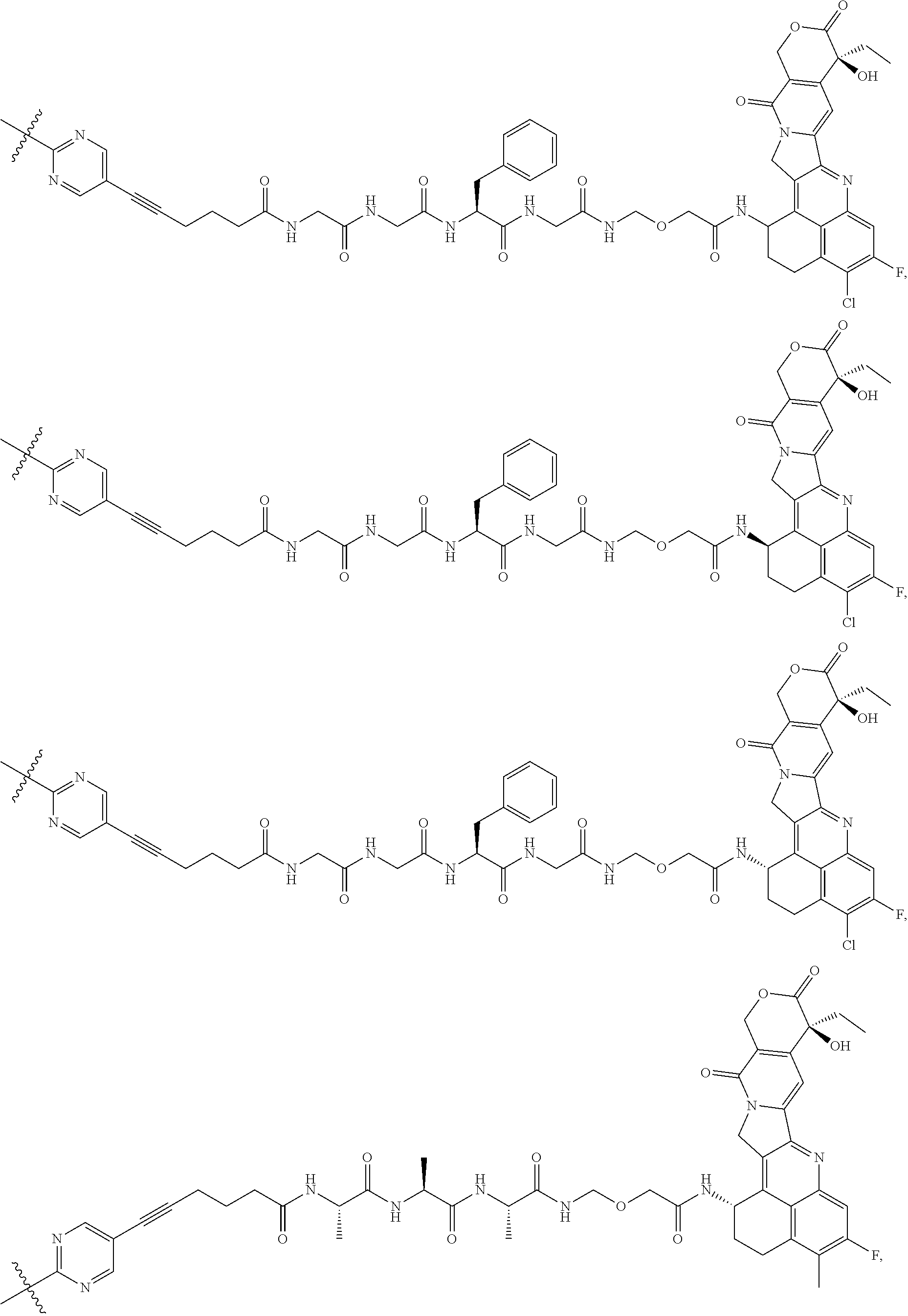

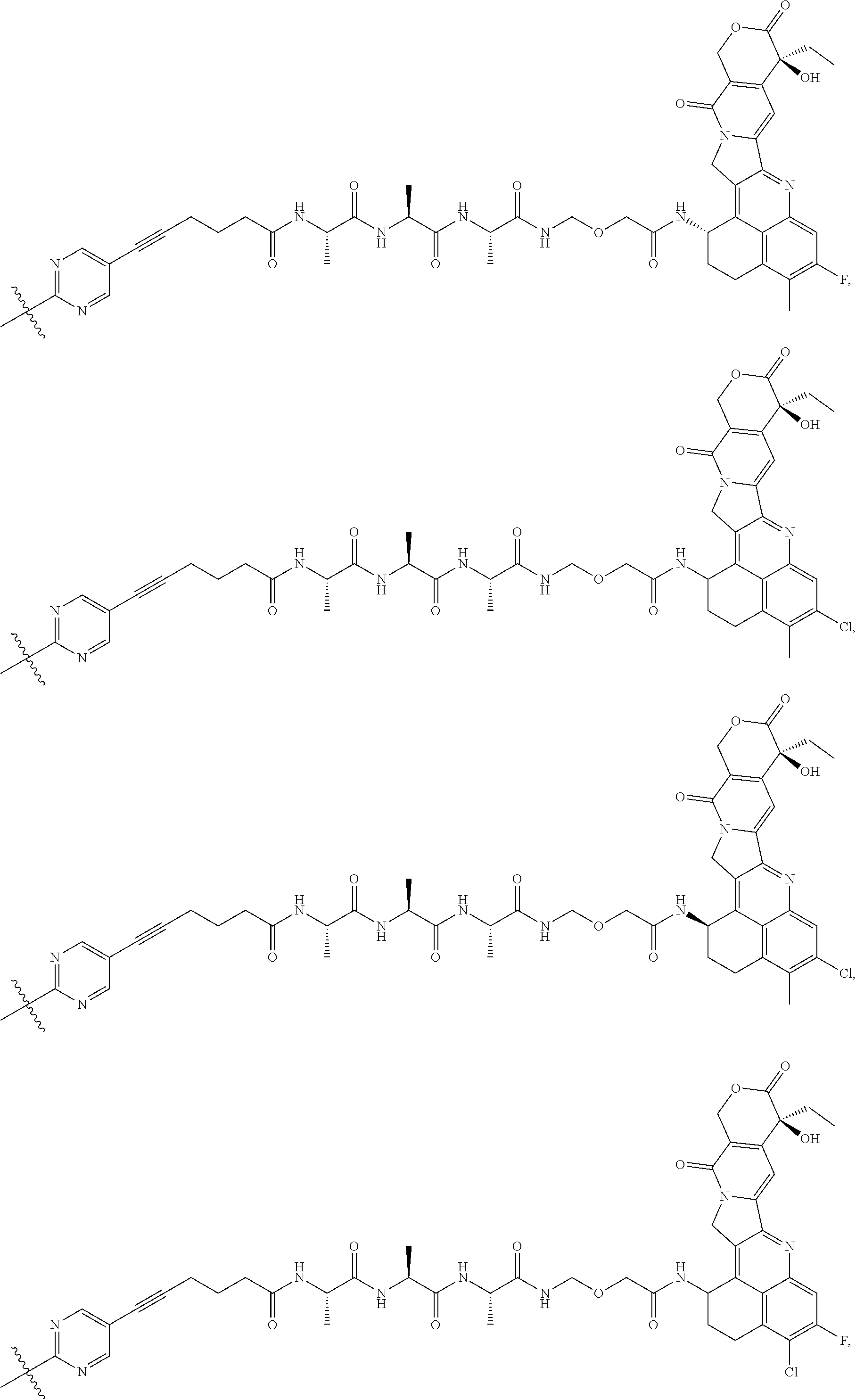

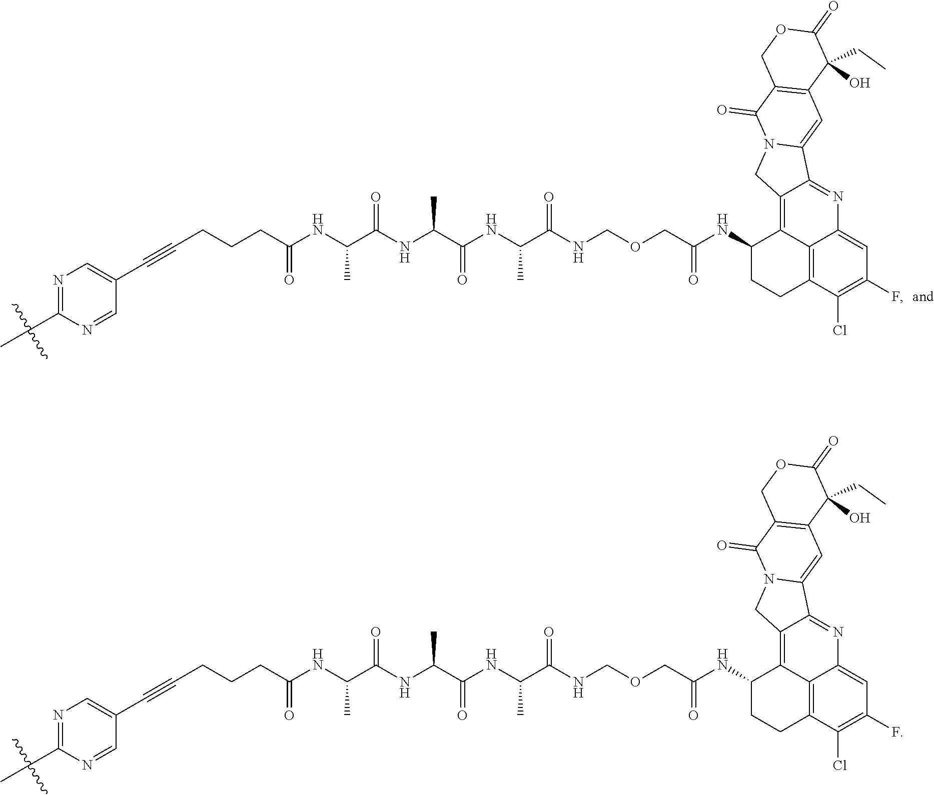 [SMILES: *c1ncc(C#CCCCC(=O)N[C@@H](C)C(=O)N[C@@H](C)C(=O)N[C@@H](C)C(=O)NCOCC(=O)N[C@@H]2CCc3c(Cl)c(F)cc4nc5c(c2c34)Cn2c-5cc3c(c2=O)COC(=O)[C@]3(O)CC)cn1 ; *c1ncc(C#CCCCC(=O)N[C@@H](C)C(=O)N[C@@H](C)C(=O)N[C@@H](C)C(=O)NCOCC(=O)N[C@H]2CCc3c(Cl)c(F)cc4nc5c(c2c34)Cn2c-5cc3c(c2=O)COC(=O)[C@]3(O)CC)cn1]

23. The antibody-drug conjugate according to any one of claims 1 to 22, selected from ADC-Aa, ADC-Aa1, ADC-Aa2, ADC-Bb, ADC-Bb1, and ADC-Bb2 shown as follows: 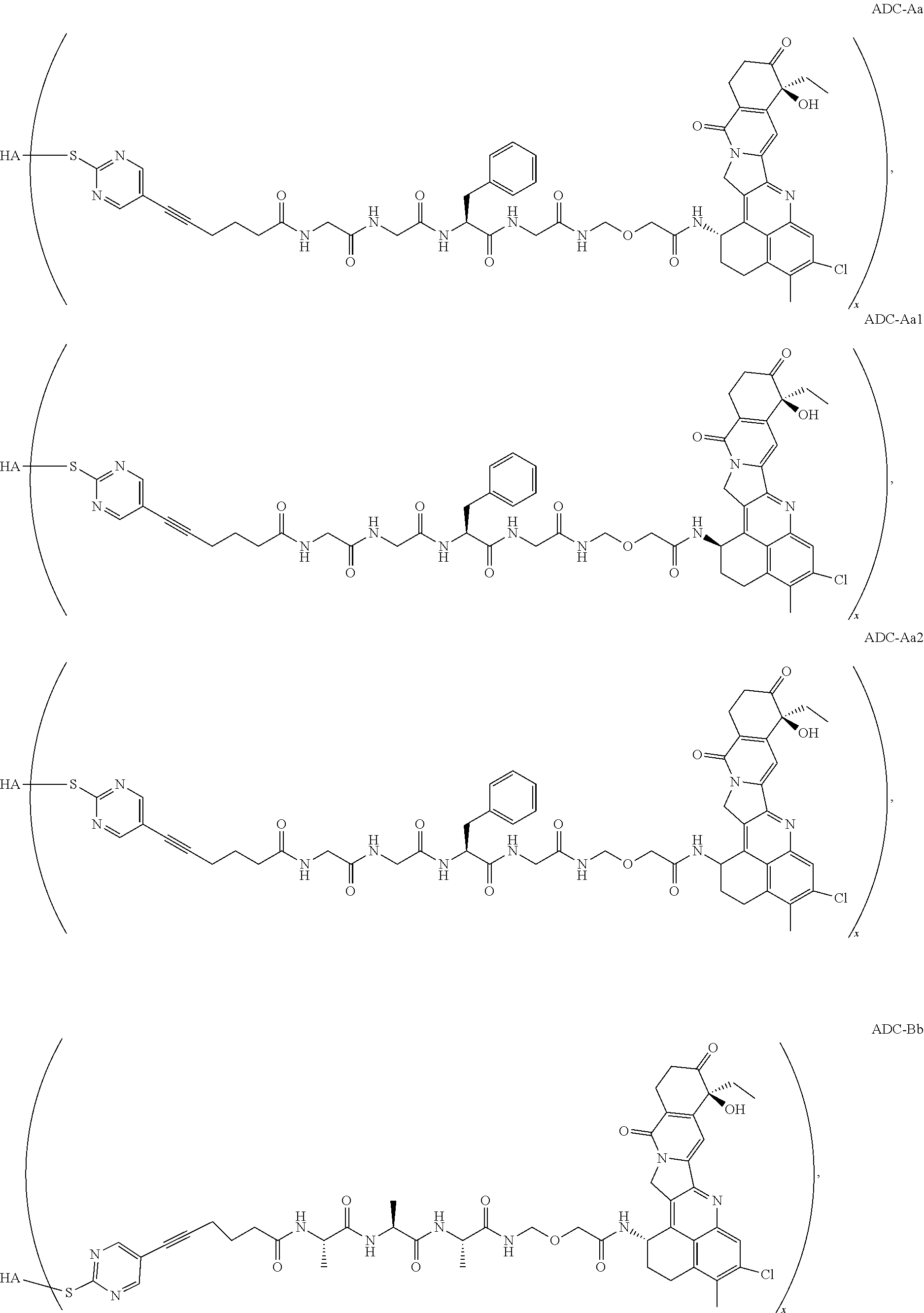 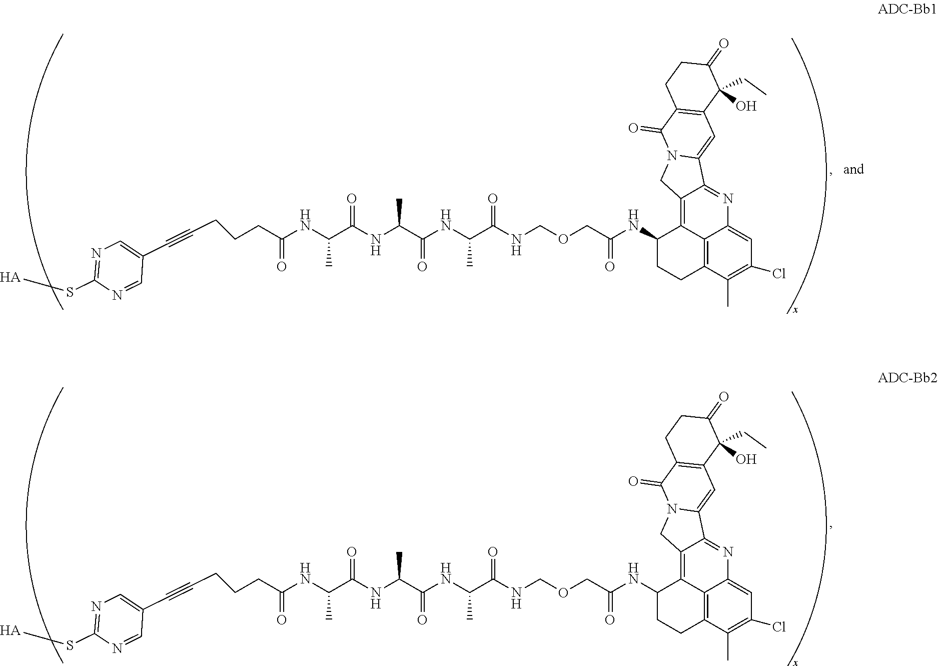
wherein HA-(Y- is HA-(S-;
HA-(S- comprises an antibody comprising a V_{H} having the amino acid sequence as set forth in SEQ ID NO: 1, and a V_{L} having the amino acid sequence as set forth in SEQ ID NO: 2;
each -(S-represents a linkage from a sulfhydryl of a cysteine residue in the antibody to each pyrimidinyl group in formulas ADC-Aa, ADC-Aa1, ADC-Aa2, ADC-Bb, ADC-Bb1, and ADC-Bb2;
and x=1-16, 1-10, 1-8, 4-8, 4 or 8.

24. The antibody-drug conjugate according to any one of claims 1 to 23, wherein the antibody-drug conjugate comprises the formula ADC-Aa or ADC-Bb: 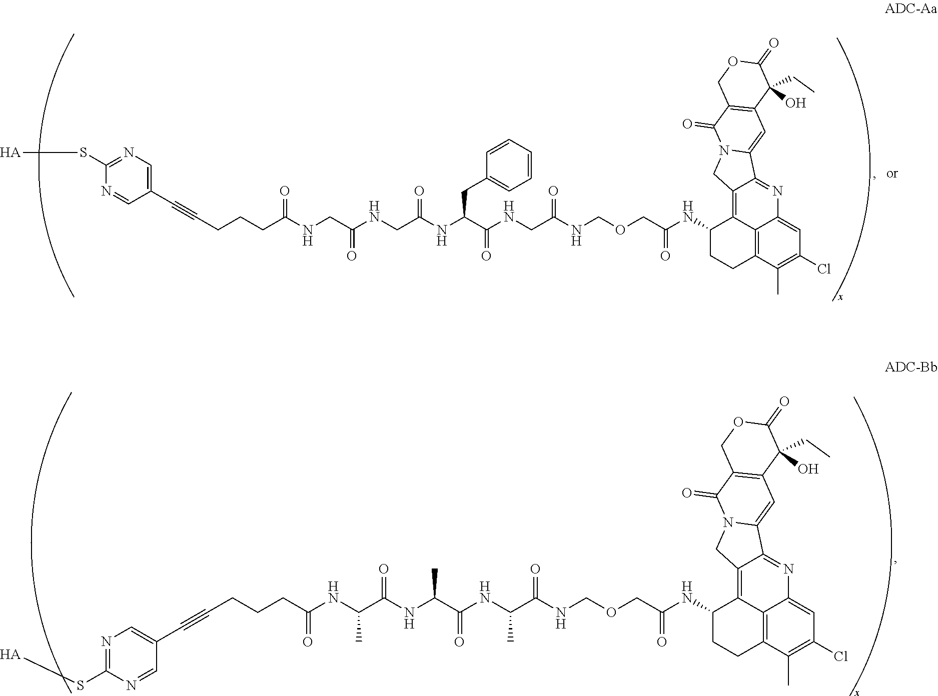
wherein HA-(Y- is HA-(S-;
HA-(S- comprises a V_{H} having the amino acid sequence as set forth in SEQ ID NO: 1, and a V_{L} having the amino acid sequence as set forth in SEQ ID NO: 2;
each -(S-represents a linkage from a sulfhydryl of a cysteine residue in the antibody to each pyrimidinyl group in formulas ADC-Aa and ADC-Ba;
and x is 1-16, 1-10, 1-8, 4-8, 4 or 8.

25. The antibody-drug conjugate according to any one of claims 1 to 24, wherein the antibody-drug conjugate comprises formula ADC-Aa or ADC-Bb: 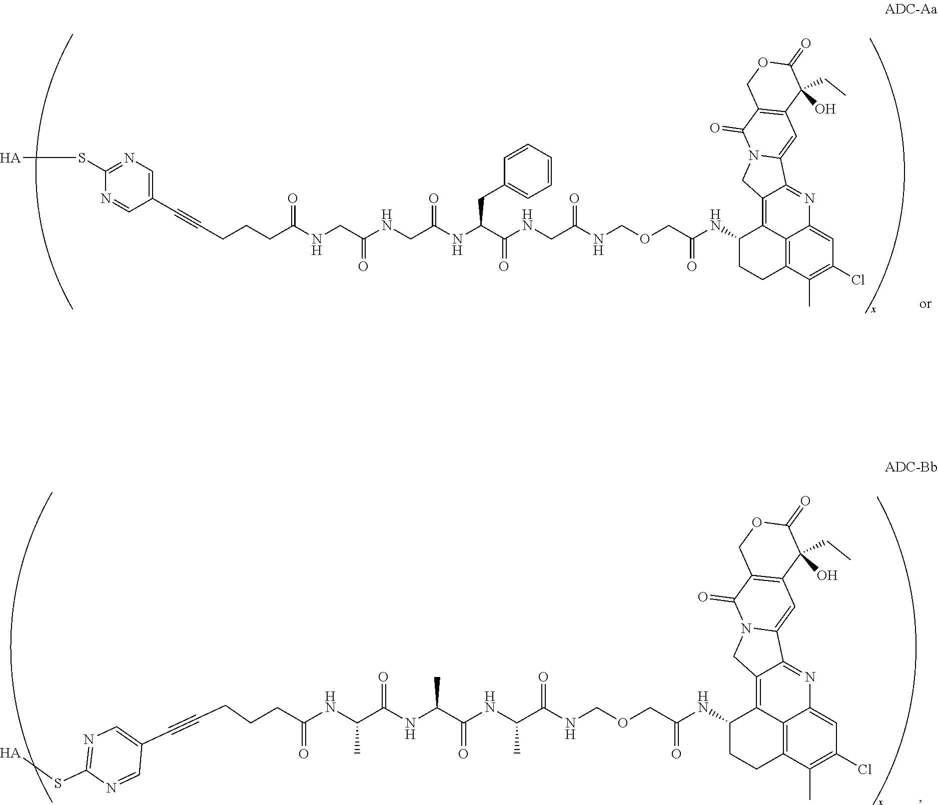
wherein HA-(Y- is HA-(S-;
HA-(S- comprises a V_{H} having the amino acid sequence as set forth in SEQ ID NO: 1 and a CH having the amino acid sequence as set forth in SEQ ID NO: 3, and a V_{L} having the amino acid sequence as set forth in SEQ ID NO: 2, and a CL having the amino acid sequence as set forth in SEQ ID NO: 4;
each -(S-represents a linkage from a sulfhydryl in the antibody to each pyrimidinyl group in formulas ADC-Aa and ADC-Bb;
and x is 1-16, 1-10, 1-8, 3-9, 4-8, 4 or 8.

26. The antibody-drug conjugate according to any one of claims 1 to 25, wherein the antibody-drug conjugate is ADC-Aa: 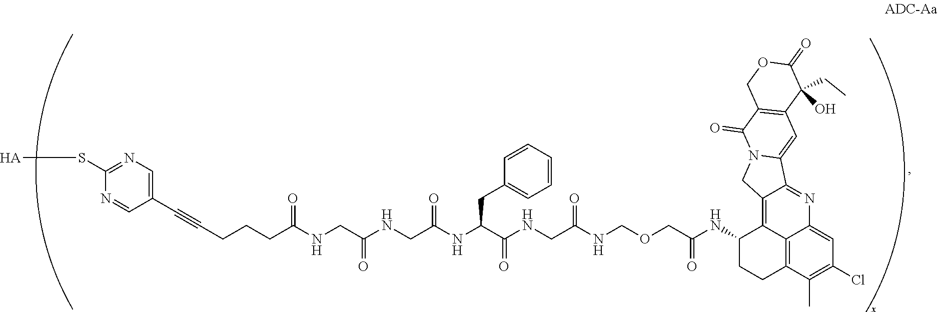
wherein HA-(Y- is HA-(S-;
HA-(S- comprises a V_{H} having the amino acid sequence as set forth in SEQ ID NO: 1, and a V_{L} having the amino acid sequence as set forth in SEQ ID NO: 2;
each -(S-represents a linkage from a sulfhydryl in the antibody to each pyrimidinyl group in formula ADC-Aa;
and x is 1-16, 1-10, 1-8, 4-8, 4 or 8.

27. The antibody-drug conjugate according to any one of claims 1 to 26, wherein the antibody-drug conjugate is ADC-Aa: 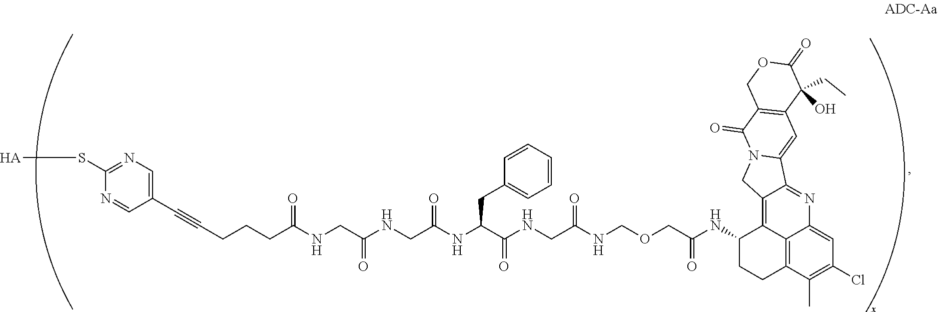
wherein HA-(Y- is HA-(S-;
HA-(S- comprises a V_{H} having the amino acid sequence as set forth in SEQ ID NO: 1 and a heavy chain constant region (CH) having the amino acid sequence as set forth in SEQ ID NO: 3, and a V_{L} having the amino acid sequence as set forth in SEQ ID NO: 2 and a light chain constant region (CL) having the amino acid sequence as set forth in SEQ ID NO: 4,
each -(S-represents a linkage from a sulfhydryl of a cysteine residue in the antibody to each pyrimidinyl group in formula ADC-Aa;
and x is 1-16, 1-10, 1-8, 4-8, 4 or 8.

28. The antibody-drug conjugate according to any one of claims 1 to 27, wherein the antibody-drug conjugate is ADC-Aa: 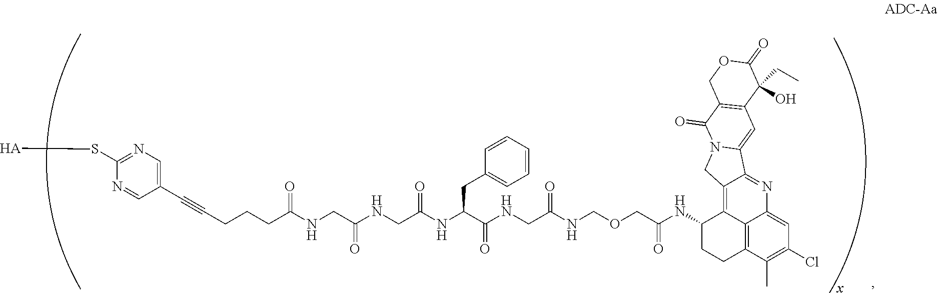
wherein HA-(Y- is HA-(S-;
HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x=1-16, 1-10, 1-8, 4-8, 4 or 8.

29. The antibody-drug conjugate according to any one of claims 1 to 28, wherein the antibody-drug conjugate is ADC-Aa: 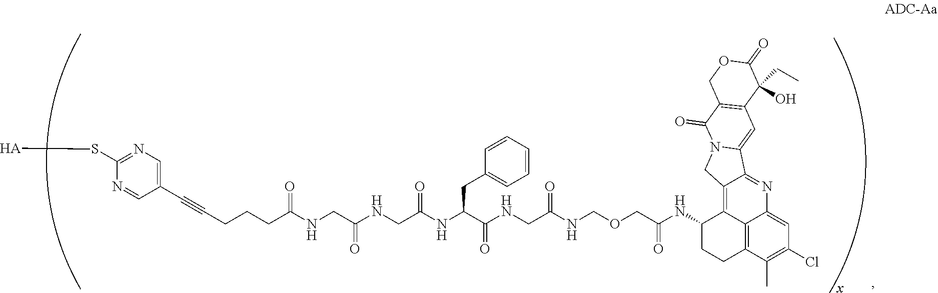
wherein HA-(Y- is HA-(S-;
HA-(S- comprises two heavy chains and two light chains, each heavy chain (HC) having the amino acid sequence as set forth in SEQ ID NO: 20 and each light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x=1-16, 1-10, 1-8, 4-8, 4 or 8.

30. The antibody-drug conjugate according to any one of claims 1 to 27, wherein the antibody-drug conjugate is ADC-Aa: 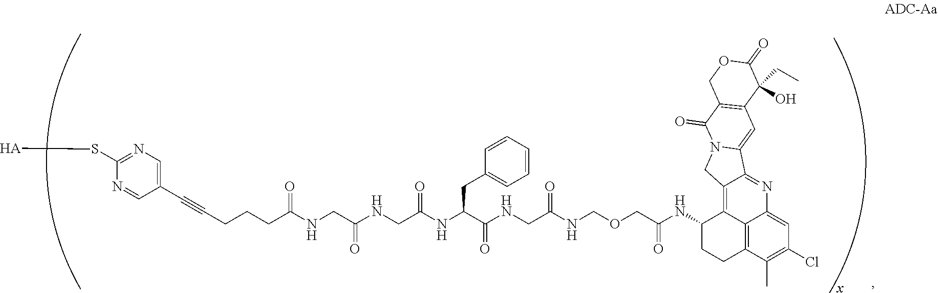
wherein HA-(Y- is HA-(S-;
HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa;
and x is 1-16, 1-10, 1-8, 4-8, 7-9, or 8.

31. The antibody-drug conjugate according to any one of claims 1 to 27, wherein the antibody-drug conjugate is ADC-Aa: 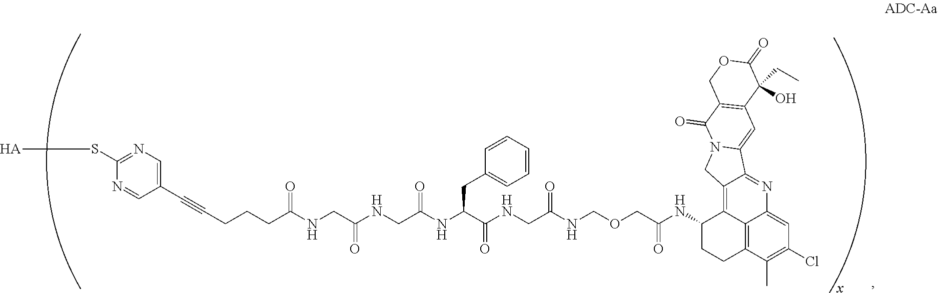
wherein HA-(Y- is HA-(S-;
HA-(S- comprises two heavy chains and two light chains, each heavy chain (HC) having the amino acid sequence as set forth in SEQ ID NO: 22 and each light chain (LC) having the amino acid sequence as set forth in SEQ ID NO: 21;
each -(S-represents a linkage from a sulfhydryl of a cysteine residue in the antibody to each pyrimidinyl group in formula ADC-Aa; and
x is 1-16, 1-8, 4-8, 7-9, or 8.

32. The antibody-drug conjugate according to any one of claims 1 to 27, wherein the antibody-drug conjugate is ADC-Aa: 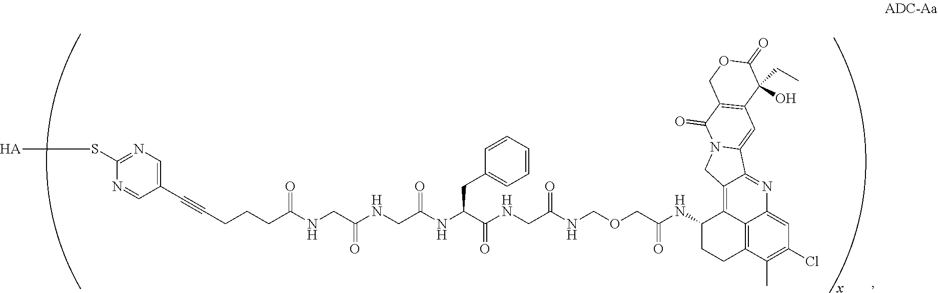
wherein HA-(Y- is HA-(S-;
HA-(S- comprises a heavy chain (HC) having the amino acid sequence as set forth in SEQ ID NO: 22 and a light chain (LC) having the amino acid sequence as set forth in SEQ ID NO: 21;
each -(S-represents a linkage from a sulfhydryl of a cysteine residue in the antibody to each pyrimidinyl group in formula ADC-Aa; and
x is 1-16; 1-8; 4-8; 7-9; 4 or 8.

33. The antibody-drug conjugate according to any one of claims 1 to 25, wherein the antibody-drug conjugate is ADC-Bb: 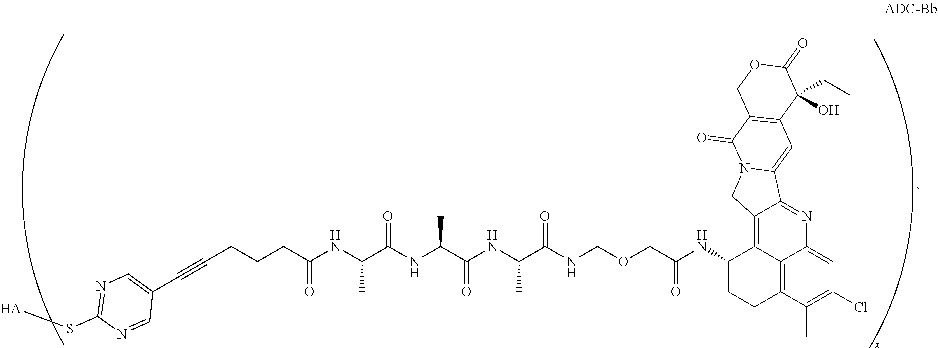
wherein HA-(Y- is HA-(S-;
HA-(S- comprises a V_{H} having the amino acid sequence as set forth in SEQ ID NO: 1, and a V_{L} having the amino acid sequence as set forth in SEQ ID NO: 2;
each -(S-represents a linkage from a sulfhydryl in the antibody to each pyrimidinyl group in formula ADC-Bb;
and x is 1 to 16, 1-10, 1-8, 4-8, 4 or 8.

34. The antibody-drug conjugate according to any one of claims 1 to 25, wherein the antibody-drug conjugate is ADC-Bb: 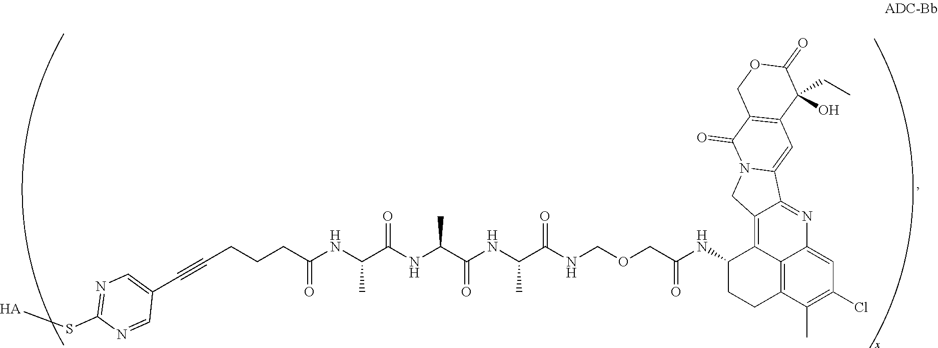
wherein HA-(Y- is HA-(S-;
HA-(S- comprises a V_{H} having the amino acid sequence as set forth in SEQ ID NO: 1 and a heavy chain constant region (CH) having the amino acid sequence as set forth in SEQ ID NO: 3, and a V_{L} having the amino acid sequence as set forth in SEQ ID NO: 2 and a light chain constant region (CL) having the amino acid sequence as set forth in SEQ ID NO: 4,
each -(S-represents a linkage from a sulfhydryl of a cysteine residue in the antibody to each pyrimidinyl group in formula ADC-Bb;
and x is 1-16, 1-10, 1-8, 4-8, 4 or 8.

35. The antibody-drug conjugate according to any one of claims 1 to 25, wherein the antibody-drug conjugate is ADC-Bb: 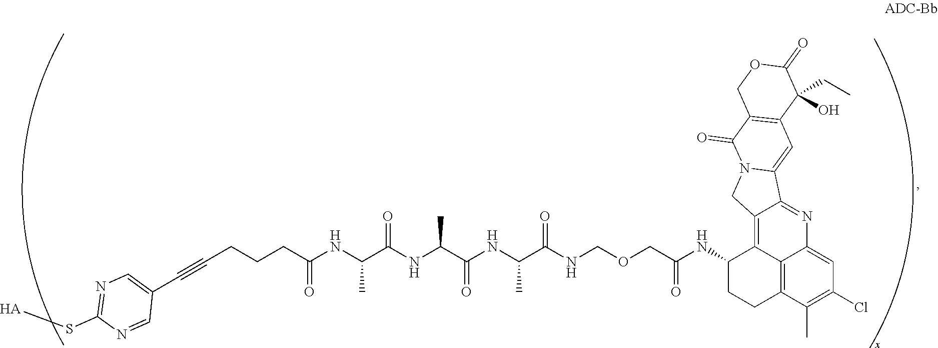
wherein HA-(Y- is HA-(S-;
HA-(S- comprises a heavy chain (HC) having the amino acid sequence as set forth in SEQ ID NO: 20 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 1-16, 1-10, 1-8, 4-8, 4 or 8.

36. The antibody-drug conjugate according to any one of claims 1 to 25, wherein the antibody-drug conjugate is ADC-Bb: 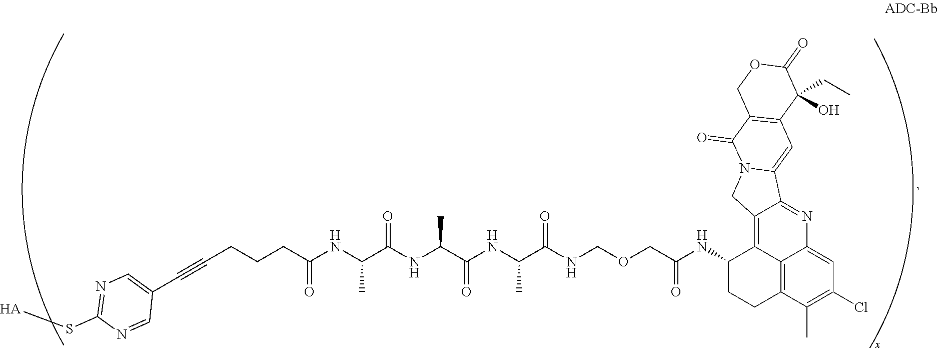
wherein HA-(Y- is HA-(S-;
HA-(S- comprises two heavy chains and two light chains, each heavy chain (HC) having the amino acid sequence as set forth in SEQ ID NO: 20 and each light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 1-16, 1-10, 1-8, 4-8, 4 or 8.

37. The antibody-drug conjugate according to any one of claims 1 to 25, wherein the antibody-drug conjugate is ADC-Bb: 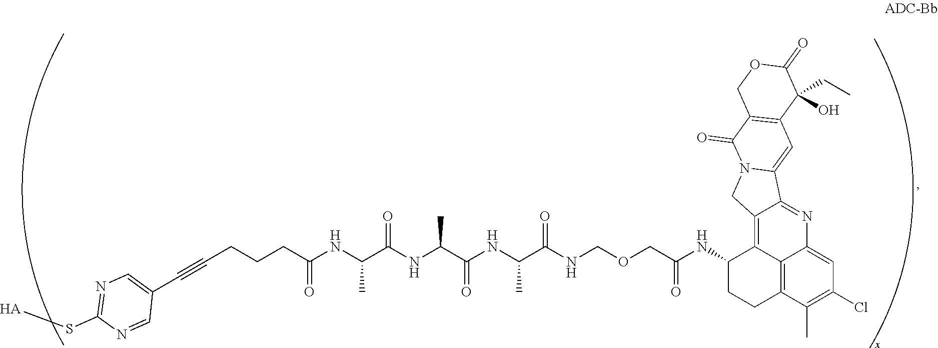
wherein HA-(Y- is HA-(S-;
HA-(S- comprises a heavy chain (HC) having the amino acid sequence as set forth in SEQ ID NO: 22, 23 or 24 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb;
and x is 1-16, 1-8, 4-8, 7-9, or 8.

38. The antibody-drug conjugate according to any one of claims 1 to 25, wherein the antibody-drug conjugate is ADC-Bb: 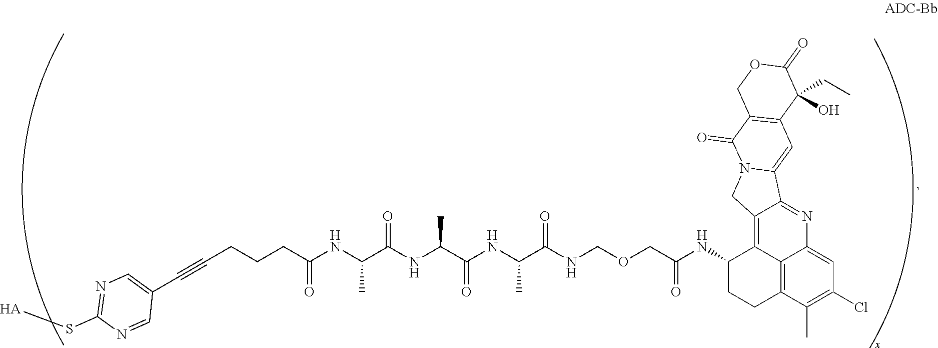
wherein HA-(Y- is HA-(S-;
HA-(S- comprises two heavy chains and two light chains, each heavy chain (HC) having the amino acid sequence as set forth in SEQ ID NO: 22 and each light chain (LC) having the amino acid sequence as set forth in SEQ ID NO: 21;
each -(S-represents a linkage from a sulfhydryl of a cysteine residue in the antibody to each pyrimidinyl group in formula ADC-Bb; and
x is 1-16, 1-10, 1-8, 4-8, 4 or 8.

39. The antibody-drug conjugate according to any one of claims 1 to 25, wherein the antibody-drug conjugate is ADC-Bb: 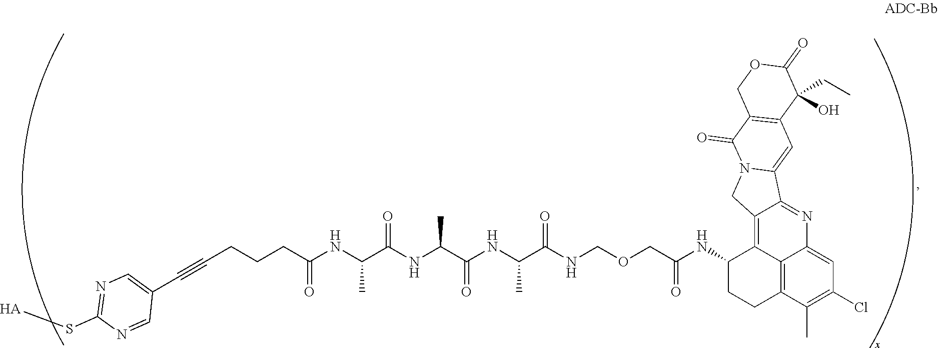
wherein HA-(Y- is HA-(S-;
HA-(S- comprises a heavy chain (HC) having the amino acid sequence as set forth in SEQ ID NO: 22 and a light chain (LC) having the amino acid sequence as set forth in SEQ ID NO: 21;
each -(S-represents a linkage from a sulfhydryl of a cysteine residue in the antibody to each pyrimidinyl group in formula ADC-Bb; and
x is 1-16; 1-8; 4-8; 7-9; 4 or 8.

40. The antibody-drug conjugate according to any one of claims 23 to 39, wherein x is 4-8.

41. The antibody-drug conjugate according to any one of claims 23 to 39, wherein x is 7-9.

42. The antibody-drug conjugate according to any one of claims 23 to 39, wherein x is 4 or 8.

43. The antibody-drug conjugate according to any one of claims 23 to 39, wherein x is 4.

44. The antibody-drug conjugate according to any one of claims 23 to 39, wherein x is 8.

45. The antibody-drug conjugate of any one of claims 1 to 27, wherein (i) the heavy chain C-terminus lacks a lysine residue; (ii) the heavy chain N-terminal amino acid is glutamine, glutamic acid, pyroglutamate or pyroglutamic acid; or, (iii) the heavy chain C-terminus lacks a lysine residue and the heavy chain N-terminal amino acid is glutamine, glutamic acid, pyroglutamate or pyroglutamic acid.

46. The antibody-drug conjugate of any one of claims 1 to 44, wherein the antibody or antigen binding fragment comprises a heavy chain comprising a V_{H} having the amino acid sequence set forth in SEQ ID NO: 1.

47. The antibody-drug conjugate of any one of claims 1 to 27, wherein the antibody or antigen binding fragment comprises a heavy chain comprising a V_{H} having the amino acid sequence set forth in SEQ ID NO: 1, wherein the N-terminal glutamine of the antibody variable region has undergone cyclization to pyroglutamate or pyroglutamic acid.

48. The antibody-drug conjugate of any one of claims 1 to 27, wherein the antibody or antigen binding fragment comprises:
(a) a heavy chain consisting of the sequence set forth in SEQ ID NO: 22 and a light chain consisting of the sequence shown as SEQ ID NO: 21;
(b) a heavy chain consisting of the sequence set forth in SEQ ID NO: 23 and a light chain consisting of the sequence shown as SEQ ID NO: 21; or
(c) a heavy chain consisting of the sequence set forth in SEQ ID NO: 24 and a light chain consisting of the sequence shown as SEQ ID NO: 21.

49. The antibody-drug conjugate of any one of claims 1 to 48, wherein the antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(1) a heavy chain comprising a V_{H} having the amino acid sequence set forth in SEQ ID NO: 1 and heavy chain constant region (CH) having the amino acid sequence set forth in SEQ ID NO: 3, and a light chain comprising a V_{L} having the amino acid sequence set forth in SEQ ID NO: 2 and light chain constant region (CL) having the amino acid sequence set forth in SEQ ID NO: 4;
(2) a heavy chain (HC) having the amino acid sequence as set forth in SEQ ID NO: 20 and a light chain (LC) having the amino acid sequence as set forth in SEQ ID NO: 21; or
(3) a V_{H} having the amino acid sequence set forth in SEQ ID NO: 1 and a V_{L} having the amino acid sequence set forth in SEQ ID NO: 2.

50. A composition comprising one or more antibody-drug conjugates according to any one of claims 1 to 49, wherein the DAR (drug-antibody conjugation ratio) of the composition is 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10.

51. An antibody-drug conjugate comprising an antibody that binds to CEACAM5, conjugated via one or more cysteine or lysine residue or non-canonical amino acid substitutions of amino acids of the antibody to a drug linker having a structure shown as the formula M-L-E-D, where
M-L-E is a linker connecting the antibody to D, wherein M is the portion of the linker connecting to the antibody, L is the central portion of the linker, and E is the portion of the linker connecting to D;
M is 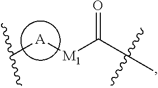
wherein ring A is a 5-6 membered aliphatic heterocyclic ring, or 5-20 membered aromatic ring system, and the aliphatic heterocyclic ring and aromatic ring systems are optionally substituted by one or more members independently selected from the group consisting of oxo (═O), halogen, cyano, amino, carboxyl, mercapto and C₁₋₆ alkyl;
M₁ is selected from a single bond, C₁₋₂₀ alkylene, C₂₋₂₀ alkenylene and C₂₋₂₀ alkynylene; and
D is a cytotoxic drug.

52. The antibody-drug conjugate of claim 51, wherein 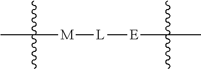
is selected from

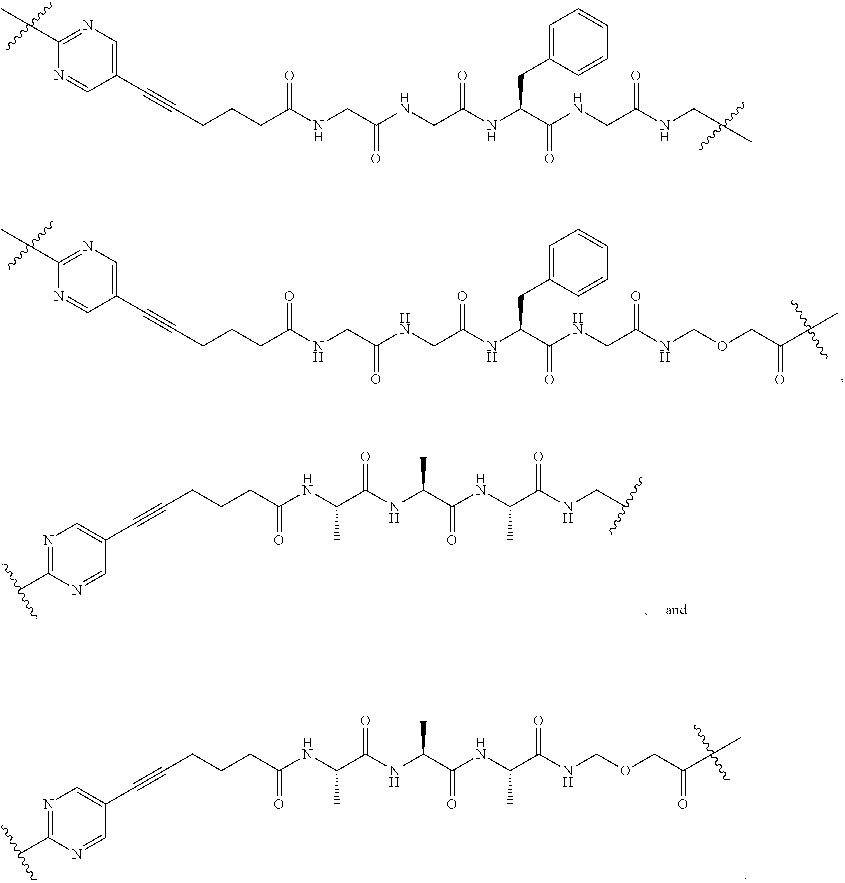 [SMILES: *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

53. The antibody-drug conjugate according to claim 51 or 52, wherein the cytotoxic drug is selected from the following compounds:

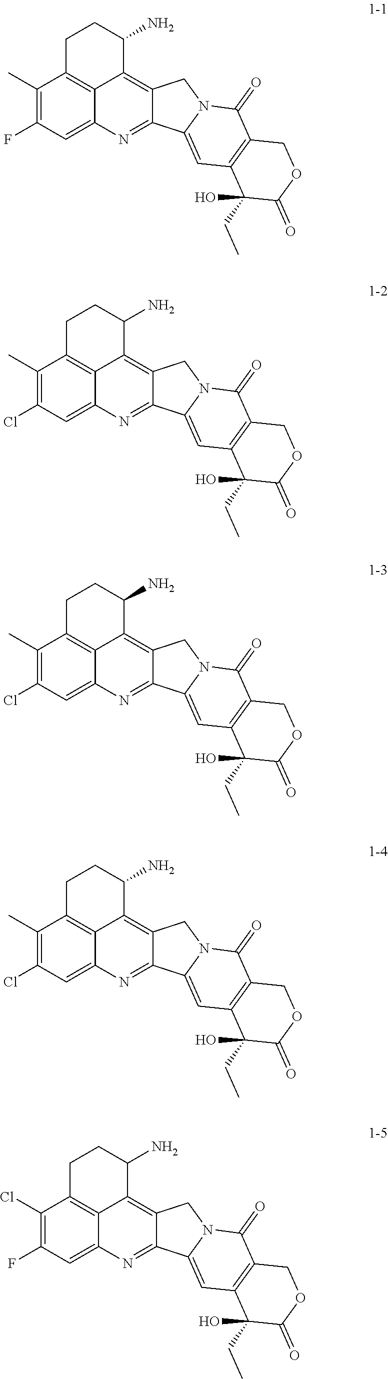 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

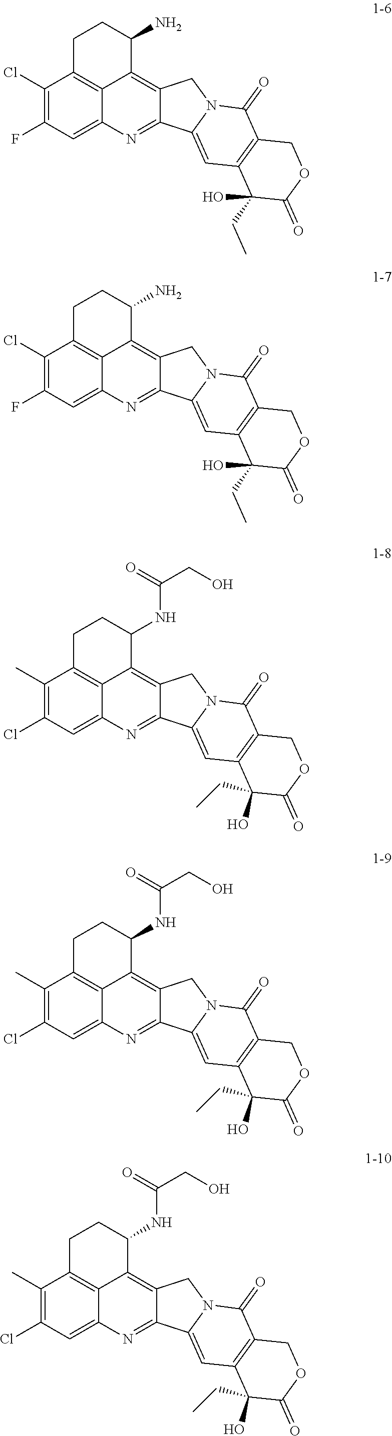 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

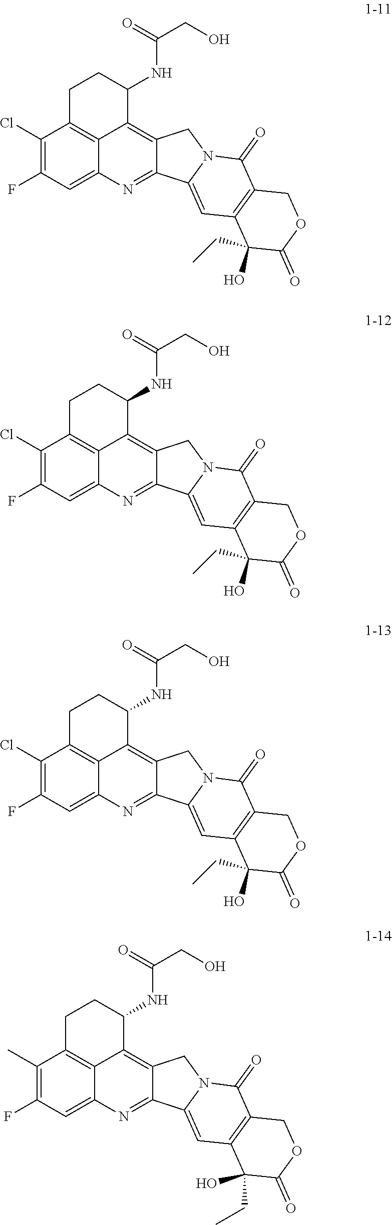 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

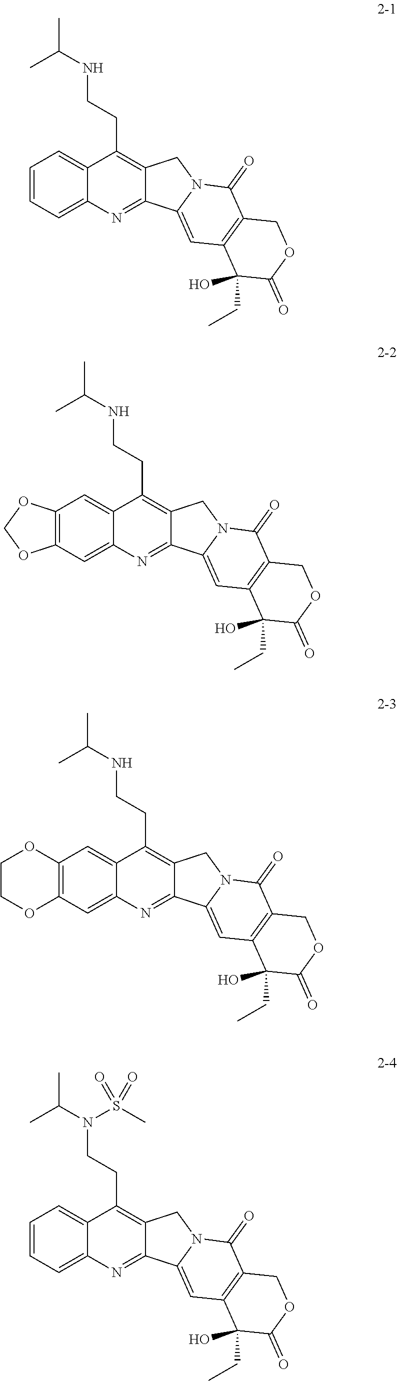 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O]

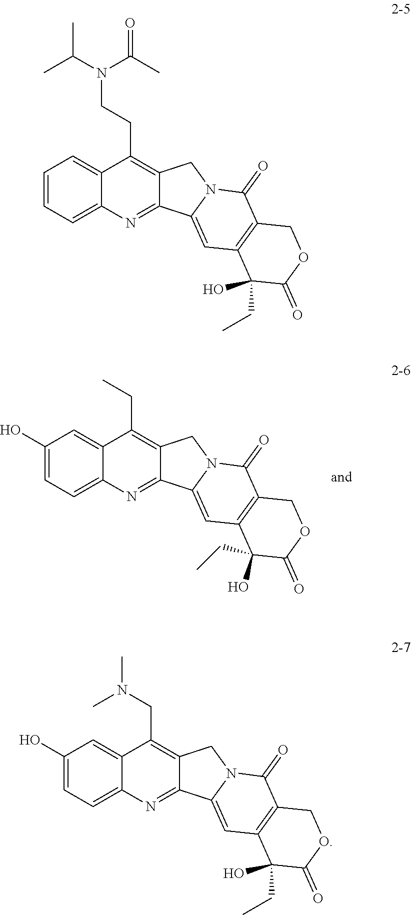 [SMILES: CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

54. The antibody-drug conjugate of any one of claims 51 to 53, wherein the antibody that binds to CEACAM5 is selected from the group consisting of:
(1) the following heavy chain variable regions (VH) and/or light chain variable regions (VL):
a heavy chain variable region (VH) comprising the following 3 CDRs: CDR-H1 having an amino acid sequence as set forth in SEQ ID NO: 14 or a variant thereof, CDR-H2 having an amino acid sequence as set forth in SEQ ID NO: 18 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
(2) the following heavy chain variable region (VH) and/or light chain variable region (VL):
a heavy chain variable region (VH) comprising the following 3 CDRs: CDR-H1 having an amino acid sequence as set forth in SEQ ID NO: 15 or a variant thereof, CDR-H2 having an amino acid sequence as set forth in SEQ ID NO: 19 or a variant thereof, CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
(3) the following heavy chain variable regions (VH) and/or light chain variable regions (VL):
a heavy chain variable region (VH) comprising the following 3 CDRs: CDR-H1 having an amino acid sequence as set forth in SEQ ID NO: 10 or a variant thereof, CDR-H2 having an amino acid sequence as set forth in SEQ ID NO: 17 or a variant thereof, and CDR-H3 having an amino acid sequence as set forth in SEQ ID NO: 11 or a variant thereof; and/or, a light chain variable region (VL) comprising the following 3 CDRs: CDR-L1 having an amino acid sequence as set forth in SEQ ID NO: 12 or a variant thereof, CDR-L2 having an amino acid sequence as set forth in SEQ ID NO: 13 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one, two, or several amino acids compared with the sequence from which the variants are derived; preferably, the replacement is a conservative replacement;
or,
(4) the following heavy chain variable regions (VH) and/or light chain variable regions (VL):
a heavy chain variable region (VH) comprising the following 3 CDRs: CDR-H1 having an amino acid sequence as set forth in SEQ ID NO: 5 or a variant thereof, CDR-H2 having an amino acid sequence as set forth in SEQ ID NO: 16 or a variant thereof, and CDR-H3 having an amino acid sequence as set forth in SEQ ID NO: 6 or a variant thereof; and/or, a light chain variable region (VL) comprising the following 3 CDRs: CDR-L1 having an amino acid sequence as set forth in SEQ ID NO: 7 or a variant thereof, CDR-L2 having an amino acid sequence as set forth in SEQ ID NO: 8 or a variant thereof, and CDR-L3 having an amino acid sequence as set forth in SEQ ID NO: 9 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived.

55. The antibody-drug conjugate of any one of claims 51 to 54, wherein the the antibody or antigen-binding fragment that binds CEACAM5 comprises a V_{H} having the amino acid sequence as set forth in SEQ ID NO: 1 or a variant thereof, and/or, a V_{L} having the amino acid sequence as set forth in SEQ ID NO: 2 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one, two, or several amino acids as compared to the sequence from which the variants are derived.

56. The antibody-drug conjugate of any one of claims 51 to 55, wherein the antibody that binds CEACAM5 comprises a heavy chain comprising a V_{H} having the amino acid sequence as set forth in SEQ ID NO: 1 and a heavy chain constant region (CH) shown as SEQ ID NO: 3, and, a light chain comprising a V_{L} having the amino acid sequence as set forth in SEQ ID NO: 2 and a light chain constant region (CL) having the amino acid sequence as set forth in SEQ ID NO: 4.

57. The antibody-drug conjugate of any one of claims 51 to 56, wherein the antibody that binds CEACAM5 comprises a heavy chain having the amino acid sequence as set forth in SEQ ID NO: 20 and a light chain having the amino acid sequence as set forth in SEQ ID NO: 21.

58. An antibody-drug conjugate of formula ADC-Aa: 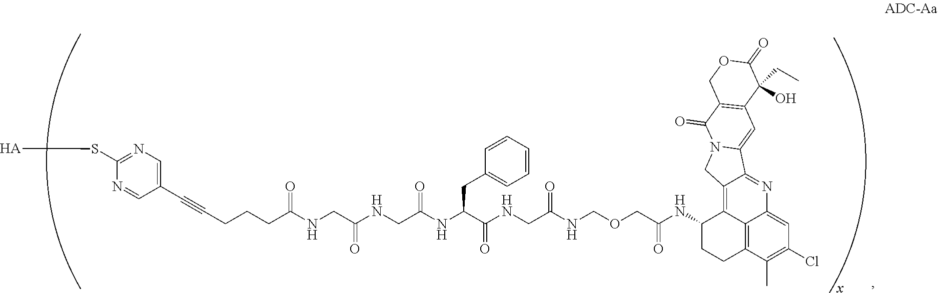
wherein HA-(S-represents an antibody that specifically binds to human CEACAM5 and comprises a heavy chain (HC) having the amino acid sequence as set forth in SEQ ID NO: 20 or 22 and a light chain (LC) having the amino acid sequence as set forth in SEQ ID NO: 21;
each -(S-represents a linkage from a sulfhydryl of a cysteine residue in the antibody to each pyrimidinyl group shown in formula ADC-Aa; and x is an integer from 1-8.

59. An antibody-drug conjugate of formula ADC-Bb: 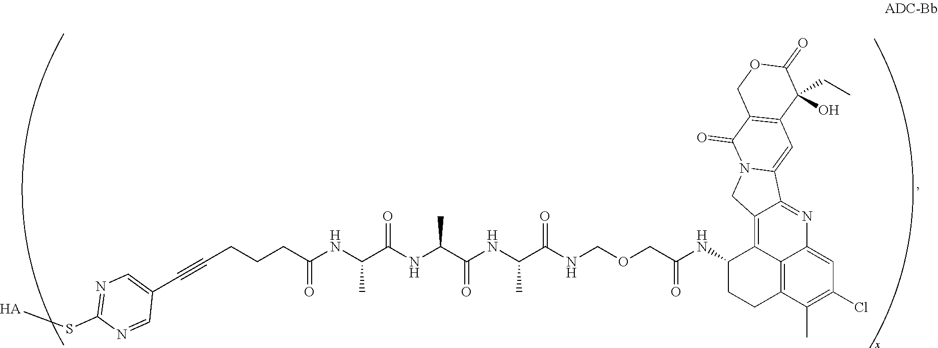
wherein HA-(S-represents an antibody that specifically binds to human CEACAM5 and comprises a heavy chain (HC) having the amino acid sequence as set forth in SEQ ID NO: 20 or 22 and a light chain (LC) having the amino acid sequence as set forth in SEQ ID NO: 21; each -(S-represents a linkage from a sulfhydryl of a cysteine residue in the antibody to each pyrimidinyl group shown in formula ADC-Bb; and x is an integer from 1-8.

60. An antibody-drug conjugate of formula ADC-Aa′: 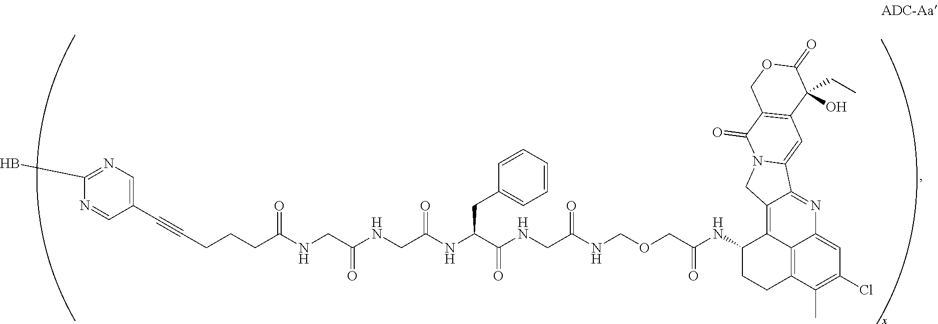
wherein HB-(- is an anti-CEACAM5 monoclonal antibody comprising a heavy chain (HC) having the amino acid sequence as set forth in SEQ ID NO: 20 or 22 and a light chain (LC) having the amino acid sequence as set forth in SEQ ID NO: 21, and x is an integer from 1-8.

61. The antibody-drug conjugate of claim 60, wherein HB-(- is linked via x number of sulfhydryl group(s) of cysteine residue(s) in the antibody.

62. An antibody-drug conjugate of formula ADC-Bb′: 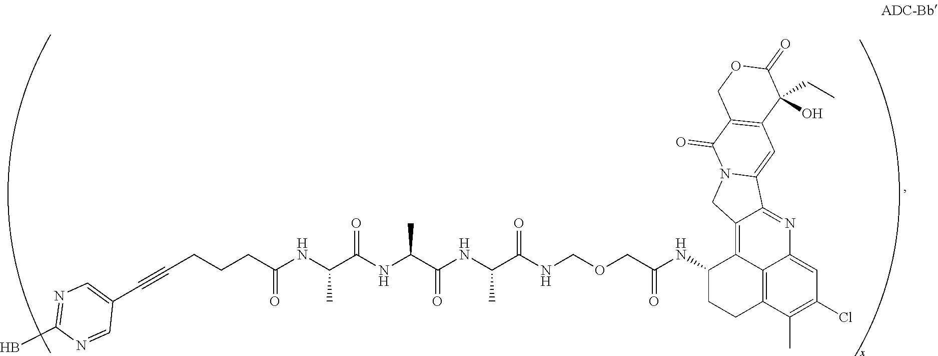
wherein HB-(- is an anti-CEACAM5 monoclonal antibody comprising a heavy chain (HC) having the amino acid sequence as set forth in SEQ ID NO: 20 or 22 and a light chain (LC) having the amino acid sequence as set forth in SEQ ID NO: 21, and x is an integer from 1-8.

63. The antibody-drug conjugate of claim 62, wherein HB-(- is linked via x number of sulfhydryl group(s) of cysteine residue(s) in the antibody.

64. The antibody-drug conjugate according to any one of claims 1 to 49 and 51 to 63, wherein the antibody or antigen binding fragment thereof is an antibody.

65. A pharmaceutical composition, comprising the antibody-drug conjugate according to any one of claims 1 to 49 and 51 to 64, and one or more pharmaceutically acceptable excipients.

66. A method of treating a cancer in a subject with high expression of CEACAM5 comprising administering a therapeutically effective amount of the antibody-drug conjugate according to any one of claims 1 to 49 and 51 to 64, the composition according to claim 50, or the pharmaceutical composition according to claim 65 to the subject.

67. The method of claim 66, wherein the cancer comprises solid tumors.

68. The method of claim 67, wherein the cancer is a cancer of the rectum, colon, stomach, pancreas, esophagus, lung, head and neck, breast, bladder, uterine, prostate, skin, testicle, ovary, liver, thyroid, kidney, or brain; or the cancer is a lymphoma, a melanoma or a leukemia.

69. The method of claim 66, wherein the cancer is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer, adenocarcinomas of the esophagogastric junction, bladder epithelial carcinoma, breast ductal carcinoma, ovarian endometriosis carcinoma, prostate transitional cell carcinoma, cervical squamous cell carcinoma, colorectal cancer, or non-small cell lung cancer.

70. Use of the antibody-drug conjugate according to any one of claims 1 to 49 and 51 to 64, the composition according to claim 50, or the pharmaceutical composition according to claim 65 in the preparation of a drug for treating cancers with high expression of CEACAM5.

71. The use of claim 70, wherein the cancer comprises solid tumors.

72. The use of claim 70, wherein the cancer is a cancer of the rectum, colon, stomach, pancreas, esophagus, lung, head and neck, breast, bladder, uterine, prostate, skin, testicle, ovary, liver, thyroid, kidney, or brain; or the cancer is a lymphoma, a melanoma or a leukemia.

73. The use of claim 70, wherein the cancer with high expression of CEACAM5 is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer, adenocarcinomas of the esophagogastric junction, bladder epithelial carcinoma, breast ductal carcinoma, ovarian endometriosis carcinoma, prostate transitional cell carcinoma, cervical squamous cell carcinoma, colorectal cancer, or non-small cell lung cancer.

74. Use of the antibody-drug conjugate according to any one of claims 1 to 49 and 51 to 64, the composition according to claim 50, or the pharmaceutical composition according to claim 65 in the treatment of a cancer with high expression of CEACAM5.

75. The use of claim 74, wherein the cancer comprises solid tumors.

76. The use of claim 74, wherein the cancer is a cancer of the rectum, colon, stomach, pancreas, esophagus, lung, head and neck, breast, bladder, uterine, prostate, skin, testicle, ovary, liver, thyroid, kidney, or brain; or the cancer is a lymphoma, a melanoma or a leukemia.

77. The use of claim 74, wherein the cancer with high expression of CEACAM5 is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer, adenocarcinomas of the esophagogastric junction, bladder epithelial carcinoma, breast ductal carcinoma, ovarian endometriosis carcinoma, prostate transitional cell carcinoma, cervical squamous cell carcinoma, colorectal cancer, or non-small cell lung cancer.

78. An antibody-drug conjugate according of any one of claims 1 to 49 and 51 to 64, the composition according to claim 50, or the pharmaceutical composition according to claim 65 for use in treatment of a cancer with high expression of CEACAM5.

79. The antibody-drug conjugate of claim 78, wherein the cancer comprises solid tumors.

80. The antibody-drug conjugate of claim 78, wherein the cancer is a cancer of the rectum, colon, stomach, pancreas, esophagus, lung, head and neck, breast, bladder, uterine, prostate, skin, testicle, ovary, liver, thyroid, kidney, or brain; or the cancer is a lymphoma, a melanoma or a leukemia.

81. The antibody-drug conjugate of claim 78, wherein the cancer is rectum adenocarcinoma, colon adenocarcinoma, stomach adenocarcinoma, pancreatic adenocarcinoma, esophageal carcinoma, lung adenocarcinoma, cervical cancer, endocervical cancer, lung squamous cell carcinoma, head and neck squamous cell carcinoma, breast invasive carcinoma, bladder urothelial carcinoma, cholangiocarcinoma, uterine corpus endometrioid carcinoma, prostate adenocarcinoma, skin cutaneous melanoma, uterine carcinosarcoma, testicular germ cell tumor, ovarian serous cystadenocarcinoma, liver hepatocellular carcinoma, thyroid carcinoma, sarcoma, kidney papillary cell carcinoma, thymoma, kidney clear cell carcinoma, brain lower grade glioma, mesothelioma, diffuse large B-cell lymphoma, glioblastoma multiforme, kidney chromophobe, uveal melanoma, adrenocortical cancer, pheochromocytoma and paraganglioma, acute myeloid leukemia, gastric cancer, adenocarcinomas of the esophagogastric junction, bladder epithelial carcinoma, breast ductal carcinoma, ovarian endometriosis carcinoma, prostate transitional cell carcinoma, cervical squamous cell carcinoma, colorectal cancer, or non-small cell lung cancer.


SEQUENCE LISTING (32 sequences)

SEQ ID NO: 1 - protein, 119 aa, synthetic construct
  source 1..119: note = Ab-hzDPmut VH amino acid sequence
QVQLVQSGAEVVKPGASVKVSCKASGYTFTRYWMHWVRQAPGQGLEWMGRIEPNAGEPTYNEKFKNRVTMTVDTSTSTVYMELSSLRSEDTAVYYCARSTTTVEAMDYWGQGTSVTVSS

SEQ ID NO: 2 - protein, 110 aa, synthetic construct
  source 1..110: note = Ab-hzDPmut VL amino acid sequence
DIQLTQSPSSLSASVGDRVTITCRASESVDNYGFTFMHWYQQKPGKAPKLLIYRASNLESGIPSRFSGSGSGTDFTLTISSVQPEDFATYYCQQSNKNPTFGGGTKLEIK

SEQ ID NO: 3 - protein, 330 aa, synthetic construct
  source 1..330: note = Human mutant IgG1 amino acid sequence of a heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 107 aa, synthetic construct [sequence omitted]

SEQ ID NO: 5 - protein, 10 aa, synthetic construct
  source 1..10: note = Ab-hzDPmut Abm CDRH1
GYTFTRYWMH

SEQ ID NO: 6 - protein, 10 aa, synthetic construct
  source 1..10: note = Ab-hzDPmut Abm/Chothia/Kabat CDRH3
STTTVEAMDY

SEQ ID NO: 7 - protein, 15 aa, synthetic construct
  source 1..15: note = Ab-hzDPmut Abm/Chothia/Kabat CDRL1
RASESVDNYGFTFMH

SEQ ID NO: 8 - protein, 7 aa, synthetic construct
  source 1..7: note = Ab-hzDPmut Abm/Chothia/Kabat CDRL2
RASNLES

SEQ ID NO: 9 - protein, 8 aa, synthetic construct
  source 1..8: note = Ab-hzDPmut Abm/Chothia/Kabat/IMGT CDRL3
QQSNKNPT

SEQ ID NO: 10 - protein, 8 aa, synthetic construct
  source 1..8: note = Ab-hzDPmut IMGT CDRH1
GYTFTRYW

SEQ ID NO: 11 - protein, 12 aa, synthetic construct
  source 1..12: note = Ab-hzDPmut IMGT CDRH3
ARSTTTVEAMDY

SEQ ID NO: 12 - protein, 10 aa, synthetic construct
  source 1..10: note = Ab-hzDPmut IMGT CDRL1
ESVDNYGFTF

SEQ ID NO: 13 - skipped

SEQ ID NO: 14 - protein, 7 aa, synthetic construct
  source 1..7: note = Ab-hzDPmut Chothia CDRH1
GYTFTRY

SEQ ID NO: 15 - protein, 5 aa, synthetic construct
  source 1..5: note = Ab-hzDPmut kabat CDRH1
RYWMH

SEQ ID NO: 16 - protein, 10 aa, synthetic construct
  source 1..10: note = Ab-hzDPmut AbM CDRH2
RIEPNAGEPT

SEQ ID NO: 17 - protein, 8 aa, synthetic construct
  source 1..8: note = Ab-hzDPmut IMGT CDRH2
IEPNAGEP

SEQ ID NO: 18 - protein, 6 aa, synthetic construct
  source 1..6: note = Ab-hzDPmut Chothia CDRH2
EPNAGE

SEQ ID NO: 19 - protein, 17 aa, synthetic construct
  source 1..17: note = Ab-hzDPmut Kabat CDRH2
RIEPNAGEPTYNEKFKN

SEQ ID NO: 20 - protein, 449 aa, synthetic construct
  source 1..449: note = Ab-hzDPmut heavy chain amino acid sequence
QVQLVQSGAEVVKPGASVKVSCKASGYTFTRYWMHWVRQAPGQGLEWMGRIEPNAGEPTYNEKFKNRVTMTVDTSTSTVYMELSSLRSEDTAVYYCARSTTTVEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 217 aa, synthetic construct
  source 1..217: note = Ab-hzDPmut light chain amino acid sequence
DIQLTQSPSSLSASVGDRVTITCRASESVDNYGFTFMHWYQQKPGKAPKLLIYRASNLESGIPSRFSGSGSGTDFTLTISSVQPEDFATYYCQQSNKNPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 448 aa, synthetic construct
  source 1..448: note = Ab-hzDPmut heavy chain amino acid sequence (Pyroglutamate or Pyroglutamic acid at N-terminus; no C-terminal lysine)
  SITE 1: note = pyroglutamate or pyroglutamic acid
XVQLVQSGAEVVKPGASVKVSCKASGYTFTRYWMHWVRQAPGQGLEWMGRIEPNAGEPTYNEKFKNRVTMTVDTSTSTVYMELSSLRSEDTAVYYCARSTTTVEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 448 aa, synthetic construct
  source 1..448: note = Ab-hzDPmut heavy chain amino acid sequence (no C-terminal lysine)
QVQLVQSGAEVVKPGASVKVSCKASGYTFTRYWMHWVRQAPGQGLEWMGRIEPNAGEPTYNEKFKNRVTMTVDTSTSTVYMELSSLRSEDTAVYYCARSTTTVEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 449 aa, synthetic construct
  source 1..449: note = Ab-hzDPmut heavy chain amino acid sequence (Pyroglutamate or Pyroglutamic acid at N-terminus)
  SITE 1: note = pyroglutamate or pyroglutamic acid
XVQLVQSGAEVVKPGASVKVSCKASGYTFTRYWMHWVRQAPGQGLEWMGRIEPNAGEPTYNEKFKNRVTMTVDTSTSTVYMELSSLRSEDTAVYYCARSTTTVEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 4 aa, synthetic construct
  source 1..4: note = Amino acid portion of linker
GGFG

SEQ ID NO: 26 - protein, 4 aa, synthetic construct
  source 1..4: note = Amino acid portion of linker
GFLG

SEQ ID NO: 27 - protein, 4 aa, synthetic construct
  source 1..4: note = Amino acid portion of linker
GGVA

SEQ ID NO: 28 - protein, 5 aa, synthetic construct
  source 1..5: note = Amino acid portion of linker
GGGGG

SEQ ID NO: 29 - protein, 5 aa, synthetic construct
  source 1..5: note = Amino acid portion of linker
GGGGS

SEQ ID NO: 30 - protein, 20 aa, synthetic construct
  source 1..20: note = Amino acid portion of linker
GGGGSGGGGSGGGGSGGGGS

SEQ ID NO: 31-32 - skipped (2 entries)